(19) United States
(12) Patent Application Publication
(10) Pub. No.: US 2015/0024969 A1
(43) Pub. Date: Jan. 22, 2015

(54) SEPSIS PROGNOSIS BIOMARKERS

(71) Applicants: Raymond Langley, Albuquerque, NM (US); Stephen Kingsmore, Overland Park, KS (US)

(72) Inventors: Raymond Langley, Albuquerque, NM (US); Stephen Kingsmore, Overland Park, KS (US)

(73) Assignee: THE CHILDREN'S MERCY HOSPITAL, Kansas City, MO (US)

(21) Appl. No.: 14/344,534

(22) PCT Filed: Sep. 12, 2012
(86) PCT No.: PCT/US12/54951

§ 371 Date: Oct. 10, 2014

Related U.S. Application Data
(60) Provisional application No. 61/533,782, filed on Sep. 12, 2011.

(51) Int. Cl. G01N 33/68; G01N 33/49

(52) U.S. Cl. CPC G01N 33/6848; G01N 33/492; G01N 2570/00; G01N 2800/26
USPC 506/12; 506/15; 436/70

(57) ABSTRACT

A method for determining prognosis, diagnosis and theronosis of a sepsis infection in a patient is disclosed. The method involves measuring the age, mean arterial pressure, hematocrit, patient temperature, and the concentration of one or more metabolites that are predictive of sepsis severity. The method can involve obtaining a blood sample from said patient and determining the concentration of the metabolite in the patient's blood; and then determining the severity of sepsis infection by analyzing the measured values in a weighted logistic regression equation.

### STATEMENT REGARDING FEDERALLY SPONSORED RESEARCH

The U.S. government retains certain rights in this invention as provided by the terms of Grant Number U01A1066569 (NIH), P20RR016480 and HHSN266200400064C, awarded by the National Institutes of Health.

### TECHNICAL FIELD

This invention is related to the area of prognosis, diagnosis and theranosis. In particular, it relates to prognosis, diagnosis, risk assessment, and monitoring of sepsis.

### BACKGROUND ART

Sepsis is the name given to infection when symptoms of inflammatory response are present. Of patients hospitalized in an intensive care unit (ICU) who have an infection, 82% have sepsis. Sepsis is defined as an infection-induced syndrome involving two or more of the following features of systemic inflammation: fever or hypothermia, leukocytosis or leukopenia, tachycardia, and tachypnea or a supranormal minute ventilation. Sepsis may be defined by the presence of any of the following ICD-9-CM codes: 038 (septicemia), 020.0 (septicemic), 790.7 (bacteremia), 117.9 (disseminated fungal infection), 112.5 (disseminated Candida infection), and 112.81 (disseminated fungal endocarditis). Sepsis is diagnosed either by clinical criteria or by culture of microorganisms from the blood of patients suspected of having sepsis plus the presence of features of systemic inflammation. Culturing some microorganisms can be tedious and time consuming, and may provide a high rate of false negatives. Bloodstream infection is diagnosed by identification of microorganisms in blood specimens from a patient suspected of having sepsis after 24 to 72 hours of laboratory culture. Currently, gram positive bacteria account for 52% of cases of sepsis, gram-negative bacteria account for 38%, polymicrobial infections for 5%, anaerobes for 1%, and fungi for 5%. For each class of infection listed, there are several different types of microorganisms that can cause sepsis. The high rate of false negative microbiologic cultures leads frequently today to empiric treatment for sepsis in the absence of definitive diagnosis. Infection at many different sites can result in sepsis. The most common sites of infection in patients with sepsis are lung, gut, urinary tract, and primary blood stream site of infection. Since sepsis can be caused by many infections with microorganisms at many different sites, sepsis is a very heterogeneous disease. The heterogeneity of sepsis increases the difficulty in devising a diagnostic test.

The number of patients with sepsis per year is increasing at 13.7% per year, and was 659,935 in 2000. The incidence of sepsis in the United States in 2000 was 240.4 cases per 100,000 population. Sepsis accounted for 1.3% of all hospitalizations in the U.S. from 1979 to 2000. During this period, there were 750 million hospitalizations in the U.S. and 10.5 million reported cases of sepsis.

Sepsis is the leading cause of death in critically ill patients, the second leading cause of death among patients in non-coronary intensive care units (ICUs), and the tenth leading cause of death overall in the United States. Overall mortality rates for sepsis are 18%. In-hospital deaths related to sepsis were 120,491 (43.9 per 100,000 population) in 2000.

Care of patients with sepsis is expensive and accounts for $17 billion annually in the United States alone. Sepsis is often lethal, killing 20 to 50 percent of severely affected patients. Furthermore, sepsis substantially reduces the quality of life of those who survive: only 56% of patients surviving sepsis are discharged home; 32% are discharged to other health care facilities (i.e., rehabilitation centers or other long-term care facilities), accruing additional costs of care.

Cost of care, morbidity and mortality related to sepsis are largely associated with delayed diagnosis and specific treatment of sepsis and the causal infection. Early diagnosis of sepsis is expected to result in decreased morbidity, mortality and cost of care. The average length of hospital stay in patients with sepsis is twelve days.

Severe sepsis is defined as sepsis associated with acute organ dysfunction. The proportion of patients with sepsis who had any organ failure is 34%, resulting in the identification of 256,033 cases of severe sepsis in 2000. Organ failure had a cumulative effect on mortality: approximately 15% of patients without organ failure died, whereas 70% of patients with 3 or more failing organs (classified as having severe sepsis and septic shock) died. Risk of death from sepsis increases with increasing severity of sepsis.

Currently determination of the severity of sepsis and determination of whether, in a patient with sepsis, the sepsis is increasing or decreasing in severity, is based upon clinical events such as failing organs. Determination that, in a patient with sepsis, the sepsis is increasing in severity, may allow more intensive therapy to be given which may increase the likelihood of the patient surviving. The availability of a diagnostic test that would allow monitoring of patients with sepsis to determine whether the sepsis is increasing or decreasing in severity may allow early detection of deterioration and earlier intensification of therapy and less risk of death or disability. Sepsis results either from community-acquired infections or hospital-acquired infections. Sepsis occurs in 1.3% of all U.S. hospitalizations. Hospital-acquired infections are a major source of sepsis, accounting for 65% of sepsis patients who are admitted to an intensive care unit. Sepsis is a major cause of admission to a hospital intensive care unit. 23-30% of patients admitted to an intensive care unit for longer than 24 hours will develop sepsis. Sepsis is a common complication of prolonged stay in an ICU. 8% of patients who remain in an ICU for longer than 24 hours will develop sepsis.

There is a need for screening diagnostic tests for sepsis and for tests to monitor sepsis severity with relatively few false negatives and high sensitivity and specificity. Sepsis is the 10th leading cause of death. Infections account for 11 million hospital visits per year. Only the patients with severe symptoms are hospitalized or receive intensive treatment. However, the evaluation and management of patients with suspected sepsis is complicated by the lack of specific diagnostic criteria, heterogeneity of presentation and outcome. Early identification of patients likely to progress to death, who are candidates for aggressive treatment to prevent such death, is particularly difficult.

Current gold standards for prognostic assessment in sepsis include APACHE II (Acute Physiology and Chronic Health Evaluation), SOFA (Sepsis-related Organ Failure Assessment), and PRISM III (Pediatric Risk of Mortality) scores (Knaus et al., 1985; Vincent et al., 1996; Pollack et al., 1996). Additional potential treatments include admission to an intensive care unit, early goal directed therapy, activated protein C therapy, intensive glycemic control, hyperbaric or supplemental oxygen, or exogenous steroids (Otero et al., 2006; Russel 2008; Calzia et al., 2006; Muth et al., 2005; Annane 2005; Lin et al., 2005; Oter et al., 2005). The decisions regarding the severity of sepsis made based upon APACHE II, SOFA, PRISM and other clinical scores or on finger stick lactate values are either subjective (clinical scores) or insensitive (lactate) or suffer from false negative results in certain subjects. Therefore a more accurate test using biomarkers or reference characteristics are needed to stratify patients at presentation and identify patient subsets that need additional or more aggressive treatment. Additionally what is needed are methods for diagnosing sepsis and differentiating those with sepsis from those patients who do not have sepsis.

### DISCLOSURE OF INVENTION

Methods and biomarker compositions are disclosed for prognosing and diagnosing sepsis in subjects, methods for prognosis of a sepsis infection and outcomes, and methods for determining the sepsis status of a subject who presents to a healthcare worker or facility as to whether the subject does or does not have sepsis, and whether there is a high risk of death. Methods comprise measurement of the amounts of one or more clinicometabolomic classifiers, which are identified clinical and metabolic changes in bodily fluids, such as plasma, of patients, for example, at time of presentation to a healthcare worker or facility, that distinguish sepsis from other disorders with similar presentation (NIS—non-infected SIRS-positive) (SIRS—systemic inflammatory response) and that differentiate sepsis patients that are likely to have uncomplicated courses from those patients that are likely to have complications, including death.

Also disclosed are novel therapeutic targets for individualized intervention. Disclosed herein are methods and compositions of diagnosing sepsis in a human subject. Methods and biomarkers of the present invention can be used to ascertain if a patient receiving treatment for sepsis is responding positively to such treatment. Additionally, methods and biomarkers of the present invention can be used to distinguish patients who should be admitted to a hospital for treatment from patients who will not require admittance for treatment.

A biomarker prognostic panel is disclosed that can distinguish and predict sepsis survival from sepsis death. The panel can include piperine, palmitoycarnitine, 3-methoxytyrosine, ocatanoylcarnitine, clinical blood lactate, X-12775 (unannotated analyte), and the single sulfated steroid X-11302 (unannotated analyte). Alternatively, the biomarker prognostic panel may comprise creatinine, 4-vinylphenol sulfate, cglycosyltryptophan, X-11261, X-12095, X-12100, 2-octenoylcarnitine and X-13553.

A biomarker diagnostic panel is disclosed that can differentiate sepsis patients from non-infected subjects. The panel can include galactonate, uridine, maltose, glutamate, creatine and X-12644 (unannotated analyte). Alternatively, the biomarker diagnostic panel may comprise citrulline, laurylcarnitine, androsterone sulfate, isoleucine, X-11838, X-12644, and X-11302 (a pregnan steroid monosulfate).

A method for sepsis prognosis in a subject is also described. The method can include the step of obtaining a biological sample from the subject; determining, in the biological sample, the level of the metabolites of a biomarker prognostic panel which can include piperine, palmitoycarnitine, 3-methoxytyrosine, ocatanoylcarnitine, clinical blood lactate, X-12775, and the single sulfated steroid X-11302 and creatinine, 4-vinylphenol sulfate, cglycosyltryptophan, X-11261, X-12095, X-12100, 2-octenoylcarnitine and X-13553; where in the correlated presence of the metabolites of the panel in the biological sample indicates that the subject has sepsis with high rate of death. In a method the biological sample subject to the method is a bodily fluid. In a method the biological sample subject to the method is plasma.

A method for sepsis diagnosis in a subject is disclosed which can include (a) obtaining a biological sample from the subject; (b) determining, in the biological sample, the concentration of the metabolites of a biomarker prognostic panel chosen from (1) galactonate, uridine, maltose, glutamate, creatine and X-12644 and (2) citrulline, laurylcarnitine, androsterone sulfate, isoleucine, X-11838, X-12644, and X-11302; where in the correlated presence of the metabolites of the panel in the biological sample indicates that the subject has sepsis. In one method the biological sample subject to the method can be a bodily fluid. In one method the biological sample subject to the method can be plasma.

A method for determining the severity of a sepsis infection in a patient is disclosed that can involve measuring the age, mean arterial pressure, hematocrit, patient temperature, and the concentration of one or more metabolites that are predictive of sepsis severity. The method can involve obtaining a blood sample from said patient and determining the concentration of the metabolite in the patient's blood; and then determining the severity of sepsis infection by analyzing the measured values in a weighted logistic regression equation. The blood sample can be taken when the patient arrives for treatment and subsequently thereafter, for example about 24 hours afterword, to determine the progress of the disease and efficacy of treatment. Not all of the markers need be assessed in every method only a sufficient number of markers to reliably determine the severity of the disease. Thus, a plurality or number of indicators can be measured which are selected from the group that includes a patient's age, mean arterial pressure, hematocrit, patient temperature, and the concentration of a metabolite selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate and their combinations. In some instances it may be possible to measure any two of these markers to assess sepsis severity. In more preferred embodiments three, four, five, six, seven, eight, ten, eleven or all twelve of the markers may be evaluated in the determination.

Preferably the accuracy of the panel in predicting day 7 sepsis survival in a known test patient population pool is about 90% or more, or more preferably about 95% or more and even more preferably about 99% or more.

The methods can also be used in the treatment of a sepsis patient. For example, to determine whether the disease is progressing and whether a therapeutic regimen is effective.

Other aspects and iterations of the invention are described in more detail below.

### BRIEF DESCRIPTION OF DRAWINGS

FIG. 1 Plasma levels of eleven metabolites in all patients showing relationships between time to death and metabolite values. Plasma metabolite concentrations were determined by targeted, quantitative MS assays and values are in pm/ml.

FIG. 2 Molecular models can predict survival and death in community-acquired sepsis. Day 7 SIRS survival (n=340) and SIRS death (n=39) by MAP, log₂MAP, log₂hexanoylcarnitine, Na⁺, log₂creatinine, log₂ pseudouridine, HPLA and 3-methoxytyrosine. a. Mosaic plot showing accuracy of death and survival prediction. b. ROC curves with AUCs 0.88, respectively. c. Overlayed plots of sensitivity (+), specificity (∘), accuracy (⋄), PPV (x) and NPV (Δ).

FIG. 3 An integrative systems survey of sepsis survival and death. a. The prevailing clinical model of sepsis progression at the outset of CAPSOD. b. Experimental design. Patients presenting to EDs with suspected community-acquired sepsis (acute infection and ≧2 SIRS criteria) were grouped according to final diagnosis (sepsis or non-infected), day 3 clinical course (septic shock, severe sepsis, and uncomplicated sepsis) and outcome at day 28 (survival or death). Groups were defined by the most severe stage of sepsis attained. MS-based metabolome and proteome analysis was performed on plasma samples obtained at t₀ and t₂₄ from 150 matched “discovery” subjects. Next generation sequencing was performed on mRNA from blood cells obtained from these subjects at t₀. Replication of metabolome findings was sought by semi-quantitative MS in an independent cohort comprising all remaining sepsis deaths and a matched group of sepsis survivors at t₀ and t₂₄ (n=52). Following molecular integration and analysis, predictive models were developed that were representative of the clinical and molecular findings. The utility of the predictive models was tested by targeted, quantitative assays of butyrylcarnitine, 2-methylbytyrylcarnitine, hexanoylcarnitine, cis-4-decenoylcarnitine, 1-arachidonoyl-glycerophosphocholine (GPC), 1-linoleoyl-GPC, pseudouridine, 3-(4-hydroxyphenyl)lactate (HPLA), 4-methyl-2-oxopentanoate, 3-methoxytyrosine and N-acetylthreonine of all 382 samples.

FIG. 4 Metabolomic profiling of plasma in sepsis. a,b. Venn diagrams of overlap of biochemicals (a) and annotated metabolites (b) measured by MS in discovery plasma samples at t₀ (n=150) and t₂₄ (n=132) and 52 replication (R) patients at at t₀ and t₂₄. 160 biochemicals were removed from analysis because they were detected in ≦50% of the patients. c. The variance in plasma metabolite levels at time of ED enrollment (t₀) that was attributable to sepsis outcome decreased with increasing days-to-death (X-axis). d,e,f. Comparison of Creatinine (c), Lactate (d) and Glucose (e) levels as determined in serum by clinical chemical analyzer and in plasma by MS in 149, 115 and 149 patients, respectively. MS values are normalized, log-transformed intensities. Chemistry values (mmol/L) are log-transformed. g. Z-score scatter plots of plasma biochemicals from non-infected SIRS-positive controls, uncomplicated sepsis, severe sepsis, septic shock or sepsis death patients. Zero on the X-axis represents the mean of the control group. Each data point is expressed as the number of standard deviations from the mean of the controls. The Y-axis shows all values for each biochemical on the same horizontal line. Z-score values are standard deviations from the control mean, revealing changes relative to control. The boxed values are IT/Scores, which are averages of the absolute values of Z-scores for all metabolites, calculated using non-truncated, non-imputed values.

FIG. 5 Principal components of variance (a) and unsupervised principal component analysis (PCA) of sepsis group membership (b) and renal function (c) in log-transformed plasma metabolites at t₀. a Variance decomposition (with Pearson product-moment correlation) for sepsis groups, chronic kidney disease/hemodialysis CKD(HD), liver disease, and immunosuppressant therapy. CKD(HD): estimated glomerular filtration rate (eGFR), and hemodialysis. b, Control (non-infected SIRS-positive), red, n=29; Uncomplicated sepsis, purple, n=26; Severe Sepsis, blue, n=25; Septic Shock, yellow, n=37; Sepsis Death, green, n=29. c, CKD1/2 (yellow, eGFR>74 mL/min, n=44); CKD3 (blue, eGFR 32-74 mL/min, n=56); CKD4/5 (green, eGRF 0-31 mL/min, n=25); hemodialysis (HD, red, n=24).

FIG. 6 B-matrices of Bayesian factor analysis (a and c) and the normalized energies (b and d) of sepsis group membership (SIRS+Outcomes), renal category (CKD(HD)) and other clinical parameters in log-transformed plasma metabolites at t₀ (a and b) and t₂₄ (c and d). Sepsis group membership (SIRS+Outcomes) was defined as non-infected SIRS-positive, sepsis survival and sepsis death. Renal function was defined as eGFR>74 mL/min=0; 32-74 mL/min=1; <31 mL/min=2; hemodialysis=3. Clinical parameters were fit to a normal distribution with mean of 0 and standard deviation of 1. Bayesian regression [cj=Byj+A(sj·zj)+εj where B is the relationship between metabolite values and the clinical parameter, A is random or undefined effects and ε is random noise] of metabolite values and clinical parameters defined the relevance of the latter. CKD(HD), liver disease and SIRS+Outcomes largely define changes in the plasma metabolome at t₀ in descending order. Normalized energy of sepsis group membership (SIRS+Outcomes) increased from 0.06 at t₀ to 0.14 at t₂₄.

FIG. 7 Variance decomposition (with Pearson correlation) of sepsis diagnosis (non-infected SIRS positive controls vs. sepsis survivor groups) at t₀ (a) and t₂₄ (b). PCA of log-transformed, scaled metabolite concentration at t₀ (c) and t₂₄ (d). Volcano plots showing significant metabolite differences between groups (points above red line) by ANOVA with non-hypothesis components of variance as fixed effects at t₀ (e, FDR 10%) and t₂₄ (f, FDR 5%).

FIG. 8 Plasma metabolite changes in sepsis outcomes (survival or death) in the discovery cohort at t₀ (a) and t₂₄ (b), and in the replication cohort at t₀ (c) and t₂₄ (d). Left, Variance decomposition (with Pearson correlation) of known parameters. Center, Unsupervised PCA of log-transformed, scaled metabolite concentration. Right, Volcano plots showing significant metabolite differences (above red line) by ANOVA with non-hypothesis variance parameters asfixed effects. FDR: t₀ and t₂₄, 5%; Replication t₀, 25%; Replication t₂₄, 15%.

FIG. 9 Variance components attibutable to sepsis survivor subgroups (uncomplicated sepsis, severe sepsis and septic shock, panel a) and etiologic agents (E. coli (n=16), S. pneumoniae (n=31) and S. aureus (n=27), panel b) at t0 were too small (1.7% and 0.2%, respectively) to detect meaningful changes (FDR-corrected (5%) ANOVAs with non-hypothesis components of variance as fixed effects).

FIG. 10 Venn diagrams of significant differences (weighted ANOVA, 5% FDR) in plasma metabolite levels between non-infected control patients (with SIRS) and sepsis survivors at t₀ and t₂₄ (a), concordance of direction of change of significantly altered metabolites (b), and concordance of direction of change of metabolites exhibiting significant differences at one of the time points (c).

FIG. 11 Bar graphs of plasma metabolite levels at t₀ (a), t₂₄ (b) and in replication patients at t₀ (c) and t₂₄ (d). Y-axis displays average scaled plasma metabolite concentrations. Error bars are SEM. Columns represent controls (non-infected, SIRS positive; blue), sepsis survivors (green) and sepsis deaths (red). Asterisks indicate significant differences from sepsis survivors (weighted ANOVA with 5% FDR (a,b), 25% FDR (c) or 15% FDR (d)). All but the relevant negatives carnitine, deoxycarnitine, 3-dehydrocarnitine, 3-dehydrocarntine, steridonate, 3-hybroxybutyrate (BHBA) and acetoacetate were significant. Abbreviations: Glycerophosphethanolamine (-GPE), glycerolphosphocholine (-GPC), 7-α-hydroxy-3-oxo-4-cholestenoate (7-HOCA), dehydroepiandrosterone sulfate (DHEA-S), 3-[4-hydroxyphenyl]lactate (HPLA), symmetric dimethylarginine (SDMA), unannotated disulfated steroids (X-11245 and X-11301).

FIG. 12 Venn diagrams of significant differences in plasma metabolite levels between sepsis survivors and deaths at t₀ and t₂₄ in the discovery and replication (R) cohorts (a), concordance of direction of change of significantly different metabolites (b and d), and concordance of direction of change of metabolites with significant differences at one of the time points (c and e). Significant differences reflect weighted ANOVAs with 5% FDR (t₀ and t₂₄ in the discovery set), 25% FDR (t₀ in the replication set) or 15% FDR (t₂₄ in the replication set).

FIG. 13 Comparisons of the plasma metabolome in community-acquired sepsis survivors and deaths. a Comparison of annotated plasma metabolite levels at t₂₄ in 132 discovery subjects (represented by columns). Individuals who died were ordered by days-to-death (decreasing from left to right as indicated by the black triangle). Rows show 82 host metabolites with statistically significant differences between groups (stratified ANOVA, p<0.05). Colors indicate log-transformed standardized values. Highlighted are 13 acyl-GPCs and -GPEs, which were decreased in sepsis survivors and further decreased in sepsis deaths (in comparison with controls), 13 RNA catabolites and 14 acyl-carnitines, both of which were decreased in sepsis survivors and increased in sepsis deaths (in comparison with controls). b, c, d. 3-dimensional scatterplots showing plasma acyl-carnitine and acyl-GPC levels in 383 samples, as measured by quantitative, targeted assays. b, c. Acylcarnitine levels were generally increased in day-28 sepsis deaths (green contour ellipsoid) and decreased in sepsis survivors (blue ellipsoid) when compared with non-infected controls (red ellipsoid). Sepsis day 28-death samples are indicated by green crosses (n=53; 4-cis-decenoylcarnitine 1825±168 mg/dL; hexanoylcarnitine 41.2±3.5 mg/dL; butyrylcarnitine 68.2±11.7 mg/dL [mean±S.E.M.]), sepsis survivors by blue dots (n=235; 4-cis-decenoylcarnitine 932±50 mg/dL; hexanoylcarnitine 20.3±1.1 mg/dL; butyrylcarnitine 31.9±2.3 mg/dL) and non-infected controls by red dots (n=54; 4-cis-decenoylcarnitine 1200±115 mg/dL; hexanoylcarnitine 24.6±2.9 mg/dL; butyrylcarnitine 35.0±3.7 mg/dL). d. 3-dimensional scatterplot showing similar trends in plasma values of two acyl-glycerophosphocholines (acyl-GPCs) and an RNA catabolite in 383 samples. Acyl-GPCs generally were highest in non-infected (red contour ellipsoid), lower in sepsis survivors (blue contour ellipsoid) and lowest in day-28 sepsis deaths (green contour ellipsoid). Sepsis day 28-deaths are shown by green crosses (n=53; 1-arachidonoyl-GPC 1.10±0.09 mg/dL; 1-linoleoyl-GPC 2.23±0.21 mg/dL; pseudouridine 954±65 mg/dL [mean±S.E.M.]), sepsis survivors by blue dots (n=235; 1-arachidonoyl-GPC 1.38±0.07 mg/dL; 1-linoleoyl-GPC 3.40±0.29 mg/dL; pseudouridine 708±43 mg/dL) and non-infected controls by red dots (n=54; 1-arachidonoyl-GPC 2.49±0.13 mg/dL; 1-linoleoyl-GPC 6.15±0.52 mg/dL; pseudouridine 628±88 mg/dL). Ellipsoids encompass 90% of sample values. e. Box and whisker plots of targeted, quantitative values (red boxes) in 383 plasma samples. Sample values are shown in black. Ranges are shown by black horizontal lines. Means are connected by blue lines. f. The variance in plasma metabolite levels at time of ED enrollment (t₀) that was attributable to sepsis outcome decreased with increasing days-to-death (X-axis). f Heatmap of hierarchical clustering of pairwise Pearson product-moment correlations of 188 log-transformed, annotated plasma metabolites in 132 subjects at t₀. Positive correlations are red; inverse correlations are blue. Metabolites measured at t₀ and t₂₄ were included. Excluded were sparse (detected in <50% of patients) or unannotated GC/MS-determined compounds. Labels are in FIG. 29. g. An identical heatmap, but at t₂₄, illustrating temporal conservation of metabolome perturbation in sepsis survival and death. Labels are in FIG. 30.

FIG. 14 Representative chromatograms of quantitative LC-MS-MS measurement of Butyrylcarnitine, 2-Methylbytyrylcarnitine, Hexanoylcarnitine and cis-4-Decenoylcarnitine (X-11234) in a subject plasma sample.

FIG. 15 Representative calibration curves of quantitative LC-MS-MS measurement of Butyrylcarnitine, 2-Methylbytyrylcarnitine, Hexanoylcarnitine and cis-4-Decenoylcarnitine (X-11234).

FIG. 16 Bar graphs of plasma levels by targeted, quantitative MS-assays of butyrylcarnitine, 2-methylbytyrylcarnitine, hexanoylcarnitine and cis-4-decenoylcarnitine at t₀, t₂₄ and in replication patients at t0 (Rt₀) and t₂₄ (Rt₂₄). Y-axis displays average plasma metabolite concentrations. Error bars are SEM. Columns represent controls (non-infected, SIRS positive), sepsis survivors and sepsis deaths.

FIG. 17 Plasma levels of eleven metabolites in all patients showing relationships between time to death and metabolite values. Plasma metabolite concentrations were determined by targeted, quantitative MS-assays and values are in pm/ml.

FIG. 18 Comparison of two methods of measuring plasma proteins in MS data. a Venn diagram showing overlap of high confidence plasma protein identifications in MS data using two approaches. Results from plasma at t₀ and t₂₄ in the discovery group (n=150) are shown. AUC: X!Tandem and SEQUEST were used to search IPI v3.48 and the non-redundant H. sapiens database and quantification was by AUC of aligned chromatogram peaks. Spectral counts: Mascot v2.0 and Scaffold v3.0 were used to search Swissprot v57.5 and quantification was by spectral counting. c Graph showing correlations between two methods of protein quantitation as a function of values with one of them (Spectral Counts). Shown are log transformed plasma levels of 200 high confidence proteins detected by the methods described above at t₀ and t₂₄. r²=0.488.

FIG. 19 Comparison of C reactive protein (CRP) (a), and albumin (ALB) (b) levels by serum immunoassay (ELISA) and plasma mass spectrometry in 19 and 98 patients, respectively. MS values are log transformed, normalized, areas-under-the-curve of ion chromatograms after background noise removal. Albumin immunoassay values are in mg/dL.

FIG. 20 Z-score scatter plots of proteins detected in human plasma from non-infected SIRS-positive controls, uncomplicated sepsis, severe sepsis (by day 3 post-enrollment), septic shock (by day 3 post-enrollment) or sepsis death (by day 28 post-enrollment) patients. Zero on the X-axis represents the mean of the control group (non-infected SIRS positive). Each data point is expressed as the number of standard deviations from the mean of the control group. The Y-axis represents individual proteins, with all data for any single protein represented on the same horizontal line. The boxed values (mScores) are averages of the absolute values of Z-scores for all proteins, calculated using non-truncated, non-imputed values.

FIG. 21 Principle components of variance (left panels) of plasma proteins in sepsis diagnosis (non-infected SIRS positive controls with sepsis survivors) at t₀ (a) and t₂₄ (b) and sepsis outcome (sepsis survivors and deaths) at t₀ (c) and t₂₄ (d). Center Panels: PCA of log transformed, scaled plasma proteinvalues. Right Panels: Volcano plots showing significant proteins (dots above red line) after ANOVA with non-hypothesis components of variance as fixed effects. Sepsis Diagnosis: t₀ & t₂₄, FDR=5%. Sepsis Outcomes: t₀, FDR=5%; t₂₄, FDR=10%.

FIG. 22 Variance decomposition of venous plasma proteins in sepsis survivor groups at t₀. The variation explicable by these groups (survivors with uncomplicated sepsis, severe sepsis and septic shock, 0.4%) was too small to detect meaningful changes in host plasma protein values.

FIG. 23 Principal components of plasma protein variation associated with etiologic agent in sepsis at t₀ and volcano plots of weighted ANOVAs. a, Principal components of variance decomposition (with Pearson product-moment correlation) for etiologic agents and clinical parameters. Volcano plots of FDRcorrected (5%) ANOVAs (with non-hypothesis components of variance as fixed effects) indicate no significant differences between host proteomic response to bacteremia with E. coli (n=16) and S. pneumoniae, n=31, b), E. coli and S. aureus (n=27, c), and S. pneumoniae and S. aureus (d).

FIG. 24 The plasma proteome in community-acquired sepsis survivors and deaths. a Comparison of annotated plasma protein levels at t₂₄ in non-infected, SIRS-positive controls, 28-day sepsis deaths and sepsis survivors in the discovery group. Columns represent 132 patients. Rows show 69 host proteins with statistically significant differences between groups (stratified ANOVA, p<0.05). Colors indicate log transformed values, standardized to means and standard deviations. 29 complement, coagulation and fibrinolytic proteins which differed among groups are indicated. b Changes in plasma proteins in the complement, coagulation and fibrinolytic cascades in sepsis survivors and deaths. Adapted from KEGG. Red boxes indicate proteins that are significantly decreased in sepsis death compared to survivors; Green boxes are significantly increased in sepsis death. c Heatmap of hierarchical clustering of pairwise Pearson product-moment correlations of 162 log-transformed, annotated plasma proteins and 203 metabolites in 132 subjects at t₀. Positive correlations are red; inverse correlations are blue. Excluded were sparse (detected in <50% of patients) or unannotated analytes. Labels are in FIG. 31. d An identical heatmap, but at t₂₄, illustrating temporal conservation of metabolome and proteome perturbation in sepsis survival and death. Labels are in FIG. 32. e Plasma metabolite correlations with Succinate Dehydrogenase Complex, Subunit D. SDHD was increased 2.44-fold in sepsis death compared with sepsis survival. Regulation of metabolite flow from the pyruvate dehydrogenase complex through the citric acid cycle is shown, with anaplerotic reactions that replenish depleted cycle intermediates and entry into FA β-oxidation. Correlation coefficients of plasma metabolite with plasma SDHD values are indicated by green integers. Plasma lactate, pyruvate, acetyl-carnitine, oxaloacetate and α-ketoglutarate were higher in sepsis deaths than sepsis survivors.

FIG. 25 Plasma proteins exhibiting differences in levels in sepsis at t₀ (a) and t₂₄ (b). Y-axis displays average, scaled log-transformed plasma protein concentrations. Error bars are SEM. Columns represent controls (non-infected SIRS-positive; blue), sepsis survivors (green) and sepsis deaths (red). Asterisks indicate significant differences from sepsis survivors by weighted ANOVA with FDR correction.

FIG. 26 Technical analyses of t₀ mRNA sequencing data of venous blood of 135 subjects. a Overlayed kernel density estimates of transcript expression by log₁₀ transformed genome-aligned mRNA sequence counts in 135 samples. The X-axis shows log transformed gene expression values while the Y-axis shows kernel densities. Samples are represented by individual traces. Group membership is indicated by colors as shown. Inset, Mahalanobis distances of transcript expression by aligned mRNA sequence counts. 135 samples are indicated by colored circles, with groups as indicated. The Y-axis shows Mahalanobis distances of log transformed gene expression values. The dotted blue line indicates the cutoff value for outliers. b Unsupervised principal component analysis of log₁₀ transformed aligned mRNA sequence counts. Three dimensional plots of principal component analysis by Pearson product-moment correlation. 135 samples are indicated by colored circles. Group membership is indicated by colors as shown. c Principal components of variance of log₁₀ transformed aligned mRNA sequence counts. Variance components decomposition of principal components (with Pearson correlation), with partitioning of variability in terms of sepsis subgroups (noninfected SIRS postive controls, NIS; sepsis deaths, SD; Severe Sepsis, SS; Septic Shock, SShock; and Uncomplicated Sepsis, UCS) at t₀.

FIG. 27 Principle components of variance of transcript abundance in peripheral blood by aligned read counts of mRNA sequencing in sepsis diagnosis (non-infected SIRS positive controls with sepsis survivors) at t₀ (a) and sepsis outcome (sepsis survivors and deaths) at t₀ (b). Principle component analysis of log-transformed transcript abundance values in non-infected SIRS positive controls (red circles) and sepsis survivors (blue circles) at t₀ (c) and in sepsis deaths (red circles) and sepsis survivors (blue circles) at t₀ (d).

FIG. 28 The peripheral blood transcriptome in community-acquired sepsis survivors and deaths. a Top panel: Volcano plot of weighted ANOVA of comparison of log-transformed levels of transcripts in sepsis survivors and SIRS-positive, non-infected controls, showing significant up regulation of 3,128 transcripts in sepsis survivors (dots above the red line on the right hand side, FDR 5%). Bottom panel: Volcano plot of weighted ANOVA of comparisons of log-transformed levels of transcripts in sepsis survivors and deaths, showing significant up regulation of 1,326 transcripts in sepsis survivors (dots above the red line on the left hand side). b Functional classification of transcripts with significantly altered levels in sepsis survivors and SIRS-positive, non-infected controls (top panel) and in sepsis survivors and deaths (bottom panel). c Comparison of peripheral blood transcript levels in non-infected, SIRS-positive controls (C), sepsis survivors (S) and sepsis deaths (D) at t₀ in the discovery group. Rows show selected transcripts with statistically significant differences between groups arranged in functional networks and pathways. Blue values are decreased relative to means. Black values are average. Yellow values are increased relative to means. Colors represent log transformed values, standardized to means and standard deviations. Columns of left panels show means of groups. Right panels show individual values in subjects at t₀.

FIG. 29 Heatmap of hierarchical clustering of Pearson-moment pairwise correlations of log-transformed t₀ values of 188 plasma metabolites in 132 patients. Excluded were sparse (detected in <50% of patients), unannotated GC/MS-determined biochemicals, and those without data at both t₀ and t₂₄.

FIG. 30 Heatmap of hierarchical clustering of Pearson correlations of log-transformed t24 values of 188 plasma metabolites in 132 patients. Excluded were sparse (detected in <50% of patients), unannotated GC/MS-determined biochemicals, and those without data at both t₀ and t₂₄.

FIG. 31 Heatmap of hierarchical clustering of Pearson correlations of 162 log-transformed, annotated plasma proteins and 204 metabolites in 138 subjects at t₀ (analytes measured with high confidence at both t₀ and t₂₄).

FIG. 32 Heatmap of hierarchical clustering of Pearson correlations of log-transformed t₂₄ values of 210 venous plasma metabolites and 162 plasma proteins (all analytes measured at both t₀ and t₂₄ in 120 patients).

FIG. 33 Plasma metabolite correlations with Fatty Acid Binding Protein (FABP4, adipocyte), plasma carrier proteins for carnitine esters and free fatty acids. Positive correlation coefficients of plasma metabolite values with plasma FABP4 values are indicated by black integers.

FIG. 34 Selected plasma metabolite correlations with Acyl-CoA synthase. ACSM6 was upregulated 1.33-fold in sepsis death compared with sepsis survival. ACSM6 attaches fatty acids to Coenzyme A for β-oxidation. Esterification of carnitine commits fatty acids to β-oxidation. Correlation coefficients of plasma metabolite values with ACSM6 values are indicated by red (inverse correlations) or blue (positive correlations) integers.

FIG. 35 Molecular models can predict survival and death in community-acquired sepsis. a. The molecular model of sepsis revealed by CAPSOD, featuring early divergence of host response that is predictive of outcome. b. 3-dimensional scatterplot showing demarcation of day-28 SIRS survivors (red ellipsoid) from day-28 SIRS deaths (blue ellipsoid) by plasma hexanoylcarnitine, butyrylcarnitine and HPLA. Survivors are indicated by red dots (n=292) and deaths by blue crosses (n=97). Values are in mg/dL. Ellipsoids encompass 90% of samples. i. 3-dimensional scatterplot showing demarcation of day-7 SIRS survivors (red ellipsoid) from day-7 SIRS deaths (blue ellipsoid) by plasma hexanoylcarnitine, butyrylcarnitine and HPLA. Survivors are indicated by red dots (n=243) and deaths by blue crosses (n=39). Values are in mg/dL. Ellipsoids encompass 90% of values. c-1 Plots and predictive models of survival and death in 379 patient samples by logistic regression with predictor reduction by K-means clusters, T-tests (−log₁₀(p)>1.6) and Forest penalization, proportional prior probabilities and using genetic algorithms for variable selection. c-e Day 28 sepsis survival (n=234) and death (n=91) by hematocrit, 3-methoxytyrosine, log₂HPLA, MAP, Na⁺, log₂4-cis-decenoylcarnitine, log₂creatinine and log₂Na⁺. f-h Day 28 SIRS survival (n=282) and SIRS death (n=97) by Na⁺, GFR-MDRD, log₂HPLA, log₂3-methoxytyrosine, creatinine, MAP and log₂hexanoylcarnitine. j-1 Day 7 SIRS survival (n=340) and SIRS death (n=39) by MAP, log₂MAP, log₂hexanoylcarnitine, Na⁺, log₂creatinine, log₂pseudouridine, HPLA and 3-methoxytyrosine. c,f,j Mosaic plots showing accuracy of death and survival prediction. d,g,k ROC curves with AUCs of 0.85, 0.84 and 0.88, respectively. e,h,l Overlayed plots of sensitivity (+), specificity (∘), accuracy (⋄), PPV (x) and NPV (Δ).

### BEST MODE FOR CARRYING OUT THE INVENTION

Clinical and metabolomic biomarker classifiers were developed to predict survival or death. Sparse models were developed at t₀ using logistic regression along with penalized predictor reduction using a max number of 10 effects in the model, log 10 regularization parameter and 5 max number of categories allowed in a predictor, and cross validation, with 10 percent random holdout and 100 iterations was performed with JMP genomics 5.0 (SAS inc., Cary, N.C.). The analyses identified four clinical factors (Age, mean arterial pressure, hematocrit and temperature) and 12 metabolites (2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate) that reflected underpinning molecular mechanisms, and were also significantly different via ANOVA and Bayesian Factor Analysis.

A seven feature logistic regression model was developed utilizing 4-cis-decenoylcarnitine, 2-methylbutyrylcarnitine, butyrylcarnitine, hexanoylcarnitine, lactate, age, hematocrit and prognostic utility was assessed in t₂₄, Rt₀, and Rt₂₄ datasets. Metabolite classifiers predicted outcomes better than proteins or clinical variables (Data not shown) with high AUCs (Table 1). Since the logistic regression model was developed utilizing all CAPSOD patients, it is possible that the model was over-fitted to best represent the CAPSOD cohort. Therefore, the finished model was independently validated against de-identified sepsis patients' metabolomic values that were graciously provided by Dr. Augustine Choi and the Brigham and Women's Hospital Registry of Critical Illness Cohort (RoCI; approved by the Partners Human Research Committee, protocol #2008-P-000495.(1)). Again, we saw similar strong prediction of sepsis survival and sepsis death utilizing our training set (Table 1). The accuracy, AUC, PPV and NPV of the current gold standards for prognostic assessment in sepsis (SOFA score ≧7, APACHE II score ≧25, and capillary lactate ≧4.0 mg/dL) were lower than most of the seven-feature logistic regression results in all datasets. AUC values at t₀ and t₂₄ of the logistic regression model were superior to the best published biomarker classifier (79% for 3-day prognosis).

TABLE 1
Predictive modeling of metabolomic training and validation datasets

| | | Accuracy | PPV | NPV |
|---|---|---|---|---|
| | | APCAHE II (≧25) | | |
| Sepsis Outcomest | t₀ | 77.2% | 90.0% | 36.4% |
| | t₂₄ | 79.1% | 87.3% | 56.5% |
| | Rt₀ | 73.9% | 93.9% | 23.1% |
| | Rt₂₄ | 75.6% | 96.7% | 18.2% |

| | SOFA (≧7) | | |
|---|---|---|---|
| t₀ | 68.5% | 70.0% | 63.6% |
| t₂₄ | 65.2% | 64.3% | 66.7% |
| Rt₀ | 61.8% | 75.0% | 30.0% |
| Rt₂₄ | 47.6% | 62.5% | 38.5% |
| | Blood Lactate (≧4.0 mg/dL) | | |
| t₀ | 75.0% | 90.8% | 37.0% |
| t₂₄ | 61.2% | 85.7% | 20.0% |
| Rt₀ | 60.6% | 85.7% | 16.7% |
| Rt₂₄ | 75.0% | 100.0% | 25.0% |

| | | Logistic Regression¹ | | | | |
|---|---|---|---|---|---|---|
| | | Accuracy | AUC | RMSE | PPV | NPV |
| Sepsis | t₀ | 85.1% | 84.7% | 35.2% | 94.4% | 58.1% |
| Outcomes | t₂₄ | 79.8% | 80.5% | 39.4% | 85.9% | 64.3% |
| | Rt₀ | 74.5% | 62.5% | 45.2% | 94.1% | 35.3% |
| | Rt₂₄ | 77.1% | 67.4% | 44.7% | 93.8% | 43.8% |
| | BWH | 71.7% | 73.4% | 44.8% | 91.4% | 44.0% |
| | TA^{1,2} | 79.8% | 76.7% | 39.6% | 94.0% | 43.0% |
| | SVM³ | 74.0% | 71.0% | | 79.0% | 63.0% |

¹4-cis-decenoylcarnitine, 2-methylbutyrylcarnitine, butyrylcarnitine, hexanoylcarnitine, lactate, age, hematocrit
²328 targeted assay values tested. All test sets and timepoints combined. Sepsis death, n = 93; sepsis survivors, n = 235.
³173 unique sepsis survivors (n = 124) and sepsis death (n = 49); 87 for training, 86 for test. 100 iterations. 4-cis-decenoylcarnitine, 2-methylbutyrylcarnitine, butyrylcarnitine, hexanoylcarnitine, age, hematocrit, MAP, temperature.

Models were refined using quantitative, targeted MS measurements of the 11 metabolites represented in the initial predictive classifiers in 378 samples and non-sparse, clinical parameters that differed significantly in survivors and deaths. First, the seven-feature logistic regression model was repeated in all sepsis death (n=93) compared to all sepsis survivors (n=235). Clinical lactate values were used in place of targeted assay measurements since the values for most patients were previously captured. Predictive performance was similar to the initially derived test and training sets (Table 1). Support vector machines were used to develop a weighted model for prediction of sepsis survival and death. Data from 173 unique sepsis survivors and deaths was used; where data from the same person was available at both t₀ and t₂₄, one time point was randomly chosen and included (87 for training and the remaining 86 for testing) to avoid testing on a trained patient. Values were normalized by subtracting the mean and dividing by the standard deviation. 100 random partitions were performed for training and test data for each setting. Parameters and weights for the linear SVM determined were 2-methylbutyrylcarnitine 0.1631, 4-cis-decenoylcarnitine 0.1629, butyrylcarnitine −0.4248, hexanoylcarnitine 0.0719, Temperature −0.2602, MAP −0.3157, Age 0.4838, Hematocrit −0.3419 and bias term −0.9959. With these weights, the AUC in 86 unique test subjects was 0.71 and accuracy was 74% (63% for 28-day sepsis death and 79% for sepsis survival).

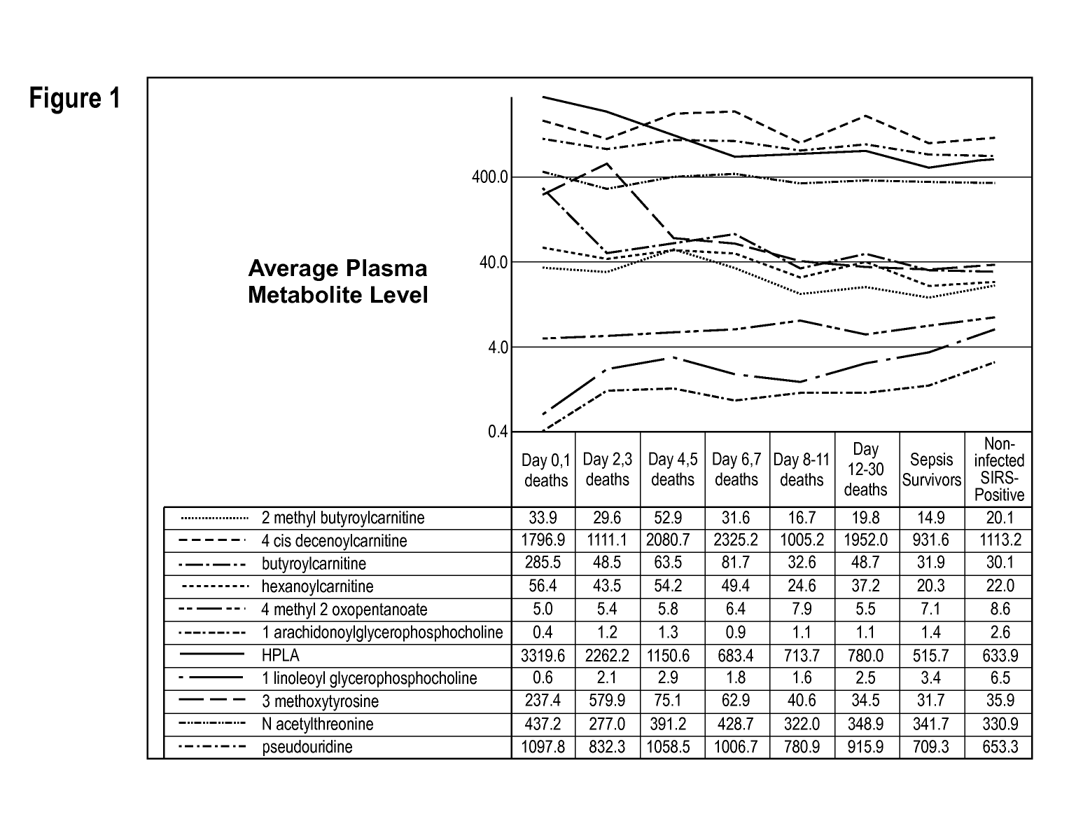

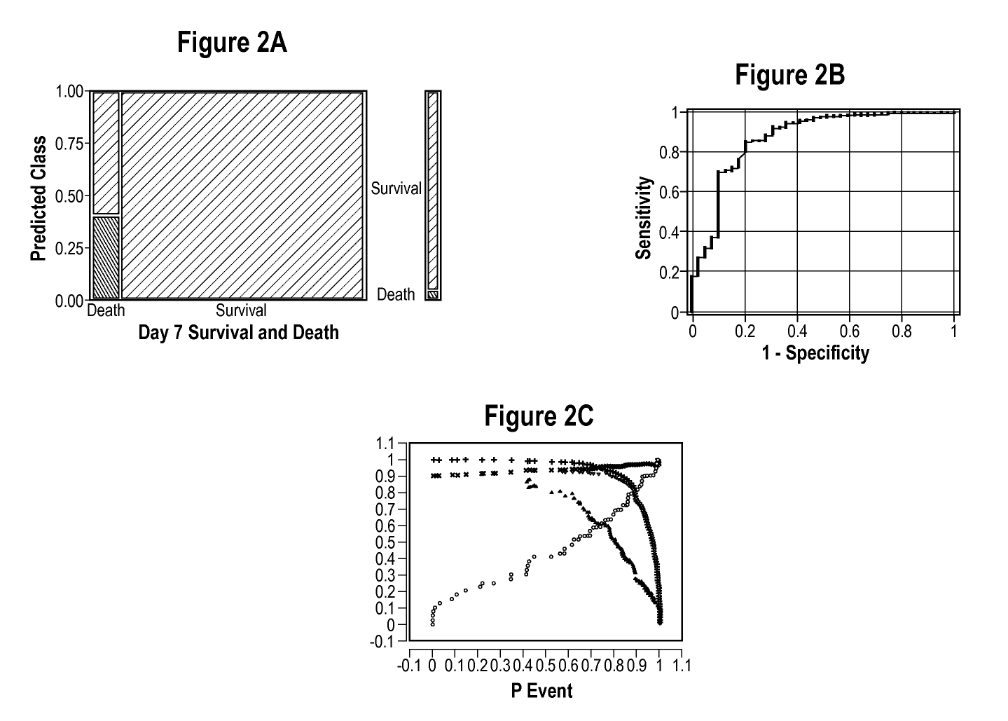

Since we noted that variance in metabolomic profiles could be partially attributed to time-to-death we used the 11 metabolites and clinical features to build a seven-day outcome prediction model to determine if it was superior to 28-day outcome since the metabolomic variance attributable to outcome decayed with increasing time-to-death. Moreover, all eleven plasma metabolite concentrations correlated well between time-to-death and metabolite value (FIG. 1). All cause survival/death included both patients with sepsis and those subsequently diagnosed with other SIRS-causing illnesses and matches the clinical scenario encountered in ED patients precisely. Upon applying a realistic prior probability of death of 10%, day-7 survival prediction was 99% accurate (FIG. 2). The factors in this model represented the observed dichotomy in host response and/or have previously shown utility in sepsis outcome prediction (Mean Arterial Pressure (MAP), hexanoylcarnitine, Na⁺, creatinine, pseudouridine, HPLA and 3-methoxytyrosine).

The strong replication in internal and external validation sets, targeted assays, SVM analysis, and predictive time-to-death models suggest that metabolomic features described will provide strong utility for sepsis death and survival prediction at presentation.

The plasma metabolome, plasma proteome and blood transcriptome of over 200 rigorously phenotyped individuals with community-acquired sepsis or controls (SIRS without infection) were analyzed by mass spectrometry and mRNA sequencing, respectively, in discovery and validation studies at ED arrival and 24 hours later. Host responses to sepsis were dichotomous and predicted 28-day sepsis outcome: Molecular divergence of sepsis survivors, sepsis deaths and controls was present at ED arrival, increased after 24 hours, and continued to diverge as death approached. Analytes differed minimally among etiologic agents or between survivors with uncomplicated sepsis, severe sepsis or septic shock. While sepsis survivors mobilized and utilized diverse energy substrates aerobically, sepsis patients who would die exhibited impaired ft-oxidation of fatty acids, with acylcarnitine accumulation and RNA degradation. Concomitant changes in transcription provided explanations for proteomic and metabolic differences. Collapsed rare and common genetic variants in 20 genes showed significant association with survival and death.

The integration of systems surveys revealed sepsis to be a complex, heterogeneous and highly dynamic pathologic state and yielded new insights into molecular mechanisms of survival or death that could potentially enable predictive differentiation and individualized patient treatment. Early accumulation of catabolic intermediates of lipids, proteins, RNA and carbohydrates in plasma of sepsis patients who would die, most notably acyl carnitines, were found, together with widespread decreases in mRNA of genes involved in glycolysis and gluconeogenesis. These changes were reversed in sepsis survivors. Therefore, the primacy of metabolism was shown to be a determinant of sepsis survival and death. The present invention also presented structural studies showing mitochondrial derangements, decreased mitochondrial number and reduced substrate utilization in sepsis death, and progressive drop in total body oxygen consumption with increasing severity of sepsis. An early differential in sepsis survival or death is the presence or absence of mitochondrial biogenesis, respectively. Finally, sepsis-induced multiple organ failure occurs despite minimal cell death in affected organs and recovery occurs relatively rapidly in sepsis survivors, ruling out other potential mechanisms of sepsis death. A causal role for elevated acylcarnitines in sepsis death is discovered by the finding that micromolar palmitoylcarnitine causes ventricular contractile dysfunction. Furthermore, adults with Mendelian mutations of acylcarnitine metabolism have similar metabolic derangements and high rates of sudden death. Alternatively, the differences observed in corticoid levels in sepsis survivors and nonsurvivors may be token neuro-hormonal control of disparate metabolic responses to sepsis.

The immediacy of the metabolic dichotomy in sepsis—before organ failure or shock became established—was very surprising. Survivors and deaths did not differ significantly in medication prior to enrollment. However, nucleotide variants in 20 genes showed evidence as risk factors for a pre-existing susceptibility and an adverse outcome. The functions of these genes concurred with the molecular differences between sepsis survival and death: single stranded DNA binding protein 1 is involved in mitochondrial biogenesis; SLC16A13 transports lactate and pyruvate; vitamin K epoxide reductase complex, subunit 1, is important for blood clotting; CCAAT/enhancer binding protein ε is important in granulocyte maturation and response to TNFα; NADH dehydrogenase 1 α2 and β8 are components of the mitochondrial electron transport chain.

Also surprising was the molecular homogeneity of uncomplicated sepsis, severe sepsis and septic shock, challenging the traditional notion of a temporal or molecular pyramid of sepsis progression. Additional longitudinal investigation of the host metabolic response to sepsis is needed to address more fully the temporal dynamics and general relevance of this dichotomy in community-acquired and nosocomial sepsis among diverse patient populations, ages and types of infection. Investigation of the relevance of host metabolic dichotomy to other SIRS-inducing conditions, such as trauma, hyperthermia and drug-induced mitochondrial damage, is also needed. The reversibility of the death phenotype by targeted interventions such as early goal-directed therapy, succinate administration or enhancement of mitochondrial biogenesis needs to be assessed. Global and temporal correlation of metabolome, proteome and transcriptome data from relevant biological fluids and well phenotyped patient groups, is suitable for understanding of intermediary metabolism, particularly with respect to poorly annotated analytes, and for characterization of homogeneous subgroups in complex traits. Combinations of transcriptome, proteome, metabolome and genetic data may establish multi-dimensional molecular models of disease that could provide insights into network responses to intrinsic and/or extrinsic perturbation.

Global correlations of plasma proteomic and metabolomic datasets recapitulated known mass action kinetic models of catalysis or physicochemical complex assembly and suggested novel models disclosed herein. Hierarchical clustering of correlations predicted class membership for unannotated biochemicals that were substantiated by structural determination. The clinicometabolomic model disclosed herein predicted day-7 survival with 99% accuracy, providing basis for individualized sepsis treatment. Therefore the invention is proved to be useful for predictive differentiation and nomination of novel potential interventions in complex pathologic states.

### EXAMPLES

The following examples set forth preferred materials and procedures in accordance with the present invention. Although any methods and materials similar or equivalent to those described herein can be used in the practice or testing of the present invention, the preferred methods, devices, and materials are now described. It is to be understood, however, that these examples are provided by way of illustration only, and nothing therein should be deemed a limitation upon the overall scope of the invention.

### Methods and Materials:

Summary:

Patients presenting at EDs at Henry Ford Hospital, Duke University Hospital, and Durham Veterans Affairs Medical Center with suspected sepsis (≧2 SIRS criteria and infection) were enrolled. The CAPSOD study was approved by institutional ethics committees and written informed consent was given by patients. Physical examination and blood sample collection were performed at enrollment and 24 hrs later. Patients were followed for 28 days. Anonymized demographic and clinical data was stored in compliance with HIPAA regulations (ProSanos Inc., Harrisburg, Pa.). Following blinded, expert audit of infection status and outcomes, 150 matched subjects were chosen for discovery studies. Patients were classified as non-infected SIRS-positive uncomplicated sepsis, severe sepsis, septic shock or sepsis death. t₀ and t₂₄ samples from another 52 matched sepsis survivors and deaths were used for validation. Plasma metabolites were prepared and analyzed by high performance liquid chromatography and linear ion trap quadrupole (LTQ) MS with electrospray ionization and by gas chromatography and fastscanning dual-stage quadrupole MS with electron impact ionization (Metabolon Inc, Durham, N.C.). Plasma proteins were immunodepleted by GenWay Seppro IgY-12 columns and analyzed by LTQ MS in triple-play mode (Monarch Life Sciences Inc.). mRNA was isolated from blood samples and sequenced on Illumina GAIIx instruments. Statistical analysis employed JMP Genomics 5.0 (SAS Institute).

CAPSOD Study Sites and Patients:

The Community Acquired Pneumonia and Sepsis Outcome Diagnostics (CAPSOD) study was approved by the Institutional Review Boards of the National Center for Genome Resources (Santa Fe, N. Mex.), Duke University Medical Center (Durham, N.C.), Durham Veteran Affairs Medical Center (Durham, N.C.) and Henry Ford Hospital (Detroit, Mich.) and filed at ClinicalTrials.gov (NCT00258869). Inclusion criteria were presentation of adults at the emergency department with known or suspected acute infection and presence of at least two of the four systemic inflammatory response syndrome (SIRS) criteria (tympanic temperature <36° C. or >38° C., tachycardia >90 beats per minute, tachypnea >20 breaths per minute or PaCO₂<32 mmHg, white cell count <4000 cells/mm³ or >12,000 cells/mm³ or >10% neutrophil band forms). Exclusion criteria were as previously described. Patients were enrolled from 2005 through 2009 in emergency departments at each institution and written informed consent was obtained by all study participants or their legal designates.

Clinical Data Collection:

Patient demographics, exposures, past medical history, results of physical examination, APACHE II score, SOFA score, development of ALI or ARDS and treatment were recorded at enrollment (t₀) and at 24 hours (t₂₄) by a nurse practitioner or physician using online electronic data capture (Prosanos Inc., Harrisburg, Pa.) as previously described. Microbiologic evaluation was as indicated clinically, supplemented by urinary pneumococcal and Legionella antigen tests. Finger-stick lactate values were obtained. After 28 days, charts were reviewed and largest deviations of clinical and laboratory parameters from normal were recorded, together with outcome measures, microbiologic results, treatment and time-to-events. Blood for metabolomic and proteomic analyses was collected in bar-coded EDTA-plasma tubes at enrollment (t₀) and the following day (t₂₄), incubated on ice, plasma separated (within 4 hours), and aliquots stored at −80° C. Blood for mRNA sequencing was collected in PaxGene tubes at enrollment (t₀) and the following day (t₂₄), incubated at room temperature and stored at −20° C.

Clinical Data Audit and Discovery Cohort Selection:

All subject records were adjudicated independently by a study physician to determine whether presenting symptoms and signs were due to infection, etiologic agent, site of infection, patient outcomes and times-to-outcomes. Patients were clinically categorized based on infection likelihood and microbial etiology: definite infection, causative organism identified; definite infection, causative organism uncertain; indeterminate, infection possible; no evidence of infection; and no evidence of infection and diagnosis of a non-infectious process accounting for SIRS. 150 patients were selected from the definite infection and non-infection categories for plasma metabolome and proteome analyses as follows: non-infected patients with >2 SIRS criteria (n=29); uncomplicated sepsis (sepsis without progression and with survival at day 28; n=27); severe sepsis (sepsis at t₀ with progression to severe sepsis by day 3, n=25); septic shock (sepsis at t₀ with progression to septic shock by day 3, n=38); sepsis deaths (sepsis with death by day 28, n=31). Patients with sepsis were further selected to enrich for confirmed infections due to E. coli, S. aureus, and S. pneumoniae. Within these constraints, groups were matched for age, race, sex and enrollment site. The estimated glomerular filtration rate (eGFR) was calculated as described.

Metabolite Sample Preparation and Gas Chromatography/Mass-Spectrometry and Liquid Chromatography/Mass-Spectrometry Analysis:

Plasma samples were thawed on ice at Metabolon Inc. (Durham, N.C.), and 100 μL was extracted using an automated MicroLab STAR system (Hamilton Company, Reno, Nev.), as described. A well characterized human plasma pool (“Matrix”, MTRX) was also included as a technical replicate, to assess variability and sensitivity in the measurement of all consistently detected chemicals. A single solvent extraction was performed with 400 μl of methanol containing recovery standards by shaking for two minutes using a Geno/Grinder 2000 (Glen Mills Inc., Clifton N.J.). After extraction, the sample was centrifuged, the supernatant removed and split into four equal aliquots: two for LC/MS, one for GC/MS, and a reserve aliquot. Aliquots were dried under vacuum overnight on a TurboVap (Zymark, Hopkinton, Mass.). Samples were maintained at 4° C. throughout the extraction process. For LC/MS analysis, aliquots were reconstituted in either 0.1% formic acid (for positive ion LC/MS), or 6.5 mM ammonium bicarbonate pH 8.0 (for negative ion LC/MS) containing internal standards for chromatographic alignment. For GC/MS analysis, aliquots were derivatized using equal parts N,O-bistrimethylsilyl-trifluoroacetamide and a mixture of acetonitrile:dichloromethane:cyclohexane (5:4:1) with 5% triethylamine at 60° C. for 1 hour. The derivatization mixture also contained a series of alkyl benzenes that served as retention time markers.

LC/MS was carried out using an Acquity UPLC (Waters Corporation, Milford, Mass.) coupled to a linear ion trap quadrupole (LTQ) mass spectrometer (Thermo-Fisher Scientific Inc., Waltham, Mass.) equipped with an electrospray ionization source. Two separate LC/MS injections were performed on each sample: the first optimized for positive ions, and the second for negative ions. The mobile phase for positive ion analysis consisted of 0.1% formic acid in H₂O (solvent A) and 0.1% formic acid in methanol (solvent B), whereas that for negative ion analysis consisted of 6.5 mM ammonium bicarbonate, pH 8.0 (solvent A) and 6.5 mM ammonium bicarbonate in 95% methanol (solvent B). The acidic and basic extracts were monitored for positive and negative ions, respectively, using separate acid/base dedicated 2.1×100 mm Waters BEH C18 1.7 μm particle columns heated to 40° C. The extracts were loaded via a Waters Acquity autosampler and gradient-eluted (0% B to 98% B, with an 11 minute runtime) directly into the mass spectrometer at a flow rate of 350 μl/min. The LTQ alternated between full scan mass spectra (99-1000 m/z) and data-dependent MS/MS scans, which used dynamic exclusion.

Derivatized samples were analyzed on a Thermo-Fisher Scientific Trace DSQ fastscanning single-quadrupole MS set at unit mass resolving power. The GC column was 20 m×0.18 mm with 0.18 μm film phase consisting of 5% phenyldimethyl silicone. The temperature program ramped from 60° C. to 340° C., with helium as the carrier gas. The MS was operated using electron impact ionization with a 50-750 amu scan range, tuned and calibrated daily for mass resolution and mass accuracy. Samples were randomized to avoid group block effects and were analyzed over five platform days (for discovery group samples) or two platform days (for replication group samples). Six MTRX aliquots, an internal standard sample (see below) and various control samples were included in each run.

Metabolites were identified by automated comparison to a reference library of purified external standards using Metabolon software developed for creating library entries from known chemical entities with automatic fitting of reference to experimental spectra. Peaks that eluted from the LC or GC methods were compared to the library at a particular retention time and associated spectra for that metabolite. Internal standards were used to calibrate retention times of metabolites across all samples. Platform variability was determined by calculating the median relative standard deviation (RSD) for the internal standard compounds that were added to every sample. Overall variability (including sample preparation) was determined by the median RSD for 261 endogenous metabolites present in all MTRX samples. Peptides were identified using standard tandem mass spectrometry sequencing.

Raw area counts for each metabolite in each sample were normalized to correct for variation resulting from instrument inter-day tuning differences. For each metabolite, the raw area counts were divided by the median value for each run-day, therefore setting the medians to 1.0 for each run. This preserved variation between samples, but allowed metabolites of widely different raw peak areas to be compared on a similar graphical scale. Missing values were imputed with the observed minimum after normalization. However, metabolites with missing values in >50% of the samples were excluded from analysis.

Identification of Unknown Biochemical X-11234:

The unknown compound X-11234 was identified as cis-4-decenoyl carnitine based on comparison of its mass spectrum and chromatographic retention time with an authentic standard.

Quantitative LC/MS/MS Measurements:

A combined internal standard working solution was made, comprising butyrylcarnitine-d3 at 400 μg/mL, 2-methylbutyrylcarnitine-d3 at 200 μg/mL, hexanoylcarnitine-d3 at 200 μg/mL and cis-4-decenoylcarnitine-d3 (Universidad Autonoma de Madrid, Spain) at 400 μg/mL in acetonitrile/water (1:1). Six calibration samples were made in acetonitrile/water (1:1): Standard A: butyrylcarnitine 2 μg/mL, 2-methylbutyrylcarnitine 4 μg/mL, hexanoylcarnitine 2 μg/mL, cis-4-decanoylcarnitine 40 μg/mL. Standard B: butyrylcarnitine 4 μg/mL, 2-methylbutyrylcarnitine 8 μg/mL, hexanoylcarnitine 4 μg/mL, cis-4-decanoylcarnitine 80 μg/mL. Standard C: butyrylcarnitine 10 μg/mL, 2-methylbutyrylcarnitine 20 μg/mL, hexanoylcarnitine 10 μg/mL, cis-4-decanoylcarnitine 200 μg/mL. Standard D: butyrylcarnitine 40 μg/mL, 2-methylbutyrylcarnitine 80 μg/mL, hexanoylcarnitine 40 μg/mL, cis-4-decanoylcarnitine 800 μg/mL. Standard E: butyrylcarnitine 100 μg/mL, 2-methylbutyrylcarnitine 200 μg/mL, hexanoylcarnitine 100 μg/mL, cis-4-decanoylcarnitine 2000 μg/mL. Standard F: butyrylcarnitine 200 μg/mL, 2-methylbutyrylcarnitine 400 μg/mL, hexanoylcarnitine 200 μg/mL, cis-4-decanoylcarnitine 4000 μg/mL. 50 μL of 393 human EDTA plasma samples, 48 quality control plasma aliquots, 6 calibration standards and a blank internal standard (H₂O) were each spiked with 20 μL of internal standard working solution and 50 μL of acetonitrile/water (1:1) and 200 μL of methanol. Samples were vortexed and centrifuged to precipitate proteins. 180 μL of the supernatant was dried under a stream of nitrogen at 40° C., reconstituted in 75 μL of water, vortexed, centrifuged and injected onto a Waters Acquity UPLC/Thermo Quantum Ultra triple quadrupole LC/MS/MS system with HESI source equipped with a reversed phase chromatographic column. The peak areas of the respective product ions were measured against the peak areas of the corresponding internal standard product ions. The monitored ion masses (SRM mode) were: as follows: for butyrylcarnitine, parent ion 232.2+0.5, product ion 85.0+0.5; For butyrylcarnitine-D₃, parent ion 235.2+0.5, product ion 85.0+0.5; For 2-methylcarnitine, parent ion 246.2+0.5, product ion 85.0+0.5; For 2-methylcarnitine-D₃, parent ion 249.2+0.5, product ion 85.0+0.5. For hexanoylcarnitine, parent ion 260.2+0.5, product ion 85.0+0.5; For hexanoylcarnitine-D₃, parent ion 263.2+0.5, product ion 85.0+0.5; For cis-4-decenenoylcarnitine, parent ion 314.2+0.5, product ion 85.0+0.5; For cis-4-decenoylcarnitine-D₃, parent ion 317.2+0.5, product ion 85.0+0.5. 1. Chromatographic conditions were: Mobile phase A, 0.1% formic acid in water; Mobile phase B, 0.5% formic acid in acetonitrile; UHPLC column, Waters Acquity C 18 BEH, 1.7 micron 2.1×100 mm; Injection volume, 10 μL. Quantitation was performed using a weighted linear least squares regression analysis generated from fortified calibration standards prepared immediately prior to each run. The dynamic range was 2.00-200 μg/mL for butyrylcarnitine, 4.00-400 μg/mL for 2-methylbutyrylcarnitine, 2.00-200 μg/mL for hexanoylcarnitine and 40.0-4000 μg/mL for cis-4-decenoylcarnitine. 48 replicate plasma quality control sample aliquots were interspersed and analyzed together with the study samples and a calibration curve at the beginning and end of each run. The interday % RSD (total of 8 analytical runs) for butyrylcarnitine was 5.1%, 2-methylcarnitine was 4.9%, hexanoylcarnitine was 5.8% and cis-4-decenoylcarnitine was 4.8%.

Proteome Sample Preparation and Mass Spectrometry Analysis (Monarch Life Sciences):

Plasma samples were thawed on ice at Monarch Life Sciences Inc. and the top-12 most abundant proteins (albumin, IgG, fibrinogen, transferrin, IgA, IgM, haptoglobin, α2-macroglobulin, α1-acid glycoprotein, α1-antitrypsin and apolipoprotein A-I and A-II) were removed using Seppro IgY-12 Columns (GenWay Biotech Inc.). Column flow-throughs were denatured by 8M urea, reduced by triethylphosphine, alkylated by iodoethanol and digested by trypsin, as described. Tryptic digests (˜20 μg) were analyzed using a Thermo-Fisher Scientific LTQ linear ion-trap mass spectrometer coupled with a Surveyor HPLC system. Peptides were separated on a C18 reverse phase column (i.d. =2.1 mm, length=50 mm) with a flow rate of 200 μl/min and eluted with a gradient from 5 to 45% acetonitrile developed over 120 min. All injections were randomized and the instrument was operated by the same operator for the study. Data were collected in the triple-play mode (MS scan, zoom scan and MS/MS scan). Data were filtered and analyzed as described. Database searches against the IPI (International Protein Index) human database (v3.48) and the non-Redundant-Homo Sapiens database (update July 2009) were carried out using both the X!Tandem and SEQUEST algorithms. Parameters were set as follows: a mass tolerance of 2 Da for precursors and 0.7 Da for fragment ions, two missed cleavage sites allowed for trypsin, carbamidomethyl cysteine as fixed modification, and oxidized methionine as optional modification. The q-value represented peptide false identification rate and was calculated by incorporating Sequest and X!Tandem results in addition to a number of other relevant factors such as Ä [M+H]+ and charge state. Observed peptide MS/MS spectrum and theoretically derived spectra were used to assign quality scores (Xcorr in SEQUEST and e-Score in X!Tandem). Protein identities were assigned priority scores (from 1 to 4): based on the peptide ID confidence (q-value) and the number of unique peptides used for protein identification: Priority 1, high peptide confidence (>90%) and multiple unique sequences; Priority 2, high peptide confidence (>90%) and single peptide sequence; Priority 3, moderate peptide confidence (between 75% and 89%) and multiple unique sequences; Priority 4, moderate peptide confidence (between 75% and 89%) and single peptide sequence. Priority 1 protein identifications were employed for analyses, except protein-metabolite correlations, which also employed Priority 2 identifications that were observed at both t₀ and t₂₄. Protein quantification was carried out using the method of Higgs et al. Briefly, raw files were acquired from the LTQ and all extracted ion chromatograms (XIC) were aligned by retention time. For protein quantification, each aligned peak must match four criteria: precursor ion, charge state, fragment ions (MS/MS data) and retention time (within a one-minute window). After alignment, area-under-the-curve (AUC) for each individually aligned peak from each sample was measured and compared for relative abundance. As an example, the XICs and ANOVA for chicken lysozyme (an external control) in 150 subjects at t₀ are appended.

Peak intensities were log 2 transformed before quantile normalization₉₀ to ensure that every sample had a peptide intensity histogram of the same scale, location and shape. Normalization removed trends introduced by sample handling, sample preparation, total protein differences and changes in instrument sensitivity while running multiple samples (data not shown). If multiple peptides had the same protein identification, then their quantile normalized log 2 intensities were averaged to obtain log 2 protein intensities.

Proteome Mass Spectrometry Analysis:

Raw LC-MS/MS data files collected on a LTQ Linear Ion Trap (ThermoFisher Scientific, Waltham. MA) were delivered to the Duke Proteomics Core Facility as .raw files with appropriate deidentified clinical data. The centroid MS/MS data was processed into .mgf files using Mascot Distiller v2.0 (Matrix Sciences, Inc Boston, Mass.), and searched with Mascot v2.2. Mascot was set up to search the Swissprot v57.5 database (www.uniprot.org) with human taxonomy and decoy database enabled, trypsin specificity with a maximum of 2 missed cleavages, and 2 Da precursor and 0.8 Da product ion mass accuracy. Iodoacetamide derivative of cysteine was specified as a fixed modification, and deamidation of asparagine, deamidation of glutamine, and oxidation of methionine were specified in Mascot as variable modifications. Scaffold version 3.0 (Proteome Software Inc., Portland, Oreg.) was used to import search results directly from Mascot and validate MS/MS based peptide and protein identifications. Because of the number of analyses, the time zero (n=150) and 24 hour (n=131) datasets were imported and validated in Scaffold independently. For both data sets, peptide identifications were accepted if they could be established at greater than 50.0% probability as specified by the Peptide Prophet algorithm, and protein identifications were accepted if they could be established at greater than 90.0% probability and contained at least 1 identified peptide. Protein probabilities were assigned by the Protein Prophet algorithm₉₂. Proteins that contained similar peptides and could not be differentiated based on MS/MS analysis alone were grouped to satisfy the principles of parsimony. Non-normalized spectral counting reports were then exported independently for each of the datasets, and compiled in Microsoft Excel 2007. Using the Protein Prophet scores, the protein search results from both datasets were compiled, sorted and curated using reverse (decoy) sequences identified to set the protein false discovery rate of the aggregate dataset to 2.5%. Proteins identified below this threshold were discarded from the dataset. Follow-up comparative quantitation between individuals and timepoints was performed using spectral counting in the form of number of identified spectra per protein.

Transcriptome Sample Preparation and mRNA Sequencing:

RNA was prepared using a PaxGene Blood RNA kit (Qiagen, Germantown, Md.) according to the manufacturer's instructions. Briefly, nucleic acids were pelleted by centrifugation, washed and treated with proteinase K. Residual cell debris was removed by centrifugation through a column. Samples were equilibrated with ethanol and total RNA was isolated using a silica membrane. Following washing and DNase I treatment, RNA was eluted. RNA integrity was determined by 2100 Bioanalyzer microfluids using RNA 600 Nano kit (Agilent). RNA samples were stored at −80° C.

mRNA sequencing libraries were prepared from total RNA according to Illumina's mRNA-Seq Sample Prep Protocol v2.0/2007. Briefly, mRNA was isolated using oligo-dT magnetic Dynabeads (Invitrogen, Carlsbad, Calif.). Random-primed cDNA was synthesized and fragments were 3′ adenylated. Illumina DNA oligonucleotides adapters for sequencing were ligated and 350-500 bp fragments were selected by gel electrophoresis. cDNA sequencing libraries were amplified by 18 cycles of PCR and quality was assessed with the Bioanalyzer. cDNA libraries were stored at −20° C.

Biological replicate cDNA libraries, prepared from whole blood extracted from an anonymous healthy individual, were sequenced on the Illumina GA_{II} instruments as 36-cycle singleton reads. CAPSOD experimental samples were sequenced on Illumina GA_{II} instruments 54-cycle singleton reads). Base calling used the Illumina Pipeline software v1.4, except for 14 samples which used v1.3. Approximately 500 million high quality reads were generated per sample. Reads were aligned to the NCBI human nuclear genome reference build 37 and the corresponding human mitochondrial genome reference using the algorithm GSNAP (Mar. 9, 2010 release). GSNAP alignment parameters were: maximum mismatches=((readlength+2)/12)−2; indel penalty=1; trim=1; indel endlength=12; maximum middle deletion size=6000 nt; maxmiddle-insertions=60. Uniquely aligned reads were enumerated on a RefSeq gene-by-gene basis and expressed as aligned reads per million. Variants were detected in reads aligned by GSNAP.

Variants were retained if present in >=4 reads of Q>=20 and >14% reads, with the exception of mitochondrial variants, which were retained if present in >10% reads. Numeric genotypes (0, homozygous reference; 1, heterozygous; 2, homozygous variant, •, nucleotide coverage <4 reads) were imputed in reads aligning to the nuclear genome; mitochondrial variants were assigned present or absent (0, absent [present in <10% reads]; 1, present [>=10% reads]; •, nucleotide coverage <4 reads). Heterozygous nuclear variants were present in 14-86% of reads; homozygotes were represented by reads with <14% or >86% variant calls, as described.

Statistical Analyses:

Overlaid kernel density estimates, univariate distribution results, correlation coefficients of pair wise sample comparisons, unsupervised principal components analysis (by Pearson productmoment correlation) and Ward hierarchal clustering of Pearson product-moment correlations were performed using log₂-transformed data as described using JMP Genomics 5.0 (SAS Institute). Decomposition of principal components of variance, including patient demographics, past medical history, laboratory and clinical values, was performed to maximize sepsis-group related components of variance and minimize residual variance. Guided by these analyses, ANOVA was performed between sepsis groups, with 5 or 10% false discovery rate (FDR) correction and inclusion of substantive non-hypothesis components of variance as fixed effects. These included renal function, as determined by the estimated glomerular filtration rate (eGFR) using the four variable modification of diet in renal disease calculation₉₆, hemodialysis (HD), cirrhosis and liver disease, hepatitis, neoplastic disease, congenital disease, administration of exogenous immunosuppressants, drug abuse, metabolic dysfunction, respiratory dysfunction, serum glucose levels and mean arterial pressure (MAP). Predictive modeling was performed with JMP Genomics 5.0 using logistic regression, K nearest neighbors, partial least squares, partition trees and radial basis machines. Cross-validation was performed using 50 iterations and 10% sample omission.

Variant associations with survival/death were performed by comparing a binary trait with numeric genotypes of both common and rare variants. Rare variants were recoded according to a dominant model and combined within genes into a single locus. Association tests were then performed using JMP Genomics 5.0 on each single locus (using Person chi-square and Fisher's exact test) and combined tests on all variants within a gene (using Hotelling's T-squared test or on the principal components representing the variants as a regression model). The significance cutoff was −log 10(p value)>8.0. Significant associations were retained if observed in at least 60 samples, had at least moderately altered odd ratios, and following manual inspection of read alignments to confirm variant calls.

Ingenuity Pathway Analysis software (version 8.7, content version 3203) was used to assign biological functions to differentially expressed genes.

Pairwise cross correlations were performed using JMP Genomics 4.0 software to compare protein and metabolite values at t₀ and t₂₄ using Pearson moment-correlation. Briefly, all proteins and all metabolites were included, with the exception of unannotated GC/MS determined compounds or redundant entries. Metabolite and protein log 2 values were transposed into a wide format and the correlations were merged based on patient identification. Protein metabolite correlations were considered significant if observed at t₀ and t₂₄ with p-values <0.05 and <0.1, or at a single time point with Bonferroni correction. To identify significant, sepsis associated correlations, the same analysis was performed but limited only to proteins or metabolites that were significant at both time points with concordant changes.

Unannotated metabolites and proteins, except the sulfated steroids X-11245 and X11302, were removed.

Support vector machines (SVM), both linear and with RBF kernels, were used for binary classification of sepsis survivors and deaths (SD). Data from 173 unique sepsis survivors and deaths was used; where data from the same person was available at both t₀ and t₂₄, one time point was randomly chosen and included. Features were either four quantitative MS-assays of acylcarnitines or the four acylcarnitines and four non-sparse, clinical parameters that showed significant differences between survivors and deaths (age, temperature, MAP and hematocrit). 100 random partitions were performed for training and test data for each setting. SVM performance was evaluated by test data scores for area under the receiver operating characteristic (ROC) curve (AUC) and accuracy. Accuracy was highly dependent on the threshold chosen for the scores. In all experiments, the scores of training samples were sorted and the N_SDth score was used as the threshold with test data. Parameter weights were derived for linear SVM.

The following examples illustrate preferred embodiments of the present invention. These examples are provided for illustration only and the invention is not limited by these examples.

### Example 1

### Clinical Synopsis

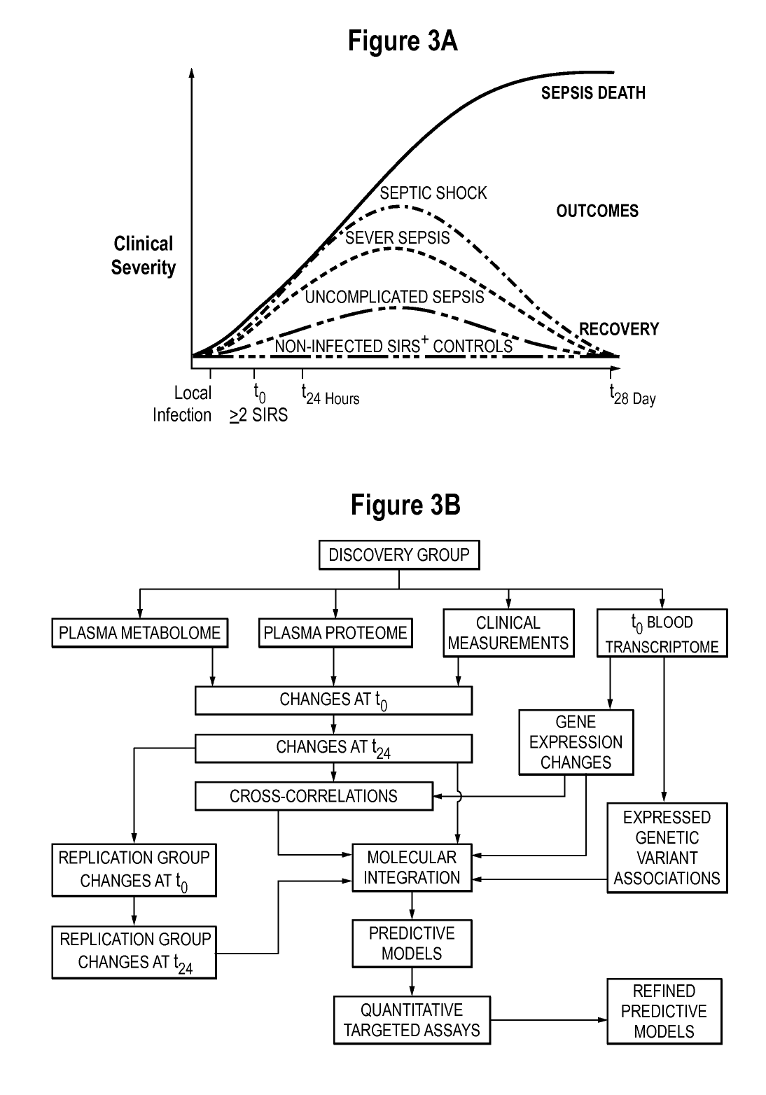

1,152 individuals with suspected, community-acquired sepsis (acute infection and ≧2 SIRS criteria¹⁶) were enrolled prospectively at three urban, tertiary-care EDs in the United States between 2005 and 2009 [Community Acquired Pneumonia and. Sepsis Outcome Diagnostics (CAPSOD), ClinicalTrials.gov NCT00258869]. Medical history, physical examination, acute illness scores (APACHE II and SOFA) and blood samples were recorded at enrollment (t₀) and 24 hours later (t₂₄; FIG. 3). APACHE II and SOFA were ascertained to provide gold standard clinical prognostic determinations. The two time points were chosen both to represent the earliest time practicable in sepsis evolution and to permit limited analysis of the temporal dynamics of molecular responses. Infection status and outcomes through day 28 were independently adjudicated. Conventional diagnosis of etiologic agent was supplemented by urinary pneumococcal antigen and PCR of blood for bacterial and fungal DNA. The cohort was distinctive in that a majority of patients were African American and 28-day mortality was 4.9%. A previous CAPSOD study found early progression to shock (systolic blood pressure <90 mm Hg) to be associated with higher 30-day mortality.

150 CAPSOD enrollees were selected for mass spectrometry (MS)-based venous plasma metabolome and proteome profiling at t₀ and t₂₄, venous blood mRNA-seq at t₀ and integrative analysis (FIG. 3). The subjects comprised 5 groups, which were chosen to reflect the conventional concept of a pyramid of progression in sepsis. They were: day 28 sepsis survivors with uncomplicated courses (n=27), sepsis survivors who developed severe sepsis or septic shock by day 3 (n=25 and 38, respectively), sepsis deaths (by day 28, n=31), and ill controls (presumed to have sepsis at enrollment but later determined to have a non-infectious SIRS etiology; n=29) (Table 2).

TABLE 2
Definitions of Severe Sepsis and Septic Shock

| Organ | | |
|---|---|---|
| Dysfunction | Measure | Range |
| Cardio- | Arterial Systolic Pressure | ≦90 mmHG (≧18 years) |
| vascular¹ | Or MAP | ≦70 mmHG (≧18 years) |
| | Or Vasopressors | Dopamine (≧5 μg/kg/min); |
| | | norepinephrine, epinephrine |
| | | or phenylephrine (any dose) |
| Renal¹ | Urine output | <0.5 mL/kg/h |
| Respiratory | PaO₂/FiO₂³ | ≦250 (≦200 if only severe |
| | | sepsis criterion met, or lung |
| | | is suspected site of infection) |
| Hematologic | Platelet count | <80,000 (≧18 years of age) or |
| | | 50% decrease over 3 days |
| Metabolic | plasma pH | ≦7.3 (≧18 years of age) |
| | Base deficit | ≧18 years: BD: ≧5.0 mEq/L; |
| | (BD) + lactate | Lactate >1.5x upper limit of |
| | | normal |

Septic shock = sepsis with acute cardiovascular dysfunction; Severe sepsis = sepsis with ≧2 acute organ dysfunctions
¹Despite adequate fluid resuscitation or adequate intravascular volume
³If SaO₂ only, PaO₂ calculated from standard oxyhemoglobin dissociation curve with assumption of normal pH

The latter were considered to be ideal molecular controls since at ED arrival they had a SIRS-associated illness that was clinically indistinguishable from sepsis (Table 3). In addition, they matched the sepsis groups in rates of progression (day 3 organ dysfunction or shock) and 28-day death, allowing a distinction to be made between the pathognomonic molecular events of sepsis progression and those common to progression in other SIRS-associated, acute illnesses (Table 3).

TABLE 3
Final Diagnosis and Progression in the 29 Definite Non-infection (SIRS positive) Control Patients

| | Day 3 Acute | | Day 3 Acute | Day 3 Acute | Day 3 Acute | Day |
|---|---|---|---|---|---|---|
| Diagnosis in Non-Infected, SIRS- | Organ | Day 3 | Renal | Hematologic | Metabolic | 28 |
| Positive Controls | Dysfunction | Shock | Dysfunction | Dysfunction | Dysfunction | Death |
| Arrhythmia | Yes | No | No | No | Yes | No |
| Arrhythmia/Malignancy | Yes | No | No | No | No | No |
| Bowel Obstruction | No | No | No | No | No | No |
| Congestive Heart Failure | Yes | No | No | No | No | No |
| Congestive Heart Failure | No | No | No | No | No | No |
| Congestive Heart Failure | Yes | Yes | No | No | Yes | Yes |
| Congestive Heart Failure/Arrhythmia | Yes | Yes | No | No | Yes | No |
| Congestive Heart Failure/Chronic | Yes | No | No | No | Yes | No |
| Obstructive Pulmonary Disease | | | | | | |
| Dehydration | No | No | No | No | No | No |
| Dehydration | Yes | Yes | Yes | No | Yes | No |
| Drug Reaction/Malignancy | Yes | No | No | No | No | No |
| Gastrointestinal Hemorrhage | Yes | No | Yes | No | Yes | Yes |
| Gastrointestinal Hemorrhage | Yes | Yes | No | Yes | No | No |
| Heroin Overdose | Yes | No | Yes | No | Yes | No |
| Hypertensive Emergency | No | No | No | No | No | No |
| Hypoglycemia | No | No | No | No | No | No |
| Lung Mass/Chronic Obstructive | Yes | Yes | Yes | No | No | No |
| Pulmonary Disease | | | | | | |
| Malignancy | No | No | No | No | No | No |
| Myocardial Infarction | Yes | Yes | No | No | No | No |
| Myocardial Infarction/Dehydration | Yes | Yes | Yes | No | No | No |
| Pancreatitis | No | No | No | No | No | No |
| Pulmonary Edema | Yes | No | Yes | No | No | No |
| Pulmonary Embolism | Yes | No | No | No | No | No |
| Pulmonary Embolism | Yes | Yes | No | No | Yes | No |
| Pulmonary Embolism | Yes | No | No | No | No | No |
| Pulmonary Fibrosis | Yes | Yes | No | No | No | No |
| Pulmonary Mass | Yes | No | No | No | No | No |
| Ruptured Aneurysm/Hypovolemic Shock | Yes | Yes | Yes | No | Yes | Yes |
| Uterine Fibroids/Pain | No | No | No | No | No | No |

Patients were selected to match groups for most material phenotypes at presentation (number of SIRS criteria, age, race, sex, enrollment site, renal function and co-morbidity) but differed in temperature, APACHE II and SOFA scores (Tables 4 and 5). All sepsis patients were independently determined by an expert physician to have definite infections. Non-consecutive patients were added to sepsis groups to increase those with Streptococcus pneumoniae (and thereby for lobar pneumonia; n=31), Escherichia coli (and thereby for urosepsis; n=16) and Staphylococcus aureus (and thereby for skin, soft tissue, and catheter associated infections; n=27) to allow limited etiologic comparisons to be undertaken. Validation studies employed in an independent CAPSOD sample of 18 sepsis deaths and 34 matched sepsis survivors (at t₀ [Rt₀] and t₂₄ [Rt₂₄]: Table 6). The validation set included all remaining sepsis deaths in CAPSOD at time of selection, and, as a result differed in median time-to-death from the discovery cohort (18.5 days vs. 10.7 days, respectively).

TABLE 4
Discovery Patient Categories and Demographics

| | | African- | | | | | | |
|---|---|---|---|---|---|---|---|---|
| | n | American | White | Male | HFHS⁴ | Duke | APACHE II | Age |
| Definite infection: Agent | 103 | 66.3% | 27.2% | 59.2% | 73.8% | 26.0% | 17.6 ± 7.9 | 57.3 ± 17.3 |
| identified | | | | | | | | |
| Definite infection: Agent | 18 | 61.0% | 39.0% | 50.0% | 66.7% | 33.3% | 22.2 ± 10.1 | 70.6 ± 17.4 |
| unidentified | | | | | | | | |
| Definite non-infection | 29 | 72.4% | 24.1% | 41.4% | 79.3% | 20.7% | 17.6 ± 7.2 | 65.8 ± 13.6 |
| (SIRS positive)¹ | | | | | | | | |
| Uncomplicated Sepsis | 27 | 66.7% | 29.6% | 66.7% | 59.3% | 40.7% | 11.6 ± 5.7 | 53.8 ± 15.4 |
| Sepsis Death^{1,3} | 31 | 74.2% | 22.6% | 55.2% | 87.1% | 12.9% | 23.5 ± 9.0 | 68.8 ± 16.7 |
| Septic Shock² | 38 | 52.6% | 36.8% | 50.0% | 65.8% | 34.2% | 19.4 ± 7.8 | 58.6 ± 18.5 |
| Severe Sepsis² | 25 | 76.0% | 24.0% | 64.0% | 84.0% | 20.0% | 18.6 ± 5.6 | 55 ± 16.8 |
| APACHE II⁴ ≧20 | 44 | 77.3% | 18.2% | 50.0% | 81.8% | 18.2% | 25.8 ± 6.1 | 67.5 ± 16.2 |
| APACHE II ≦19 | 67 | 61.2% | 34.3% | 58.2% | 70.1% | 29.9% | 13.2 ± 4.7 | 57.1 ± 17.0 |
| S. aureus bacteremia | 27 | 70.4% | 29.6% | 77.7% | 62.9% | 37.0% | 16.7 ± 8.8 | 52.4 ± 15.3 |
| S. pneumonia bacteremia | 31 | 71.0% | 19.4% | 51.6% | 87.1% | 12.9% | 17.6 ± 7.1 | 57.4 ± 17.2 |
| E. coli bacteremia | 16 | 68.8% | 25.0% | 62.5% | 56.3% | 43.8% | 16.8 ± 8.8 | 59.6 ± 16.0 |
| Community acquired | 44 | 63.6% | 27.3% | 65.9% | 81.8% | 18.2% | 20.7 ± 9.5 | 60.7 ± 18.0 |
| pneumonia | | | | | | | | |

¹Constrained - little or no choice; ≧2 SIRS;
²Day 0-3;
³Day 1-28;
⁴Henry Ford Hospital System

TABLE 5
Clinical and Laboratory Values of the 150 Discovery Patients (Mean ± Standard Error)

| | Non-infected | Uncomplicated | | | |
|---|---|---|---|---|---|
| | SIRS-positive | Sepsis | Severe Sepsis | Septic Shock | Sepsis Death |
| Total | 29 | 27 | 25 | 38 | 31 |
| Patient, History | | | | | |
| Heart Failure | 3.4% | 14.8% | 28.0% | 23.7% | 25.8% |
| Liver Failure | 6.9% | 0.0% | 8.0% | 5.3% | 19.4% |
| Diabetes Mellitus | 34.5% | 25.9% | 24.0% | 34.2% | 41.9% |
| Neoplastic Disease | 24.1% | 3.7% | 8.0% | 5.3% | 22.6% |
| Chronic Lung Disease | 37.9% | 14.8% | 36.0% | 28.9% | 25.8% |
| Renal Failure | 20.7% | 14.8% | 40.0% | 15.9% | 22.6% |
| Hemodialysis | 13.8% | 14.8% | 32.0% | 13.2% | 9.7% |
| Late HIV | 0.0% | 0.0% | 8.0% | 0.0% | 3.2% |
| Immunosuppressants | 3.4% | 0.0% | 12.0% | 5.3% | 6.5% |
| Smoker | 17.2% | 37.0% | 28.0% | 23.7% | 25.8% |
| Alcohol Use | 17.2% | 11.1% | 16.0% | 18.4% | 12.9% |
| Drug Abuse | 10.3% | 29.6% | 28.0% | 15.8% | 12.9% |
| Clinical Variables | | | | | |
| Age (years)* | 65.8 ± 13.6 | 53.8 ± 15.4 | 55.0 ± 16.8 | 58.6 ± 18.5 | 68.8 ± 16.7 |
| Heart Rate (/min) | 107.1 ± 20.1 | 110.0 ± 19.9 | 123.0 ± 20.3 | 115.7 ± 24.9 | 114.2 ± 25.3 |
| Respiratory Rate (/min) | 25.2 ± 6.4 | 23.1 ± 5.3 | 27.4 ± 9.7 | 24.7 ± 6.4 | 28.9 ± 10.4 |
| Temperature (° C.)* | 36.8 ± 1.1 | 38.2 ± 1.8 | 38.5 ± 1.0 | 37.7 ± 2.0 | 37.4 ± 1.7 |
| MAP (mmHg)* | 89.3 ± 20.1 | 90.4 ± 13.6 | 82.5 ± 15.8 | 71.3 ± 15.8 | 69.0 ± 13.5 |
| SOFA | 4.4 ± 2.9 | 3.7 ± 1.2 | 4.7 ± 2.2 | 5.7 ± 3.1 | 7.0 ± 3.6 |
| APACHE II* | 17.6 ± 7.2 | 11.1 ± 5.9 | 18.6 ± 5.6 | 19.1 ± 7.1 | 23.5 ± 9.0 |
| Lab Values | | | | | |
| Sodium (mMol/L)* | 136.9 ± 4.4 | 137.2 ± 3.2 | 134.2 ± 5.9 | 136.7 ± 5.1 | 141.6 ± 10.6 |
| Potassium (mMol/L) | 4.7 ± 1.3 | 3.9 ± 0.8 | 4.5 ± 1.2 | 4.3 ± 1.0 | 4.3 ± 1.1 |
| Creatinine (mg/100 ml) | 2.7 ± 3.7 | 2.6 ± 3.5 | 3.9 ± 4.3 | 2.7 ± 2.8 | 2.7 ± 2.9 |
| BUN (mg/dL) | 34.8 ± 27.2 | 20.6 ± 17.6 | 43.1 ± 41.9 | 31.0 ± 22.4 | 47.5 ± 40.0 |
| Glucose (mg/dL) | 151.0 ± 96.1 | 142.1 ± 82.0 | 190.5 ± 192.3 | 157.3 ± 107.1 | 164.3 ± 157.9 |
| Hematocrit (%)* | 34.8 ± 6.6 | 38.4 ± 5.2 | 37.5 ± 4.8 | 33.9 ± 7.7 | 30.6 ± 7.4 |
| Leucocytes (1000 s/mm³) | 10.8 ± 4.2 | 12.9 ± 4.4 | 15.1 ± 8.7 | 16.4 ± 8.5 | 18.6 ± 18.3 |
| Platelet (10³/mm³) | 275.5 ± 98.9 | 240.3 ± 77.8 | 214.9 ± 163.5 | 235.6 ± 126.0 | 232.0 ± 151 |
| eGFR (ml/min) | 65.7 ± 53.0 | 75.5 ± 24.6 | 77.7 ± 48.6 | 75.7 ± 77.5 | 55.8 ± 40.3 |

*Significant group difference (ANOVA with Bonferroni correction, p ≦ 0.0031);
BUN: blood urea nitrogen.

TABLE 6
Validation Cohort (n = 52) Demographics and Characteristics

| | | African- | | | | | eGFR | | |
|---|---|---|---|---|---|---|---|---|---|
| | n | American | White | Male | HFHS | Duke | (ml/min) | APACHE II | Age (years) |
| Definite infection: Agent identified | 34 | 52.9% | 38.2% | 61.8% | 58.8% | 41.2% | 61.7 ± 41.2 | 16.7 ± 6.8 | 58.8 ± 18.9 |
| Definite infection: Agent unidentified | 18 | 44.4% | 50.0% | 72.2% | 38.9% | 61.1% | 61.6 ± 44.4 | 16.1 ± 6.2 | 58.2 ± 20.1 |
| Uncomplicated Sepsis | 11 | 63.6% | 27.3% | 72.7% | 45.5% | 54.5% | 68.5 ± 22.4 | 11.7 ± 3.4 | 56.4 ± 14.5 |
| Severe Sepsis | 12 | 41.7% | 50.0% | 75.0% | 50.0% | 50.0% | 49.8 ± 51.8 | 17.2 ± 5.8 | 56.3 ± 18.6 |
| Septic Shock | 11 | 45.5% | 45.5% | 54.5% | 45.5% | 55.5% | 43.5 ± 22.5 | 17.6 ± 6.2 | 64.3 ± 21.1 |
| Sepsis Survivors | 34 | 50.0% | 41.2% | 67.6% | 47.1% | 52.9% | 58.7 ± 34.4 | 15.7 ± 6.5 | 58.9 ± 18.1 |
| Sepsis Death | 18^{A} | 50.0% | 44.4% | 61.1% | 61.1% | 38.9% | 67.9 ± 45.3 | 18.3 ± 8.2 | 58.0 ± 18.1 |

### Example 2

### Plasma Metabolomics

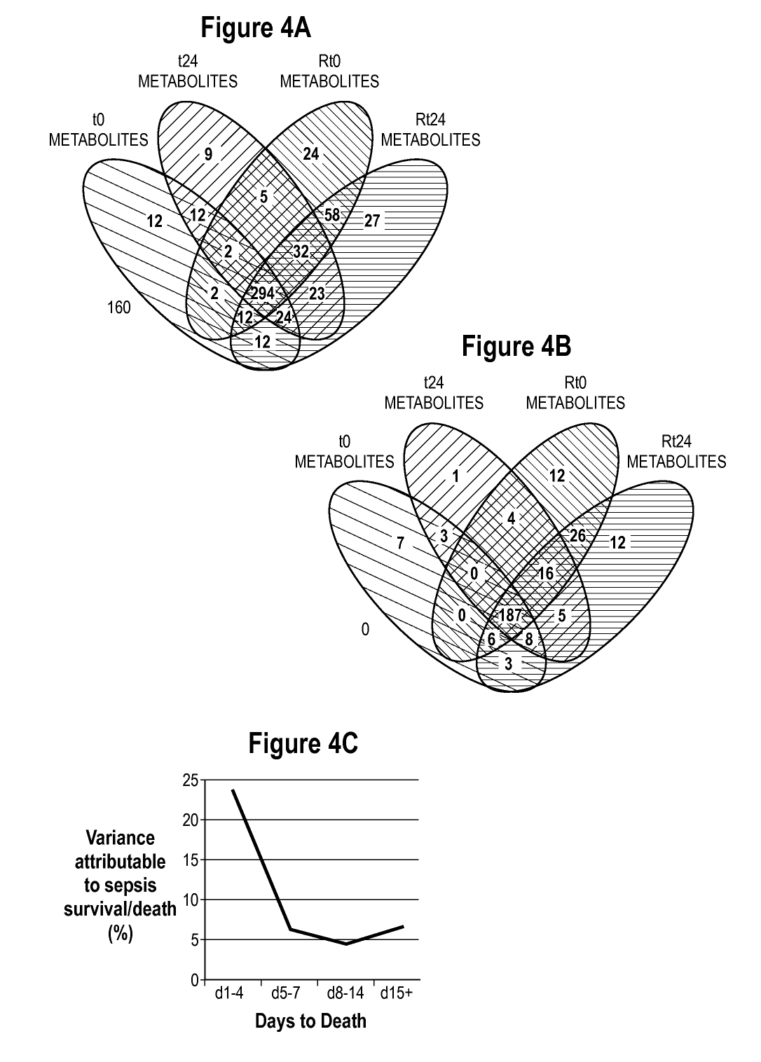

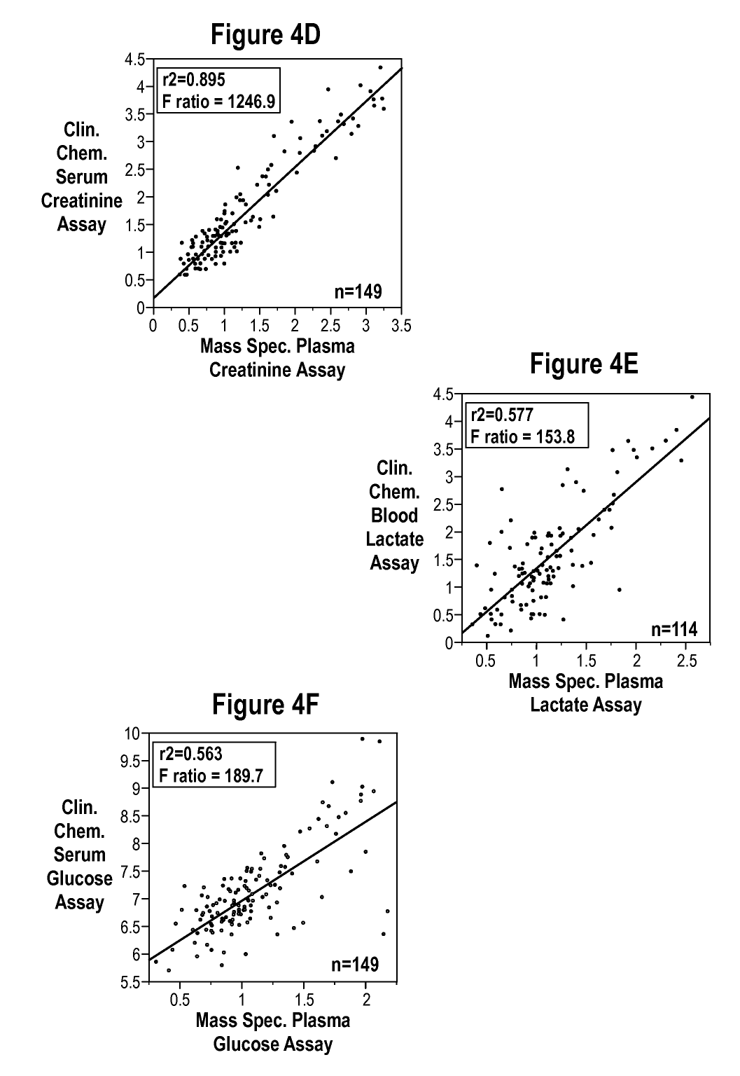

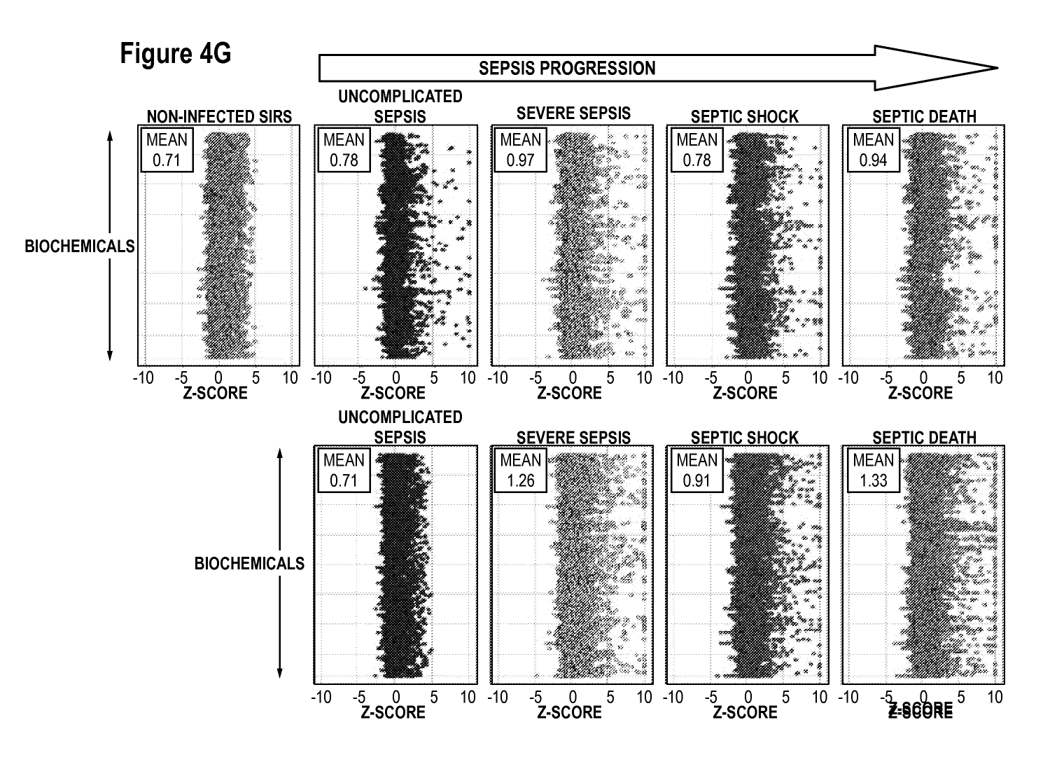

Plasma biochemicals of mass-to-charge (m/z) ratio 100-1000 Da were measured in 150 discovery patients using label-free, liquid and gas chromatography and MS. Of approximately 4,413 biochemicals detectable in human tissues, 439 were measured at t₀ or t₂₄ and 332 were detected at both times. 215 and 224 of the biochemicals detected at t₀ and t₂₄, respectively, were annotated metabolites (FIG. 4 a,b). After signal intensity normalization to batch medians, median relative standard deviation of repeated measurements of standards was 10%. Clinical assays of serum creatinine, capillary lactate and serum glucose correlated well with log-transformed normalized plasma intensities (FIG. 4 d, e, f), indicating that MS-measurements were semi-quantitative. Z-score plots showed right-skewed metabolite distributions at t₀, with increased skewing in severe sepsis and sepsis death (FIG. 4 g), indicative of greater metabolite variance in these groups.

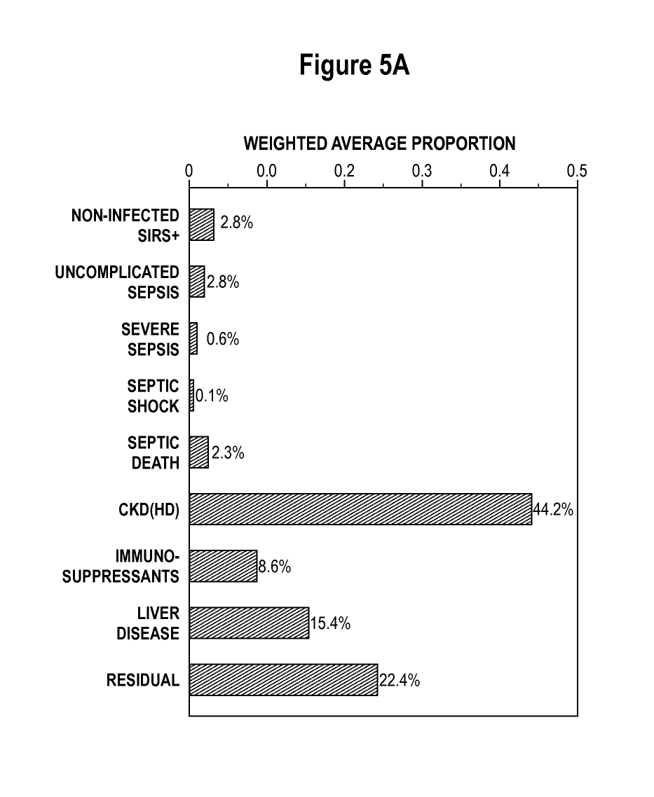

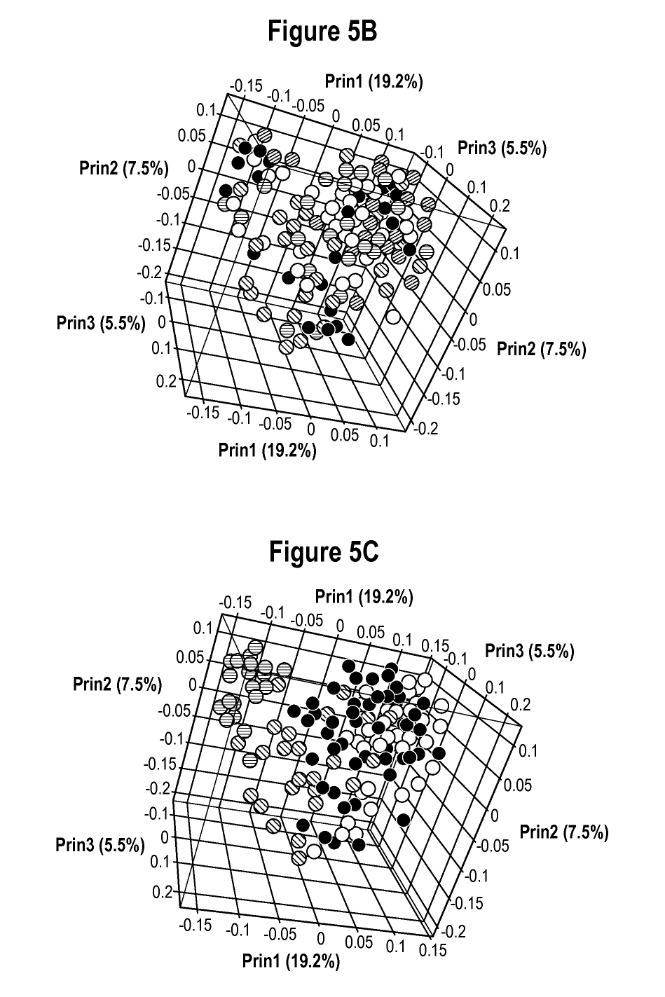

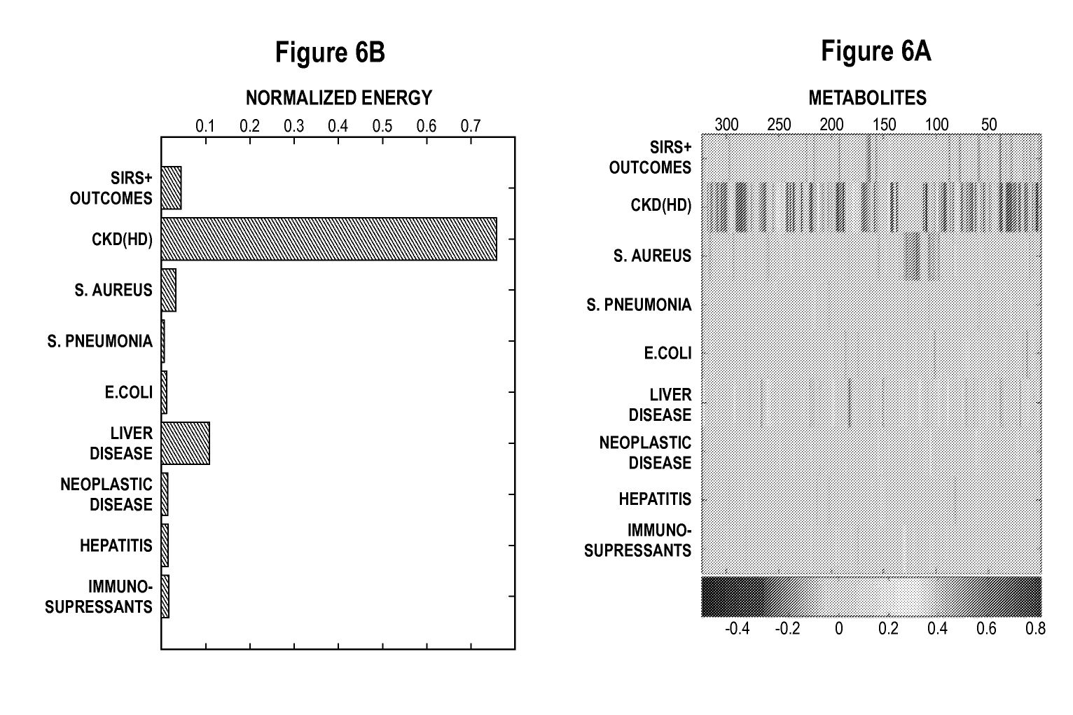

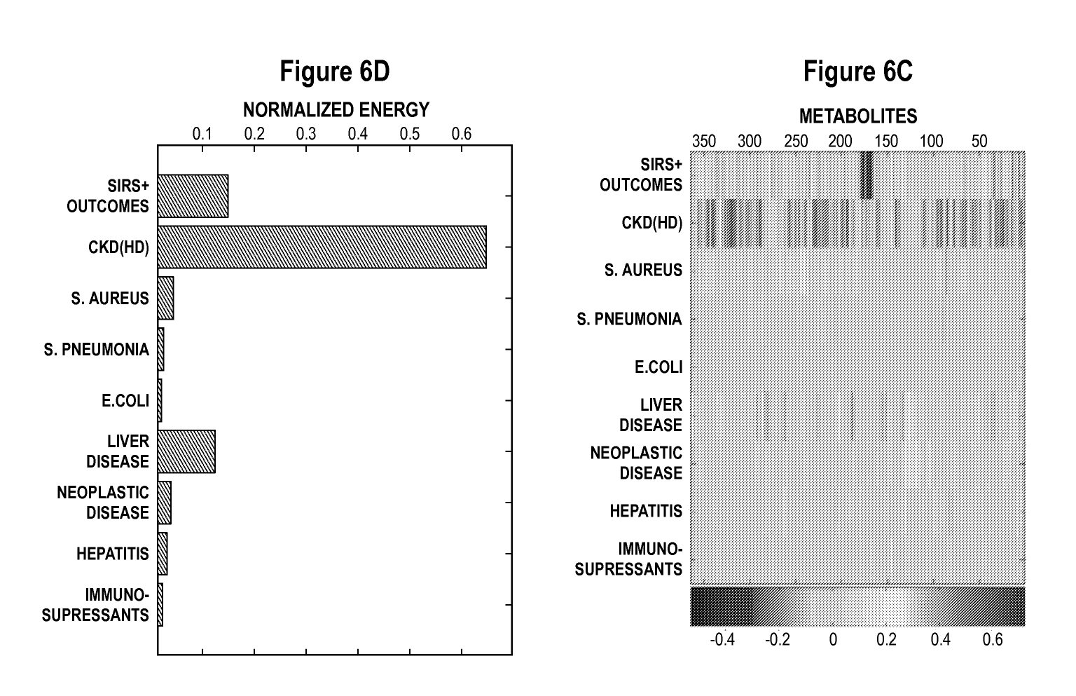

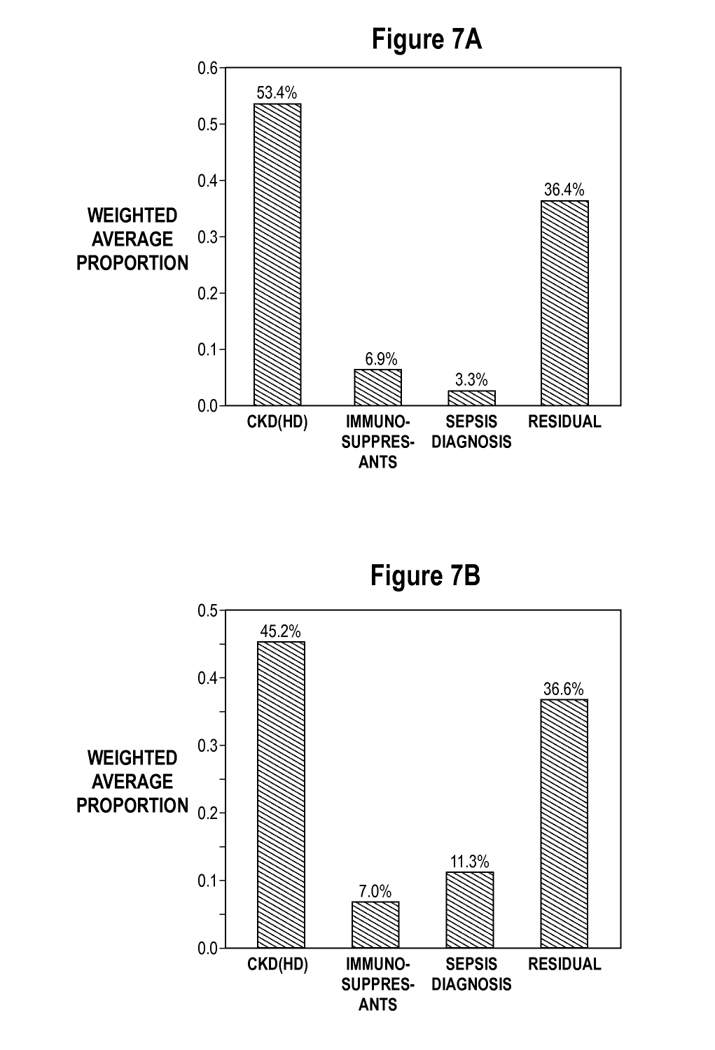

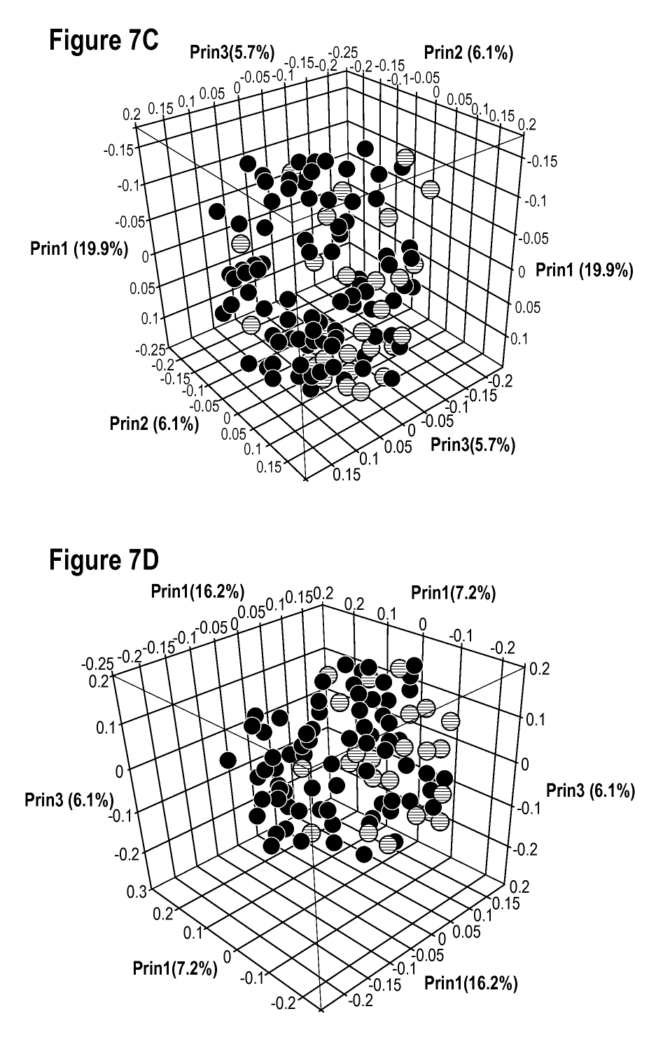

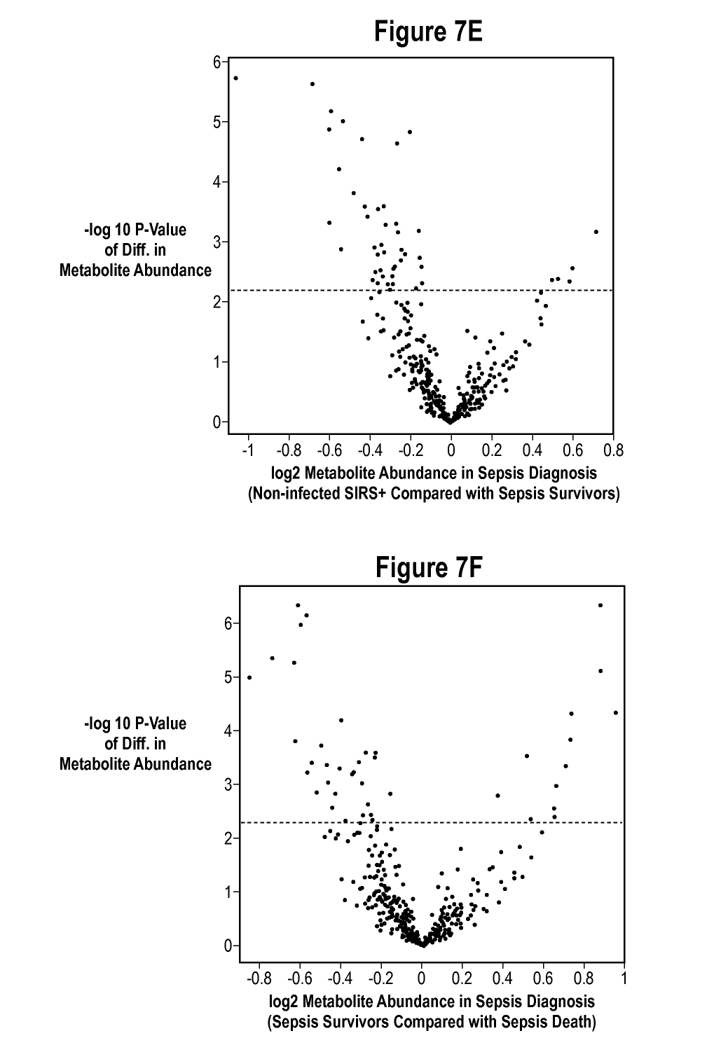

Group differences between mean plasma metabolite values were sought in cross-sectional studies at t₀ or t₂₄. Principal component analysis (PCA) and Bayesian factor analysis with normalized energy plots both demonstrated the main sources of inter-individual variation in the plasma metabolome to be renal function, liver disease and sepsis group membership (FIGS. 5 and 6). Of these, only variation attributable to sepsis groups increased with time (FIGS. 6-8). In sepsis deaths, the variance in the plasma metabolome explained by sepsis outcome increased as death approached (FIG. 2g).

Differences between groups were sought by analysis of variance (ANOVA). Non-sepsis-related effects were minimized by inclusion of renal function and liver disease as fixed effects and/or by separating renal and sepsis group effects. Since acute renal dysfunction partially co-segregated with sepsis death this strategy may have been too conservative in sepsis outcome comparisons (Table 7, 8).

TABLE 7
Comparison of Average eGFR at t₀ and
t₂₄ in the Major Sepsis Groups

| Sepsis Group | t₀ eGFR | t₂₄ eGFR | p-value |
|---|---|---|---|
| Non-infected SIRS Positive | 69.3 ± 55.5 | 68.1 ± 48.9 | 0.83 |
| Sepsis Survivors | 71.2 ± 56.9 | 75.9 ± 55.6 | 0.16 |
| Sepsis Death | 60.2 ± 40.2 | 59.6 ± 31.6 | 1 |

TABLE 8
Partial Overlap of eGFR Group and Sepsis Group Membership

| Estimated | | | | | | |
|---|---|---|---|---|---|---|
| Glomerular | | Non-infected | Uncom- | | | |
| Filtration | | SIRS- | plicated | Severe | Septic | Sepsis |
| Rate | N | Positive | Sepsis | Sepsis | Shock | Death |
| >74 ml/min | 45 | 13.3% | 26.7% | 15.6% | 24.4% | 20% |
| 32-74 ml/min | 54 | 27.8% | 20.4% | 14.8% | 16.7% | 20% |
| 0-31 ml/min | 28 | 17.9% | 3.6% | 10.7% | 35.7% | 32.1% |
| Chronic | 22 | 13.6% | 13.6% | 36.4% | 27.3% | 9.1% |
| hemodialysis | | | | | | |

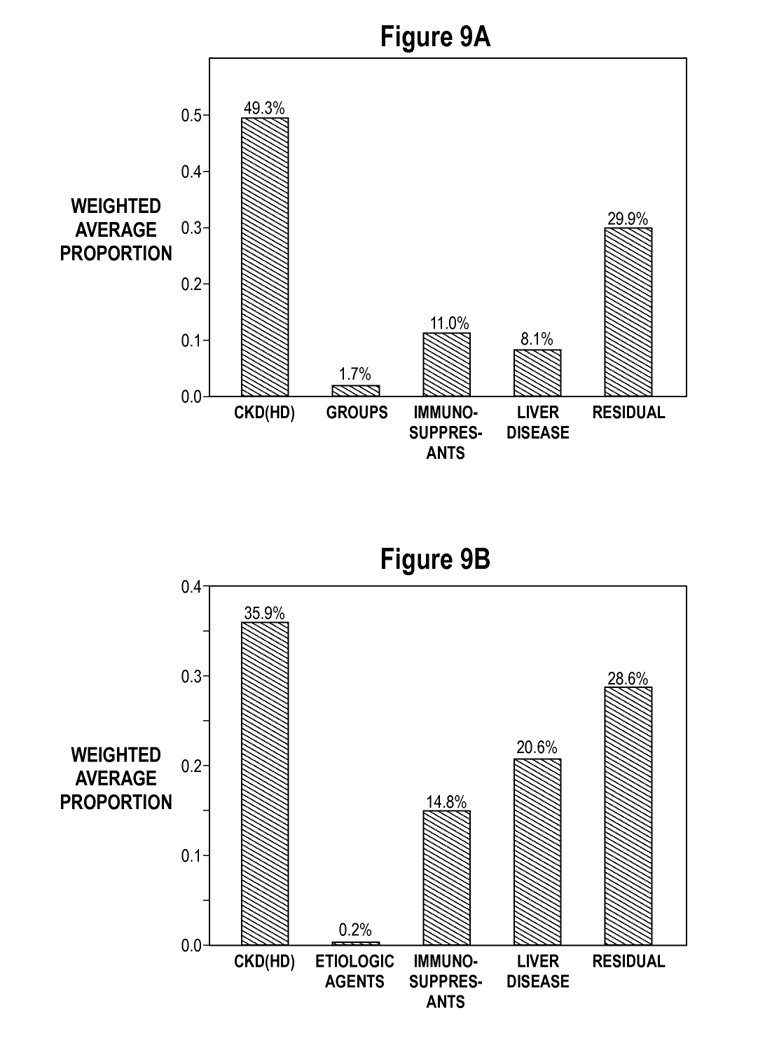

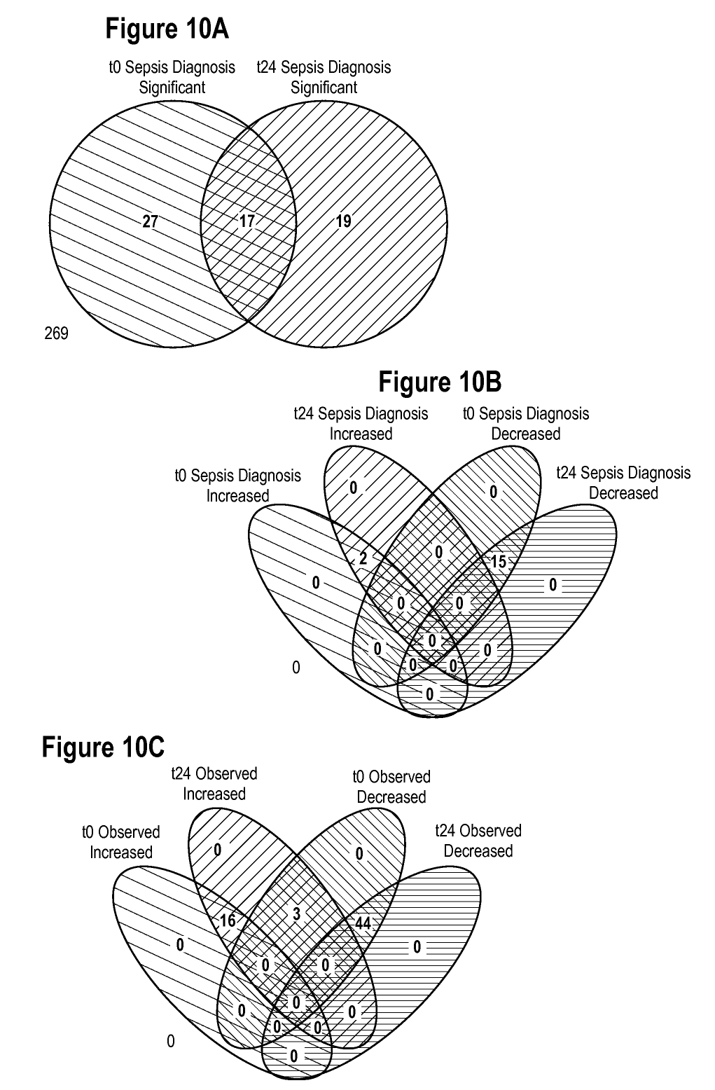

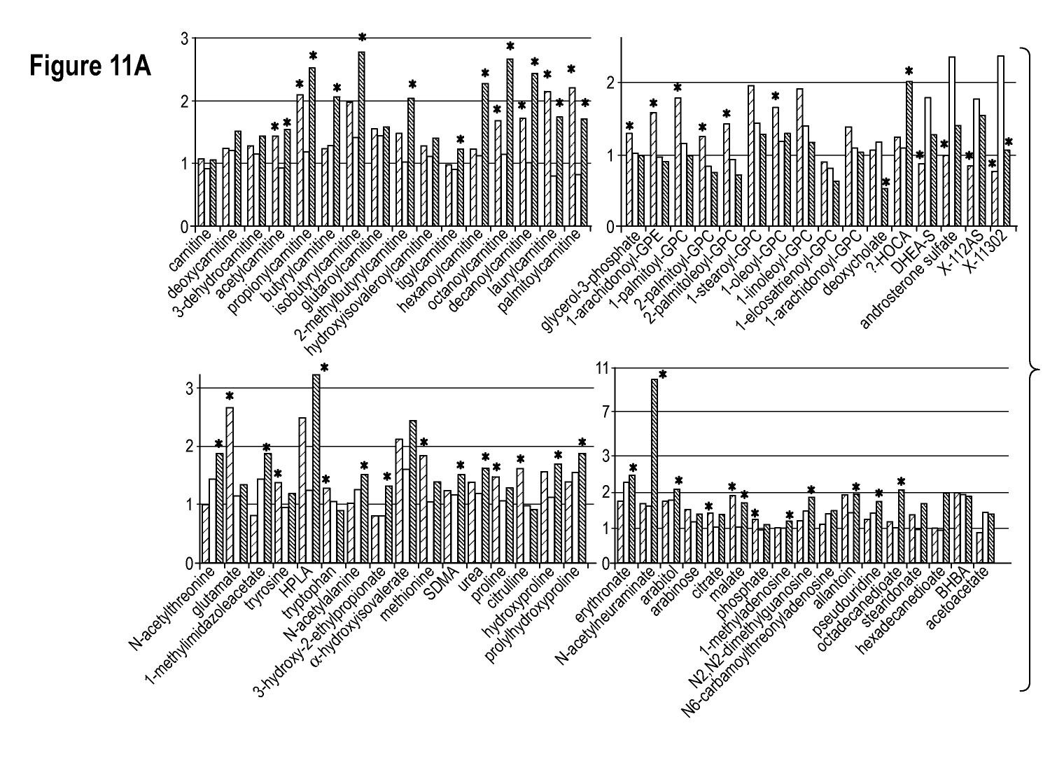

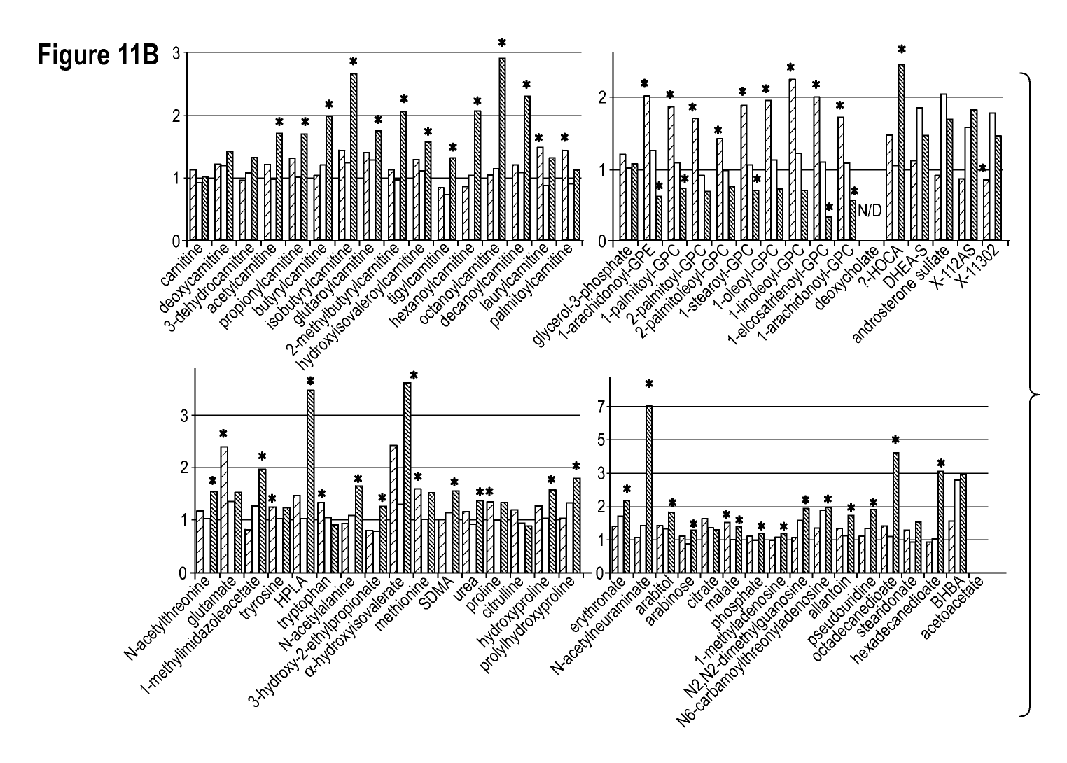

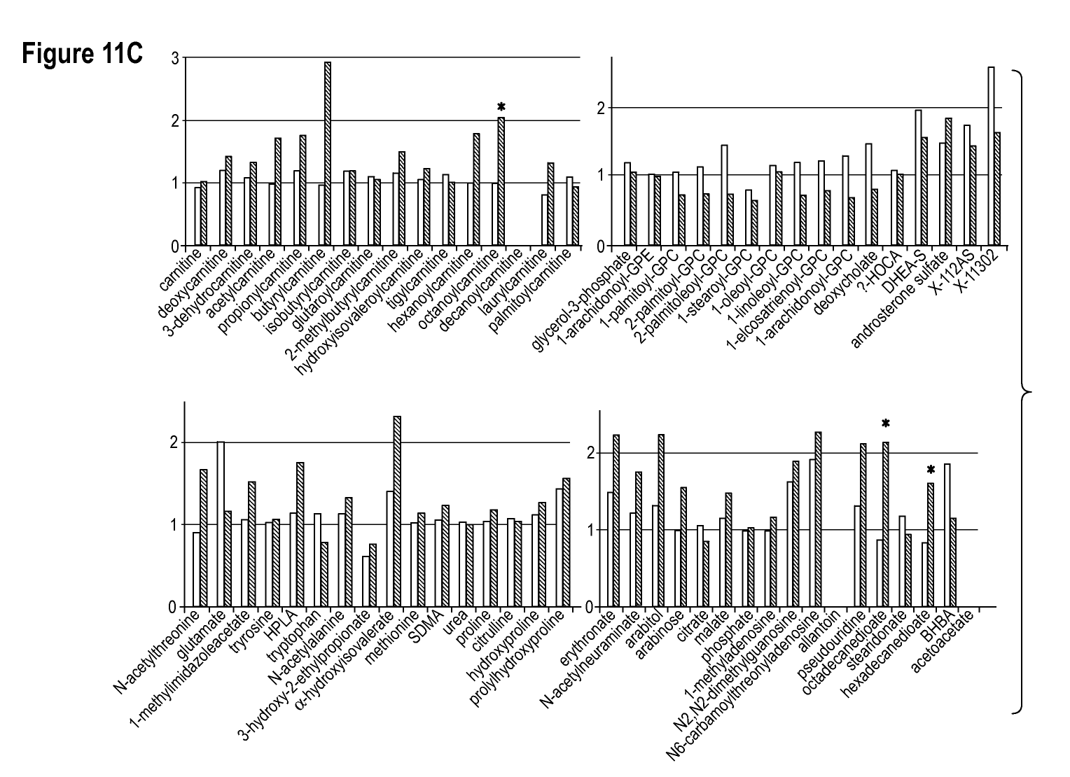

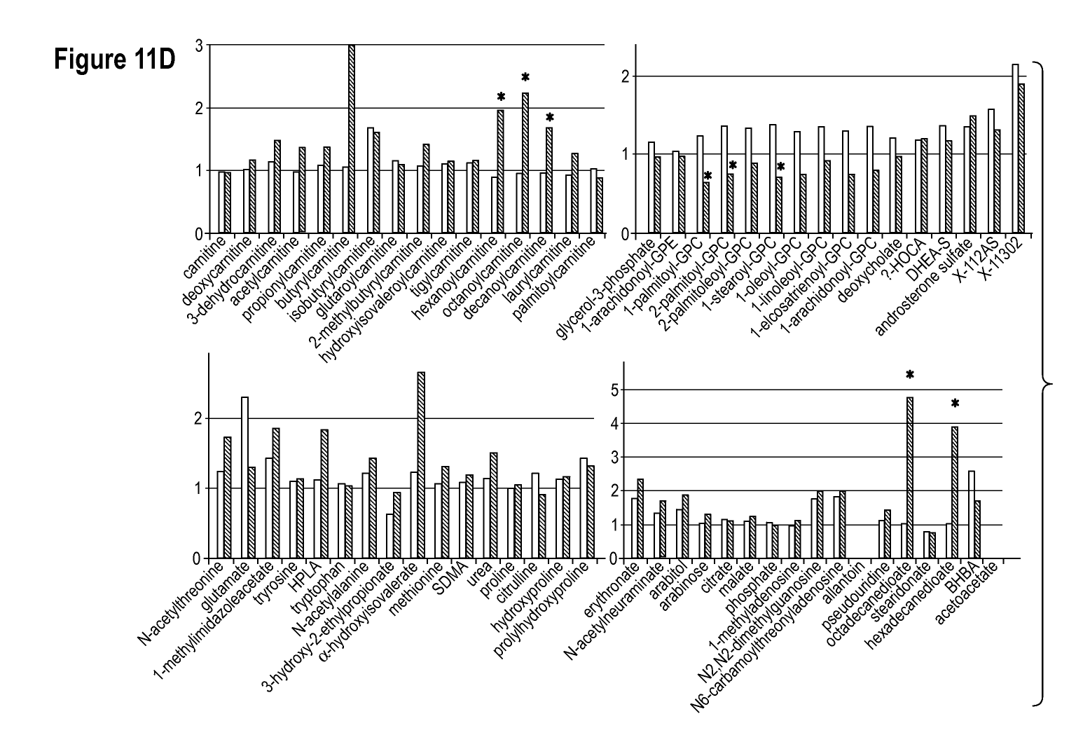

No plasma metabolite differed significantly between sepsis survivor subgroups (uncomplicated sepsis, day 3 severe sepsis, day 3 septic shock) or between infectious agents (S. pneumoniae, S. aureus or E. coli; FIG. 9) at either t₀ or t₂₄. In contrast, plasma levels of 49 and 42 metabolites differed between sepsis survivors and uninfected, SIRS-positive controls at t₀ and t₂₄, respectively (FIG. 2a; ANOVA with inclusion of renal function and liver disease as fixed effects and FDR 5%; Tables 9, 10). 60 of 63 metabolites that were significantly altered at one time and detected at the other had concordant direction of change, indicating a singular, rather than multiphasic, metabolic response in sepsis survivors (FIG. 10). Decreased in sepsis survivors relative to controls were citrate, malate, glycerol, glycerol 3-phosphate, phosphate, 21 amino acids and their catabolites, 12 glycerophospho-choline and -ethanolamine esters (acyl GPC/E) and 6 carnitine esters (FIG. 3a, FIG. 11, Tables 9, 10). The latter have previously been reported in sepsis. Six acetaminophen catabolites and two androgenic steroids were increased. Notably, lactate, ketone bodies and carnitine were unchanged.

TABLE 9
Direction of Change of Significant Plasma
Metabolite Differences by Weighted ANOVA

| | Sepsis Diagnosis | | Sepsis Outcome | |
|---|---|---|---|---|
| | Increased | Decreased | Increased | Decreased |
| Discovery Set t₀ | 7 | 42 | 61 | 15 |
| Discovery Set t₂₄ | 12 | 30 | 112 | 16 |
| Replication Set t₀ | | | 12 | 6 |
| Replication Set t₂₄ | | | 13 | 7 |

Sepsis diagnosis: comparison of sepsis survivors with non-infected SIRS-positive patients. Sepsis outcome: comparison of sepsis survivors and deaths. Significant differences reflect weighted ANOVAs with 5% FDR (t0 and t24 in the discovery set), 25% FDR (t0 in the replication set) or 15% FDR (t24 in the replication set).

TABLE 10
Average, log-transformed, scaled, plasma metabolite concentrations in
non-infected, SIRS-positive patients, sepsis survivors and sepsis deaths at t₀ and t24 in
discovery and replication cohorts, showing significant differences from sepsis survivors by
weighted ANOVAs (denoted *) with 5% FDR (t₀ and t₂₄ discovery samples), 25% FDR (t₀
replication samples) or 15% FDR (t₂₄ replication samples).

| | t0 Non- | | | t24 Non- | | | Replication |
|---|---|---|---|---|---|---|---|
| | Infected | t0 Sepsis | t0 Sepsis | Infected | t24 Sepsis | t24 Sepsis | t0 Sepsis |
| Biochemical | SIRS+ | Survivors | Deaths | SIRS+ | Survivors | Deaths | Survivors |
| 1,5-anhydroglucitol | 0.95 ± 0.02 | 0.90 ± 0.01 | 0.83 ± 0.02 | 1.06 ± 0.02 | 0.92 ± 0.01 | 0.78 ± 0.01 | 0.73 ± 0.02 |
| 1,6-anhydroglucose | 1.34 ± 0.09 | 1.21 ± 0.03 | 0.97 ± 0.04 | N/D | N/D | N/D | 1.36 ± 0.06 |
| 10-heptadecenoate | 1.24 ± 0.03 | 1.12 ± 0.01 | 1.21 ± 0.02 | 1.15 ± 0.02 | 0.98 ± 0.00 | 1.17 ± 0.02 | 0.99 ± 0.01 |
| 10-nonadecenoate | 1.25 ± 0.02 | 1.05 ± 0.01 | 1.34 ± 0.03 | 1.25 ± 0.03 | 1.03 ± 0.01 | 1.47 ± 0.04 | 0.95 ± 0.01 |
| 1-arachidoyl-GPC | 1.42 ± 0.04 | 1.08 ± 0.01 | 1.03 ± 0.04 | 1.66 ± 0.04* | 1.05 ± 0.01 | 0.55 ± 0.02* | 1.29 ± 0.03 |
| 1-arachidoyl-GPE | 1.59 ± 0.04* | 0.96 ± 0.01 | 0.90 ± 0.03 | 1.93 ± 0.04* | 1.22 ± 0.01 | 0.66 ± 0.01* | 1.02 ± 0.01 |
| 1-arachidoyl-GPI | 1.10 ± 0.02 | 1.04 ± 0.01 | 0.93 ± 0.02 | 1.25 ± 0.02 | 0.99 ± 0.01 | 0.83 ± 0.01 | 1.26 ± 0.02 |
| 1-docosahexaenoyl-GPC | N/D | N/D | N/D | N/D | N/D | N/D | 1.44 ± 0.05 |
| 1-eicosadienoyl-GPC | N/D | N/D | N/D | N/D | N/D | N/D | 1.09 ± 0.02 |
| 1-eicosatrienoyl-GPC | 0.90 ± 0.02 | 0.82 ± 0.01 | 0.62 ± 0.02 | 2.00 ± 0.06* | 1.11 ± 0.02 | 0.38 ± 0.01* | 1.22 ± 0.02 |
| 1-heptadecanoyl-GPC | N/D | N/D | N/D | N/D | N/D | N/D | 1.16 ± 0.03 |
| 1-linoleoyl-GPC | 1.92 ± 0.05 | 1.43 ± 0.02 | 1.20 ± 0.04 | 2.20 ± 0.05* | 1.19 ± 0.02 | 0.67 ± 0.02 | 1.19 ± 0.02 |
| 1-linoleoyl-GPE | N/D | N/D | N/D | 2.23 ± 0.06* | 1.40 ± 0.02 | 0.73 ± 0.02* | N/D |
| 1-linoleoyl-GPI | N/D | N/D | N/D | N/D | N/D | N/D | 0.88 ± 0.02 |
| 1-methyladenosine | 1.01 ± 0.01 | 0.98 ± 0.00 | 1.18 ± 0.01* | 0.95 ± 0.01 | 1.04 ± 0.00 | 1.18 ± 0.01* | 0.99 ± 0.01 |
| 1-methylimidazoleacetate | 0.80 ± 0.03 | 1.23 ± 0.02 | 1.79 ± 0.08* | 0.84 ± 0.04 | 1.27 ± 0.03 | 1.97 ± 0.05* | 1.05 ± 0.03 |
| 1-methylurate | N/D | N/D | N/D | 0.84 ± 0.04 | 1.22 ± 0.02 | 1.49 ± 0.05* | 1.16 ± 0.03 |
| 1-myristoyl-GPC | N/D | N/D | N/D | 1.10 ± 0.03* | 0.76 ± 0.01 | 0.59 ± 0.01 | 1.14 ± 0.02 |
| 1-oleoylglycerol | N/D | N/D | N/D | N/D | N/D | N/D | 0.75 ± 0.02 |
| 1-oleoylglycerophosphate | N/D | N/D | N/D | 1.62 ± 0.03* | 1.00 ± 0.01 | 0.80 ± 0.02 | N/D |
| 1-oleoyl-GPC | 1.67 ± 0.03* | 1.21 ± 0.01 | 1.30 ± 0.05 | 1.98 ± 0.04* | 1.14 ± 0.01 | 0.74 ± 0.02 | 1.09 ± 0.02 |
| 1-oleoyl-GPE | 1.06 ± 0.03 | 0.93 ± 0.01 | 0.87 ± 0.03 | 1.52 ± 0.05 | 1.07 ± 0.02 | 0.64 ± 0.02 | 1.14 ± 0.02 |
| 1-palmitoleoyl-GPC | 1.43 ± 0.04* | 0.94 ± 0.01 | 0.73 ± 0.03 | 1.44 ± 0.03* | 1.00 ± 0.01 | 0.70 ± 0.02 | 1.44 ± 0.03 |
| 1-palmitoleoyl-GPI | N/D | N/D | N/D | N/D | N/D | N/D | 1.26 ± 0.04 |
| 1-palmitoylglycerol | 1.18 ± 0.02 | 1.19 ± 0.01 | 1.21 ± 0.03 | 2.82 ± 0.30 | 1.25 ± 0.02 | 0.71 ± 0.01 | N/D |
| 1-palmitoyl-GPC | 1.80 ± 0.03* | 1.17 ± 0.01 | 0.99 ± 0.03 | 1.89 ± 0.03* | 1.10 ± 0.01 | 0.76 ± 0.02* | 1.07 ± 0.02 |
| 1-palmitoyl-GPE | N/D | N/D | N/D | N/D | N/D | N/D | 1.17 ± 0.02 |
| 1-palmitoyl-GPI | N/D | N/D | N/D | N/D | N/D | N/D | 1.62 ± 0.05 |
| 1-stearoylglycerol | 1.10 ± 0.01 | 0.97 ± 0.00 | 0.91 ± 0.01 | 0.90 ± 0.02 | 0.91 ± 0.01 | 0.71 ± 0.02 | 0.90 ± 0.01 |
| 1-stearoyl-GPC | 1.99 ± 0.05 | 1.46 ± 0.02 | 1.27 ± 0.04 | 1.92 ± 0.03* | 1.07 ± 0.01 | 0.70 ± 0.02* | 1.28 ± 0.03 |
| 1-stearoyl-GPE | N/D | N/D | N/D | N/D | N/D | N/D | 0.82 ± 0.02 |
| 1-stearoyl-GPI | 1.09 ± 0.02 | 0.97 ± 0.01 | 1.24 ± 0.04 | N/D | N/D | N/D | 1.26 ± 0.03 |
| 2-aminobutyrate | 1.43 ± 0.04 | 1.18 ± 0.01 | 1.12 ± 0.02 | 1.66 ± 0.05 | 1.18 ± 0.01 | 1.01 ± 0.02 | 1.13 ± 0.02 |
| 2-arachidonoyl-GPE | N/D | N/D | N/D | N/D | N/D | N/D | 1.07 ± 0.02 |
| 2-hydroxyacetaminophen | 0.77 ± 0.04 | 1.05 ± 0.01 | 0.67 ± 0.03 | 0.35 ± 0.03* | 1.79 ± 0.04 | 1.51 ± 0.08 | 1.82 ± 0.07 |
| 2-hydroxybutyrate | 1.39 ± 0.04 | 1.13 ± 0.01 | 1.39 ± 0.02 | 1.16 ± 0.03 | 1.18 ± 0.01 | 1.64 ± 0.04 | 1.29 ± 0.04 |
| 2-hydroxyhippurate | N/D | N/D | N/D | N/D | N/D | N/D | 2.20 ± 0.17 |
| 2-hydroxypalmitate | 1.15 ± 0.01 | 1.13 ± 0.01 | 1.58 ± 0.04 | 1.33 ± 0.04 | 1.14 ± 0.01 | 1.51 ± 0.04 | 1.09 ± 0.02 |
| 2-hydroxystearate | 1.19 ± 0.02 | 1.15 ± 0.01 | 1.20 ± 0.03 | 1.23 ± 0.03 | 1.08 ± 0.01 | 1.16 ± 0.02 | 1.16 ± 0.02 |
| 2-linoleoyl-GPC | N/D | N/D | N/D | N/D | N/D | N/D | 1.04 ± 0.03 |
| 2-methoxyacetaminophen | N/D | N/D | N/D | N/D | N/D | N/D | 1.79 ± 0.09 |
| glucuronide | | | | | | | |
| 2-methoxyacetaminophen | 0.64 ± 0.04 | 1.11 ± 0.01 | 0.75 ± 0.05 | 0.16 ± 0.01* | 1.48 ± 0.02 | 0.98 ± 0.06 | 1.62 ± 0.07 |
| 2-methylbutyroylcarnitine | 1.46 ± 0.06 | 1.01 ± 0.01 | 2.05 ± 0.07* | 1.13 ± 0.05 | 1.00 ± 0.01 | 2.12 ± 0.09* | 1.17 ± 0.02 |
| 2-octenoylcarnitine | N/D | N/D | N/D | 0.77 ± 0.02 | 0.79 ± 0.01 | 1.47 ± 0.04* | N/D |
| 2-oleoyl-GPC | N/D | N/D | N/D | N/D | N/D | N/D | 0.96 ± 0.02 |
| 2-palmitoyl-GPC | 1.26 ± 0.03* | 0.85 ± 0.01 | 0.76 ± 0.02 | 1.72 ± 0.04* | 0.91 ± 0.01 | 0.70 ± 0.02 | 1.14 ± 0.02 |
| 2-stearoyl-GPC | N/D | N/D | N/D | 1.48 ± 0.03* | 1.19 ± 0.03 | 0.52 ± 0.01* | 1.19 ± 0.03 |
| 3-(4-hydroxyphenyl)lactate | 2.48 ± 0.13 | 1.26 ± 0.01 | 3.21 ± 0.11* | 1.48 ± 0.06 | 1.04 ± 0.01 | 3.47 ± 0.18* | 1.15 ± 0.03 |
| 3-(cystein-S-yl)acetaminophen | 0.68 ± 0.04 | 1.28 ± 0.02 | 1.41 ± 0.08 | 0.25 ± 0.03* | 2.00 ± 0.04 | 1.41 ± 0.12 | 1.48 ± 0.05 |
| 3-aminoisobutyrate | N/D | N/D | N/D | N/D | N/D | N/D | N/D |
| 3-carboxy-4-methyl-5-propyl-2- | 1.88 ± 0.07 | 2.38 ± 0.05 | 1.63 ± 0.10 | 2.55 ± 0.16 | 2.49 ± 0.07 | 1.76 ± 0.10 | 3.55 ± 0.17 |
| furanpropanoate | | | | | | | |
| 3-dehydrocarnitine | 1.26 ± 0.02 | 1.18 ± 0.01 | 1.43 ± 0.04 | 0.98 ± 0.02 | 1.09 ± 0.01 | 1.33 ± 0.03 | 0.97 ± 0.02 |
| 3-hydroxy-2-ethylpropionate | 0.80 ± 0.02 | 0.82 ± 0.01 | 1.32 ± 0.04* | 0.80 ± 0.02 | 0.74 ± 0.01 | 1.26 ± 0.05* | 0.59 ± 0.02 |
| 3-hydroxybutyrate | 1.99 ± 0.10 | 1.97 ± 0.03 | 1.85 ± 0.06 | 1.54 ± 0.08 | 2.80 ± 0.07 | 2.98 ± 0.11 | 1.88 ± 0.12 |
| 3-hydroxydecanoate | N/D | N/D | N/D | 1.03 ± 0.03 | 0.86 ± 0.01 | 1.97 ± 0.09* | 0.79 ± 0.01 |
| 3-hydroxyisobutyrate | N/D | N/D | N/D | N/D | N/D | N/D | 1.21 ± 0.03 |
| 3-hydroxykynurenine | N/D | N/D | N/D | N/D | N/D | N/D | N/D |
| 3-hydroxyoctanoate | N/D | N/D | N/D | N/D | N/D | N/D | 0.88 ± 0.02 |
| 3-indoxyl sulfate | 1.45 ± 0.05 | 2.00 ± 0.03 | 1.72 ± 0.07 | 1.29 ± 0.05 | 2.07 ± 0.04 | 2.19 ± 0.09* | 2.09 ± 0.08 |
| 3-methoxytyrosine | 0.95 ± 0.02 | 0.81 ± 0.01 | 5.57 ± 0.74* | 1.13 ± 0.02 | 0.96 ± 0.01 | 2.41 ± 0.15* | 1.14 ± 0.02 |
| 3-methyl-2-oxobutyrate | 1.18 ± 0.02 | 1.07 ± 0.00 | 1.01 ± 0.02 | 1.04 ± 0.01 | 1.06 ± 0.00 | 1.07 ± 0.01 | 1.07 ± 0.01 |
| 3-methyl-2-oxovalerate | 1.38 ± 0.02* | 1.00 ± 0.01 | 1.02 ± 0.02 | 1.26 ± 0.01 | 1.07 ± 0.01 | 0.80 ± 0.02* | 1.08 ± 0.02 |
| 3-methylhistidine | 1.37 ± 0.06 | 1.06 ± 0.02 | 0.60 ± 0.04 | 3.08 ± 0.15* | 1.17 ± 0.02 | 0.57 ± 0.02 | 1.21 ± 0.05 |
| 4-acetamidobutanoate | 1.76 ± 0.09 | 2.91 ± 0.05 | 3.26 ± 0.18 | 1.56 ± 0.10 | 2.39 ± 0.06 | 2.65 ± 0.10* | 1.97 ± 0.07 |
| 4-acetamidophenol | 0.56 ± 0.05* | 1.48 ± 0.02 | 0.63 ± 0.04* | 0.22 ± 0.01* | 2.00 ± 0.03 | 0.94 ± 0.07* | 1.12 ± 0.03 |
| 4-acetaminophen sulfate | 0.74 ± 0.04 | 1.22 ± 0.02 | 0.84 ± 0.04 | 0.46 ± 0.04* | 1.76 ± 0.03 | 1.11 ± 0.06 | 1.41 ± 0.07 |
| 4-ethylphenyl sulfate | N/D | N/D | N/D | N/D | N/D | N/D | N/D |
| 4-hydroxyphenylacetate | N/D | N/D | N/D | N/D | N/D | N/D | N/D |
| 4-methyl-2-oxopentanoate | 1.31 ± 0.03 | 1.17 ± 0.01 | 0.87 ± 0.02* | 1.16 ± 0.02 | 1.15 ± 0.01 | 0.93 ± 0.02 | 1.28 ± 0.02 |
| 4-vinylphenol sulfate | 1.84 ± 0.14 | 2.57 ± 0.09 | 0.52 ± 0.02 | 2.21 ± 0.15 | 2.89 ± 0.13 | 0.79 ± 0.03 | N/D |
| 5-dodecenoate | 1.46 ± 0.06 | 1.62 ± 0.02 | 1.66 ± 0.09 | 1.12 ± 0.02 | 1.22 ± 0.01 | 1.31 ± 0.04 | 1.04 ± 0.03 |
| 5-methylthioadenosine | N/D | N/D | N/D | 0.83 ± 0.03 | 1.02 ± 0.01 | 1.39 ± 0.04* | 0.98 ± 0.02 |
| 5-oxoproline | 1.03 ± 0.02 | 1.26 ± 0.01 | 1.43 ± 0.04 | 1.23 ± 0.04 | 1.13 ± 0.01 | 1.36 ± 0.02 | 1.01 ± 0.01 |
| 7-alpha-hydroxy-3-oxo-4- | 1.25 ± 0.04 | 1.10 ± 0.01 | 2.03 ± 0.07* | 1.49 ± 0.05 | 1.06 ± 0.01 | 2.45 ± 0.09* | 1.09 ± 0.02 |
| cholestenoate | | | | | | | |
| acetoacetate | 0.87 ± 0.04 | 1.47 ± 0.03 | 1.41 ± 0.06 | N/D | N/D | N/D | N/D |
| acetylcarnitine | 1.42 ± 0.04* | 0.94 ± 0.01 | 1.53 ± 0.03* | 1.22 ± 0.03 | 0.99 ± 0.01 | 1.72 ± 0.04* | 1.18 ± 0.03 |
| adenosine 5′-monophosphate | 1.17 ± 0.03 | 1.18 ± 0.01 | 1.05 ± 0.02 | 1.03 ± 0.03 | 1.34 ± 0.01 | 1.44 ± 0.11 | 1.14 ± 0.02 |
| adrenate | 1.27 ± 0.03 | 1.09 ± 0.01 | 1.31 ± 0.03 | 1.36 ± 0.04 | 1.01 ± 0.01 | 1.30 ± 0.04 | 1.15 ± 0.01 |
| alanine | 1.10 ± 0.03 | 0.96 ± 0.01 | 0.80 ± 0.01 | 1.39 ± 0.03* | 0.99 ± 0.01 | 0.85 ± 0.01 | 1.05 ± 0.02 |
| allantoin | 1.92 ± 0.09 | 1.43 ± 0.02 | 1.92 ± 0.05* | 1.33 ± 0.06 | 1.13 ± 0.02 | 1.71 ± 0.05* | N/D |
| alpha-hydroxyisovalerate | 2.10 ± 0.09 | 1.61 ± 0.02 | 2.42 ± 0.08 | 2.41 ± 0.12 | 1.31 ± 0.02 | 3.61 ± 0.29* | 1.40 ± 0.05 |
| alpha-ketobutyrate | 1.05 ± 0.03 | 1.00 ± 0.01 | 1.13 ± 0.03 | 0.94 ± 0.02 | 0.86 ± 0.01 | 1.04 ± 0.02 | 1.17 ± 0.03 |
| alpha-ketoglutarate | 1.33 ± 0.03 | 1.12 ± 0.01 | 1.50 ± 0.05 | 1.34 ± 0.06 | 0.98 ± 0.01 | 1.34 ± 0.06 | N/D |
| alpha-tocopherol | 1.09 ± 0.01 | 1.04 ± 0.01 | 1.14 ± 0.02 | 1.18 ± 0.02 | 0.97 ± 0.01 | 1.11 ± 0.02 | 1.17 ± 0.02 |
| androsterone sulfate | 1.00 ± 0.06* | 2.40 ± 0.03 | 1.40 ± 0.06 | 0.93 ± 0.05 | 2.05 ± 0.04 | 1.74 ± 0.08 | 1.49 ± 0.05 |
| arabinose | 1.52 ± 0.07 | 1.15 ± 0.01 | 1.39 ± 0.03 | 1.07 ± 0.03 | 0.89 ± 0.01 | 1.24 ± 0.03* | 0.99 ± 0.02 |
| arabitol | 1.77 ± 0.06 | 1.80 ± 0.03 | 2.08 ± 0.07* | 1.41 ± 0.05 | 1.34 ± 0.02 | 1.82 ± 0.05* | 1.33 ± 0.03 |
| arachidonate | 1.22 ± 0.01 | 1.09 ± 0.01 | 1.11 ± 0.02 | 1.43 ± 0.03 | 1.17 ± 0.01 | 1.02 ± 0.02 | 1.06 ± 0.02 |
| arginine | 1.28 ± 0.02 | 1.28 ± 0.03 | 1.32 ± 0.05 | 1.28 ± 0.02 | 0.98 ± 0.01 | 0.96 ± 0.02 | 1.10 ± 0.02 |
| asparagine | 1.14 ± 0.03 | 1.00 ± 0.01 | 1.13 ± 0.02 | 1.13 ± 0.03 | 1.00 ± 0.01 | 1.08 ± 0.03 | 0.89 ± 0.01 |
| aspartate | 1.38 ± 0.03 | 0.99 ± 0.01 | 1.05 ± 0.02 | 1.25 ± 0.03 | 1.06 ± 0.01 | 1.55 ± 0.08 | 1.13 ± 0.03 |
| beta-hydroxyisovalerate | 1.36 ± 0.04 | 1.28 ± 0.01 | 1.44 ± 0.03 | 0.80 ± 0.02 | 0.80 ± 0.01 | 1.03 ± 0.02 | 1.12 ± 0.02 |
| beta-hydroxypyruvate | N/D | N/D | N/D | N/D | N/D | N/D | N/D |
| betaine | 1.26 ± 0.02 | 1.08 ± 0.01 | 1.25 ± 0.03 | 1.14 ± 0.03 | 1.09 ± 0.01 | 1.12 ± 0.02 | 0.95 ± 0.02 |
| beta-sitosterol | 0.94 ± 0.04 | 0.92 ± 0.01 | 1.14 ± 0.05 | N/D | N/D | N/D | N/D |
| bilirubin | 1.13 ± 0.03 | 1.38 ± 0.01 | 2.09 ± 0.14 | 0.80 ± 0.03 | 1.43 ± 0.02 | 3.26 ± 0.17 | 2.08 ± 0.06 |
| bilirubin (E,E) | N/D | N/D | N/D | N/D | N/D | N/D | 1.22 ± 0.04 |
| bilirubin(E,Z or Z,E) | N/D | N/D | N/D | N/D | N/D | N/D | 1.18 ± 0.03 |
| biliverdin | 1.06 ± 0.04 | 1.20 ± 0.02 | 1.00 ± 0.03 | 0.93 ± 0.03 | 1.05 ± 0.01 | 1.14 ± 0.03 | 1.42 ± 0.04 |
| butyrylcarnitine | 1.26 ± 0.03 | 1.28 ± 0.02 | 2.06 ± 0.06* | 1.04 ± 0.02 | 1.22 ± 0.01 | 1.96 ± 0.06* | 0.96 ± 0.02 |
| caffeine | 1.45 ± 0.08 | 2.81 ± 0.08 | 4.40 ± 0.42 | 1.53 ± 0.09 | 2.22 ± 0.05 | 2.96 ± 0.21 | 1.97 ± 0.08 |
| caprate | 1.03 ± 0.01 | 1.25 ± 0.02 | 1.42 ± 0.04 | 1.12 ± 0.02 | 1.03 ± 0.01 | 1.06 ± 0.02 | 1.48 ± 0.04 |
| caproate | 1.00 ± 0.02 | 1.17 ± 0.01 | 1.11 ± 0.02 | 1.30 ± 0.04 | 1.34 ± 0.01 | 0.95 ± 0.02 | 0.98 ± 0.00 |
| caprylate | 0.94 ± 0.01 | 1.17 ± 0.02 | 3.75 ± 0.46 | 1.18 ± 0.03 | 1.10 ± 0.01 | 1.05 ± 0.02 | 1.18 ± 0.02 |
| carnitine | 1.06 ± 0.01 | 0.93 ± 0.00 | 1.06 ± 0.01 | 1.13 ± 0.01 | 0.94 ± 0.00 | 1.03 ± 0.01 | 0.93 ± 0.01 |
| catechol sulfate | 2.23 ± 0.09 | 2.10 ± 0.04 | 1.86 ± 0.11 | 1.47 ± 0.06 | 1.62 ± 0.03 | 1.19 ± 0.04 | 1.97 ± 0.10 |
| C-glycosyltryptophan | N/D | N/D | N/D | N/D | N/D | N/D | 1.69 ± 0.06 |
| chenodeoxycholate | N/D | N/D | N/D | N/D | N/D | N/D | N/D |
| cholate | N/D | N/D | N/D | 2.02 ± 0.20 | 1.38 ± 0.04 | 5.93 ± 0.72 | 4.55 ± 0.47 |
| cholesterol | 1.08 ± 0.01 | 1.01 ± 0.00 | 1.09 ± 0.01 | 1.02 ± 0.01 | 1.09 ± 0.01 | 1.03 ± 0.02 | 1.01 ± 0.01 |
| choline | 1.31 ± 0.03 | 1.11 ± 0.01 | 1.16 ± 0.02 | 1.06 ± 0.02 | 1.13 ± 0.01 | 1.07 ± 0.01 | 0.99 ± 0.01 |
| citrate | 1.40 ± 0.02* | 1.03 ± 0.00 | 1.35 ± 0.03 | 1.64 ± 0.04 | 1.36 ± 0.03 | 1.29 ± 0.03 | 1.06 ± 0.02 |
| citrulline | 1.60 ± 0.03* | 0.98 ± 0.01 | 0.91 ± 0.02 | 1.20 ± 0.02 | 0.93 ± 0.01 | 0.89 ± 0.03 | 1.07 ± 0.01 |
| cortisol | 0.79 ± 0.01 | 1.16 ± 0.01 | 1.45 ± 0.03 | 0.69 ± 0.02 | 1.51 ± 0.03 | 1.84 ± 0.04 | 1.41 ± 0.06 |
| cortisone | N/D | N/D | N/D | 0.88 ± 0.02 | 0.92 ± 0.01 | 1.20 ± 0.02* | 0.96 ± 0.01 |
| creatine | 1.15 ± 0.04 | 1.76 ± 0.02 | 2.65 ± 0.09 | 1.15 ± 0.04 | 1.49 ± 0.02 | 2.44 ± 0.08* | 1.48 ± 0.05 |
| creatinine | 1.53 ± 0.06 | 1.88 ± 0.02 | 1.38 ± 0.04 | 1.59 ± 0.05 | 1.60 ± 0.02 | 1.51 ± 0.03 | 1.33 ± 0.03 |
| cysteine | 1.49 ± 0.04 | 1.15 ± 0.01 | 1.26 ± 0.03 | 1.26 ± 0.04 | 1.20 ± 0.01 | 1.44 ± 0.03 | 1.18 ± 0.02 |
| cystine | 2.38 ± 0.11* | 1.67 ± 0.08 | 2.56 ± 0.14 | N/D | N/D | N/D | N/D |
| decanoylcarnitine | 1.74 ± 0.05* | 0.99 ± 0.01 | 2.43 ± 0.07* | 1.21 ± 0.04 | 1.12 ± 0.01 | 2.30 ± 0.06* | N/D |
| dehydroisoandrosterone sulfate | 0.87 ± 0.04* | 1.82 ± 0.02 | 1.28 ± 0.04 | 1.07 ± 0.05 | 1.79 ± 0.03 | 1.54 ± 0.05 | 1.95 ± 0.06 |
| deoxycarnitine | 1.24 ± 0.03 | 1.25 ± 0.01 | 1.51 ± 0.03 | 1.24 ± 0.03 | 1.20 ± 0.01 | 1.46 ± 0.03 | 0.96 ± 0.01 |
| deoxycholate | 1.08 ± 0.08 | 1.19 ± 0.02 | 0.53 ± 0.02* | N/D | N/D | N/D | 1.48 ± 0.06 |
| dihomolinoleate | 1.16 ± 0.02 | 0.98 ± 0.01 | 1.09 ± 0.02 | 1.40 ± 0.04 | 1.05 ± 0.01 | 1.35 ± 0.04 | 0.96 ± 0.01 |
| dihomolinolenate | 1.27 ± 0.02 | 1.12 ± 0.01 | 1.09 ± 0.02 | 1.35 ± 0.03 | 1.08 ± 0.01 | 0.98 ± 0.02 | 1.02 ± 0.01 |
| dihydroxyacetone | 1.65 ± 0.05 | 1.44 ± 0.02 | 0.66 ± 0.01* | 0.84 ± 0.03 | 0.95 ± 0.01 | 0.73 ± 0.02 | N/D |
| docosahexaenoate | 1.55 ± 0.03 | 1.15 ± 0.01 | 1.35 ± 0.03 | 1.50 ± 0.04 | 1.16 ± 0.01 | 1.20 ± 0.03 | 1.18 ± 0.02 |
| docosapentaenoate | 1.35 ± 0.03 | 1.09 ± 0.01 | 1.38 ± 0.04 | 1.40 ± 0.05 | 1.15 ± 0.01 | 1.45 ± 0.05 | 1.25 ± 0.03 |
| dodecanedioate | N/D | N/D | N/D | N/D | N/D | N/D | 1.05 ± 0.03 |
| eicosapentaenoate | 1.53 ± 0.04 | 1.27 ± 0.01 | 1.44 ± 0.09 | 1.48 ± 0.03 | 1.31 ± 0.02 | 1.33 ± 0.08 | 1.29 ± 0.03 |
| eicosenoate | 1.10 ± 0.02 | 1.04 ± 0.01 | 1.34 ± 0.03 | 1.33 ± 0.04 | 1.06 ± 0.01 | 1.58 ± 0.05 | 0.92 ± 0.01 |
| epiandrosterone sulfate | 1.30 ± 0.07 | 2.21 ± 0.03 | 1.21 ± 0.04 | 1.15 ± 0.06 | 1.85 ± 0.05 | 1.20 ± 0.04 | 1.28 ± 0.03 |
| erythritol | 1.48 ± 0.07 | 1.65 ± 0.02 | 2.19 ± 0.07* | 1.18 ± 0.04 | 1.37 ± 0.02 | 2.27 ± 0.07* | 1.22 ± 0.03 |
| erythronate | 1.76 ± 0.09 | 2.32 ± 0.04 | 2.46 ± 0.09* | 1.44 ± 0.07 | 1.71 ± 0.04 | 2.23 ± 0.06* | 1.49 ± 0.05 |
| erythrose | 1.42 ± 0.03 | 1.23 ± 0.01 | 0.89 ± 0.01 | 1.08 ± 0.02 | 1.11 ± 0.01 | 0.99 ± 0.01 | 1.28 ± 0.03 |
| estrone 3-sulfate | N/D | N/D | N/D | N/D | N/D | N/D | N/D |
| fructose | 1.78 ± 0.05 | 1.58 ± 0.02 | 1.15 ± 0.03 | 1.42 ± 0.04 | 1.53 ± 0.02 | 0.86 ± 0.01 | 1.40 ± 0.04 |
| galactonate | 1.62 ± 0.05 | 1.35 ± 0.02 | 1.08 ± 0.03 | 1.17 ± 0.04 | 1.39 ± 0.03 | 0.89 ± 0.02 | 1.50 ± 0.05 |
| gamma-glutamylglutamine | 1.19 ± 0.03 | 1.03 ± 0.01 | 0.95 ± 0.03 | 1.21 ± 0.04 | 0.99 ± 0.01 | 1.09 ± 0.04 | 0.84 ± 0.01 |
| gamma-glutamylisoleucine | N/D | N/D | N/D | 0.93 ± 0.01* | 0.74 ± 0.00 | 0.87 ± 0.02 | 1.00 ± 0.02 |
| gamma-glutamylleucine | 1.19 ± 0.02 | 1.13 ± 0.01 | 1.06 ± 0.02 | 1.03 ± 0.02 | 1.06 ± 0.01 | 1.24 ± 0.04 | 1.21 ± 0.02 |
| gamma-glutamylmethionine | N/D | N/D | N/D | N/D | N/D | N/D | 0.68 ± 0.01 |
| gamma-glutamylphenylalanine | 0.98 ± 0.02 | 1.09 ± 0.01 | 1.23 ± 0.02 | 1.00 ± 0.02 | 1.19 ± 0.01 | 1.59 ± 0.06 | 1.29 ± 0.02 |
| gamma-glutamyltyrosine | 1.24 ± 0.04 | 0.99 ± 0.01 | 1.23 ± 0.03 | 1.13 ± 0.02 | 0.85 ± 0.01 | 1.34 ± 0.05* | 0.98 ± 0.01 |
| gluconate | 2.17 ± 0.08 | 4.02 ± 0.13 | 1.99 ± 0.08 | 1.79 ± 0.08 | 5.21 ± 0.19 | 3.16 ± 0.32 | 2.03 ± 0.11 |
| glucose | 1.22 ± 0.02 | 1.11 ± 0.01 | 1.12 ± 0.02 | 1.09 ± 0.01 | 1.03 ± 0.00 | 1.04 ± 0.01 | 1.05 ± 0.01 |
| glucuronate | 1.95 ± 0.12 | 2.21 ± 0.04 | 2.49 ± 0.10* | N/D | N/D | N/D | 1.24 ± 0.03 |
| glutamate | 2.65 ± 0.10* | 1.16 ± 0.01 | 1.33 ± 0.06 | 2.39 ± 0.09* | 1.35 ± 0.02 | 1.53 ± 0.07 | 2.01 ± 0.09 |
| glutamine | 1.20 ± 0.01* | 0.97 ± 0.00 | 1.07 ± 0.01 | 1.15 ± 0.01 | 0.99 ± 0.00 | 1.11 ± 0.02 | 1.02 ± 0.01 |
| glutamylvaline | 1.10 ± 0.02 | 1.17 ± 0.01 | 1.24 ± 0.02 | 1.10 ± 0.02 | 1.03 ± 0.01 | 1.35 ± 0.03* | 1.17 ± 0.02 |
| glutaroylcarnitine | 1.56 ± 0.07 | 1.45 ± 0.02 | 1.60 ± 0.05 | 1.38 ± 0.05 | 1.29 ± 0.02 | 1.71 ± 0.04* | 1.10 ± 0.02 |
| glycerate | 1.25 ± 0.03 | 1.01 ± 0.00 | 1.22 ± 0.02 | 1.23 ± 0.02 | 1.00 ± 0.01 | 1.11 ± 0.02 | 1.21 ± 0.02 |
| glycerol | 1.42 ± 0.03 | 0.99 ± 0.01 | 1.23 ± 0.02 | 1.44 ± 0.04 | 1.04 ± 0.00 | 1.29 ± 0.03 | 1.06 ± 0.02 |
| glycerol 2-phosphate | N/D | N/D | N/D | N/D | N/D | N/D | 0.91 ± 0.01 |
| glycerol 3-phosphate | 1.29 ± 0.02* | 1.03 ± 0.00 | 1.01 ± 0.01 | 1.24 ± 0.01 | 1.05 ± 0.00 | 1.07 ± 0.02 | 1.19 ± 0.02 |
| glycerophosphorylcholine | N/D | N/D | N/D | 1.55 ± 0.05 | 1.28 ± 0.02 | 0.76 ± 0.02* | 1.12 ± 0.03 |
| glycine | 1.17 ± 0.03 | 1.00 ± 0.01 | 1.18 ± 0.02 | 1.25 ± 0.02* | 0.96 ± 0.01 | 0.98 ± 0.01 | 1.05 ± 0.02 |
| glycochenodeoxycholate | 4.41 ± 0.40 | 2.46 ± 0.09 | 9.17 ± 0.78 | 1.27 ± 0.05 | 2.29 ± 0.06 | 3.53 ± 0.22 | 1.54 ± 0.05 |
| glycocholate | 2.65 ± 0.16 | 3.64 ± 0.13 | 8.88 ± 0.82 | 1.14 ± 0.06 | 2.53 ± 0.07 | 2.32 ± 0.09 | 2.78 ± 0.13 |
| glycodeoxycholate | N/D | N/D | N/D | 0.79 ± 0.04 | 1.30 ± 0.03 | 0.58 ± 0.03 | 1.03 ± 0.05 |
| glycolate | N/D | N/D | N/D | 1.40 ± 0.06 | 1.09 ± 0.01 | 1.02 ± 0.02 | 1.02 ± 0.01 |
| glycylproline | N/D | N/D | N/D | N/D | N/D | N/D | N/D |
| gulono-1,4-lactone | 2.25 ± 0.15 | 2.53 ± 0.06 | 1.37 ± 0.07 | 1.36 ± 0.11 | 2.29 ± 0.08 | 1.22 ± 0.04 | N/D |
| heme | 2.52 ± 0.14 | 2.04 ± 0.03 | 1.26 ± 0.07 | 1.62 ± 0.10 | 2.05 ± 0.04 | 2.37 ± 0.22 | 2.95 ± 0.18 |
| heptanoate | 0.86 ± 0.01 | 0.96 ± 0.00 | 0.93 ± 0.02 | 1.26 ± 0.03 | 1.21 ± 0.01 | 0.83 ± 0.02 | 1.01 ± 0.00 |
| hexadecanedioate | 0.99 ± 0.07 | 0.94 ± 0.01 | 1.91 ± 0.10 | 0.93 ± 0.04 | 1.04 ± 0.01 | 3.11 ± 0.14* | 0.83 ± 0.02 |
| hexanoylcarnitine | 1.24 ± 0.03 | 1.14 ± 0.01 | 2.27 ± 0.06* | 0.91 ± 0.02 | 1.08 ± 0.01 | 2.14 ± 0.05* | 0.99 ± 0.02 |
| hippurate | 4.73 ± 0.26 | 9.91 ± 0.48 | 7.02 ± 0.66 | 3.09 ± 0.20 | 4.24 ± 0.13 | 5.60 ± 0.47 | 7.24 ± 0.39 |
| histidine | 1.20 ± 0.01* | 0.98 ± 0.00 | 1.06 ± 0.01 | 1.00 ± 0.01 | 1.03 ± 0.00 | 1.08 ± 0.02 | 1.04 ± 0.01 |
| homocitrulline | 1.07 ± 0.04* | 0.70 ± 0.01 | 0.84 ± 0.02 | N/D | N/D | N/D | N/D |
| homostachydrine | N/D | N/D | N/D | 0.96 ± 0.02 | 0.97 ± 0.01 | 0.84 ± 0.03 | 0.92 ± 0.02 |
| hydroquinone sulfate | N/D | N/D | N/D | 1.41 ± 0.14 | 1.35 ± 0.03 | 2.78 ± 0.25* | N/D |
| hydroxyisovaleroylcarnitine | 1.28 ± 0.04 | 1.11 ± 0.01 | 1.39 ± 0.03 | 1.33 ± 0.07 | 1.14 ± 0.02 | 1.58 ± 0.04* | 1.07 ± 0.03 |
| hydroxyproline | 1.56 ± 0.05 | 1.11 ± 0.01 | 1.69 ± 0.05* | 1.27 ± 0.03 | 1.04 ± 0.01 | 1.57 ± 0.04* | 1.11 ± 0.02 |
| hyodeoxycholate | N/D | N/D | N/D | 1.04 ± 0.04 | 0.76 ± 0.01 | 0.86 ± 0.05 | 0.84 ± 0.03 |
| hypoxanthine | 1.11 ± 0.03 | 1.15 ± 0.01 | 1.18 ± 0.03 | 0.92 ± 0.04 | 1.35 ± 0.02 | 0.92 ± 0.02 | 1.00 ± 0.01 |
| ibuprofen | N/D | N/D | N/D | 3.44 ± 0.47 | 4.03 ± 0.22 | 1.62 ± 0.15 | N/D |
| iminodiacetate | 1.13 ± 0.03 | 1.22 ± 0.01 | 0.96 ± 0.01 | 1.31 ± 0.04 | 1.18 ± 0.01 | 0.98 ± 0.02 | 1.72 ± 0.05 |
| indoleacetate | N/D | N/D | N/D | 1.44 ± 0.04 | 1.17 ± 0.01 | 1.23 ± 0.03 | 1.38 ± 0.04 |
| indolelactate | 1.47 ± 0.04 | 1.28 ± 0.01 | 1.22 ± 0.03 | 1.29 ± 0.04 | 1.10 ± 0.01 | 1.51 ± 0.05 | 1.17 ± 0.02 |
| indolepropionate | N/D | N/D | N/D | 0.96 ± 0.02 | 1.01 ± 0.01 | 0.92 ± 0.02 | 0.96 ± 0.02 |
| isobutyrylcarnitine | 1.99 ± 0.08 | 1.44 ± 0.02 | 2.80 ± 0.09* | 1.35 ± 0.05 | 1.15 ± 0.01 | 2.49 ± 0.09* | 1.18 ± 0.03 |
| isoleucine | 1.35 ± 0.02* | 0.98 ± 0.01 | 1.14 ± 0.02 | 1.21 ± 0.01 | 1.05 ± 0.00 | 0.92 ± 0.02 | 1.05 ± 0.02 |
| isovalerate | 0.82 ± 0.02 | 0.79 ± 0.00 | 0.77 ± 0.01 | N/D | N/D | N/D | 0.96 ± 0.01 |
| isovalerylcarnitine | 1.37 ± 0.04 | 1.21 ± 0.01 | 1.27 ± 0.03 | 1.01 ± 0.03 | 1.12 ± 0.01 | 1.16 ± 0.03 | 1.46 ± 0.03 |
| kynurenate | 0.93 ± 0.04 | 0.99 ± 0.01 | 1.01 ± 0.02 | N/D | N/D | N/D | N/D |
| kynurenine | 1.16 ± 0.02 | 1.18 ± 0.01 | 1.06 ± 0.02 | 0.99 ± 0.02 | 1.19 ± 0.01 | 1.43 ± 0.04 | 1.24 ± 0.02 |
| lactate | 1.19 ± 0.03 | 1.09 ± 0.01 | 1.52 ± 0.03 | 1.07 ± 0.02 | 1.07 ± 0.01 | 1.50 ± 0.04* | 1.13 ± 0.01 |
| lathosterol | 0.61 ± 0.02 | 0.75 ± 0.01 | 0.72 ± 0.03 | N/D | N/D | N/D | N/D |
| laurate | 1.08 ± 0.01 | 1.16 ± 0.01 | 1.17 ± 0.02 | 1.03 ± 0.01 | 1.04 ± 0.00 | 1.06 ± 0.01 | 1.22 ± 0.03 |
| laurylcarnitine | 2.18 ± 0.13* | 0.81 ± 0.01 | 1.76 ± 0.06* | 1.50 ± 0.04* | 0.89 ± 0.01 | 1.32 ± 0.04 | 0.81 ± 0.02 |
| leucine | 1.11 ± 0.02 | 1.07 ± 0.00 | 1.05 ± 0.02 | 1.04 ± 0.02 | 1.04 ± 0.00 | 1.00 ± 0.02 | 1.08 ± 0.02 |
| linoleate | 1.08 ± 0.02 | 0.95 ± 0.01 | 1.02 ± 0.01 | 1.14 ± 0.02 | 0.96 ± 0.01 | 1.05 ± 0.02 | 0.94 ± 0.01 |
| linolenate | 1.22 ± 0.03 | 1.08 ± 0.01 | 0.97 ± 0.02 | 1.32 ± 0.03 | 1.10 ± 0.01 | 1.08 ± 0.02 | 1.18 ± 0.02 |
| lysine | 5.03 ± 0.29* | 1.26 ± 0.03 | 2.22 ± 0.13 | 2.36 ± 0.13 | 1.61 ± 0.03 | 6.41 ± 0.80 | 1.01 ± 0.01 |
| malate | 1.89 ± 0.08* | 1.02 ± 0.01 | 1.71 ± 0.06* | 1.50 ± 0.05* | 0.98 ± 0.01 | 1.37 ± 0.03* | 1.15 ± 0.02 |
| maltose | 0.85 ± 0.03 | 1.36 ± 0.02 | 1.84 ± 0.09 | N/D | N/D | N/D | N/D |
| mannitol | 1.85 ± 0.11 | 3.18 ± 0.17 | 3.49 ± 0.21 | 1.65 ± 0.08 | 5.40 ± 0.28 | 10.58 ± 0.48* | 3.61 ± 0.40 |
| mannose | 1.04 ± 0.02 | 1.31 ± 0.01 | 1.14 ± 0.02 | 0.98 ± 0.02 | 1.19 ± 0.01 | 1.09 ± 0.02 | 1.55 ± 0.04 |
| margarate | 1.30 ± 0.03 | 1.19 ± 0.01 | 1.21 ± 0.02 | 1.11 ± 0.02 | 1.00 ± 0.01 | 1.14 ± 0.02 | 1.03 ± 0.02 |
| methionine | 1.84 ± 0.10* | 1.06 ± 0.01 | 1.37 ± 0.03 | 1.60 ± 0.07* | 1.04 ± 0.01 | 1.51 ± 0.07 | 1.01 ± 0.01 |
| methyl linoleate | N/D | N/D | N/D | N/D | N/D | N/D | 0.76 ± 0.02 |
| methylglutaroylcarnitine | N/D | N/D | N/D | N/D | N/D | N/D | 1.18 ± 0.03 |
| myo-inositol | 2.40 ± 0.10 | 2.07 ± 0.03 | 2.38 ± 0.11 | 1.71 ± 0.06 | 2.17 ± 0.04 | 2.26 ± 0.07 | 1.49 ± 0.03 |
| myristate | 1.14 ± 0.02 | 1.11 ± 0.01 | 1.18 ± 0.02 | 1.10 ± 0.01 | 1.03 ± 0.00 | 1.10 ± 0.01 | 1.02 ± 0.02 |
| myristoleate | 1.35 ± 0.03 | 1.21 ± 0.01 | 1.45 ± 0.04 | 1.20 ± 0.02 | 1.13 ± 0.01 | 1.36 ± 0.04 | 1.28 ± 0.03 |
| N2,N2-dimethylguanosine | 1.23 ± 0.06 | 1.49 ± 0.02 | 1.84 ± 0.07* | 1.06 ± 0.07 | 1.57 ± 0.03 | 1.93 ± 0.06* | 1.63 ± 0.06 |
| N6- | 1.12 ± 0.05 | 1.38 ± 0.02 | 1.49 ± 0.06 | 1.36 ± 0.09 | 1.87 ± 0.04 | 1.96 ± 0.06* | 1.92 ± 0.07 |
| N-acetylalanine | 1.02 ± 0.02 | 1.26 ± 0.01 | 1.51 ± 0.02* | 0.94 ± 0.02 | 1.10 ± 0.01 | 1.62 ± 0.03* | 1.13 ± 0.02 |
| N-acetylaspartate | 1.22 ± 0.04 | 0.82 ± 0.01 | 1.04 ± 0.02 | N/D | N/D | N/D | N/D |
| N-acetylglucosamine 6-sulfate | N/D | N/D | N/D | 1.01 ± 0.04 | 1.21 ± 0.02 | 1.59 ± 0.04* | N/D |
| N-acetylglycine | 1.22 ± 0.03 | 1.13 ± 0.01 | 1.53 ± 0.04 | 0.95 ± 0.03 | 0.89 ± 0.01 | 1.05 ± 0.02 | 1.08 ± 0.02 |
| N-acetylmethionine | N/D | N/D | N/D | N/D | N/D | N/D | 1.31 ± 0.03 |
| N-acetylneuraminate | 1.69 ± 0.10 | 1.64 ± 0.03 | 9.83 ± 1.15* | 1.05 ± 0.06 | 1.43 ± 0.03 | 6.97 ± 0.83* | 1.21 ± 0.03 |
| N-acetylornithine | 0.94 ± 0.02 | 1.00 ± 0.01 | 0.80 ± 0.02 | 1.08 ± 0.03 | 0.87 ± 0.01 | 0.77 ± 0.02 | 1.05 ± 0.03 |
| N-acetylthreonine | 1.01 ± 0.02 | 1.42 ± 0.02 | 1.86 ± 0.06* | 1.18 ± 0.03 | 1.02 ± 0.01 | 1.56 ± 0.03* | 0.89 ± 0.02 |
| N-formylmethionine | N/D | N/D | N/D | N/D | N/D | N/D | N/D |
| nonadecanoate | N/D | N/D | N/D | 1.11 ± 0.01 | 0.98 ± 0.01 | 1.15 ± 0.02 | 1.00 ± 0.01 |
| octadecanedioate | 1.16 ± 0.05 | 1.01 ± 0.01 | 2.06 ± 0.07* | 1.41 ± 0.10 | 1.11 ± 0.01 | 4.21 ± 0.33* | 0.87 ± 0.02 |
| octanoylcarnitine | 1.69 ± 0.04* | 1.17 ± 0.02 | 2.67 ± 0.08* | 1.05 ± 0.03 | 1.18 ± 0.01 | 2.89 ± 0.11* | 0.98 ± 0.02 |
| oleate | 1.09 ± 0.02 | 0.98 ± 0.01 | 1.14 ± 0.02 | 1.03 ± 0.02 | 1.01 ± 0.01 | 1.31 ± 0.03 | 1.01 ± 0.01 |
| oleoylcarnitine | N/D | N/D | N/D | N/D | N/D | N/D | 1.11 ± 0.02 |
| ornithine | 2.98 ± 0.15* | 1.25 ± 0.02 | 2.25 ± 0.11 | 1.81 ± 0.08 | 1.47 ± 0.03 | 3.42 ± 0.36 | N/D |
| oxaloacetate | 1.09 ± 0.04 | 1.52 ± 0.02 | 2.04 ± 0.07 | 1.10 ± 0.05 | 1.60 ± 0.03 | 1.98 ± 0.09 | 2.06 ± 0.08 |
| p-acetamidophenylglucuronide | 0.92 ± 0.06 | 1.14 ± 0.02 | 0.85 ± 0.05 | 0.73 ± 0.10* | 1.82 ± 0.03 | 1.00 ± 0.06 | 1.41 ± 0.05 |
| palmitate | 1.13 ± 0.02 | 1.01 ± 0.01 | 1.11 ± 0.02 | 1.06 ± 0.02 | 0.95 ± 0.00 | 1.03 ± 0.01 | 0.97 ± 0.01 |
| palmitoleate | 1.30 ± 0.03 | 1.16 ± 0.01 | 1.32 ± 0.03 | 1.15 ± 0.03 | 1.05 ± 0.01 | 1.25 ± 0.03 | 1.08 ± 0.02 |
| palmitoylcarnitine | 2.22 ± 0.16* | 0.84 ± 0.01 | 1.75 ± 0.07* | 1.48 ± 0.04* | 0.90 ± 0.01 | 1.12 ± 0.03 | 1.08 ± 0.02 |
| pantothenate | 0.86 ± 0.02 | 1.21 ± 0.02 | 1.24 ± 0.05 | 0.95 ± 0.04 | 1.42 ± 0.02 | 1.49 ± 0.05 | 1.96 ± 0.07 |
| paraxanthine | N/D | N/D | N/D | N/D | N/D | N/D | 0.98 ± 0.05 |
| p-cresol sulfate | 1.23 ± 0.04 | 1.38 ± 0.01 | 1.39 ± 0.03 | 1.22 ± 0.04 | 1.48 ± 0.02 | 2.03 ± 0.06* | 1.39 ± 0.04 |
| pelargonate | 0.89 ± 0.01 | 0.97 ± 0.00 | 0.86 ± 0.01 | 1.12 ± 0.02 | 1.08 ± 0.01 | 0.81 ± 0.02 | 1.05 ± 0.00 |
| pentadecanoate | N/D | N/D | N/D | N/D | N/D | N/D | 1.11 ± 0.02 |
| phenol sulfate | 2.02 ± 0.08 | 1.45 ± 0.02 | 1.81 ± 0.09 | 1.36 ± 0.07 | 1.12 ± 0.01 | 1.52 ± 0.04* | 1.32 ± 0.05 |
| phenylacetate | 1.21 ± 0.06 | 0.94 ± 0.01 | 1.32 ± 0.06 | N/D | N/D | N/D | N/D |
| phenylacetylglutamine | 2.32 ± 0.12 | 4.27 ± 0.10 | 4.12 ± 0.24 | 1.77 ± 0.09 | 3.56 ± 0.10 | 5.30 ± 0.27* | 2.69 ± 0.16 |
| phenylalanine | 1.04 ± 0.01 | 1.08 ± 0.00 | 1.08 ± 0.01 | 0.94 ± 0.01 | 1.10 ± 0.00 | 1.21 ± 0.03 | 1.09 ± 0.01 |
| phenyllactate | N/D | N/D | N/D | 1.18 ± 0.07 | 1.03 ± 0.01 | 2.81 ± 0.15* | 1.15 ± 0.02 |
| phosphate | 1.23 ± 0.01* | 0.97 ± 0.00 | 1.12 ± 0.01 | 1.11 ± 0.01 | 1.05 ± 0.01 | 1.20 ± 0.01* | 0.99 ± 0.01 |
| pipecolate | 2.15 ± 0.09 | 1.45 ± 0.02 | 2.08 ± 0.07 | 2.26 ± 0.10 | 1.50 ± 0.03 | 2.00 ± 0.07 | 1.37 ± 0.07 |
| piperine | 1.25 ± 0.05 | 1.06 ± 0.02 | 0.42 ± 0.02* | 3.02 ± 0.15 | 2.13 ± 0.04 | 0.61 ± 0.03* | 1.69 ± 0.05 |
| proline | 1.48 ± 0.03* | 1.06 ± 0.01 | 1.28 ± 0.02 | 1.34 ± 0.02* | 0.99 ± 0.01 | 1.33 ± 0.04 | 1.03 ± 0.01 |
| prolylhydroxyproline | 1.41 ± 0.08 | 1.54 ± 0.02 | 1.86 ± 0.04* | 1.06 ± 0.02 | 1.34 ± 0.02 | 1.79 ± 0.03* | 1.43 ± 0.04 |
| propionylcarnitine | 2.10 ± 0.07* | 1.20 ± 0.01 | 2.53 ± 0.09* | 1.28 ± 0.03 | 0.98 ± 0.01 | 1.65 ± 0.04* | 1.20 ± 0.03 |
| pseudouridine | 1.25 ± 0.04 | 1.43 ± 0.01 | 1.74 ± 0.04* | 1.13 ± 0.04 | 1.29 ± 0.02 | 1.89 ± 0.04* | 1.31 ± 0.03 |
| pyridoxate | 1.48 ± 0.11 | 3.89 ± 0.11 | 2.36 ± 0.14 | 2.91 ± 0.34 | 6.20 ± 0.26 | 4.57 ± 0.38 | 4.36 ± 0.32 |
| pyroglutamine | 2.05 ± 0.07 | 1.32 ± 0.01 | 1.90 ± 0.05 | 2.16 ± 0.10 | 1.22 ± 0.02 | 2.10 ± 0.06* | 1.02 ± 0.02 |
| pyruvate | 0.98 ± 0.03 | 1.29 ± 0.01 | 1.65 ± 0.05 | 1.22 ± 0.03 | 1.10 ± 0.01 | 1.77 ± 0.05* | 1.62 ± 0.04 |
| quinate | 2.21 ± 0.11 | 2.25 ± 0.06 | 2.65 ± 0.26 | 1.47 ± 0.07 | 1.19 ± 0.03 | 1.00 ± 0.06 | N/D |
| riboflavin | N/D | N/D | N/D | N/D | N/D | N/D | 1.25 ± 0.05 |
| saccharin | N/D | N/D | N/D | N/D | N/D | N/D | N/D |
| salicylate | N/D | N/D | N/D | N/D | N/D | N/D | 7.39 ± 0.85 |
| salicyluric glucuronide | N/D | N/D | N/D | 1.01 ± 0.06 | 3.13 ± 0.15 | 2.46 ± 0.24 | 4.97 ± 0.51 |
| scyllo-inositol | 2.05 ± 0.09 | 1.37 ± 0.02 | 2.07 ± 0.07 | 1.33 ± 0.05 | 1.38 ± 0.03 | 2.02 ± 0.07 | 0.99 ± 0.02 |
| serine | 2.46 ± 0.08* | 1.24 ± 0.01 | 1.46 ± 0.04 | 1.66 ± 0.05 | 1.09 ± 0.01 | 1.27 ± 0.07 | 1.69 ± 0.09 |
| sphingomyelin | N/D | N/D | N/D | N/D | N/D | N/D | 1.09 ± 0.02 |
| sphingosine | N/D | N/D | N/D | N/D | N/D | N/D | 0.63 ± 0.01 |
| stachydrine | 2.12 ± 0.10 | 1.72 ± 0.03 | 2.36 ± 0.12 | 1.34 ± 0.04 | 0.92 ± 0.01 | 1.15 ± 0.05 | 1.33 ± 0.05 |
| stearate | 1.16 ± 0.02 | 1.07 ± 0.01 | 1.10 ± 0.02 | 1.10 ± 0.02 | 1.01 ± 0.00 | 1.12 ± 0.02 | 0.98 ± 0.01 |
| stearidonate | 1.36 ± 0.04 | 0.98 ± 0.01 | 1.65 ± 0.13 | 1.27 ± 0.05 | 0.90 ± 0.02 | 1.52 ± 0.08 | 1.18 ± 0.02 |
| stearoylcarnitine | N/D | N/D | N/D | N/D | N/D | N/D | 0.77 ± 0.01 |
| succinate | N/D | N/D | N/D | N/D | N/D | N/D | 1.05 ± 0.01 |
| succinoylcarnitine | N/D | N/D | N/D | N/D | N/D | N/D | 1.08 ± 0.02 |
| sucrose | 1.11 ± 0.08 | 1.15 ± 0.02 | 3.13 ± 0.23* | 1.95 ± 0.11 | 2.18 ± 0.06 | 2.10 ± 0.09 | 3.77 ± 0.24 |
| symmetric dimethylarginine | 1.22 ± 0.03 | 1.16 ± 0.01 | 1.50 ± 0.03* | 1.01 ± 0.02 | 1.14 ± 0.01 | 1.55 ± 0.03* | 1.05 ± 0.01 |
| taurochenodeoxycholate | 3.59 ± 0.34 | 3.53 ± 0.15 | 12.44 ± 0.89 | 1.66 ± 0.11 | 5.37 ± 0.28 | 11.03 ± 0.73 | 3.10 ± 0.14 |
| taurocholate | 2.28 ± 0.15 | 3.94 ± 0.15 | 14.80 ± 1.27 | 1.53 ± 0.11 | 4.15 ± 0.16 | 8.60 ± 0.68 | 4.19 ± 0.28 |
| taurolithocholate 3-sulfate | 1.02 ± 0.04 | 1.52 ± 0.02 | 1.54 ± 0.08 | 1.03 ± 0.03 | 2.46 ± 0.07 | 2.24 ± 0.13 | 2.60 ± 0.14 |
| tetradecanedioate | N/D | N/D | N/D | N/D | N/D | N/D | 1.00 ± 0.03 |
| theobromine | N/D | N/D | N/D | N/D | N/D | N/D | 1.18 ± 0.05 |
| theophylline | N/D | N/D | N/D | N/D | N/D | N/D | N/D |
| threitol | 1.93 ± 0.08 | 2.03 ± 0.04 | 2.14 ± 0.10 | 1.98 ± 0.10 | 1.76 ± 0.05 | 2.07 ± 0.09* | 1.50 ± 0.04 |
| threonate | 1.35 ± 0.04 | 1.57 ± 0.02 | 1.34 ± 0.04 | 1.46 ± 0.07 | 1.42 ± 0.02 | 1.26 ± 0.03 | 1.42 ± 0.05 |
| threonine | 1.47 ± 0.04 | 1.03 ± 0.01 | 1.10 ± 0.03 | 1.24 ± 0.02 | 1.03 ± 0.01 | 1.15 ± 0.03 | 1.56 ± 0.07 |
| thymol sulfate | N/D | N/D | N/D | N/D | N/D | N/D | N/D |
| tiglyl carnitine | 0.97 ± 0.03 | 0.87 ± 0.01 | 1.22 ± 0.03* | 0.86 ± 0.03 | 0.74 ± 0.01 | 1.34 ± 0.04* | 1.12 ± 0.03 |
| trigonelline | N/D | N/D | N/D | 1.14 ± 0.04 | 1.16 ± 0.02 | 0.75 ± 0.06 | 1.01 ± 0.04 |
| tryptophan | 1.27 ± 0.02* | 1.05 ± 0.01 | 0.88 ± 0.02 | 1.32 ± 0.02* | 1.06 ± 0.01 | 0.89 ± 0.02 | 1.12 ± 0.01 |
| tyrosine | 1.37 ± 0.03* | 0.94 ± 0.00 | 1.18 ± 0.02 | 1.26 ± 0.01* | 1.02 ± 0.01 | 1.24 ± 0.04 | 1.01 ± 0.01 |
| urate | 1.11 ± 0.01 | 0.99 ± 0.00 | 1.14 ± 0.01 | 1.24 ± 0.02* | 0.94 ± 0.00 | 1.15 ± 0.02 | 1.03 ± 0.01 |
| urea | 1.38 ± 0.04 | 1.18 ± 0.01 | 1.61 ± 0.04* | 1.14 ± 0.02 | 0.94 ± 0.01 | 1.36 ± 0.02* | 1.01 ± 0.01 |
| uridine | 1.45 ± 0.03* | 1.03 ± 0.01 | 0.97 ± 0.01 | 1.10 ± 0.02 | 1.00 ± 0.01 | 0.82 ± 0.01 | 0.97 ± 0.01 |
| urobilinogen | N/D | N/D | N/D | N/D | N/D | N/D | 0.82 ± 0.03 |
| ursodeoxycholate | N/D | N/D | N/D | 1.47 ± 0.09 | 2.40 ± 0.17 | 1.17 ± 0.06 | 0.82 ± 0.03 |
| vaccenate | 0.68 ± 0.02 | 0.69 ± 0.01 | 0.60 ± 0.02 | N/D | N/D | N/D | 0.86 ± 0.02 |
| valine | 1.24 ± 0.01* | 1.03 ± 0.00 | 0.95 ± 0.01 | 1.05 ± 0.01 | 1.05 ± 0.00 | 0.98 ± 0.02 | 1.08 ± 0.01 |
| vanillylmandelate | 1.44 ± 0.07 | 1.27 ± 0.02 | 1.80 ± 0.08* | N/D | N/D | N/D | N/D |
| X-01327 | 1.22 ± 0.03 | 1.62 ± 0.02 | 1.49 ± 0.05 | 1.87 ± 0.07 | 1.48 ± 0.02 | 1.87 ± 0.07 | N/D |
| X-01911 | N/D | N/D | N/D | 1.41 ± 0.09 | 1.31 ± 0.02 | 0.44 ± 0.02* | 1.57 ± 0.05 |
| X-02249 | 1.51 ± 0.05 | 1.61 ± 0.02 | 1.03 ± 0.05 | 1.59 ± 0.05 | 1.59 ± 0.02 | 1.19 ± 0.04 | 1.67 ± 0.05 |
| X-02269 | N/D | N/D | N/D | N/D | N/D | N/D | 1.58 ± 0.07 |
| X-02973 | 1.06 ± 0.01 | 1.05 ± 0.00 | 1.01 ± 0.01 | 1.05 ± 0.01 | 1.08 ± 0.00 | 0.96 ± 0.01 | 1.18 ± 0.02 |
| X-03002 | N/D | N/D | N/D | N/D | N/D | N/D | N/D |
| X-03056 | 0.99 ± 0.02 | 1.46 ± 0.02 | 1.86 ± 0.05 | 1.02 ± 0.03 | 1.26 ± 0.01 | 1.77 ± 0.04* | 1.29 ± 0.03 |
| X-03090 | 1.11 ± 0.02 | 1.06 ± 0.00 | 1.04 ± 0.01 | N/D | N/D | N/D | 1.09 ± 0.02 |
| X-03091 | N/D | N/D | N/D | N/D | N/D | N/D | 1.53 ± 0.04 |
| X-03094 | 1.22 ± 0.02 | 1.09 ± 0.01 | 1.05 ± 0.02 | 1.09 ± 0.02 | 1.11 ± 0.01 | 1.01 ± 0.02 | 1.18 ± 0.02 |
| X-03951 | 1.81 ± 0.09 | 1.79 ± 0.02 | 2.21 ± 0.08* | 1.55 ± 0.07 | 1.64 ± 0.03 | 2.25 ± 0.05* | N/D |
| X-04015 | N/D | N/D | N/D | N/D | N/D | N/D | 1.02 ± 0.01 |
| X-04272 | N/D | N/D | N/D | N/D | N/D | N/D | 0.94 ± 0.01 |
| X-04357 | 1.56 ± 0.04* | 1.08 ± 0.01 | 1.86 ± 0.06* | 1.46 ± 0.04* | 0.90 ± 0.01 | 1.91 ± 0.06* | 1.07 ± 0.02 |
| X-04494 | N/D | N/D | N/D | N/D | N/D | N/D | 1.04 ± 0.02 |
| X-04495 | 1.45 ± 0.04 | 1.31 ± 0.01 | 1.71 ± 0.06 | 1.28 ± 0.04 | 1.01 ± 0.01 | 1.44 ± 0.03* | 1.23 ± 0.03 |
| X-04498 | 1.17 ± 0.03 | 1.13 ± 0.01 | 1.78 ± 0.04* | 1.10 ± 0.03 | 1.02 ± 0.01 | 1.96 ± 0.06* | 1.28 ± 0.03 |
| X-04499 | 1.06 ± 0.03 | 1.06 ± 0.01 | 1.99 ± 0.05* | 0.87 ± 0.02 | 0.94 ± 0.01 | 1.59 ± 0.04* | 1.00 ± 0.02 |
| X-04504 | 2.94 ± 0.27 | 4.53 ± 0.17 | 2.74 ± 0.15 | 1.02 ± 0.07 | 4.46 ± 0.33 | 2.05 ± 0.15 | N/D |
| X-04507 | 2.17 ± 0.14 | 1.85 ± 0.03 | 1.71 ± 0.08 | 1.27 ± 0.08 | 1.52 ± 0.04 | 1.69 ± 0.07* | 1.59 ± 0.07 |
| X-04515 | 0.70 ± 0.03 | 1.80 ± 0.08 | 1.38 ± 0.08 | 1.50 ± 0.07 | 0.83 ± 0.02 | 0.88 ± 0.05 | N/D |
| X-04595 | 0.81 ± 0.02 | 0.64 ± 0.01 | 1.01 ± 0.02* | 0.95 ± 0.02 | 0.85 ± 0.01 | 1.22 ± 0.03* | 0.92 ± 0.02 |
| X-04598 | N/D | N/D | N/D | 1.09 ± 0.03 | 0.79 ± 0.01 | 1.23 ± 0.03* | 0.97 ± 0.03 |
| X-04629 | 1.11 ± 0.05 | 1.71 ± 0.03 | 1.45 ± 0.05 | 0.76 ± 0.05 | 1.03 ± 0.03 | 1.07 ± 0.03 | 1.03 ± 0.02 |
| X-05415 | N/D | N/D | N/D | N/D | N/D | N/D | 1.07 ± 0.05 |
| X-05426 | 2.50 ± 0.16 | 2.64 ± 0.07 | 1.37 ± 0.10 | 2.06 ± 0.13 | 2.36 ± 0.08 | 1.39 ± 0.05 | 1.40 ± 0.06 |
| X-05491 | 1.17 ± 0.04 | 0.98 ± 0.01 | 1.23 ± 0.03 | N/D | N/D | N/D | 1.41 ± 0.05 |
| X-05522 | 2.05 ± 0.14 | 3.27 ± 0.09 | 3.01 ± 0.13 | 1.13 ± 0.07 | 2.13 ± 0.07 | 1.79 ± 0.06 | 0.86 ± 0.03 |
| X-05907 | 1.20 ± 0.02 | 1.07 ± 0.01 | 0.67 ± 0.02* | 1.05 ± 0.02 | 1.07 ± 0.01 | 0.76 ± 0.01 | 1.05 ± 0.02 |
| X-06126 | 1.01 ± 0.05 | 2.05 ± 0.03 | 2.99 ± 0.18 | 0.78 ± 0.04 | 2.17 ± 0.05 | 4.32 ± 0.27 | 1.41 ± 0.06 |
| X-06246 | 1.22 ± 0.03 | 0.96 ± 0.01 | 0.64 ± 0.01* | 1.08 ± 0.02* | 0.82 ± 0.01 | 0.65 ± 0.01 | N/D |
| X-06267 | 1.29 ± 0.05 | 0.99 ± 0.02 | 0.95 ± 0.03 | 0.96 ± 0.03 | 0.85 ± 0.01 | 0.74 ± 0.02 | N/D |
| X-06268 | 1.07 ± 0.03 | 0.98 ± 0.01 | 1.17 ± 0.03 | 1.00 ± 0.02 | 0.72 ± 0.01 | 0.66 ± 0.02 | N/D |
| X-06346 | 1.22 ± 0.02 | 1.17 ± 0.01 | 0.94 ± 0.01 | 1.11 ± 0.02 | 1.10 ± 0.01 | 0.84 ± 0.01 | 0.87 ± 0.01 |
| X-06350 | 0.98 ± 0.02 | 0.93 ± 0.01 | 0.78 ± 0.02 | N/D | N/D | N/D | 0.88 ± 0.01 |
| X-06351 | 1.08 ± 0.05 | 1.09 ± 0.01 | 1.45 ± 0.06 | N/D | N/D | N/D | N/D |
| X-06906 | N/D | N/D | N/D | N/D | N/D | N/D | 1.14 ± 0.04 |
| X-07765 | 1.18 ± 0.04 | 2.01 ± 0.04 | 0.62 ± 0.04* | 1.61 ± 0.06 | 2.39 ± 0.06 | 0.84 ± 0.06 | 2.12 ± 0.10 |
| X-08402 | 1.26 ± 0.01 | 1.11 ± 0.01 | 0.85 ± 0.01 | 1.18 ± 0.02 | 1.17 ± 0.01 | 0.94 ± 0.01 | 1.08 ± 0.02 |
| X-08889 | 1.43 ± 0.04 | 1.15 ± 0.01 | 1.28 ± 0.03 | 1.19 ± 0.03 | 1.18 ± 0.01 | 1.26 ± 0.03 | 0.86 ± 0.02 |
| X-08988 | N/D | N/D | N/D | N/D | N/D | N/D | 1.04 ± 0.01 |
| X-09026 | 1.49 ± 0.03 | 1.39 ± 0.01 | 0.65 ± 0.01* | N/D | N/D | N/D | N/D |
| X-09044 | 2.23 ± 0.09* | 1.03 ± 0.01 | 1.34 ± 0.04 | N/D | N/D | N/D | N/D |
| X-09108 | 1.23 ± 0.03 | 1.01 ± 0.01 | 1.21 ± 0.03 | N/D | N/D | N/D | N/D |
| X-09789 | 1.81 ± 0.09 | 1.19 ± 0.01 | 0.68 ± 0.02 | 1.74 ± 0.10 | 1.09 ± 0.02 | 0.83 ± 0.04 | 0.92 ± 0.02 |
| X-10266 | 1.55 ± 0.06 | 2.01 ± 0.06 | 1.37 ± 0.03 | 1.08 ± 0.05 | 1.28 ± 0.03 | 1.19 ± 0.04 | 0.89 ± 0.03 |
| X-10346 | N/D | N/D | N/D | N/D | N/D | N/D | N/D |
| X-10359 | 2.05 ± 0.12 | 1.98 ± 0.03 | 1.79 ± 0.08 | 1.75 ± 0.09 | 1.75 ± 0.04 | 1.90 ± 0.09* | 1.22 ± 0.04 |
| X-10395 | 1.65 ± 0.04 | 1.32 ± 0.01 | 0.50 ± 0.01* | 1.50 ± 0.03 | 1.30 ± 0.01 | 0.76 ± 0.01* | 1.16 ± 0.02 |
| X-10429 | 1.35 ± 0.03 | 1.14 ± 0.01 | 0.56 ± 0.01* | N/D | N/D | N/D | N/D |
| X-10438 | 1.26 ± 0.04* | 0.78 ± 0.01 | 0.96 ± 0.03 | N/D | N/D | N/D | N/D |
| X-10439 | 2.08 ± 0.09* | 0.98 ± 0.01 | 1.50 ± 0.05 | 1.22 ± 0.05 | 0.67 ± 0.01 | 0.83 ± 0.03 | N/D |
| X-10483 | 1.01 ± 0.04 | 1.28 ± 0.02 | 1.76 ± 0.05* | 0.70 ± 0.02 | 1.10 ± 0.02 | 1.60 ± 0.04* | 1.08 ± 0.03 |
| X-10500 | 1.15 ± 0.01 | 1.04 ± 0.00 | 0.91 ± 0.01 | 1.04 ± 0.01 | 0.96 ± 0.01 | 0.84 ± 0.02 | 1.13 ± 0.01 |
| X-10510 | 1.31 ± 0.02 | 1.14 ± 0.01 | 1.04 ± 0.02 | 1.14 ± 0.02 | 1.10 ± 0.01 | 0.96 ± 0.02 | 1.02 ± 0.02 |
| X-10593 | N/D | N/D | N/D | N/D | N/D | N/D | N/D |
| X-10595 | N/D | N/D | N/D | N/D | N/D | N/D | 0.94 ± 0.01 |
| X-10609 | N/D | N/D | N/D | N/D | N/D | N/D | 1.07 ± 0.03 |
| X-10744 | 1.19 ± 0.01 | 1.03 ± 0.00 | 0.99 ± 0.01 | 1.06 ± 0.01 | 1.01 ± 0.00 | 0.97 ± 0.01 | 1.05 ± 0.01 |
| X-10747 | N/D | N/D | N/D | N/D | N/D | N/D | 0.97 ± 0.02 |
| X-10752 | 1.80 ± 0.06 | 1.41 ± 0.02 | 0.81 ± 0.01* | 1.14 ± 0.03 | 1.27 ± 0.03 | 0.96 ± 0.02 | 1.38 ± 0.04 |
| X-10876 | N/D | N/D | N/D | N/D | N/D | N/D | 0.99 ± 0.01 |
| X-10933 | N/D | N/D | N/D | 0.91 ± 0.02 | 0.94 ± 0.01 | 1.03 ± 0.02 | N/D |
| X-10964 | 1.31 ± 0.03* | 0.78 ± 0.01 | 0.75 ± 0.02 | N/D | N/D | N/D | 0.71 ± 0.02 |
| X-11168 | 1.49 ± 0.09 | 1.20 ± 0.01 | 1.09 ± 0.03 | N/D | N/D | N/D | N/D |
| X-11175 | 2.01 ± 0.07* | 1.20 ± 0.01 | 1.57 ± 0.04 | 0.95 ± 0.03 | 1.14 ± 0.02 | 2.20 ± 0.14* | 1.23 ± 0.02 |
| X-11204 | 1.02 ± 0.01 | 1.02 ± 0.00 | 0.93 ± 0.01 | 1.07 ± 0.01 | 0.99 ± 0.00 | 0.91 ± 0.01 | 0.94 ± 0.01 |
| X-11206 | 1.00 ± 0.01 | 0.93 ± 0.00 | 0.91 ± 0.01 | 0.97 ± 0.01 | 0.97 ± 0.00 | 0.92 ± 0.01 | N/D |
| X-11231 | N/D | N/D | N/D | 1.34 ± 0.04 | 1.26 ± 0.02 | 1.16 ± 0.03 | N/D |
| X-11244 | 1.36 ± 0.07 | 2.02 ± 0.03 | 1.85 ± 0.07 | 1.53 ± 0.11 | 1.76 ± 0.03 | 2.35 ± 0.13 | 2.16 ± 0.09 |
| X-11245 | 0.86 ± 0.03* | 1.82 ± 0.02 | 1.54 ± 0.05 | 0.82 ± 0.03 | 1.51 ± 0.02 | 1.61 ± 0.04 | 1.72 ± 0.04 |
| X-11255 | 1.86 ± 0.11 | 1.91 ± 0.03 | 0.81 ± 0.04 | 1.61 ± 0.09 | 1.33 ± 0.02 | 0.74 ± 0.04 | 0.99 ± 0.03 |
| X-11261 | 1.54 ± 0.04 | 1.48 ± 0.02 | 1.46 ± 0.05 | 1.27 ± 0.04 | 1.24 ± 0.01 | 1.11 ± 0.03 | 1.10 ± 0.02 |
| X-11273 | 0.75 ± 0.03* | 2.07 ± 0.03 | 1.92 ± 0.07 | 1.10 ± 0.10 | 2.11 ± 0.04 | 2.45 ± 0.10 | 1.66 ± 0.06 |
| X-11282 | 0.95 ± 0.03 | 1.16 ± 0.01 | 1.21 ± 0.04 | 1.24 ± 0.04 | 1.24 ± 0.01 | 2.43 ± 0.11* | 1.29 ± 0.03 |
| X-11299 | 1.50 ± 0.04 | 1.20 ± 0.02 | 1.62 ± 0.10 | 2.03 ± 0.06 | 1.66 ± 0.03 | 2.33 ± 0.18 | 2.30 ± 0.15 |
| X-11302 | 0.78 ± 0.02* | 2.40 ± 0.04 | 1.07 ± 0.03* | 0.86 ± 0.04* | 1.80 ± 0.03 | 1.44 ± 0.04 | 2.58 ± 0.07 |
| X-11303 | 1.07 ± 0.04 | 1.73 ± 0.03 | 1.78 ± 0.09 | 1.17 ± 0.06 | 2.61 ± 0.06 | 2.55 ± 0.13 | 2.57 ± 0.15 |
| X-11308 | 1.35 ± 0.06 | 1.10 ± 0.02 | 1.00 ± 0.03 | 1.18 ± 0.04 | 0.99 ± 0.01 | 1.14 ± 0.02 | 1.27 ± 0.03 |
| X-11315 | 0.96 ± 0.02 | 0.92 ± 0.01 | 1.05 ± 0.01 | 1.01 ± 0.02 | 0.88 ± 0.01 | 0.98 ± 0.02 | 0.93 ± 0.02 |
| X-11317 | 1.13 ± 0.02 | 1.11 ± 0.01 | 0.99 ± 0.02 | 1.03 ± 0.02 | 1.17 ± 0.01 | 1.02 ± 0.02 | N/D |
| X-11327 | 1.09 ± 0.01 | 1.00 ± 0.00 | 0.97 ± 0.02 | 1.03 ± 0.01 | 1.00 ± 0.00 | 0.88 ± 0.01 | 0.82 ± 0.01 |
| X-11333 | N/D | N/D | N/D | 1.38 ± 0.08 | 1.71 ± 0.07 | 2.38 ± 0.19* | 1.05 ± 0.05 |
| X-11334 | 1.26 ± 0.06 | 1.49 ± 0.03 | 1.31 ± 0.05 | 1.58 ± 0.11 | 1.63 ± 0.03 | 1.64 ± 0.04* | 1.84 ± 0.06 |
| X-11341 | N/D | N/D | N/D | N/D | N/D | N/D | 0.82 ± 0.02 |
| X-11372 | N/D | N/D | N/D | N/D | N/D | N/D | 1.23 ± 0.03 |
| X-11381 | 1.22 ± 0.03 | 1.05 ± 0.01 | 1.69 ± 0.04* | 1.01 ± 0.02 | 0.83 ± 0.01 | 1.67 ± 0.05* | 0.92 ± 0.01 |
| X-11400 | N/D | N/D | N/D | 3.06 ± 0.33 | 2.71 ± 0.06 | 2.31 ± 0.19 | N/D |
| X-11412 | N/D | N/D | N/D | 1.04 ± 0.02 | 1.14 ± 0.01 | 0.82 ± 0.02* | N/D |
| X-11421 (an acyl carnitine) | 1.34 ± 0.03 | 1.04 ± 0.01 | 2.21 ± 0.07* | 1.16 ± 0.05 | 1.19 ± 0.01 | 2.30 ± 0.07* | 0.96 ± 0.02 |
| X-11422 | N/D | N/D | N/D | 1.08 ± 0.03 | 1.09 ± 0.01 | 1.09 ± 0.03 | N/D |
| X-11423 | 1.70 ± 0.07 | 1.74 ± 0.02 | 1.81 ± 0.04 | 1.55 ± 0.06 | 1.69 ± 0.03 | 2.27 ± 0.06* | 1.41 ± 0.04 |
| X-11429 | 1.18 ± 0.06 | 1.12 ± 0.02 | 1.64 ± 0.07* | 1.38 ± 0.07 | 1.41 ± 0.02 | 2.01 ± 0.06* | 1.61 ± 0.06 |
| X-11431 | N/D | N/D | N/D | 1.38 ± 0.04 | 1.27 ± 0.02 | 1.20 ± 0.03 | N/D |
| X-11437 | 1.87 ± 0.09 | 3.05 ± 0.08 | 2.30 ± 0.12 | 7.12 ± 0.86 | 3.86 ± 0.11 | 2.65 ± 0.17 | 3.20 ± 0.18 |
| X-11438 | 0.96 ± 0.02 | 0.91 ± 0.01 | 1.05 ± 0.03 | 0.71 ± 0.02 | 0.81 ± 0.01 | 1.32 ± 0.04* | 0.88 ± 0.02 |
| X-11440 | 1.00 ± 0.04* | 2.25 ± 0.03 | 1.66 ± 0.08 | 0.95 ± 0.04 | 2.03 ± 0.04 | 2.20 ± 0.08 | 2.13 ± 0.05 |
| X-11441 | N/D | N/D | N/D | 1.21 ± 0.05 | 0.98 ± 0.01 | 2.05 ± 0.10* | 1.11 ± 0.02 |
| X-11442 | N/D | N/D | N/D | 1.31 ± 0.05 | 1.07 ± 0.02 | 2.26 ± 0.10* | 1.05 ± 0.02 |
| X-11443 | 1.01 ± 0.06 | 1.35 ± 0.02 | 1.16 ± 0.06 | 1.65 ± 0.11 | 1.91 ± 0.04 | 2.32 ± 0.13 | 1.38 ± 0.06 |
| X-11444 | 1.80 ± 0.13 | 3.33 ± 0.09 | 1.43 ± 0.08 | 1.73 ± 0.13 | 3.89 ± 0.14 | 1.72 ± 0.07 | 1.71 ± 0.04 |
| X-11445 | 0.90 ± 0.04 | 1.84 ± 0.03 | 0.96 ± 0.03 | 0.98 ± 0.04* | 2.26 ± 0.04 | 1.55 ± 0.05 | 1.01 ± 0.04 |
| X-11450 | 1.09 ± 0.03 | 1.53 ± 0.02 | 1.47 ± 0.04 | 1.23 ± 0.05 | 1.47 ± 0.02 | 1.77 ± 0.05 | 1.42 ± 0.04 |
| X-11452 | 0.99 ± 0.05 | 0.88 ± 0.01 | 0.53 ± 0.02 | 1.43 ± 0.07 | 1.02 ± 0.02 | 0.60 ± 0.03 | N/D |
| X-11469 | N/D | N/D | N/D | 1.67 ± 0.10 | 1.41 ± 0.03 | 0.88 ± 0.03 | 1.86 ± 0.09 |
| X-11470 | 0.84 ± 0.04 | 1.83 ± 0.04 | 0.73 ± 0.03* | 1.30 ± 0.07 | 2.13 ± 0.05 | 1.07 ± 0.04 | 1.91 ± 0.06 |
| X-11476 | N/D | N/D | N/D | 0.96 ± 0.01 | 1.01 ± 0.00 | 1.00 ± 0.01 | N/D |
| X-11478 | 0.87 ± 0.02 | 0.92 ± 0.01 | 0.68 ± 0.02 | 1.03 ± 0.02 | 1.04 ± 0.01 | 1.00 ± 0.02 | 1.44 ± 0.03 |
| X-11483 | N/D | N/D | N/D | 1.22 ± 0.04 | 1.02 ± 0.01 | 0.85 ± 0.03 | 1.19 ± 0.04 |
| X-11490 | 1.33 ± 0.06 | 1.84 ± 0.03 | 1.92 ± 0.08 | 1.51 ± 0.06 | 1.74 ± 0.04 | 6.05 ± 0.58 | 1.64 ± 0.06 |
| X-11491 | 1.02 ± 0.04 | 1.21 ± 0.04 | 1.90 ± 0.10 | 1.36 ± 0.04 | 1.37 ± 0.03 | 2.78 ± 0.15 | 1.60 ± 0.06 |
| X-11497 | 1.12 ± 0.05 | 1.20 ± 0.01 | 1.09 ± 0.03 | 2.16 ± 0.09 | 1.78 ± 0.03 | 0.92 ± 0.03 | 1.25 ± 0.03 |
| X-11510 | 1.02 ± 0.03 | 1.16 ± 0.02 | 1.29 ± 0.04 | 0.91 ± 0.03 | 1.12 ± 0.02 | 1.48 ± 0.05 | 1.59 ± 0.06 |
| X-11513 | N/D | N/D | N/D | 1.03 ± 0.04 | 0.92 ± 0.02 | 0.82 ± 0.05 | 0.81 ± 0.03 |
| X-11521 | 1.24 ± 0.03 | 1.30 ± 0.02 | 1.95 ± 0.06 | 1.18 ± 0.03 | 1.15 ± 0.02 | 2.17 ± 0.07* | 1.02 ± 0.02 |
| X-11522 | N/D | N/D | N/D | 1.10 ± 0.05 | 0.91 ± 0.01 | 3.37 ± 0.21* | N/D |
| X-11529 | 1.52 ± 0.06 | 1.50 ± 0.02 | 1.68 ± 0.09 | 1.46 ± 0.06 | 1.63 ± 0.02 | 1.47 ± 0.07 | 2.48 ± 0.12 |
| X-11530 | N/D | N/D | N/D | 1.10 ± 0.04 | 0.98 ± 0.02 | 3.00 ± 0.16* | 0.99 ± 0.02 |
| X-11533 | N/D | N/D | N/D | 1.01 ± 0.00 | 0.99 ± 0.00 | 1.03 ± 0.00 | N/D |
| X-11537 | N/D | N/D | N/D | N/D | N/D | N/D | 0.91 ± 0.03 |
| X-11538 | 1.74 ± 0.08 | 1.64 ± 0.02 | 4.37 ± 0.17* | 1.67 ± 0.11 | 1.38 ± 0.01 | 4.69 ± 0.29* | 1.08 ± 0.02 |
| X-11542 | N/D | N/D | N/D | 1.01 ± 0.01 | 0.96 ± 0.00 | 1.01 ± 0.00 | N/D |
| X-11546 | 1.28 ± 0.06 | 3.86 ± 0.27 | 1.49 ± 0.06 | 1.63 ± 0.10 | 4.57 ± 0.30 | 1.71 ± 0.07 | 1.47 ± 0.06 |
| X-11550 | 0.96 ± 0.01 | 1.04 ± 0.00 | 0.92 ± 0.01 | 1.06 ± 0.02 | 1.11 ± 0.01 | 1.03 ± 0.03 | 1.09 ± 0.01 |
| X-11560 | N/D | N/D | N/D | N/D | N/D | N/D | 1.01 ± 0.02 |
| X-11564 | N/D | N/D | N/D | 1.21 ± 0.06 | 1.16 ± 0.02 | 1.25 ± 0.03* | 1.10 ± 0.03 |
| X-11593 | 1.16 ± 0.04 | 1.81 ± 0.03 | 1.63 ± 0.04 | 1.18 ± 0.04 | 1.32 ± 0.02 | 1.49 ± 0.02* | 1.35 ± 0.03 |
| X-11687 | 1.25 ± 0.08 | 1.10 ± 0.01 | 1.46 ± 0.05* | 1.40 ± 0.08 | 1.66 ± 0.04 | 2.83 ± 0.14* | 1.54 ± 0.04 |
| X-11727 | 1.31 ± 0.03 | 1.26 ± 0.01 | 0.97 ± 0.02 | 1.05 ± 0.02 | 1.13 ± 0.01 | 1.27 ± 0.02 | 1.26 ± 0.03 |
| X-11786 | 1.27 ± 0.03 | 0.88 ± 0.01 | 0.87 ± 0.03 | 1.44 ± 0.03* | 0.94 ± 0.01 | 0.90 ± 0.02 | 1.04 ± 0.02 |
| X-11787 | 1.11 ± 0.02 | 0.99 ± 0.00 | 1.04 ± 0.02 | 1.09 ± 0.01 | 0.98 ± 0.01 | 1.05 ± 0.02 | 1.09 ± 0.02 |
| X-11793 | 0.90 ± 0.02 | 1.19 ± 0.01 | 0.89 ± 0.02 | 1.02 ± 0.03 | 1.14 ± 0.01 | 1.24 ± 0.03 | 1.33 ± 0.02 |
| X-11795 | 0.85 ± 0.03 | 0.87 ± 0.01 | 1.45 ± 0.12 | 1.17 ± 0.05 | 0.90 ± 0.01 | 1.22 ± 0.08 | 0.90 ± 0.02 |
| X-11799 | N/D | N/D | N/D | 1.78 ± 0.08 | 1.11 ± 0.02 | 1.18 ± 0.08 | 1.83 ± 0.07 |
| X-11809 | 1.12 ± 0.02 | 1.18 ± 0.01 | 1.20 ± 0.02 | 0.98 ± 0.02 | 1.15 ± 0.01 | 0.94 ± 0.02 | 0.96 ± 0.01 |
| X-11818 | N/D | N/D | N/D | 1.08 ± 0.03 | 0.85 ± 0.01 | 0.78 ± 0.01 | 1.02 ± 0.02 |
| X-11826 | 1.19 ± 0.06 | 6.17 ± 0.28 | 3.51 ± 0.25 | 3.07 ± 0.42 | 4.92 ± 0.22 | 3.38 ± 0.32 | 2.70 ± 0.15 |
| X-11832 | N/D | N/D | N/D | 0.52 ± 0.04 | 1.61 ± 0.05 | 2.49 ± 0.19 | N/D |
| X-11837 | N/D | N/D | N/D | N/D | N/D | N/D | 1.55 ± 0.09 |
| X-11838 | 0.92 ± 0.06 | 1.51 ± 0.03 | 1.62 ± 0.09 | 0.55 ± 0.05* | 1.90 ± 0.03 | 1.37 ± 0.08 | 3.22 ± 0.16 |
| X-11843 | 0.69 ± 0.03 | 1.62 ± 0.05 | 1.85 ± 0.13 | 1.14 ± 0.08 | 1.31 ± 0.04 | 3.68 ± 0.22* | 0.96 ± 0.03 |
| X-11845 | N/D | N/D | N/D | N/D | N/D | N/D | N/D |
| X-11847 | N/D | N/D | N/D | N/D | N/D | N/D | N/D |
| X-11849 | N/D | N/D | N/D | N/D | N/D | N/D | N/D |
| X-11850 | 1.50 ± 0.13 | 1.91 ± 0.07 | 1.20 ± 0.07 | 1.48 ± 0.10 | 1.52 ± 0.05 | 2.28 ± 0.11 | 1.73 ± 0.11 |
| X-11853 | 0.96 ± 0.01 | 0.99 ± 0.00 | 1.00 ± 0.01 | 0.96 ± 0.01 | 0.99 ± 0.00 | 0.96 ± 0.01 | N/D |
| X-11859 | 0.99 ± 0.01 | 1.01 ± 0.00 | 0.93 ± 0.01 | 1.01 ± 0.02 | 0.99 ± 0.01 | 0.86 ± 0.01 | N/D |
| X-11861 | N/D | N/D | N/D | 1.07 ± 0.01 | 0.99 ± 0.00 | 1.03 ± 0.01 | N/D |
| X-11868 | N/D | N/D | N/D | 0.95 ± 0.01 | 0.94 ± 0.00 | 1.06 ± 0.01 | N/D |
| X-11880 | 1.40 ± 0.03 | 1.28 ± 0.01 | 1.16 ± 0.05 | 1.44 ± 0.06 | 1.12 ± 0.02 | 0.94 ± 0.04 | 1.37 ± 0.03 |
| X-11903 | N/D | N/D | N/D | N/D | N/D | N/D | 1.34 ± 0.08 |
| X-11945 | N/D | N/D | N/D | 0.79 ± 0.03 | 1.24 ± 0.02 | 1.44 ± 0.04* | 1.69 ± 0.06 |
| X-11977 | N/D | N/D | N/D | 0.97 ± 0.04 | 1.28 ± 0.01 | 1.52 ± 0.05 | 1.47 ± 0.04 |
| X-12007 | N/D | N/D | N/D | 2.33 ± 0.20 | 1.84 ± 0.05 | 1.03 ± 0.04 | N/D |
| X-12029 | 0.90 ± 0.01 | 1.00 ± 0.00 | 0.88 ± 0.01 | 1.09 ± 0.01 | 1.01 ± 0.00 | 0.97 ± 0.01 | N/D |
| X-12038 | 1.18 ± 0.02 | 1.15 ± 0.01 | 0.92 ± 0.02 | 1.24 ± 0.03 | 1.29 ± 0.01 | 0.84 ± 0.02* | N/D |
| X-12051 | 1.84 ± 0.05 | 2.07 ± 0.05 | 1.52 ± 0.06 | 1.21 ± 0.02 | 0.96 ± 0.01 | 0.85 ± 0.01 | N/D |
| X-12063 | N/D | N/D | N/D | N/D | N/D | N/D | 0.90 ± 0.02 |
| X-12092 | 1.28 ± 0.05 | 1.67 ± 0.02 | 2.17 ± 0.11 | 1.28 ± 0.06 | 1.66 ± 0.03 | 1.82 ± 0.07 | 1.40 ± 0.04 |
| X-12094 | N/D | N/D | N/D | 1.34 ± 0.05 | 1.20 ± 0.02 | 2.38 ± 0.09* | N/D |
| X-12095 | 1.48 ± 0.04* | 0.98 ± 0.01 | 2.04 ± 0.06* | 1.36 ± 0.05 | 1.07 ± 0.02 | 1.97 ± 0.07* | 1.71 ± 0.06 |
| X-12096 | N/D | N/D | N/D | N/D | N/D | N/D | 1.07 ± 0.05 |
| X-12099 | 1.00 ± 0.02 | 1.06 ± 0.01 | 1.36 ± 0.04* | 1.17 ± 0.02 | 1.06 ± 0.01 | 1.44 ± 0.04* | N/D |
| X-12100 | 1.51 ± 0.06 | 1.70 ± 0.02 | 1.74 ± 0.07 | 1.11 ± 0.04 | 1.44 ± 0.02 | 1.53 ± 0.05 | 1.18 ± 0.03 |
| X-12101 | 1.63 ± 0.06 | 2.00 ± 0.04 | 2.05 ± 0.09 | 0.92 ± 0.03 | 1.32 ± 0.02 | 1.75 ± 0.07* | 1.75 ± 0.07 |
| X-12104 | 1.00 ± 0.03 | 1.28 ± 0.02 | 1.19 ± 0.03 | 0.90 ± 0.02 | 1.11 ± 0.01 | 1.29 ± 0.04* | 1.17 ± 0.03 |
| X-12117 | 1.84 ± 0.11 | 2.77 ± 0.05 | 2.35 ± 0.11 | 1.65 ± 0.11 | 2.47 ± 0.06 | 2.51 ± 0.11* | 3.12 ± 0.14 |
| X-12119 | N/D | N/D | N/D | N/D | N/D | N/D | 0.78 ± 0.02 |
| X-12125 | N/D | N/D | N/D | 1.37 ± 0.08 | 1.47 ± 0.04 | 1.32 ± 0.06 | 1.05 ± 0.05 |
| X-12127 | N/D | N/D | N/D | N/D | N/D | N/D | N/D |
| X-12128 | N/D | N/D | N/D | N/D | N/D | N/D | N/D |
| X-12170 | N/D | N/D | N/D | N/D | N/D | N/D | 1.16 ± 0.03 |
| X-12173 | N/D | N/D | N/D | N/D | N/D | N/D | 0.98 ± 0.04 |
| X-12199 | 1.40 ± 0.08 | 1.38 ± 0.02 | 1.03 ± 0.05 | N/D | N/D | N/D | N/D |
| X-12206 | 1.04 ± 0.06 | 1.01 ± 0.02 | 1.17 ± 0.03 | 1.39 ± 0.07 | 1.23 ± 0.02 | 1.58 ± 0.04* | 0.91 ± 0.03 |
| X-12216 | N/D | N/D | N/D | N/D | N/D | N/D | N/D |
| X-12217 | 2.75 ± 0.21 | 2.11 ± 0.05 | 1.13 ± 0.06 | 2.12 ± 0.23 | 5.02 ± 0.18 | 3.69 ± 0.27 | N/D |
| X-12231 | N/D | N/D | N/D | N/D | N/D | N/D | N/D |
| X-12244 | N/D | N/D | N/D | 1.35 ± 0.06 | 1.44 ± 0.02 | 1.17 ± 0.05 | N/D |
| X-12261 | N/D | N/D | N/D | 0.72 ± 0.04 | 0.67 ± 0.01 | 3.07 ± 0.21* | N/D |
| X-12262 | N/D | N/D | N/D | N/D | N/D | N/D | N/D |
| X-12358 | N/D | N/D | N/D | N/D | N/D | N/D | 2.07 ± 0.11 |
| X-12405 | 1.60 ± 0.10 | 1.96 ± 0.03 | 1.30 ± 0.06 | 0.99 ± 0.05 | 1.82 ± 0.04 | 2.04 ± 0.11 | 1.46 ± 0.05 |
| X-12421 | N/D | N/D | N/D | 1.24 ± 0.04 | 0.90 ± 0.01 | 0.78 ± 0.02 | N/D |
| X-12422 | 1.34 ± 0.05 | 1.25 ± 0.02 | 1.11 ± 0.05 | 1.43 ± 0.04 | 1.11 ± 0.01 | 0.94 ± 0.02 | N/D |
| X-12428 | N/D | N/D | N/D | 1.05 ± 0.07 | 1.86 ± 0.07 | 1.56 ± 0.11 | 2.03 ± 0.09 |
| X-12440 | 0.99 ± 0.01 | 0.96 ± 0.00 | 1.01 ± 0.01 | N/D | N/D | N/D | N/D |
| X-12442 | 1.81 ± 0.06 | 1.09 ± 0.01 | 1.76 ± 0.06 | 1.38 ± 0.07 | 1.09 ± 0.01 | 1.89 ± 0.07 | 0.99 ± 0.02 |
| X-12443 | N/D | N/D | N/D | 1.65 ± 0.19 | 1.50 ± 0.04 | 0.57 ± 0.02 | N/D |
| X-12450 | 1.54 ± 0.06 | 1.07 ± 0.01 | 1.03 ± 0.03 | N/D | N/D | N/D | N/D |
| X-12458 | 0.79 ± 0.02 | 0.74 ± 0.01 | 1.01 ± 0.02* | 1.06 ± 0.04 | 0.95 ± 0.01 | 1.32 ± 0.03* | N/D |
| X-12459 | N/D | N/D | N/D | N/D | N/D | N/D | 1.33 ± 0.07 |
| X-12465 (an acyl carnitine) | 3.20 ± 0.30 | 1.19 ± 0.02 | 1.98 ± 0.06* | 1.35 ± 0.08 | 1.20 ± 0.02 | 2.19 ± 0.08* | 1.41 ± 0.08 |
| X-12510 | 1.35 ± 0.04* | 0.93 ± 0.01 | 0.89 ± 0.03 | 1.08 ± 0.04 | 0.80 ± 0.01 | 0.83 ± 0.03 | 0.91 ± 0.02 |
| X-12537 | 3.11 ± 0.17 | 1.58 ± 0.03 | 1.04 ± 0.03 | N/D | N/D | N/D | 1.09 ± 0.03 |
| X-12542 | 0.73 ± 0.02 | 0.89 ± 0.01 | 0.67 ± 0.01 | N/D | N/D | N/D | N/D |
| X-12556 | 1.02 ± 0.02 | 1.03 ± 0.01 | 1.35 ± 0.03* | N/D | N/D | N/D | 1.02 ± 0.02 |
| X-12611 | 1.62 ± 0.10 | 1.76 ± 0.02 | 2.43 ± 0.09* | 1.09 ± 0.06 | 1.43 ± 0.02 | 2.55 ± 0.07* | N/D |
| X-12644 | 1.21 ± 0.04* | 0.64 ± 0.01 | 0.80 ± 0.02 | 1.50 ± 0.04* | 0.96 ± 0.01 | 0.59 ± 0.02 | 1.26 ± 0.02 |
| X-12660 | 1.24 ± 0.03 | 1.17 ± 0.02 | 0.85 ± 0.03 | 1.50 ± 0.06 | 1.04 ± 0.01 | 0.94 ± 0.03 | N/D |
| X-12681 | N/D | N/D | N/D | N/D | N/D | N/D | 0.72 ± 0.02 |
| X-12683 | N/D | N/D | N/D | N/D | N/D | N/D | 1.13 ± 0.05 |
| X-12686 | N/D | N/D | N/D | N/D | N/D | N/D | 0.95 ± 0.02 |
| X-12688 | 1.02 ± 0.05 | 1.54 ± 0.02 | 1.67 ± 0.05 | 0.94 ± 0.04 | 1.30 ± 0.02 | 1.66 ± 0.06* | 1.36 ± 0.07 |
| X-12690 | 0.79 ± 0.04 | 0.84 ± 0.01 | 1.10 ± 0.04* | N/D | N/D | N/D | 1.00 ± 0.02 |
| X-12695 | 1.76 ± 0.13 | 2.35 ± 0.07 | 1.60 ± 0.06 | 1.68 ± 0.09 | 2.21 ± 0.07 | 1.97 ± 0.06* | N/D |
| X-12707 | 1.04 ± 0.06 | 0.77 ± 0.01 | 1.06 ± 0.03* | N/D | N/D | N/D | N/D |
| X-12728 | N/D | N/D | N/D | N/D | N/D | N/D | N/D |
| X-12739 | N/D | N/D | N/D | N/D | N/D | N/D | 0.80 ± 0.02 |
| X-12742 | 1.60 ± 0.10 | 2.73 ± 0.10 | 1.97 ± 0.13 | 1.24 ± 0.11 | 2.71 ± 0.08 | 2.41 ± 0.17 | 2.02 ± 0.09 |
| X-12749 | 0.88 ± 0.04 | 0.90 ± 0.01 | 1.01 ± 0.03 | 0.89 ± 0.04 | 0.90 ± 0.02 | 1.12 ± 0.04 | 1.29 ± 0.04 |
| X-12756 | N/D | N/D | N/D | 0.33 ± 0.03* | 1.08 ± 0.02 | 0.61 ± 0.03 | N/D |
| X-12765 | N/D | N/D | N/D | N/D | N/D | N/D | 3.12 ± 0.19 |
| X-12775 | 0.95 ± 0.04 | 1.06 ± 0.01 | 1.19 ± 0.04 | 0.72 ± 0.02 | 1.30 ± 0.02 | 1.27 ± 0.03 | 2.15 ± 0.19 |
| X-12776 | 0.87 ± 0.02 | 1.06 ± 0.01 | 1.01 ± 0.02 | N/D | N/D | N/D | 0.99 ± 0.00 |
| X-12786 | 1.44 ± 0.04* | 0.89 ± 0.01 | 1.84 ± 0.07* | 0.79 ± 0.02 | 0.76 ± 0.01 | 1.41 ± 0.05* | 0.72 ± 0.02 |
| X-12792 | N/D | N/D | N/D | 1.10 ± 0.01 | 1.10 ± 0.01 | 1.02 ± 0.02 | N/D |
| X-12794 | 0.66 ± 0.04 | 1.08 ± 0.02 | 1.57 ± 0.10 | 0.54 ± 0.03* | 1.34 ± 0.03 | 1.54 ± 0.07 | N/D |
| X-12802 | 1.04 ± 0.04 | 1.63 ± 0.02 | 3.10 ± 0.11* | 0.92 ± 0.04 | 1.93 ± 0.04 | 3.97 ± 0.13* | 1.21 ± 0.03 |
| X-12822 | N/D | N/D | N/D | 0.99 ± 0.04 | 0.94 ± 0.01 | 1.44 ± 0.05* | N/D |
| X-12824 | N/D | N/D | N/D | N/D | N/D | N/D | 1.18 ± 0.03 |
| X-12844 | 1.58 ± 0.12 | 1.49 ± 0.02 | 1.04 ± 0.03 | 1.30 ± 0.08 | 1.64 ± 0.03 | 1.42 ± 0.04 | 1.47 ± 0.04 |
| X-12846 | N/D | N/D | N/D | 0.85 ± 0.06 | 2.97 ± 0.14 | 2.16 ± 0.09 | 1.63 ± 0.07 |
| X-12847 | N/D | N/D | N/D | N/D | N/D | N/D | N/D |
| X-12849 | 1.56 ± 0.10 | 1.94 ± 0.04 | 1.21 ± 0.04 | N/D | N/D | N/D | N/D |
| X-12850 | 2.36 ± 0.17 | 1.57 ± 0.04 | 4.55 ± 0.26 | 2.90 ± 0.21 | 1.76 ± 0.03 | 4.44 ± 0.22 | 3.47 ± 0.20 |
| X-12851 | N/D | N/D | N/D | 0.96 ± 0.07 | 1.46 ± 0.05 | 3.22 ± 0.17* | N/D |
| X-12855 | 1.53 ± 0.07 | 0.92 ± 0.01 | 1.82 ± 0.05* | 1.17 ± 0.04 | 0.99 ± 0.01 | 2.22 ± 0.06* | 0.74 ± 0.01 |
| X-12860 | 1.11 ± 0.05 | 0.79 ± 0.01 | 1.20 ± 0.03* | 1.01 ± 0.04 | 1.01 ± 0.01 | 1.64 ± 0.05* | 0.60 ± 0.02 |
| X-12990 | 1.27 ± 0.03 | 1.02 ± 0.01 | 1.21 ± 0.02 | 1.50 ± 0.05 | 0.97 ± 0.01 | 1.10 ± 0.04 | 0.78 ± 0.02 |
| X-13152 | N/D | N/D | N/D | N/D | N/D | N/D | 0.94 ± 0.02 |
| X-13429 | 0.76 ± 0.05 | 1.54 ± 0.03 | 0.77 ± 0.04 | 0.80 ± 0.04 | 2.00 ± 0.04 | 1.85 ± 0.15 | 4.69 ± 0.39 |
| X-13435 | 1.54 ± 0.04 | 1.18 ± 0.01 | 1.99 ± 0.05* | N/D | N/D | N/D | N/D |
| X-13465 | N/D | N/D | N/D | 1.28 ± 0.07 | 2.06 ± 0.05 | 3.17 ± 0.14* | N/D |
| X-13543 | N/D | N/D | N/D | N/D | N/D | N/D | 1.20 ± 0.04 |
| X-13553 | 1.64 ± 0.07 | 1.25 ± 0.02 | 2.88 ± 0.09* | 1.22 ± 0.05 | 1.62 ± 0.04 | 4.35 ± 0.16* | 1.25 ± 0.05 |
| X-13619 | 0.99 ± 0.02 | 1.06 ± 0.01 | 0.91 ± 0.01 | 1.07 ± 0.02 | 0.96 ± 0.00 | 0.81 ± 0.01 | 0.96 ± 0.01 |
| X-13684 | N/D | N/D | N/D | N/D | N/D | N/D | N/D |
| X-13687 | N/D | N/D | N/D | N/D | N/D | N/D | 1.09 ± 0.05 |
| X-13727 | N/D | N/D | N/D | 1.57 ± 0.13 | 2.44 ± 0.10 | 5.23 ± 0.43 | N/D |
| X-13751 | 1.03 ± 0.01 | 0.98 ± 0.00 | 0.97 ± 0.01 | 0.97 ± 0.01 | 0.97 ± 0.00 | 1.03 ± 0.01 | N/D |
| X-13852 | N/D | N/D | N/D | N/D | N/D | N/D | 2.69 ± 0.11 |
| X-13871 | N/D | N/D | N/D | N/D | N/D | N/D | 1.02 ± 0.02 |
| X-14056 | N/D | N/D | N/D | N/D | N/D | N/D | 1.06 ± 0.02 |
| X-14091 | N/D | N/D | N/D | N/D | N/D | N/D | 1.22 ± 0.03 |
| X-14318 | N/D | N/D | N/D | N/D | N/D | N/D | 0.87 ± 0.03 |
| X-14473 | N/D | N/D | N/D | N/D | N/D | N/D | 1.18 ± 0.04 |
| X-14561 | N/D | N/D | N/D | N/D | N/D | N/D | 2.19 ± 0.10 |
| X-14584 | N/D | N/D | N/D | N/D | N/D | N/D | 0.86 ± 0.03 |
| X-14588 | 0.94 ± 0.01 | 0.98 ± 0.00 | 1.16 ± 0.01* | 0.91 ± 0.01 | 0.98 ± 0.00 | 1.14 ± 0.01* | 1.01 ± 0.01 |
| X-14625 | N/D | N/D | N/D | 1.33 ± 0.03 | 1.13 ± 0.01 | 1.00 ± 0.02 | N/D |
| X-14626 | 0.84 ± 0.08 | 1.41 ± 0.03 | 0.46 ± 0.03* | 0.54 ± 0.02* | 2.23 ± 0.06 | 1.36 ± 0.09 | 4.74 ± 0.38 |
| X-14632 | N/D | N/D | N/D | N/D | N/D | N/D | N/D |
| X-14658 | 2.36 ± 0.16 | 2.34 ± 0.07 | 9.79 ± 0.71 | 4.29 ± 0.58 | 2.35 ± 0.07 | 11.58 ± 0.65* | 3.17 ± 0.24 |
| X-14662 | 1.29 ± 0.06 | 5.33 ± 0.42 | 2.79 ± 0.13 | 2.28 ± 0.20 | 11.40 ± 1.15 | 3.36 ± 0.26 | 1.78 ± 0.12 |
| X-14663 | 1.21 ± 0.13 | 2.30 ± 0.13 | 2.67 ± 0.17 | 1.84 ± 0.16 | 12.04 ± 1.22 | 3.65 ± 0.27 | 1.15 ± 0.06 |
| X-14837 | N/D | N/D | N/D | N/D | N/D | N/D | 1.33 ± 0.12 |
| X-14842 | N/D | N/D | N/D | N/D | N/D | N/D | 0.98 ± 0.01 |
| xanthine | 1.40 ± 0.03 | 1.04 ± 0.01 | 1.35 ± 0.03 | 0.90 ± 0.02 | 0.87 ± 0.01 | 0.90 ± 0.02 | 1.10 ± 0.04 |
| xylonate | 2.04 ± 0.10 | 2.48 ± 0.05 | 2.47 ± 0.10 | 1.63 ± 0.09 | 1.70 ± 0.04 | 1.99 ± 0.07* | N/D |
| xylose | 2.13 ± 0.13 | 1.28 ± 0.02 | 1.53 ± 0.07 | 1.14 ± 0.05 | 0.98 ± 0.02 | 0.96 ± 0.03 | N/D |

| | Replication | Replication | Replication | | | |
|---|---|---|---|---|---|---|
| | t0 Sepsis | t24 Sepsis | t24 Sepsis | | | |
| Biochemical | Deaths | Survivors | Deaths | PLATFORM | KEGG ID | HMDB ID |
| 1,5-anhydroglucitol | 1.07 ± 0.03* | 0.93 ± 0.02 | 1.14 ± 0.04 | LC/MS neg | C07326 | HMDB02712 |
| 1,6-anhydroglucose | 1.75 ± 0.12 | 1.32 ± 0.06 | 1.24 ± 0.09 | GC/MS | | HMDB00640 |
| 10-heptadecenoate | 1.02 ± 0.03 | 1.01 ± 0.02 | 1.10 ± 0.04 | LC/MS neg | | |
| 10-nonadecenoate | 1.04 ± 0.02 | 1.04 ± 0.02 | 1.30 ± 0.06 | LC/MS neg | | |
| 1-arachidoyl-GPC | 0.69 ± 0.03 | 1.36 ± 0.03 | 0.79 ± 0.04 | LC/MS pos | (C05208) | |
| 1-arachidoyl-GPE | 0.99 ± 0.03 | 1.02 ± 0.02 | 0.97 ± 0.04 | LC/MS neg | | |
| 1-arachidoyl-GPI | 1.11 ± 0.04 | 1.13 ± 0.01 | 1.06 ± 0.03 | LC/MS neg | (C03819) | |
| 1-docosahexaenoyl-GPC | 0.83 ± 0.04 | 1.32 ± 0.02 | 1.02 ± 0.04 | LC/MS pos | | |
| 1-eicosadienoyl-GPC | 0.76 ± 0.03 | 0.90 ± 0.02 | 0.70 ± 0.02 | LC/MS pos | | |
| 1-eicosatrienoyl-GPC | 0.78 ± 0.05 | 1.31 ± 0.03 | 0.73 ± 0.04 | LC/MS pos | | |
| 1-heptadecanoyl-GPC | 0.72 ± 0.04 | 0.93 ± 0.02 | 0.68 ± 0.03 | LC/MS pos | | |
| 1-linoleoyl-GPC | 0.73 ± 0.03 | 1.35 ± 0.03 | 0.91 ± 0.05 | LC/MS pos | C04100 | |
| 1-linoleoyl-GPE | N/D | 1.18 ± 0.03 | 1.04 ± 0.05 | LC/MS neg | | |
| 1-linoleoyl-GPI | 0.95 ± 0.04 | N/D | N/D | LC/MS neg | (C03819) | |
| 1-methyladenosine | 1.16 ± 0.02 | 0.99 ± 0.01 | 1.13 ± 0.02 | LC/MS pos | C02494 | HMDB03331 |
| 1-methylimidazoleacetate | 1.51 ± 0.12 | 1.43 ± 0.06 | 1.87 ± 0.16 | LC/MS pos | C05828 | HMDB02820 |
| 1-methylurate | 1.26 ± 0.09 | 1.09 ± 0.04 | 1.09 ± 0.08 | LC/MS pos | | HMDB03099 |
| 1-myristoyl-GPC | 0.68 ± 0.04 | N/D | N/D | LC/MS pos | | |
| 1-oleoylglycerol | 0.80 ± 0.08 | 1.20 ± 0.04 | 1.14 ± 0.10 | LC/MS pos | (C01885) | |
| 1-oleoylglycerophosphate | N/D | 1.12 ± 0.02 | 0.90 ± 0.04 | LC/MS neg | | HMDB00443 |
| 1-oleoyl-GPC | 0.81 ± 0.04 | 1.30 ± 0.02 | 0.75 ± 0.03 | LC/MS pos | C03916 | HMDB02815 |
| 1-oleoyl-GPE | 1.05 ± 0.04 | 1.24 ± 0.02 | 1.05 ± 0.04 | LC/MS neg | | |
| 1-palmitoleoyl-GPC | 0.74 ± 0.03 | 1.33 ± 0.03 | 0.88 ± 0.03 | LC/MS pos | | |
| 1-palmitoleoyl-GPI | 0.91 ± 0.04 | N/D | N/D | LC/MS neg | (C03819) | |
| 1-palmitoylglycerol | N/D | N/D | N/D | GC/MS | | |
| 1-palmitoyl-GPC | 0.73 ± 0.03 | 1.25 ± 0.02 | 0.64 ± 0.03* | LC/MS pos | C04102 | |
| 1-palmitoyl-GPE | 0.95 ± 0.03 | 1.38 ± 0.03 | 1.16 ± 0.05 | LC/MS neg | | |
| 1-palmitoyl-GPI | 1.45 ± 0.09 | N/D | N/D | LC/MS neg | (C03819) | |
| 1-stearoylglycerol | 0.74 ± 0.02 | 1.06 ± 0.02 | 0.89 ± 0.02 | GC/MS | (C01885) | |
| 1-stearoyl-GPC | 0.79 ± 0.04 | 1.38 ± 0.03 | 0.71 ± 0.03* | LC/MS pos | | |
| 1-stearoyl-GPE | 0.63 ± 0.02 | N/D | N/D | LC/MS pos | | |
| 1-stearoyl-GPI | 1.23 ± 0.05 | 1.24 ± 0.02 | 1.41 ± 0.05 | LC/MS neg | (C03819) | |
| 2-aminobutyrate | 1.24 ± 0.06 | 1.24 ± 0.02 | 1.24 ± 0.07 | GC/MS | C02261 | HMDB00452 |
| 2-arachidonoyl-GPE | 0.98 ± 0.04 | 0.83 ± 0.02 | 0.76 ± 0.03 | LC/MS neg | | |
| 2-hydroxyacetaminophen | 0.89 ± 0.06 | 1.70 ± 0.08 | 0.91 ± 0.08 | LC/MS neg | | |
| 2-hydroxybutyrate | 1.20 ± 0.05 | 1.10 ± 0.02 | 1.18 ± 0.05 | GC/MS | C05984 | HMDB00008 |
| 2-hydroxyhippurate | 1.26 ± 0.13 | N/D | N/D | LC/MS neg | C07588 | HMDB00840 |
| 2-hydroxypalmitate | 1.33 ± 0.03 | 0.99 ± 0.01 | 1.22 ± 0.04 | LC/MS neg | | |
| 2-hydroxystearate | 1.20 ± 0.04 | 0.99 ± 0.01 | 1.08 ± 0.03 | LC/MS neg | C03045 | |
| 2-linoleoyl-GPC | 0.42 ± 0.03 | 0.89 ± 0.02 | 0.73 ± 0.03 | LC/MS pos | | |
| 2-methoxyacetaminophen | 0.73 ± 0.04 | N/D | N/D | LC/MS pos | | |
| glucuronide | | | | | | |
| 2-methoxyacetaminophen | 0.93 ± 0.06 | 1.59 ± 0.07 | 1.00 ± 0.08 | LC/MS neg | | |
| 2-methylbutyroylcarnitine | 1.50 ± 0.08 | 1.07 ± 0.02 | 1.41 ± 0.07 | LC/MS pos | | HMDB00378 |
| 2-octenoylcarnitine | N/D | 0.60 ± 0.01 | 1.01 ± 0.05* | LC/MS pos | | |
| 2-oleoyl-GPC | 0.79 ± 0.03 | 1.12 ± 0.03 | 0.84 ± 0.03 | LC/MS pos | | |
| 2-palmitoyl-GPC | 0.74 ± 0.04 | 1.37 ± 0.03 | 0.74 ± 0.03* | LC/MS pos | | |
| 2-stearoyl-GPC | 0.64 ± 0.03 | 1.20 ± 0.02 | 0.75 ± 0.03 | LC/MS pos | | |
| 3-(4-hydroxyphenyl)lactate | 1.75 ± 0.11 | 1.11 ± 0.03 | 1.83 ± 0.12 | LC/MS neg | C03672 | HMDB00755 |
| 3-(cystein-S-yl)acetaminophen | 0.73 ± 0.05 | 2.93 ± 0.11 | 1.81 ± 0.16 | LC/MS pos | | |
| 3-aminoisobutyrate | N/D | 1.29 ± 0.11 | 1.51 ± 0.14 | GC/MS | C05145 | HMDB03911 |
| 3-carboxy-4-methyl-5-propyl-2- | 1.57 ± 0.15 | 2.74 ± 0.14 | 1.14 ± 0.12 | LC/MS neg | | |
| furanpropanoate | | | | | | |
| 3-dehydrocarnitine | 1.61 ± 0.07 | 1.13 ± 0.02 | 1.47 ± 0.05 | LC/MS pos | C02636 | |
| 3-hydroxy-2-ethylpropionate | 0.76 ± 0.03 | 0.62 ± 0.01 | 0.94 ± 0.04 | GC/MS | | HMDB00396 |
| 3-hydroxybutyrate | 1.17 ± 0.06 | 2.60 ± 0.13 | 1.70 ± 0.14 | GC/MS | C01089 | HMDB00357 |
| 3-hydroxydecanoate | 0.87 ± 0.03 | N/D | N/D | LC/MS neg | | HMDB02203 |
| 3-hydroxyisobutyrate | 1.00 ± 0.04 | 1.51 ± 0.06 | 1.58 ± 0.07 | LC/MS pos | C01188 | HMDB00336 |
| | | | | | C06001 | HMDB00023 |
| 3-hydroxykynurenine | N/D | 1.15 ± 0.05 | 1.34 ± 0.10 | LC/MS pos | C02794 | HMDB00732 |
| 3-hydroxyoctanoate | 0.98 ± 0.05 | 0.81 ± 0.02 | 1.15 ± 0.09 | LC/MS neg | | HMDB01954 |
| 3-indoxyl sulfate | 2.04 ± 0.13 | 1.39 ± 0.04 | 1.15 ± 0.05 | LC/MS neg | | HMDB00682 |
| 3-methoxytyrosine | 1.15 ± 0.04 | 1.09 ± 0.02 | 1.16 ± 0.05 | LC/MS pos | | HMDB01434 |
| 3-methyl-2-oxobutyrate | 0.96 ± 0.01 | 0.99 ± 0.01 | 0.92 ± 0.01 | LC/MS neg | C00141 | HMDB00019 |
| 3-methyl-2-oxovalerate | 0.95 ± 0.03 | 1.17 ± 0.01 | 0.85 ± 0.02 | LC/MS neg | C00671 | HMDB03736 |
| 3-methylhistidine | 0.60 ± 0.04 | 2.02 ± 0.12 | 0.72 ± 0.08 | LC/MS neg | C01152 | HMDB00479 |
| 4-acetamidobutanoate | 3.81 ± 0.31 | 2.16 ± 0.08 | 4.00 ± 0.32 | LC/MS pos | C02946 | HMDB03681 |
| 4-acetamidophenol | 0.89 ± 0.07 | 2.47 ± 0.11 | 1.25 ± 0.11 | LC/MS pos | C06804 | HMDB01859 |
| 4-acetaminophen sulfate | 0.85 ± 0.08 | 2.12 ± 0.09 | 1.47 ± 0.12 | LC/MS neg | | |
| 4-ethylphenyl sulfate | N/D | 2.46 ± 0.14 | 1.57 ± 0.22 | LC/MS neg | | |
| 4-hydroxyphenylacetate | N/D | 0.86 ± 0.03 | 1.46 ± 0.15 | LC/MS neg | C00642 | HMDB00020 |
| 4-methyl-2-oxopentanoate | 0.98 ± 0.03 | 1.18 ± 0.02 | 0.94 ± 0.02 | LC/MS neg | C00233 | HMDB00695 |
| 4-vinylphenol sulfate | N/D | 1.72 ± 0.13 | 2.03 ± 0.14 | LC/MS neg | | |
| 5-dodecenoate | 1.25 ± 0.05 | 1.48 ± 0.09 | 2.10 ± 0.17 | LC/MS neg | | HMDB00529 |
| 5-methylthioadenosine | 1.20 ± 0.06 | 0.74 ± 0.02 | 0.97 ± 0.04 | LC/MS pos | C00170 | HMDB01173 |
| 5-oxoproline | 1.12 ± 0.02 | 1.08 ± 0.02 | 1.14 ± 0.02 | LC/MS pos | C01879 | HMDB00267 |
| 7-alpha-hydroxy-3-oxo-4- | 1.01 ± 0.02 | 1.19 ± 0.02 | 1.20 ± 0.04 | LC/MS neg | C17337 | |
| cholestenoate | | | | | | |
| acetoacetate | N/D | N/D | N/D | LC/MS neg | C00164 | HMDB00060 |
| acetylcarnitine | 2.01 ± 0.13 | 0.95 ± 0.01 | 1.35 ± 0.06 | LC/MS pos | C02571 | HMDB00201 |
| adenosine 5′-monophosphate | 0.92 ± 0.04 | 2.43 ± 0.17 | 1.05 ± 0.04 | LC/MS pos | C00020 | HMDB00045 |
| adrenate | 1.01 ± 0.02 | 1.06 ± 0.02 | 0.99 ± 0.03 | LC/MS neg | C16527 | HMDB02226 |
| alanine | 1.02 ± 0.04 | 1.16 ± 0.02 | 0.92 ± 0.03 | GC/MS | C00041 | HMDB00161 |
| allantoin | N/D | N/D | N/D | GC/MS | C02350 | HMDB00462 |
| alpha-hydroxyisovalerate | 2.34 ± 0.13 | 1.23 ± 0.04 | 2.63 ± 0.20 | LC/MS neg | | HMDB00407 |
| alpha-ketobutyrate | 1.02 ± 0.03 | 1.12 ± 0.02 | 0.97 ± 0.03 | LC/MS neg | C00109 | HMDB00005 |
| alpha-ketoglutarate | N/D | 0.53 ± 0.02 | 0.82 ± 0.05 | GC/MS | C00026 | HMDB00208 |
| alpha-tocopherol | 1.09 ± 0.03 | 1.24 ± 0.02 | 1.15 ± 0.02 | GC/MS | C02477 | HMDB01893 |
| androsterone sulfate | 1.84 ± 0.13 | 1.36 ± 0.04 | 1.49 ± 0.09 | LC/MS neg | (C00523) | HMDB02759 |
| arabinose | 1.55 ± 0.08 | 1.06 ± 0.03 | 1.33 ± 0.07 | GC/MS | C00181 | HMDB00646 |
| arabitol | 2.23 ± 0.12 | 1.45 ± 0.04 | 1.90 ± 0.13 | GC/MS | C00474 | HMDB01851 |
| arachidonate | 0.98 ± 0.03 | 1.18 ± 0.01 | 0.95 ± 0.03 | LC/MS neg | C00219 | HMDB01043 |
| arginine | 1.05 ± 0.02 | 1.02 ± 0.01 | 0.97 ± 0.02 | LC/MS pos | C00062 | HMDB03416 |
| asparagine | 1.05 ± 0.03 | 1.07 ± 0.02 | 1.08 ± 0.04 | GC/MS | C00152 | HMDB00168 |
| aspartate | 0.85 ± 0.03 | 1.90 ± 0.15 | 1.19 ± 0.03 | GC/MS | C00049 | HMDB00191 |
| beta-hydroxyisovalerate | 1.48 ± 0.05 | 1.20 ± 0.02 | 2.10 ± 0.18 | LC/MS neg | | HMDB00754 |
| beta-hydroxypyruvate | N/D | 0.94 ± 0.01 | 0.84 ± 0.02 | GC/MS | C00168 | HMDB01352 |
| betaine | 1.36 ± 0.04 | 0.96 ± 0.01 | 1.17 ± 0.03 | LC/MS pos | C00719 | HMDB00043 |
| beta-sitosterol | N/D | N/D | N/D | GC/MS | C01753 | HMDB00852 |
| bilirubin | 3.15 ± 0.28 | 1.28 ± 0.04 | 3.26 ± 0.34 | LC/MS neg | C00486 | HMDB00054 |
| bilirubin (E,E) | 0.95 ± 0.03 | 1.38 ± 0.04 | 1.17 ± 0.04 | LC/MS pos | C00486 | HMDB00054 |
| bilirubin(E,Z or Z,E) | 1.06 ± 0.05 | N/D | N/D | LC/MS pos | C00486 | HMDB00054 |
| biliverdin | 0.76 ± 0.03 | 1.31 ± 0.04 | 1.05 ± 0.05 | LC/MS pos | C00500 | HMDB01008 |
| butyrylcarnitine | 2.92 ± 0.32 | 1.05 ± 0.01 | 3.00 ± 0.30* | LC/MS pos | C02862 | HMDB02013 |
| caffeine | 6.33 ± 0.84 | 1.17 ± 0.04 | 3.65 ± 0.42 | LC/MS pos | C07481 | HMDB01847 |
| caprate | 1.09 ± 0.03 | 1.10 ± 0.02 | 0.91 ± 0.02 | LC/MS neg | C01571 | HMDB00511 |
| caproate | 1.01 ± 0.01 | 1.09 ± 0.01 | 0.93 ± 0.02 | LC/MS neg | C01585 | HMDB00535 |
| caprylate | 1.23 ± 0.05 | 1.12 ± 0.01 | 0.91 ± 0.02 | LC/MS neg | C06423 | HMDB00482 |
| carnitine | 1.03 ± 0.04 | 0.98 ± 0.01 | 0.97 ± 0.02 | LC/MS pos | C00487 | HMDB00062 |
| catechol sulfate | 1.63 ± 0.07 | 2.29 ± 0.11 | 1.20 ± 0.07 | LC/MS neg | (C00090) | |
| C-glycosyltryptophan | 2.57 ± 0.16 | 1.96 ± 0.07 | 2.40 ± 0.17 | LC/MS pos | | |
| chenodeoxycholate | N/D | 1.56 ± 0.05 | 1.08 ± 0.06 | LC/MS neg | C02528 | HMDB00518 |
| cholate | 1.33 ± 0.10 | 4.04 ± 0.49 | 1.05 ± 0.06 | LC/MS neg | C00695 | HMDB00619 |
| cholesterol | 0.94 ± 0.01 | 1.16 ± 0.02 | 1.04 ± 0.02 | GC/MS | C00187 | HMDB00067 |
| choline | 1.09 ± 0.04 | 1.08 ± 0.01 | 0.97 ± 0.02 | LC/MS pos | C00114 | HMDB00097 |
| citrate | 0.86 ± 0.02 | 1.19 ± 0.01 | 1.12 ± 0.05 | GC/MS | C00158 | HMDB00094 |
| citrulline | 1.03 ± 0.03 | 1.22 ± 0.02 | 0.93 ± 0.02 | LC/MS pos | C00327 | HMDB00904 |
| cortisol | 1.35 ± 0.03 | 1.19 ± 0.03 | 1.70 ± 0.10 | LC/MS pos | C00735 | HMDB00063 |
| cortisone | 0.92 ± 0.03 | 0.91 ± 0.01 | 0.99 ± 0.02 | LC/MS pos | C00762 | HMDB02802 |
| creatine | 2.87 ± 0.23 | 1.71 ± 0.05 | 3.04 ± 0.21 | LC/MS pos | C00300 | HMDB00064 |
| creatinine | 1.08 ± 0.05* | 1.45 ± 0.03 | 1.04 ± 0.05 | LC/MS pos | C00791 | HMDB00562 |
| cysteine | 1.15 ± 0.03 | 1.26 ± 0.02 | 1.20 ± 0.05 | GC/MS | C00097 | HMDB00574 |
| cystine | N/D | N/D | N/D | GC/MS | C00491 | HMDB00192 |
| decanoylcarnitine | N/D | 0.96 ± 0.01 | 1.66 ± 0.06* | LC/MS pos | C03299 | HMDB00651 |
| dehydroisoandrosterone sulfate | 1.54 ± 0.09 | 1.37 ± 0.04 | 1.17 ± 0.07 | LC/MS neg | (C01227) | HMDB01032 |
| deoxycarnitine | 1.43 ± 0.06 | 1.01 ± 0.01 | 1.17 ± 0.05 | LC/MS pos | C01181 | HMDB01161 |
| deoxycholate | 0.81 ± 0.04 | 1.20 ± 0.03 | 0.98 ± 0.06 | LC/MS neg | C04483 | HMDB00626 |
| dihomolinoleate | 1.05 ± 0.02 | 0.98 ± 0.02 | 1.14 ± 0.06 | LC/MS neg | | |
| dihomolinolenate | 1.01 ± 0.04 | 1.10 ± 0.01 | 0.97 ± 0.03 | LC/MS neg | C03242 | HMDB02925 |
| dihydroxyacetone | N/D | 1.36 ± 0.03 | 1.26 ± 0.05 | GC/MS | C00184 | HMDB01882 |
| docosahexaenoate | 1.05 ± 0.03 | 1.10 ± 0.02 | 1.01 ± 0.04 | LC/MS neg | C06429 | HMDB02183 |
| docosapentaenoate | 1.09 ± 0.03 | 1.14 ± 0.02 | 1.02 ± 0.04 | LC/MS neg | C16513 | HMDB01976 |
| dodecanedioate | 0.87 ± 0.03 | 0.87 ± 0.01 | 1.08 ± 0.05 | LC/MS neg | C02678 | HMDB00623 |
| eicosapentaenoate | 1.00 ± 0.03 | 1.24 ± 0.01 | 1.03 ± 0.03 | LC/MS neg | C06428 | HMDB01999 |
| eicosenoate | 1.11 ± 0.02 | 1.06 ± 0.02 | 1.49 ± 0.07 | LC/MS neg | | HMDB02231 |
| epiandrosterone sulfate | 1.07 ± 0.06 | 1.31 ± 0.04 | 1.24 ± 0.07 | LC/MS neg | (C07635) | (HMDB00365) |
| erythritol | 2.03 ± 0.11 | 1.31 ± 0.04 | 1.90 ± 0.12 | GC/MS | C00503 | HMDB02994 |
| erythronate | 2.22 ± 0.13 | 1.78 ± 0.07 | 2.34 ± 0.16 | GC/MS | C01620 | HMDB00613 |
| erythrose | 1.05 ± 0.02 | 1.11 ± 0.01 | 1.17 ± 0.03 | GC/MS | C01796 | HMDB02649 |
| estrone 3-sulfate | N/D | 0.78 ± 0.03 | 1.70 ± 0.27 | LC/MS neg | C02538 | HMDB01425 |
| fructose | 1.18 ± 0.04 | 2.05 ± 0.08 | 1.91 ± 0.15 | GC/MS | C00095 | HMDB00660 |
| galactonate | 1.04 ± 0.03 | 1.08 ± 0.01 | 1.03 ± 0.03 | GC/MS | C00257 | HMDB03290 |
| gamma-glutamylglutamine | 0.95 ± 0.03 | 1.04 ± 0.01 | 0.88 ± 0.03 | LC/MS pos | | |
| gamma-glutamylisoleucine | 0.82 ± 0.03 | 1.10 ± 0.02 | 0.85 ± 0.03 | LC/MS pos | | |
| gamma-glutamylleucine | 0.83 ± 0.02* | 1.13 ± 0.02 | 0.92 ± 0.02 | LC/MS pos | | |
| gamma-glutamylmethionine | 0.88 ± 0.03 | 0.91 ± 0.01 | 1.09 ± 0.04 | LC/MS pos | | |
| gamma-glutamylphenylalanine | 1.12 ± 0.04 | 1.10 ± 0.02 | 1.11 ± 0.03 | LC/MS pos | | HMDB00594 |
| gamma-glutamyltyrosine | 1.01 ± 0.02 | 1.03 ± 0.01 | 1.27 ± 0.04 | LC/MS pos | | |
| gluconate | 4.52 ± 0.81 | 6.68 ± 0.64 | 6.63 ± 0.96 | GC/MS | C00257 | HMDB00625 |
| glucose | 1.05 ± 0.02 | 1.07 ± 0.01 | 0.98 ± 0.02 | GC/MS | C00267 | HMDB00122 |
| glucuronate | 1.76 ± 0.12 | 1.47 ± 0.06 | 1.65 ± 0.10 | GC/MS | C00191 | HMDB00127 |
| glutamate | 1.16 ± 0.06 | 2.29 ± 0.14 | 1.30 ± 0.06 | GC/MS | C00025 | HMDB03339 |
| glutamine | 1.06 ± 0.02 | 1.02 ± 0.01 | 1.02 ± 0.02 | LC/MS pos | C00064 | HMDB00641 |
| glutamylvaline | 0.90 ± 0.02 | 1.24 ± 0.02 | 1.03 ± 0.02 | LC/MS pos | | |
| glutaroylcarnitine | 1.06 ± 0.05 | 1.17 ± 0.02 | 1.08 ± 0.04 | LC/MS pos | | |
| glycerate | 1.28 ± 0.04 | 1.28 ± 0.03 | 1.22 ± 0.03 | GC/MS | C00258 | HMDB00139 |
| glycerol | 1.18 ± 0.03 | 1.15 ± 0.02 | 1.41 ± 0.06 | GC/MS | C00116 | HMDB00131 |
| glycerol 2-phosphate | 1.09 ± 0.04 | N/D | N/D | GC/MS | C02979 | HMDB02520 |
| glycerol 3-phosphate | 1.06 ± 0.03 | 1.16 ± 0.02 | 0.97 ± 0.01 | GC/MS | C00093 | HMDB00126 |
| glycerophosphorylcholine | 1.12 ± 0.07 | 1.27 ± 0.05 | 0.93 ± 0.06 | LC/MS pos | C00670 | HMDB00086 |
| glycine | 1.39 ± 0.09 | 1.10 ± 0.02 | 0.97 ± 0.02 | GC/MS | C00037 | HMDB00123 |
| glycochenodeoxycholate | 3.51 ± 0.38 | 1.46 ± 0.05 | 3.38 ± 0.31 | LC/MS neg | C05466 | HMDB00637 |
| glycocholate | 4.28 ± 0.47 | 2.34 ± 0.12 | 4.25 ± 0.49 | LC/MS neg | C01921 | HMDB00138 |
| glycodeoxycholate | 1.03 ± 0.13 | 1.30 ± 0.06 | 0.71 ± 0.08 | LC/MS pos | C05464 | HMDB00631 |
| glycolate | 1.24 ± 0.08 | 1.03 ± 0.01 | 1.11 ± 0.02 | GC/MS | C00160 | HMDB00115 |
| glycylproline | N/D | 0.83 ± 0.01 | 0.87 ± 0.05 | LC/MS pos | | HMDB00721 |
| gulono-1,4-lactone | N/D | 1.65 ± 0.07 | 1.55 ± 0.13 | GC/MS | C01040 | HMDB03466 |
| heme | 1.12 ± 0.12 | 6.73 ± 0.73 | 1.16 ± 0.07 | LC/MS pos | C00032 | HMDB03178 |
| heptanoate | 0.96 ± 0.01 | 1.15 ± 0.01 | 0.89 ± 0.02 | LC/MS neg | | HMDB00666 |
| hexadecanedioate | 1.60 ± 0.08* | 1.06 ± 0.04 | 3.90 ± 0.46* | LC/MS neg | | HMDB00672 |
| hexanoylcarnitine | 1.79 ± 0.08 | 0.89 ± 0.01 | 1.96 ± 0.10* | LC/MS pos | | HMDB00705 |
| hippurate | 4.25 ± 0.72 | 8.02 ± 0.47 | 7.84 ± 1.31 | LC/MS neg | C01586 | HMDB00714 |
| histidine | 0.95 ± 0.02 | 0.98 ± 0.01 | 0.96 ± 0.02 | LC/MS neg | C00135 | HMDB00177 |
| homocitrulline | N/D | 0.86 ± 0.02 | 1.17 ± 0.08 | LC/MS pos | C02427 | HMDB00679 |
| homostachydrine | 1.30 ± 0.04 | 1.04 ± 0.02 | 1.17 ± 0.06 | LC/MS pos | C08283 | HMDB04827 |
| hydroquinone sulfate | N/D | 1.26 ± 0.06 | 4.05 ± 0.52 | LC/MS neg | (C00530) | (HMDB02434) |
| hydroxyisovaleroylcarnitine | 1.24 ± 0.06 | 1.10 ± 0.02 | 1.14 ± 0.04 | LC/MS pos | | |
| hydroxyproline | 1.26 ± 0.05 | 1.14 ± 0.03 | 1.16 ± 0.04 | GC/MS | C01157 | HMDB00725 |
| hyodeoxycholate | 1.25 ± 0.11 | 1.02 ± 0.05 | 1.10 ± 0.14 | LC/MS neg | C15517 | HMDB00733 |
| hypoxanthine | 0.94 ± 0.03 | 1.80 ± 0.09 | 1.60 ± 0.09 | LC/MS pos | C00262 | HMDB00157 |
| ibuprofen | N/D | N/D | N/D | LC/MS neg | C01588 | HMDB01925 |
| iminodiacetate | 0.81 ± 0.04 | 1.44 ± 0.03 | 1.07 ± 0.05 | GC/MS | | HMDB11753 |
| indoleacetate | 1.47 ± 0.08 | 1.26 ± 0.03 | 1.04 ± 0.03 | LC/MS pos | C00954 | HMDB00197 |
| indolelactate | 0.91 ± 0.03 | 1.36 ± 0.04 | 1.14 ± 0.04 | GC/MS | C02043 | HMDB00671 |
| indolepropionate | 1.17 ± 0.06 | 0.99 ± 0.01 | 0.93 ± 0.04 | LC/MS pos | | HMDB02302 |
| isobutyrylcarnitine | 1.19 ± 0.06 | 1.67 ± 0.06 | 1.60 ± 0.10 | LC/MS pos | | HMDB00736 |
| isoleucine | 0.96 ± 0.03 | 1.10 ± 0.01 | 0.92 ± 0.03 | LC/MS pos | C00407 | HMDB00172 |
| isovalerate | 1.02 ± 0.01 | 1.29 ± 0.03 | 0.85 ± 0.02 | LC/MS neg | C08262 | HMDB00718 |
| isovalerylcarnitine | 1.06 ± 0.04 | 1.10 ± 0.02 | 1.08 ± 0.04 | LC/MS pos | | HMDB00688 |
| kynurenate | N/D | N/D | N/D | LC/MS neg | C01717 | HMDB00715 |
| kynurenine | 0.92 ± 0.04 | 1.11 ± 0.02 | 1.13 ± 0.04 | LC/MS pos | C00328 | HMDB00183 |
| lactate | 1.25 ± 0.05 | 1.10 ± 0.03 | 1.24 ± 0.04 | GC/MS | C00186 | HMDB00190 |
| lathosterol | N/D | N/D | N/D | GC/MS | C01189 | HMDB01170 |
| laurate | 1.25 ± 0.04 | 1.05 ± 0.01 | 1.00 ± 0.02 | LC/MS neg | C02679 | HMDB00638 |
| laurylcarnitine | 1.31 ± 0.05 | 0.92 ± 0.02 | 1.27 ± 0.06 | LC/MS pos | | |
| leucine | 0.81 ± 0.02 | 1.15 ± 0.01 | 0.97 ± 0.02 | LC/MS pos | C00123 | HMDB00687 |
| linoleate | 1.04 ± 0.02 | 0.93 ± 0.01 | 1.06 ± 0.03 | LC/MS neg | C01595 | HMDB00673 |
| linolenate | 1.09 ± 0.03 | 1.12 ± 0.02 | 1.15 ± 0.04 | LC/MS neg | C06427 | HMDB01388 |
| lysine | 1.03 ± 0.02 | 0.99 ± 0.00 | 1.05 ± 0.01 | LC/MS pos | C00047 | HMDB00182 |
| malate | 1.47 ± 0.07 | 1.12 ± 0.02 | 1.26 ± 0.04 | GC/MS | C00149 | HMDB00156 |
| maltose | N/D | 1.24 ± 0.05 | 1.01 ± 0.05 | GC/MS | C00208 | HMDB00163 |
| mannitol | 6.21 ± 1.00 | 4.52 ± 0.35 | 14.79 ± 2.13 | GC/MS | C00392 | HMDB00765 |
| mannose | 1.27 ± 0.04 | 1.30 ± 0.03 | 0.97 ± 0.02 | GC/MS | C00159 | HMDB00169 |
| margarate | 1.15 ± 0.02 | 1.00 ± 0.02 | 1.08 ± 0.04 | LC/MS neg | | HMDB02259 |
| methionine | 1.13 ± 0.02 | 1.07 ± 0.01 | 1.31 ± 0.08 | LC/MS neg | C00073 | HMDB00696 |
| methyl linoleate | 0.83 ± 0.03 | N/D | N/D | GC/MS | | |
| methylglutaroylcarnitine | 2.05 ± 0.12 | 1.29 ± 0.03 | 1.93 ± 0.14 | LC/MS pos | | |
| myo-inositol | 2.72 ± 0.14 | 1.34 ± 0.04 | 1.89 ± 0.11 | GC/MS | C00137 | HMDB00211 |
| myristate | 1.22 ± 0.03 | 1.02 ± 0.01 | 1.08 ± 0.03 | LC/MS neg | C06424 | HMDB00806 |
| myristoleate | 1.28 ± 0.05 | 1.22 ± 0.04 | 1.35 ± 0.06 | LC/MS neg | C08322 | HMDB02000 |
| N2,N2-dimethylguanosine | 1.90 ± 0.15 | 1.75 ± 0.07 | 1.98 ± 0.16 | LC/MS pos | | HMDB04824 |
| N6- | 2.28 ± 0.18 | 1.85 ± 0.07 | 2.01 ± 0.15 | LC/MS pos | | |
| N-acetylalanine | 1.31 ± 0.04 | 1.21 ± 0.02 | 1.42 ± 0.06 | LC/MS neg | C02847 | HMDB00766 |
| N-acetylaspartate | N/D | N/D | N/D | GC/MS | C01042 | HMDB00812 |
| N-acetylglucosamine 6-sulfate | N/D | 1.27 ± 0.04 | 1.69 ± 0.12 | LC/MS neg | C04132 | HMDB00814 |
| N-acetylglycine | 1.33 ± 0.08 | 1.18 ± 0.03 | 1.03 ± 0.05 | GC/MS | | HMDB00532 |
| N-acetylmethionine | 1.40 ± 0.07 | 1.06 ± 0.03 | 1.39 ± 0.07 | LC/MS pos | C02712 | HMDB11745 |
| N-acetylneuraminate | 1.77 ± 0.08 | 1.37 ± 0.05 | 1.70 ± 0.11 | GC/MS | C00270 | HMDB00230 |
| N-acetylornithine | 1.24 ± 0.05 | 0.93 ± 0.02 | 0.92 ± 0.03 | LC/MS pos | C00437 | HMDB03357 |
| N-acetylthreonine | 1.65 ± 0.07* | 1.23 ± 0.03 | 1.72 ± 0.09 | LC/MS neg | | |
| N-formylmethionine | N/D | 0.83 ± 0.02 | 1.14 ± 0.04 | LC/MS neg | C03145 | HMDB01015 |
| nonadecanoate | 1.10 ± 0.02 | 0.99 ± 0.01 | 1.33 ± 0.04 | LC/MS neg | C16535 | HMDB00772 |
| octadecanedioate | 2.14 ± 0.13* | 1.05 ± 0.04 | 4.74 ± 0.77 | LC/MS neg | | HMDB00782 |
| octanoylcarnitine | 2.04 ± 0.09* | 0.94 ± 0.02 | 2.25 ± 0.10* | LC/MS pos | | HMDB00791 |
| oleate | 1.03 ± 0.02 | 0.98 ± 0.02 | 1.13 ± 0.04 | GC/MS | C00712 | HMDB00207 |
| oleoylcarnitine | 0.99 ± 0.04 | 1.18 ± 0.02 | 0.94 ± 0.03 | LC/MS pos | | |
| ornithine | N/D | 1.97 ± 0.07 | 2.04 ± 0.17 | GC/MS | C00077 | HMDB03374 |
| oxaloacetate | 2.42 ± 0.22 | 1.20 ± 0.03 | 1.88 ± 0.13 | GC/MS | C00036 | HMDB00223 |
| p-acetamidophenylglucuronide | 0.82 ± 0.05 | 1.44 ± 0.05 | 0.79 ± 0.06 | LC/MS neg | | |
| palmitate | 1.03 ± 0.02 | 0.92 ± 0.01 | 0.97 ± 0.02 | LC/MS neg | C00249 | HMDB00220 |
| palmitoleate | 1.14 ± 0.04 | 1.18 ± 0.03 | 1.26 ± 0.05 | LC/MS neg | C08362 | HMDB03229 |
| palmitoylcarnitine | 0.93 ± 0.03 | 1.02 ± 0.02 | 0.87 ± 0.03 | LC/MS pos | C02990 | HMDB00222 |
| pantothenate | 2.17 ± 0.18 | 2.53 ± 0.11 | 1.90 ± 0.17 | LC/MS pos | C00854 | HMDB00210 |
| paraxanthine | 1.21 ± 0.11 | 0.98 ± 0.03 | 1.26 ± 0.08 | LC/MS pos | C13747 | HMDB01860 |
| p-cresol sulfate | 1.62 ± 0.11 | 1.49 ± 0.04 | 1.29 ± 0.06 | LC/MS neg | (C01468) | |
| pelargonate | 0.97 ± 0.01 | 1.11 ± 0.01 | 0.84 ± 0.02 | LC/MS neg | C01601 | HMDB00847 |
| pentadecanoate | 1.28 ± 0.03 | 1.11 ± 0.01 | 1.11 ± 0.03 | LC/MS neg | C16537 | HMDB00826 |
| phenol sulfate | 3.26 ± 0.27 | 1.30 ± 0.03 | 2.36 ± 0.20 | LC/MS neg | C02180 | |
| phenylacetate | N/D | 0.76 ± 0.02 | 0.76 ± 0.04 | LC/MS neg | C07086 | HMDB00209 |
| phenylacetylglutamine | 3.37 ± 0.38 | 4.13 ± 0.24 | 3.78 ± 0.51 | LC/MS neg | C05597 | HMDB06344 |
| phenylalanine | 0.95 ± 0.02 | 1.08 ± 0.01 | 1.00 ± 0.02 | LC/MS pos | C00079 | HMDB00159 |
| phenyllactate | 1.49 ± 0.08 | 1.11 ± 0.03 | 1.67 ± 0.11 | LC/MS neg | C05607 | HMDB00779 |
| phosphate | 1.01 ± 0.01 | 1.11 ± 0.01 | 1.03 ± 0.02 | GC/MS | C00009 | HMDB01429 |
| pipecolate | 3.32 ± 0.22* | 1.19 ± 0.03 | 2.78 ± 0.18* | LC/MS pos | C00408 | HMDB00070 |
| piperine | 0.50 ± 0.03* | 1.79 ± 0.06 | 0.56 ± 0.03* | LC/MS pos | C03882 | |
| proline | 1.16 ± 0.05 | 1.01 ± 0.01 | 1.06 ± 0.03 | LC/MS pos | C00148 | HMDB00162 |
| prolylhydroxyproline | 1.56 ± 0.06 | 1.44 ± 0.04 | 1.31 ± 0.05 | LC/MS pos | | HMDB06695 |
| propionylcarnitine | 1.76 ± 0.12 | 1.07 ± 0.02 | 1.36 ± 0.06 | LC/MS pos | C03017 | HMDB00824 |
| pseudouridine | 2.11 ± 0.10 | 1.15 ± 0.03 | 1.45 ± 0.07 | LC/MS pos | C02067 | HMDB00767 |
| pyridoxate | 3.56 ± 0.51 | 7.49 ± 0.59 | 6.38 ± 0.78 | LC/MS neg | C00847 | HMDB00017 |
| pyroglutamine | 1.72 ± 0.07 | 0.94 ± 0.02 | 1.49 ± 0.07 | LC/MS pos | | |
| pyruvate | 1.68 ± 0.09 | 1.18 ± 0.04 | 1.41 ± 0.07 | LC/MS neg | C00022 | HMDB00243 |
| quinate | N/D | N/D | N/D | GC/MS | C00296 | HMDB03072 |
| riboflavin | 1.30 ± 0.10 | N/D | N/D | LC/MS pos | C00255 | HMDB00244 |
| saccharin | N/D | 1.38 ± 0.08 | 20.78 ± 4.19 | LC/MS neg | C12283 | |
| salicylate | 1.29 ± 0.20 | N/D | N/D | LC/MS neg | C00805 | HMDB01895 |
| salicyluric glucuronide | 7.44 ± 0.94 | N/D | N/D | LC/MS neg | | |
| scyllo-inositol | 2.85 ± 0.29* | 0.73 ± 0.02 | 1.65 ± 0.13 | GC/MS | C06153 | HMDB06088 |
| serine | 1.13 ± 0.04 | 1.42 ± 0.04 | 1.12 ± 0.05 | GC/MS | C00065 | HMDB03406 |
| sphingomyelin | 1.03 ± 0.04 | 1.23 ± 0.02 | 1.10 ± 0.03 | GC/MS | C00550 | HMDB01348 |
| sphingosine | 0.77 ± 0.03 | 0.99 ± 0.01 | 0.87 ± 0.04 | LC/MS pos | C00319 | HMDB00252 |
| stachydrine | 5.16 ± 0.47 | 0.96 ± 0.03 | 2.20 ± 0.16 | LC/MS pos | C10172 | HMDB04827 |
| stearate | 1.03 ± 0.01 | 0.97 ± 0.01 | 1.05 ± 0.03 | LC/MS neg | C01530 | HMDB00827 |
| stearidonate | 0.93 ± 0.05 | 0.83 ± 0.02 | 0.81 ± 0.03 | LC/MS neg | C16300 | HMDB06547 |
| stearoylcarnitine | 0.85 ± 0.04 | 0.92 ± 0.02 | 0.75 ± 0.02 | LC/MS pos | | HMDB00848 |
| succinate | 1.03 ± 0.01 | 1.05 ± 0.02 | 0.95 ± 0.02 | GC/MS | C00042 | HMDB00254 |
| succinoylcarnitine | 1.39 ± 0.06 | 1.10 ± 0.02 | 1.28 ± 0.07 | LC/MS pos | | |
| sucrose | 1.83 ± 0.11 | 3.95 ± 0.28 | 3.09 ± 0.41 | LC/MS neg | C00089 | HMDB00258 |
| symmetric dimethylarginine | 1.22 ± 0.04 | 1.09 ± 0.02 | 1.19 ± 0.05 | LC/MS pos | | HMDB03334 |
| taurochenodeoxycholate | 10.74 ± 1.42 | 1.61 ± 0.06 | 4.95 ± 0.55 | LC/MS neg | C05465 | HMDB00951 |
| taurocholate | 8.06 ± 0.95 | 4.07 ± 0.39 | 6.26 ± 0.77 | LC/MS neg | C05122 | HMDB00036 |
| taurolithocholate 3-sulfate | 6.06 ± 0.86 | 1.45 ± 0.05 | 2.78 ± 0.30 | LC/MS neg | C03642 | HMDB02580 |
| tetradecanedioate | 0.93 ± 0.04 | 1.01 ± 0.01 | 1.30 ± 0.04 | LC/MS neg | | HMDB00872 |
| theobromine | 2.05 ± 0.16 | 1.20 ± 0.05 | 2.15 ± 0.23 | LC/MS pos | C07480 | HMDB02825 |
| theophylline | N/D | 0.77 ± 0.03 | 1.18 ± 0.09 | LC/MS neg | C07130 | |
| threitol | 2.28 ± 0.14 | 1.80 ± 0.06 | 2.19 ± 0.16 | GC/MS | | HMDB04136 |
| threonate | 1.40 ± 0.07 | 1.77 ± 0.07 | 1.26 ± 0.07 | GC/MS | C01620 | HMDB00943 |
| threonine | 1.06 ± 0.03 | 1.37 ± 0.03 | 1.42 ± 0.09 | GC/MS | C00188 | HMDB00167 |
| thymol sulfate | N/D | 1.35 ± 0.05 | 1.49 ± 0.15 | LC/MS neg | (C09908) | (HMDB01878) |
| tiglyl carnitine | 1.01 ± 0.04 | 1.12 ± 0.02 | 1.15 ± 0.04 | LC/MS pos | | |
| trigonelline | 1.44 ± 0.13 | 1.40 ± 0.06 | 1.23 ± 0.12 | LC/MS pos | C01004 | HMDB00875 |
| tryptophan | 0.78 ± 0.02 | 1.07 ± 0.01 | 1.03 ± 0.04 | LC/MS pos | C00078 | HMDB00929 |
| tyrosine | 1.04 ± 0.02 | 1.10 ± 0.01 | 1.13 ± 0.03 | LC/MS pos | C00082 | HMDB00158 |
| urate | 1.26 ± 0.03 | 0.98 ± 0.01 | 1.07 ± 0.03 | LC/MS neg | C00366 | HMDB00289 |
| urea | 0.99 ± 0.02 | 1.14 ± 0.02 | 1.49 ± 0.07 | GC/MS | C00086 | HMDB00294 |
| uridine | 0.98 ± 0.02 | 1.08 ± 0.01 | 0.96 ± 0.02 | LC/MS neg | C00299 | HMDB00296 |
| urobilinogen | 2.00 ± 0.16 | N/D | N/D | LC/MS neg | C05791 | HMDB04158 |
| ursodeoxycholate | 1.08 ± 0.07 | N/D | N/D | LC/MS neg | C07880 | HMDB00946 |
| vaccenate | 0.92 ± 0.02 | 1.30 ± 0.02 | 0.88 ± 0.04 | GC/MS | C08367 | |
| valine | 0.87 ± 0.02 | 1.02 ± 0.01 | 0.90 ± 0.02 | LC/MS pos | C00183 | HMDB00883 |
| vanillylmandelate | N/D | N/D | N/D | GC/MS | C05584 | HMDB00291 |
| X-01327 | N/D | N/D | N/D | LC/MS pos | | |
| X-01911 | 0.58 ± 0.03* | 1.26 ± 0.04 | 0.41 ± 0.02* | LC/MS pos | | |
| X-02249 | 1.25 ± 0.08 | 2.06 ± 0.07 | 1.19 ± 0.08 | LC/MS neg | | |
| X-02269 | 0.77 ± 0.03 | 1.58 ± 0.08 | 0.58 ± 0.02 | LC/MS neg | | |
| X-02973 | 1.00 ± 0.02 | 1.11 ± 0.01 | 1.10 ± 0.03 | GC/MS | | |
| X-03002 | N/D | 2.72 ± 0.17 | 1.01 ± 0.07 | GC/MS | | |
| X-03056 | 1.60 ± 0.08 | 1.21 ± 0.03 | 1.32 ± 0.07 | LC/MS pos | | |
| X-03090 | 0.63 ± 0.02 | 1.00 ± 0.03 | 0.79 ± 0.05 | GC/MS | | |
| X-03091 | 0.74 ± 0.04* | 0.81 ± 0.03 | 0.67 ± 0.06 | GC/MS | | |
| X-03094 | 0.89 ± 0.03 | 1.15 ± 0.02 | 0.94 ± 0.03 | GC/MS | | |
| X-03951 | N/D | N/D | N/D | LC/MS pos | | |
| X-04015 | 0.99 ± 0.02 | 0.76 ± 0.02 | 0.62 ± 0.03 | GC/MS | | |
| X-04272 | 0.73 ± 0.02 | 1.01 ± 0.01 | 0.96 ± 0.02 | GC/MS | | |
| X-04357 | 1.26 ± 0.06 | 1.21 ± 0.03 | 1.52 ± 0.06 | GC/MS | | |
| X-04494 | 1.23 ± 0.04 | 0.82 ± 0.01 | 1.14 ± 0.04 | GC/MS | | |
| X-04495 | 1.39 ± 0.06 | 1.10 ± 0.02 | 1.25 ± 0.05 | GC/MS | | |
| X-04498 | 2.27 ± 0.16 | 1.28 ± 0.03 | 1.58 ± 0.08 | GC/MS | | |
| X-04499 | 1.39 ± 0.08 | 1.85 ± 0.09 | 2.47 ± 0.35 | GC/MS | | |
| X-04504 | N/D | 2.85 ± 0.19 | 2.04 ± 0.13 | GC/MS | | |
| X-04507 | 2.50 ± 0.21 | 1.37 ± 0.06 | 1.46 ± 0.10 | GC/MS | | |
| X-04515 | N/D | 0.74 ± 0.03 | 1.54 ± 0.12 | GC/MS | | |
| X-04595 | 1.23 ± 0.05 | 0.80 ± 0.02 | 1.20 ± 0.05 | GC/MS | | |
| X-04598 | 1.41 ± 0.06 | 1.15 ± 0.03 | 1.54 ± 0.07 | GC/MS | | |
| X-04629 | 0.95 ± 0.04 | 1.21 ± 0.06 | 1.08 ± 0.07 | GC/MS | | |
| X-05415 | 1.07 ± 0.09 | 1.86 ± 0.12 | 0.82 ± 0.09 | GC/MS | | |
| X-05426 | 1.52 ± 0.14 | 1.84 ± 0.11 | 1.72 ± 0.16 | GC/MS | | |
| X-05491 | 1.10 ± 0.02 | 1.27 ± 0.04 | 1.15 ± 0.04 | GC/MS | | |
| X-05522 | 1.21 ± 0.07 | 1.22 ± 0.05 | 1.59 ± 0.13 | GC/MS | | |
| X-05907 | 0.62 ± 0.03* | 1.10 ± 0.02 | 0.78 ± 0.03 | GC/MS | | |
| X-06126 | 1.68 ± 0.16 | 1.93 ± 0.07 | 1.46 ± 0.13 | LC/MS neg | | |
| X-06246 | N/D | 0.96 ± 0.01 | 0.79 ± 0.03 | GC/MS | | |
| X-06267 | N/D | 0.94 ± 0.02 | 0.70 ± 0.02 | GC/MS | | |
| X-06268 | N/D | N/D | N/D | GC/MS | | |
| X-06346 | 0.83 ± 0.02 | 1.13 ± 0.02 | 0.94 ± 0.02 | GC/MS | | |
| X-06350 | 0.69 ± 0.02 | 0.99 ± 0.02 | 1.01 ± 0.04 | GC/MS | | |
| X-06351 | N/D | N/D | N/D | GC/MS | | |
| X-06906 | 1.38 ± 0.10 | 1.36 ± 0.06 | 1.33 ± 0.08 | GC/MS | | |
| X-07765 | 1.78 ± 0.13 | 2.07 ± 0.11 | 1.37 ± 0.10 | LC/MS neg | | |
| X-08402 | 0.93 ± 0.03 | 1.23 ± 0.03 | 0.98 ± 0.03 | GC/MS | | |
| X-08889 | 0.78 ± 0.03 | 0.96 ± 0.03 | 0.90 ± 0.04 | GC/MS | | |
| X-08988 | 1.03 ± 0.03 | 1.03 ± 0.01 | 1.08 ± 0.02 | GC/MS | | |
| X-09026 | N/D | 1.13 ± 0.02 | 0.89 ± 0.03 | GC/MS | | |
| X-09044 | N/D | N/D | N/D | GC/MS | | |
| X-09108 | N/D | 1.01 ± 0.01 | 0.79 ± 0.03 | GC/MS | | |
| X-09789 | 1.27 ± 0.08 | 0.90 ± 0.03 | 0.96 ± 0.06 | LC/MS neg | | |
| X-10266 | 0.84 ± 0.04 | 1.09 ± 0.04 | 0.96 ± 0.04 | GC/MS | | |
| X-10346 | N/D | 1.78 ± 0.11 | 7.90 ± 0.92 | LC/MS neg | | |
| X-10359 | 1.62 ± 0.11 | 2.10 ± 0.09 | 2.26 ± 0.17 | GC/MS | | |
| X-10395 | 0.79 ± 0.04 | 1.25 ± 0.02 | 0.85 ± 0.04 | GC/MS | | |
| X-10429 | N/D | 0.87 ± 0.02 | 0.63 ± 0.04 | GC/MS | | |
| X-10438 | N/D | N/D | N/D | GC/MS | | |
| X-10439 | N/D | 0.87 ± 0.02 | 1.06 ± 0.04 | GC/MS | | |
| X-10483 | 1.72 ± 0.10 | 1.04 ± 0.03 | 1.41 ± 0.08 | GC/MS | | |
| X-10500 | 0.89 ± 0.02 | 1.15 ± 0.02 | 1.00 ± 0.02 | GC/MS | | |
| X-10510 | 1.05 ± 0.04 | 1.20 ± 0.03 | 0.99 ± 0.03 | GC/MS | | |
| X-10593 | N/D | 0.89 ± 0.01 | 1.00 ± 0.06 | LC/MS pos | | |
| X-10595 | 0.97 ± 0.02 | N/D | N/D | GC/MS | | |
| X-10609 | 1.03 ± 0.04 | N/D | N/D | GC/MS | | |
| X-10744 | 0.92 ± 0.02 | N/D | N/D | GC/MS | | |
| X-10747 | 3.99 ± 0.78 | 1.32 ± 0.04 | 0.99 ± 0.05 | GC/MS | | |
| X-10752 | 1.02 ± 0.02 | 1.20 ± 0.03 | 1.01 ± 0.04 | GC/MS | | |
| X-10876 | 0.94 ± 0.02 | 1.13 ± 0.01 | 1.00 ± 0.02 | GC/MS | | |
| X-10933 | N/D | 1.10 ± 0.02 | 0.87 ± 0.02 | GC/MS | | |
| X-10964 | 0.59 ± 0.03 | N/D | N/D | GC/MS | | |
| X-11168 | N/D | 0.99 ± 0.04 | 0.81 ± 0.04 | GC/MS | | |
| X-11175 | 1.20 ± 0.06 | 1.14 ± 0.03 | 1.25 ± 0.07 | GC/MS | | |
| X-11204 | 0.90 ± 0.02 | 1.09 ± 0.01 | 0.99 ± 0.01 | LC/MS pos | | |
| X-11206 | N/D | N/D | N/D | LC/MS pos | | |
| X-11231 | N/D | N/D | N/D | LC/MS neg | | |
| X-11244 | 1.69 ± 0.16 | 2.00 ± 0.06 | 1.82 ± 0.14 | LC/MS neg | | |
| X-11245 | 1.44 ± 0.12 | 1.58 ± 0.04 | 1.31 ± 0.07 | LC/MS neg | | |
| X-11255 | 0.74 ± 0.05 | 1.37 ± 0.06 | 0.83 ± 0.07 | LC/MS pos | | |
| X-11261 | 1.45 ± 0.08 | 1.28 ± 0.03 | 1.50 ± 0.09 | LC/MS pos | | |
| X-11273 | 1.09 ± 0.04 | 1.46 ± 0.05 | 1.71 ± 0.12 | LC/MS neg | | |
| X-11282 | 1.67 ± 0.11 | 1.21 ± 0.03 | 1.48 ± 0.07 | LC/MS neg | | |
| X-11299 | 1.40 ± 0.08 | 1.93 ± 0.11 | 1.34 ± 0.08 | LC/MS neg | | |
| X-11302 | 1.62 ± 0.12 | 2.15 ± 0.08 | 1.90 ± 0.16 | LC/MS neg | | |
| X-11303 | 3.42 ± 0.41 | 1.79 ± 0.07 | 2.80 ± 0.32 | LC/MS neg | | |
| X-11308 | 1.03 ± 0.04 | 1.20 ± 0.03 | 1.16 ± 0.07 | LC/MS neg | | |
| X-11315 | 1.14 ± 0.03 | 0.87 ± 0.02 | 0.98 ± 0.03 | LC/MS pos | | |
| X-11317 | N/D | 1.14 ± 0.01 | 0.95 ± 0.02 | LC/MS neg | | |
| X-11327 | 1.00 ± 0.03 | 1.11 ± 0.01 | 1.00 ± 0.01 | LC/MS pos | | |
| X-11333 | 1.77 ± 0.16 | 1.35 ± 0.07 | 1.29 ± 0.12 | LC/MS pos | | |
| X-11334 | 2.20 ± 0.16 | 1.78 ± 0.06 | 1.57 ± 0.11 | LC/MS pos | | |
| X-11341 | 1.45 ± 0.09 | 1.18 ± 0.03 | 1.24 ± 0.05 | LC/MS pos | | |
| X-11372 | 1.01 ± 0.04 | 1.14 ± 0.02 | 1.09 ± 0.05 | LC/MS neg | | |
| X-11381 | 1.27 ± 0.07 | 1.00 ± 0.01 | 1.14 ± 0.04 | LC/MS pos | | |
| X-11400 | N/D | 3.83 ± 0.24 | 1.56 ± 0.11 | LC/MS pos | | |
| X-11412 | N/D | N/D | N/D | LC/MS pos | | |
| X-11421 (an acyl carnitine) | 1.66 ± 0.06* | 0.99 ± 0.02 | 1.91 ± 0.09* | LC/MS pos | | |
| X-11422 | N/D | 1.68 ± 0.08 | 1.17 ± 0.04 | LC/MS neg | | |
| X-11423 | 1.83 ± 0.09 | 1.78 ± 0.06 | 2.20 ± 0.18 | LC/MS neg | | |
| X-11429 | 2.24 ± 0.12 | 1.43 ± 0.05 | 1.83 ± 0.11 | LC/MS neg | | |
| X-11431 | N/D | N/D | N/D | LC/MS neg | | |
| X-11437 | 7.24 ± 1.58 | 6.25 ± 0.44 | 9.55 ± 0.98 | LC/MS neg | | |
| X-11438 | 0.93 ± 0.03 | 1.04 ± 0.02 | 1.30 ± 0.09 | LC/MS neg | | |
| X-11440 | 1.13 ± 0.04 | 2.33 ± 0.08 | 1.24 ± 0.05 | LC/MS neg | | |
| X-11441 | 1.29 ± 0.11 | 1.01 ± 0.03 | 1.46 ± 0.08 | LC/MS neg | | |
| X-11442 | 1.10 ± 0.09 | 0.94 ± 0.03 | 1.36 ± 0.07 | LC/MS neg | | |
| X-11443 | 1.18 ± 0.10 | 1.26 ± 0.04 | 1.80 ± 0.15 | LC/MS neg | | |
| X-11444 | 1.00 ± 0.06 | 1.56 ± 0.04 | 1.79 ± 0.21 | LC/MS neg | | |
| X-11445 | 0.82 ± 0.04 | 1.41 ± 0.05 | 1.51 ± 0.09 | LC/MS neg | | |
| X-11450 | 1.30 ± 0.08 | 1.33 ± 0.03 | 1.25 ± 0.07 | LC/MS neg | | |
| X-11452 | N/D | 1.29 ± 0.05 | 0.42 ± 0.02* | LC/MS neg | | |
| X-11469 | 0.87 ± 0.03 | N/D | N/D | LC/MS pos | | |
| X-11470 | 1.20 ± 0.10 | 1.74 ± 0.07 | 1.77 ± 0.20 | LC/MS neg | | |
| X-11476 | N/D | 1.05 ± 0.01 | 0.84 ± 0.02 | LC/MS pos | | |
| X-11478 | 0.94 ± 0.03 | 1.08 ± 0.03 | 0.89 ± 0.04 | LC/MS neg | | |
| X-11483 | 0.84 ± 0.04 | 0.88 ± 0.03 | 0.71 ± 0.03 | LC/MS neg | | |
| X-11490 | 2.53 ± 0.17 | 1.24 ± 0.04 | 2.22 ± 0.14 | LC/MS neg | | |
| X-11491 | 2.04 ± 0.21 | 1.19 ± 0.03 | 1.50 ± 0.10 | LC/MS neg | | |
| X-11497 | 1.08 ± 0.05 | N/D | N/D | LC/MS neg | | |
| X-11510 | 1.46 ± 0.08 | 1.39 ± 0.06 | 1.22 ± 0.07 | LC/MS neg | | |
| X-11513 | 3.40 ± 0.40 | 0.60 ± 0.02 | 1.41 ± 0.11 | LC/MS pos | | |
| X-11521 | 1.28 ± 0.07 | 1.28 ± 0.03 | 1.50 ± 0.09 | LC/MS pos | | |
| X-11522 | N/D | 1.35 ± 0.04 | 2.50 ± 0.19 | LC/MS neg | | |
| X-11529 | 2.56 ± 0.18 | 2.43 ± 0.12 | 2.50 ± 0.19 | LC/MS neg | | |
| X-11530 | 1.42 ± 0.11 | 1.24 ± 0.03 | 2.71 ± 0.29 | LC/MS neg | | |
| X-11533 | N/D | 1.09 ± 0.01 | 1.00 ± 0.00 | LC/MS neg | | |
| X-11537 | 0.74 ± 0.03 | 0.83 ± 0.02 | 0.66 ± 0.03 | LC/MS pos | | |
| X-11538 | 3.54 ± 0.37* | 1.38 ± 0.07 | 8.24 ± 1.38* | LC/MS neg | | |
| X-11542 | N/D | N/D | N/D | LC/MS pos | | |
| X-11546 | 4.83 ± 0.36* | 1.35 ± 0.09 | 4.60 ± 0.43* | LC/MS neg | | |
| X-11550 | 0.91 ± 0.03 | 1.03 ± 0.01 | 0.83 ± 0.02 | LC/MS neg | | |
| X-11560 | 0.95 ± 0.03 | N/D | N/D | LC/MS neg | | |
| X-11564 | 1.16 ± 0.07 | 1.77 ± 0.06 | 2.20 ± 0.13 | LC/MS neg | | |
| X-11593 | 1.35 ± 0.06 | 1.25 ± 0.03 | 1.17 ± 0.06 | LC/MS neg | | |
| X-11687 | 2.60 ± 0.18 | 1.40 ± 0.06 | 1.54 ± 0.10 | LC/MS pos | | |
| X-11727 | 1.22 ± 0.04 | 1.31 ± 0.04 | 1.08 ± 0.04 | LC/MS pos | | |
| X-11786 | 1.24 ± 0.04 | 1.08 ± 0.02 | 1.18 ± 0.04 | LC/MS pos | | |
| X-11787 | 0.89 ± 0.03 | 1.11 ± 0.01 | 0.81 ± 0.02 | LC/MS pos | | |
| X-11793 | 1.11 ± 0.06 | 1.13 ± 0.02 | 1.33 ± 0.06 | LC/MS pos | | |
| X-11795 | 1.39 ± 0.09 | 1.14 ± 0.03 | 1.66 ± 0.11 | LC/MS pos | | |
| X-11799 | 6.80 ± 0.80 | 0.94 ± 0.03 | 2.78 ± 0.39 | LC/MS pos | | |
| X-11809 | 0.91 ± 0.03 | 1.09 ± 0.01 | 0.86 ± 0.02 | LC/MS pos | | |
| X-11818 | 0.80 ± 0.02 | 1.01 ± 0.01 | 0.97 ± 0.03 | LC/MS pos | | |
| X-11826 | 2.38 ± 0.31 | 3.86 ± 0.23 | 3.43 ± 0.52 | LC/MS neg | | |
| X-11832 | N/D | 1.17 ± 0.07 | 1.03 ± 0.08 | LC/MS neg | | |
| X-11837 | 2.50 ± 0.36 | 1.56 ± 0.09 | 1.41 ± 0.20 | LC/MS pos | | |
| X-11838 | 1.74 ± 0.13 | 3.59 ± 0.16 | 2.34 ± 0.24 | LC/MS neg | | |
| X-11843 | 1.97 ± 0.30 | 1.77 ± 0.09 | 1.80 ± 0.28 | LC/MS neg | | |
| X-11845 | N/D | 1.84 ± 0.19 | 0.57 ± 0.04 | LC/MS neg | | |
| X-11847 | N/D | 2.93 ± 0.20 | 1.52 ± 0.21 | LC/MS neg | | |
| X-11849 | N/D | 5.73 ± 0.64 | 0.49 ± 0.03 | LC/MS neg | | |
| X-11850 | 1.27 ± 0.16 | 1.89 ± 0.10 | 1.12 ± 0.13 | LC/MS neg | | |
| X-11853 | N/D | N/D | N/D | LC/MS neg | | |
| X-11859 | N/D | 1.08 ± 0.01 | 0.87 ± 0.02 | LC/MS neg | | |
| X-11861 | N/D | 1.15 ± 0.02 | 0.98 ± 0.01 | LC/MS neg | | |
| X-11868 | N/D | 0.81 ± 0.01 | 0.78 ± 0.02 | LC/MS neg | | |
| X-11880 | 1.23 ± 0.05 | 1.28 ± 0.03 | 1.22 ± 0.08 | LC/MS neg | | |
| X-11903 | 1.81 ± 0.25 | 1.53 ± 0.09 | 1.64 ± 0.30 | LC/MS neg | | |
| X-11945 | 1.55 ± 0.10 | 1.66 ± 0.06 | 1.57 ± 0.11 | LC/MS pos | | |
| X-11977 | 1.28 ± 0.04 | 0.97 ± 0.02 | 0.75 ± 0.02 | LC/MS pos | | |
| X-12007 | N/D | 2.54 ± 0.17 | 1.26 ± 0.11 | LC/MS neg | | |
| X-12029 | N/D | 1.02 ± 0.01 | 0.93 ± 0.01 | LC/MS neg | | |
| X-12038 | N/D | 1.06 ± 0.02 | 0.87 ± 0.05 | LC/MS neg | | |
| X-12051 | N/D | 1.65 ± 0.04 | 1.30 ± 0.06 | LC/MS pos | | |
| X-12063 | 0.58 ± 0.02 | 1.17 ± 0.03 | 1.36 ± 0.06 | LC/MS neg | | |
| X-12092 | 1.77 ± 0.09 | 1.44 ± 0.04 | 1.64 ± 0.10 | LC/MS pos | | |
| X-12094 | N/D | 1.58 ± 0.05 | 2.23 ± 0.17 | LC/MS pos | | |
| X-12095 | 2.24 ± 0.15 | 1.36 ± 0.04 | 1.87 ± 0.13 | LC/MS pos | | |
| X-12096 | 1.64 ± 0.15 | 1.19 ± 0.05 | 1.11 ± 0.08 | LC/MS pos | | |
| X-12099 | N/D | N/D | N/D | LC/MS pos | | |
| X-12100 | 1.09 ± 0.05 | 1.12 ± 0.03 | 1.17 ± 0.05 | LC/MS pos | | |
| X-12101 | 1.52 ± 0.08 | 1.74 ± 0.06 | 1.33 ± 0.08 | LC/MS pos | | |
| X-12104 | 1.32 ± 0.09 | 1.31 ± 0.03 | 1.41 ± 0.09 | LC/MS pos | | |
| X-12117 | 4.18 ± 0.36 | 2.04 ± 0.10 | 2.08 ± 0.17 | LC/MS pos | | |
| X-12119 | 0.82 ± 0.04 | 1.22 ± 0.04 | 1.09 ± 0.06 | LC/MS pos | | |
| X-12125 | 1.91 ± 0.20 | 1.26 ± 0.08 | 1.91 ± 0.29 | LC/MS pos | | |
| X-12127 | N/D | 1.24 ± 0.04 | 1.03 ± 0.05 | LC/MS pos | | |
| X-12128 | N/D | 0.77 ± 0.01 | 0.88 ± 0.04 | LC/MS pos | | |
| X-12170 | 1.13 ± 0.09 | N/D | N/D | LC/MS pos | | |
| X-12173 | 1.31 ± 0.09 | 1.07 ± 0.04 | 0.79 ± 0.05 | LC/MS pos | | |
| X-12199 | N/D | 1.02 ± 0.02 | 0.60 ± 0.02 | LC/MS pos | | |
| X-12206 | 1.00 ± 0.04 | 1.90 ± 0.08 | 1.74 ± 0.13 | LC/MS neg | | |
| X-12216 | N/D | 0.81 ± 0.04 | 1.59 ± 0.23 | LC/MS neg | | |
| X-12217 | N/D | 4.78 ± 0.34 | 2.67 ± 0.41 | LC/MS neg | | |
| X-12231 | N/D | 1.04 ± 0.04 | 0.35 ± 0.02* | LC/MS neg | | |
| X-12244 | N/D | 1.28 ± 0.03 | 0.83 ± 0.02 | LC/MS pos | | |
| X-12261 | N/D | 1.69 ± 0.09 | 1.64 ± 0.21 | LC/MS neg | | |
| X-12262 | N/D | 1.06 ± 0.03 | 1.73 ± 0.20 | LC/MS neg | | |
| X-12358 | 2.95 ± 0.44 | 1.44 ± 0.06 | 1.72 ± 0.17 | LC/MS pos | | |
| X-12405 | 1.58 ± 0.11 | 2.19 ± 0.11 | 1.34 ± 0.09 | LC/MS neg | | |
| X-12421 | N/D | N/D | N/D | LC/MS pos | | |
| X-12422 | N/D | 0.76 ± 0.02 | 0.61 ± 0.05 | LC/MS pos | | |
| X-12428 | 1.29 ± 0.15 | 2.09 ± 0.11 | 1.91 ± 0.24 | LC/MS neg | | |
| X-12440 | N/D | 1.48 ± 0.03 | 0.91 ± 0.04 | LC/MS neg | | |
| X-12442 | 1.21 ± 0.03 | 1.28 ± 0.05 | 1.70 ± 0.12 | LC/MS neg | | |
| X-12443 | N/D | 0.61 ± 0.02 | 0.62 ± 0.03 | LC/MS neg | | |
| X-12450 | N/D | 1.10 ± 0.01 | 1.07 ± 0.02 | LC/MS neg | | |
| X-12458 | N/D | 0.89 ± 0.02 | 1.36 ± 0.06 | LC/MS pos | | |
| X-12459 | 1.10 ± 0.10 | N/D | N/D | LC/MS pos | | |
| X-12465 (an acyl carnitine) | 1.68 ± 0.10 | 1.47 ± 0.05 | 1.78 ± 0.12 | LC/MS pos | | |
| X-12510 | 1.26 ± 0.05 | 1.17 ± 0.03 | 1.63 ± 0.11 | LC/MS pos | | |
| X-12537 | 1.04 ± 0.06 | N/D | N/D | GC/MS | | |
| X-12542 | N/D | N/D | N/D | LC/MS pos | | |
| X-12556 | 1.11 ± 0.03 | 1.06 ± 0.02 | 1.23 ± 0.05 | GC/MS | | |
| X-12611 | N/D | N/D | N/D | LC/MS pos | | |
| X-12644 | 1.10 ± 0.04 | 1.18 ± 0.02 | 1.13 ± 0.05 | LC/MS neg | | |
| X-12660 | N/D | 2.33 ± 0.09 | 1.45 ± 0.10 | LC/MS pos | | |
| X-12681 | 1.19 ± 0.07 | 0.82 ± 0.01 | 1.13 ± 0.06 | LC/MS pos | | |
| X-12683 | 1.57 ± 0.14 | N/D | N/D | LC/MS pos | | |
| X-12686 | 1.14 ± 0.06 | 0.88 ± 0.02 | 0.68 ± 0.03 | LC/MS pos | | |
| X-12688 | 1.92 ± 0.16 | 1.35 ± 0.07 | 1.05 ± 0.07 | LC/MS pos | | |
| X-12690 | 1.30 ± 0.06 | 0.82 ± 0.02 | 1.06 ± 0.04 | LC/MS pos | | |
| X-12695 | N/D | N/D | N/D | LC/MS neg | | |
| X-12707 | N/D | 1.59 ± 0.09 | 2.03 ± 0.14 | LC/MS neg | | |
| X-12728 | N/D | 0.96 ± 0.02 | 0.96 ± 0.02 | LC/MS neg | | |
| X-12739 | 1.37 ± 0.14 | N/D | N/D | LC/MS neg | | |
| X-12742 | 1.82 ± 0.15 | 2.13 ± 0.10 | 2.27 ± 0.26 | LC/MS neg | | |
| X-12749 | 1.88 ± 0.08 | 1.36 ± 0.04 | 1.72 ± 0.09 | LC/MS pos | | |
| X-12756 | N/D | 2.57 ± 0.14 | 1.40 ± 0.11 | LC/MS pos | | |
| X-12765 | 2.34 ± 0.21 | 2.20 ± 0.16 | 1.41 ± 0.12 | LC/MS pos | | |
| X-12775 | 1.45 ± 0.09 | 1.57 ± 0.05 | 1.41 ± 0.10 | LC/MS pos | | |
| X-12776 | 1.06 ± 0.01 | 1.03 ± 0.00 | 1.01 ± 0.01 | LC/MS neg | | |
| X-12786 | 0.89 ± 0.05 | 1.10 ± 0.03 | 1.42 ± 0.05 | GC/MS | | |
| X-12792 | N/D | N/D | N/D | LC/MS pos | | |
| X-12794 | N/D | 0.84 ± 0.04 | 0.83 ± 0.06 | LC/MS pos | | |
| X-12802 | 2.66 ± 0.16 | 1.27 ± 0.05 | 2.13 ± 0.14 | LC/MS pos | | |
| X-12822 | N/D | 1.24 ± 0.04 | 1.46 ± 0.07 | LC/MS neg | | |
| X-12824 | 1.14 ± 0.07 | 1.19 ± 0.05 | 1.61 ± 0.14 | LC/MS neg | | |
| X-12844 | 1.13 ± 0.04 | 1.76 ± 0.05 | 1.61 ± 0.14 | LC/MS neg | | |
| X-12846 | 1.88 ± 0.18 | 2.09 ± 0.10 | 2.06 ± 0.20 | LC/MS neg | | |
| X-12847 | N/D | 1.20 ± 0.05 | 0.72 ± 0.03 | LC/MS neg | | |
| X-12849 | N/D | N/D | N/D | LC/MS neg | | |
| X-12850 | 5.51 ± 0.42 | 2.16 ± 0.13 | 3.17 ± 0.18 | LC/MS neg | | |
| X-12851 | N/D | N/D | N/D | LC/MS neg | | |
| X-12855 | 1.20 ± 0.07 | 0.96 ± 0.02 | 1.40 ± 0.07 | LC/MS pos | | |
| X-12860 | 1.28 ± 0.07 | 0.99 ± 0.02 | 1.75 ± 0.13 | LC/MS pos | | |
| X-12990 | 0.83 ± 0.05 | 0.97 ± 0.01 | 0.92 ± 0.04 | LC/MS neg | | |
| X-13152 | 1.89 ± 0.16 | 0.91 ± 0.02 | 1.55 ± 0.10 | LC/MS pos | | |
| X-13429 | 3.32 ± 0.36 | 1.86 ± 0.12 | 1.53 ± 0.11 | LC/MS neg | | |
| X-13435 | N/D | 0.99 ± 0.02 | 1.91 ± 0.09* | LC/MS pos | | |
| X-13465 | N/D | N/D | N/D | LC/MS neg | | |
| X-13543 | 0.56 ± 0.01 | N/D | N/D | LC/MS pos | | |
| X-13553 | 1.78 ± 0.09 | 2.09 ± 0.10 | 4.01 ± 0.26 | LC/MS neg | | |
| X-13619 | 0.98 ± 0.03 | 1.03 ± 0.01 | 0.92 ± 0.02 | GC/MS | | |
| X-13684 | N/D | 0.87 ± 0.02 | 0.89 ± 0.04 | LC/MS pos | | |
| X-13687 | 1.25 ± 0.13 | 1.55 ± 0.06 | 1.67 ± 0.16 | LC/MS pos | | |
| X-13727 | N/D | 1.98 ± 0.10 | 1.17 ± 0.07 | LC/MS neg | | |
| X-13751 | N/D | N/D | N/D | LC/MS pos | | |
| X-13852 | 3.37 ± 0.26 | 1.58 ± 0.05 | 2.23 ± 0.17 | LC/MS pos | | |
| X-13871 | 1.28 ± 0.07 | 1.08 ± 0.03 | 1.28 ± 0.09 | LC/MS pos | | |
| X-14056 | 1.32 ± 0.07 | 0.96 ± 0.02 | 1.17 ± 0.05 | LC/MS pos | | |
| X-14091 | 1.60 ± 0.08 | N/D | N/D | LC/MS neg | | |
| X-14318 | 0.89 ± 0.07 | N/D | N/D | LC/MS pos | | |
| X-14473 | 1.22 ± 0.08 | 1.02 ± 0.02 | 1.11 ± 0.07 | LC/MS pos | | |
| X-14561 | 0.83 ± 0.02 | N/D | N/D | LC/MS neg | | |
| X-14584 | 1.37 ± 0.06 | 0.90 ± 0.03 | 0.81 ± 0.04 | LC/MS neg | | |
| X-14588 | 1.04 ± 0.01 | 1.01 ± 0.01 | 1.06 ± 0.01 | LC/MS neg | | |
| X-14625 | N/D | 1.09 ± 0.01 | 1.14 ± 0.03 | LC/MS neg | | |
| X-14626 | 4.29 ± 0.64 | 2.29 ± 0.16 | 3.15 ± 0.39 | LC/MS neg | | |
| X-14632 | N/D | 0.98 ± 0.04 | 1.16 ± 0.07 | LC/MS neg | | |
| X-14658 | 10.16 ± 1.16 | 2.06 ± 0.11 | 6.48 ± 0.52 | LC/MS neg | | |
| X-14662 | 6.92 ± 0.56* | 1.13 ± 0.05 | 4.43 ± 0.37* | LC/MS neg | | |
| X-14663 | 5.73 ± 0.54* | 1.46 ± 0.09 | 7.65 ± 0.71* | LC/MS neg | | |
| X-14837 | 1.00 ± 0.06 | 3.01 ± 0.40 | 1.43 ± 0.13 | LC/MS pos | | |
| X-14842 | 0.91 ± 0.04 | N/D | N/D | GC/MS | | |
| xanthine | 0.86 ± 0.02 | 1.24 ± 0.04 | 0.88 ± 0.03 | LC/MS pos | C00385 | HMDB00292 |
| xylonate | N/D | 1.79 ± 0.07 | 1.79 ± 0.13 | GC/MS | C00502 | |
| xylose | N/D | 1.32 ± 0.04 | 1.23 ± 0.08 | GC/MS | C00181 | HMDB00098 |

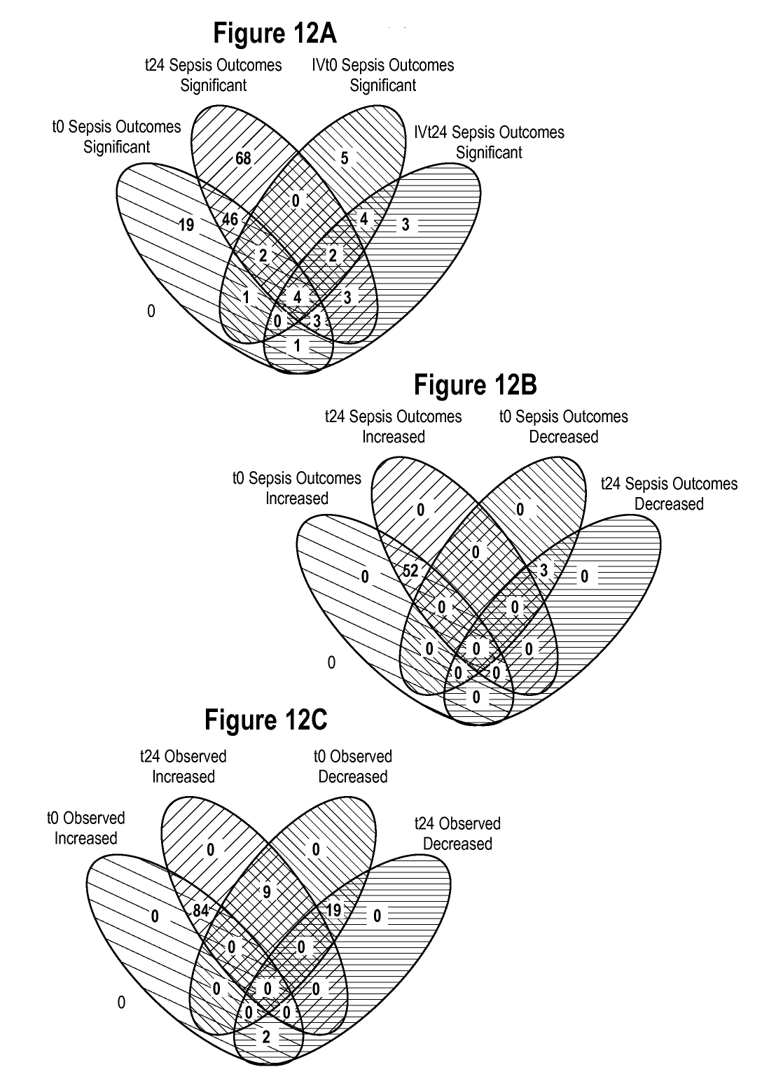

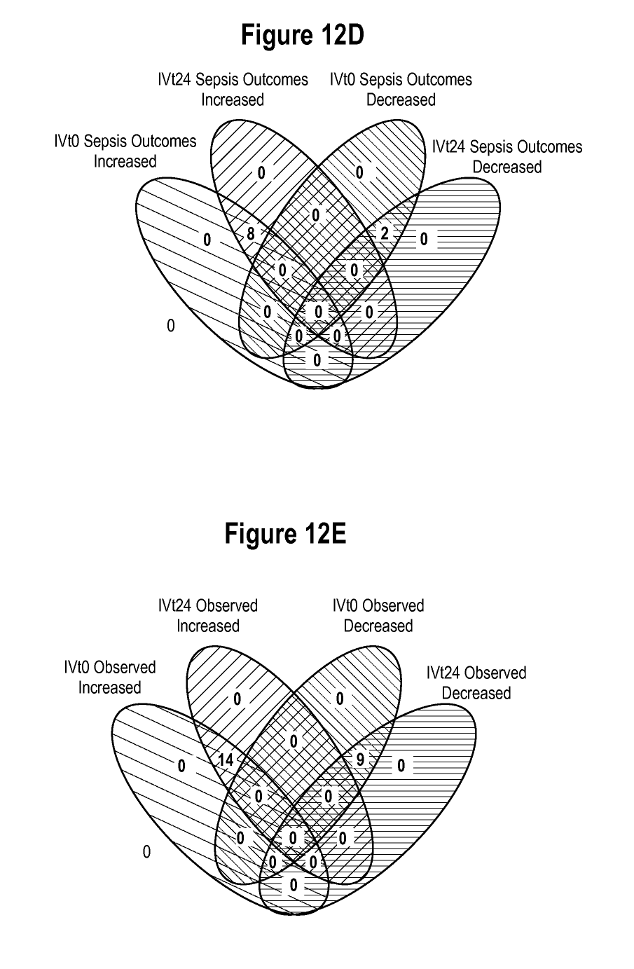

The metabolic differences of sepsis survivors from controls were reversed in sepsis deaths. 76 plasma metabolites differed between sepsis survivors and deaths at t₀, increasing to 128 at t₂₄ (FDR 5%; FIG. 2a; FIGS. 11 and 12; Tables 9, 10). Metabolic divergence of sepsis survivors and deaths was temporally consistent—84 metabolites that were significant at one time point and detected at the other had concordant direction of change. Inter-individual variability in individual metabolites was high. The significance of the biochemical differences detected, however, was strengthened by finding multiple related metabolites exhibiting the same pattern of change, including 17 amino acid catabolites, 16 carnitine esters, 11 nucleic acid catabolites, 5 glycolysis and citric acid cycle components (citrate and malate, pyruvate, dihydroxyacetone, phosphate) and 4 fatty acids (FA); FIG. 11). All were elevated in sepsis deaths (by ANOVA). In contrast, 7 acyl-GPC/E were decreased in sepsis survivors and more so in sepsis deaths, in agreement with previous studies. Lactate, an established sepsis severity marker, was elevated in sepsis death. Carnitine and ketones were unchanged. A clinical correlate of depressed exergonic metabolism in sepsis deaths was significantly lower core temperature than survivors (Table 5), as previously described. Given their role in metabolic regulation, it was notable that anabolic steroids were decreased in sepsis deaths while cortisone was increased.

### Example 3

### Validation of Metabolomic Findings

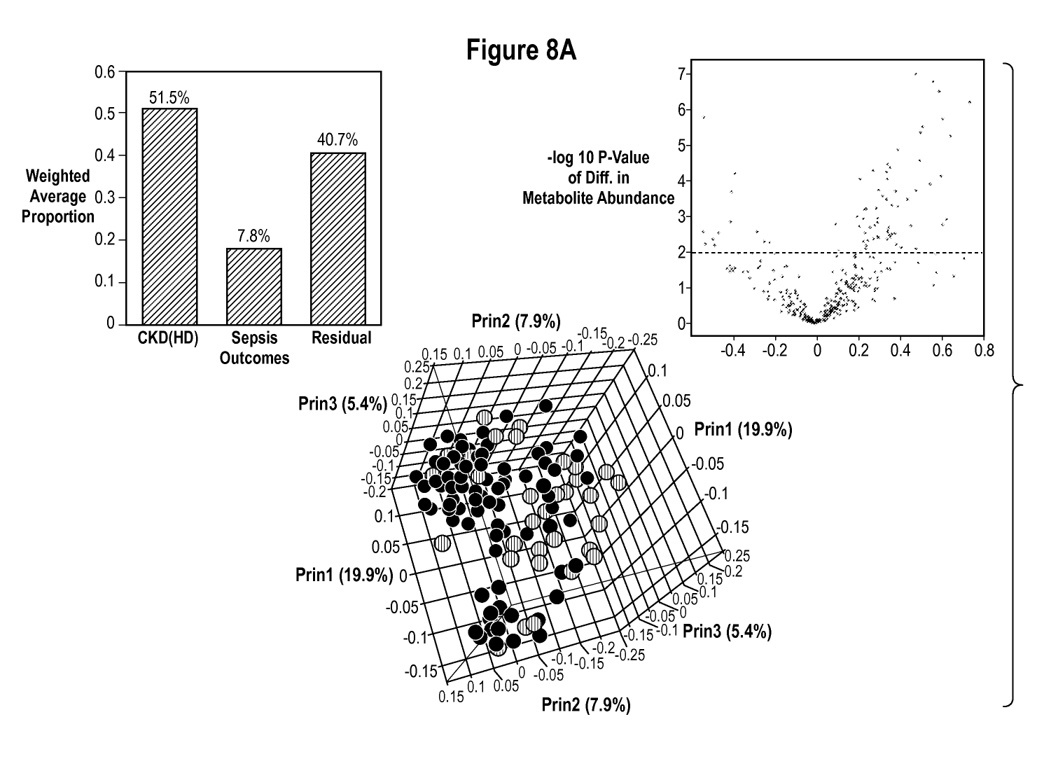

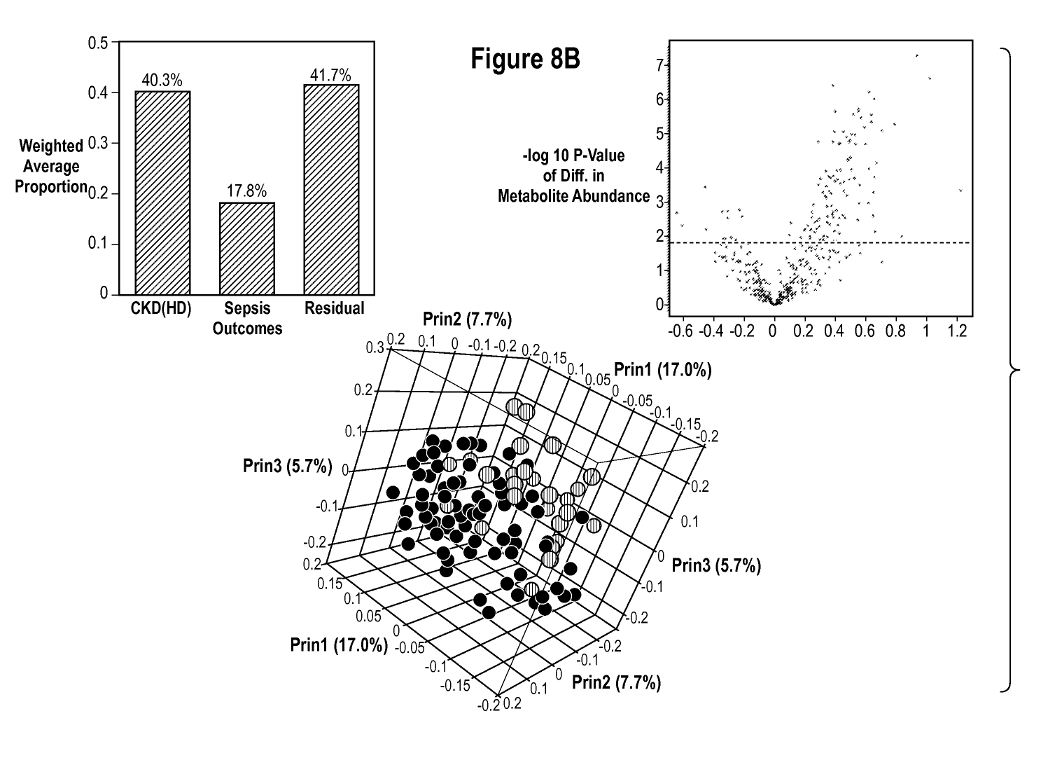

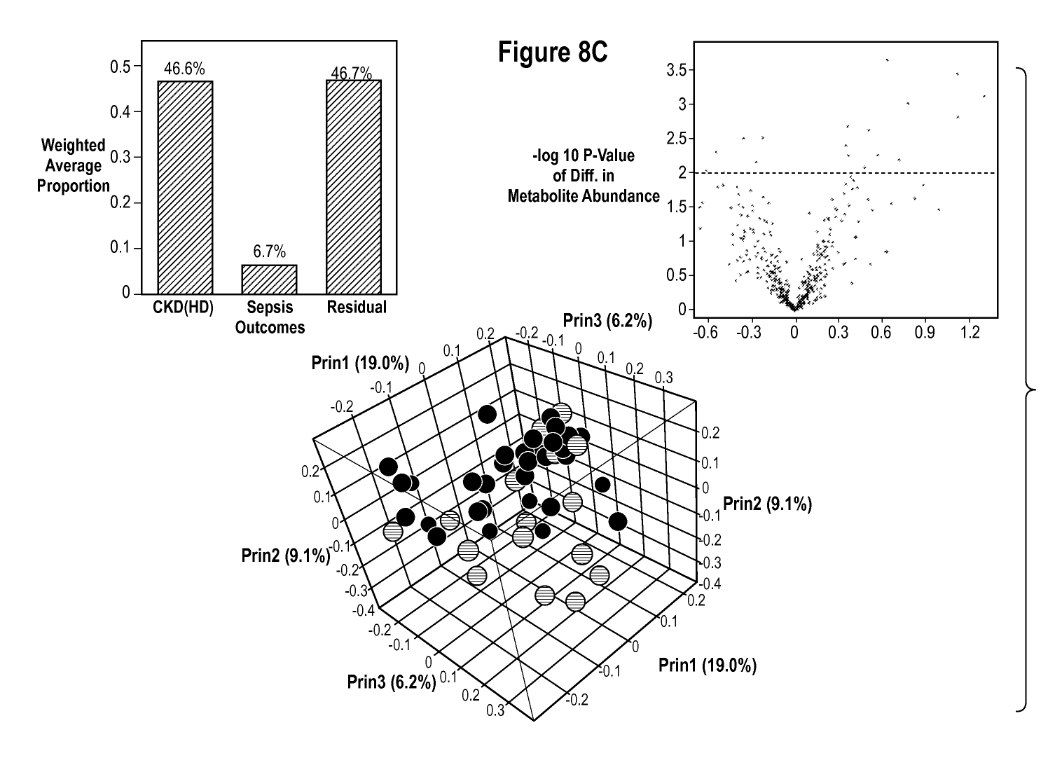

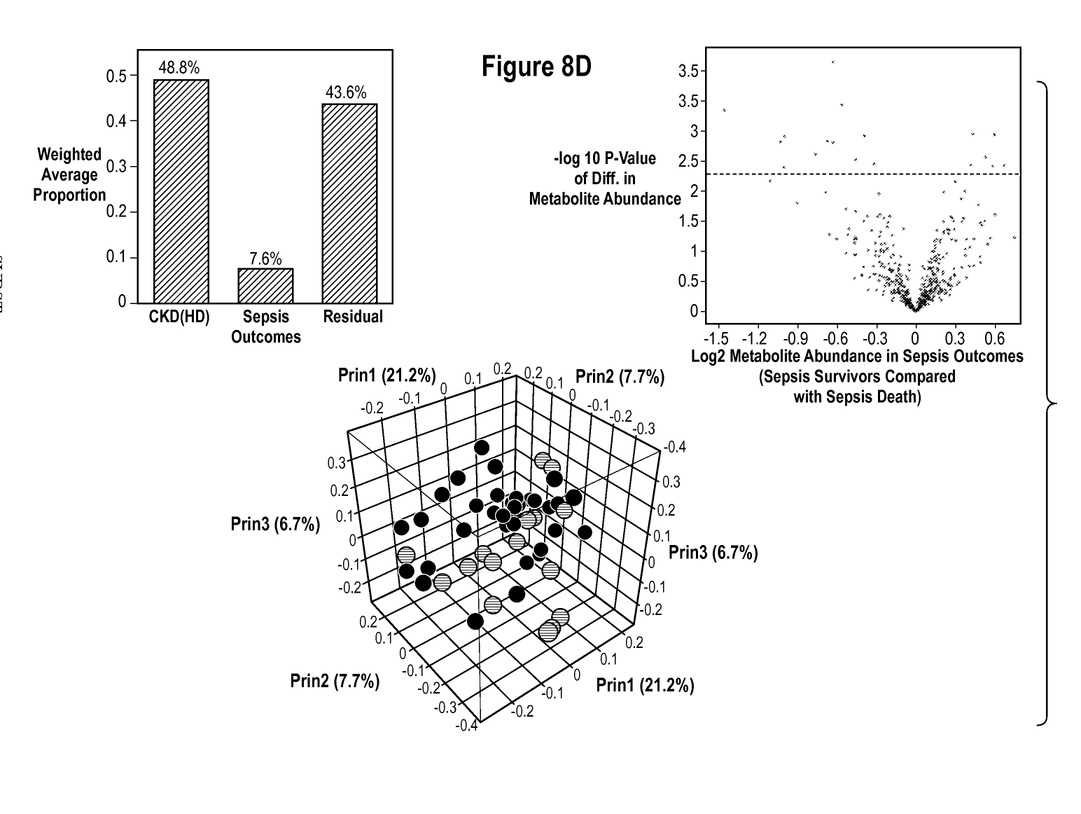

Plasma metabolites were assayed in all remaining CAPSOD sepsis deaths (n=18) and 34 additional, matched sepsis survivors to seek confirmation of the discovery findings. (FIG. 3). The median time-to-death of the validation group was much longer than the discovery group (18.5 days vs. 10.7 days, respectively), and the metabolic variance attributable to sepsis outcome was less (FIG. 8). Consequently, the validation cohort exhibited fewer differences and of smaller magnitude between sepsis survivors and deaths (18 differences at t₀ and 20 at t₂₄; FIG. 11, 12; Tables 9, 10 and 11). Nevertheless, the major discovery cohort findings were recapitulated (elevated amino acid and RNA catabolites, citrate, malate and fatty acids, decreased anabolic steroids and GPC esters). The most consistently altered biochemical class was carnitine esters, with significant increases in 19 of 21 compounds in sepsis death in at least one time point.

TABLE 11
Concordant differences between sepsis deaths and survivors
at t₂₄ in the discovery set and t₀ in the replication set

| | | Discovery t₂₄ | Replication t₀ |
|---|---|---|---|
| | | Fold Change | Fold Change |
| | Metabolic | Sepsis Death | Sepsis Death |
| Biochemical | Pathway | (vs. Survival) | (vs. Survival) |
| propionylcarnitine | Amino acid | 1.69 | 1.16 |
| (C3) | metabolism | | |
| butyrylcarnitine | Amino acid | 1.61 | 1.42 |
| (C4) | metabolism | | |
| 2-methylbutyroyl- | Amino acid | 2.12 | 1.07 |
| carnitine (C5) | metabolism | | |
| hydroxyisovaleroyl- | Amino acid | 1.39 | 1.10 |
| carnitine (C5) | metabolism | | |
| Pyruvate | Glycolysis, | 1.61 | 1.06 |
| | gluconeogenesis | | |
| Lactate | Anaerobic | 1.40 | 1.06 |
| | glycolysis | | |
| Malate | Krebs cycle | 1.40 | 1.13 |
| Phosphate | Oxidative | 1.15 | 1.01 |
| | phosphorylation | | |
| 3-hydroxydecanoate | Fatty acid | 2.30 | 1.07 |
| hexadecanedioate | Fatty acid | 3.00 | 1.39 |
| (C16) | | | |
| octadecanedioate | Fatty acid | 3.81 | 1.56 |
| (C18) | | | |
| acetylcarnitine (C2) | Fatty acid | 1.75 | 1.20 |
| | metabolism | | |
| hexanoylcarnitine | Fatty acid | 1.98 | 1.32 |
| (C6) | metabolism | | |
| octanoylcarnitine | Fatty acid | 2.46 | 1.42 |
| (C8) | metabolism | | |
| glycerophos- | Glycerolipid | 0.59 | 0.97 |
| phorylcholine | metabolism, | | |
| (GPC) | immune function | | |
| 1-arachidoyl-GPE* | Glycerolipid | 0.54 | 0.96 |
| (20:4) | metabolism, | | |
| | immune function | | |
| 1-palmitoyl-GPC | Glycerolipid | 0.69 | 0.85 |
| (16:0) | metabolism, | | |
| | immune function | | |
| 1-stearoyl-GPC | Glycerolipid | 0.65 | 0.84 |
| (18:0) | metabolism, | | |
| | immune function | | |
| 2-stearoyl-GPC* | Glycerolipid | 0.44 | 0.80 |
| (18:0) | metabolism, | | |
| | immune function | | |
| 1-eicosatrienoyl- | Glycerolipid | 0.34 | 0.81 |
| GPC* (20:3) | metabolism, | | |
| | immune function | | |
| 1-arachidoyl-GPC* | Glycerolipid | 0.52 | 0.78 |
| (20:4) | metabolism, | | |
| | immune function | | |
| Piperine | Food component/ | 0.29 | 0.66 |
| | Plant | | |

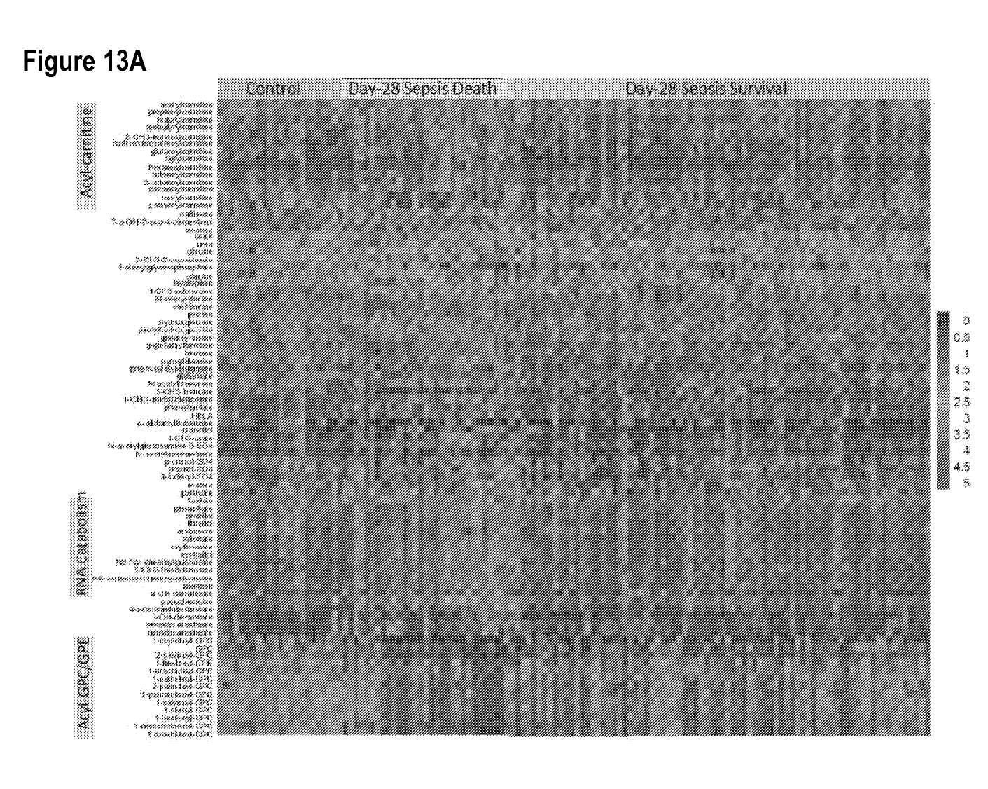

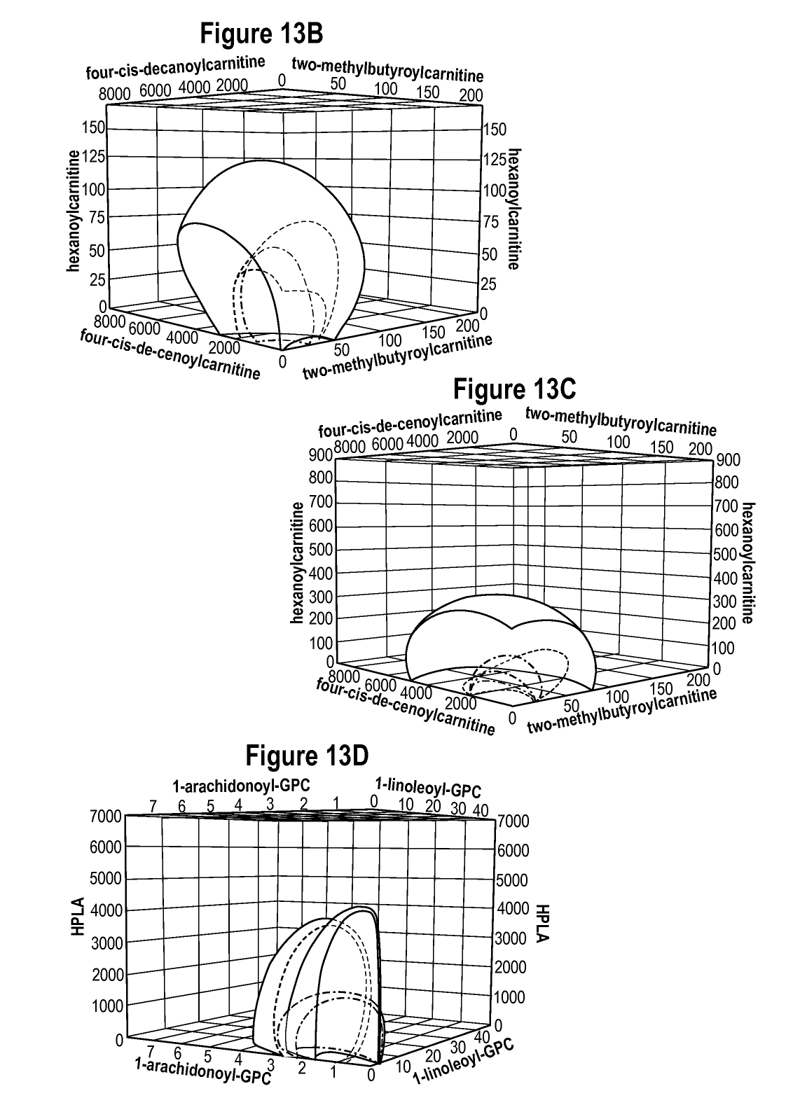

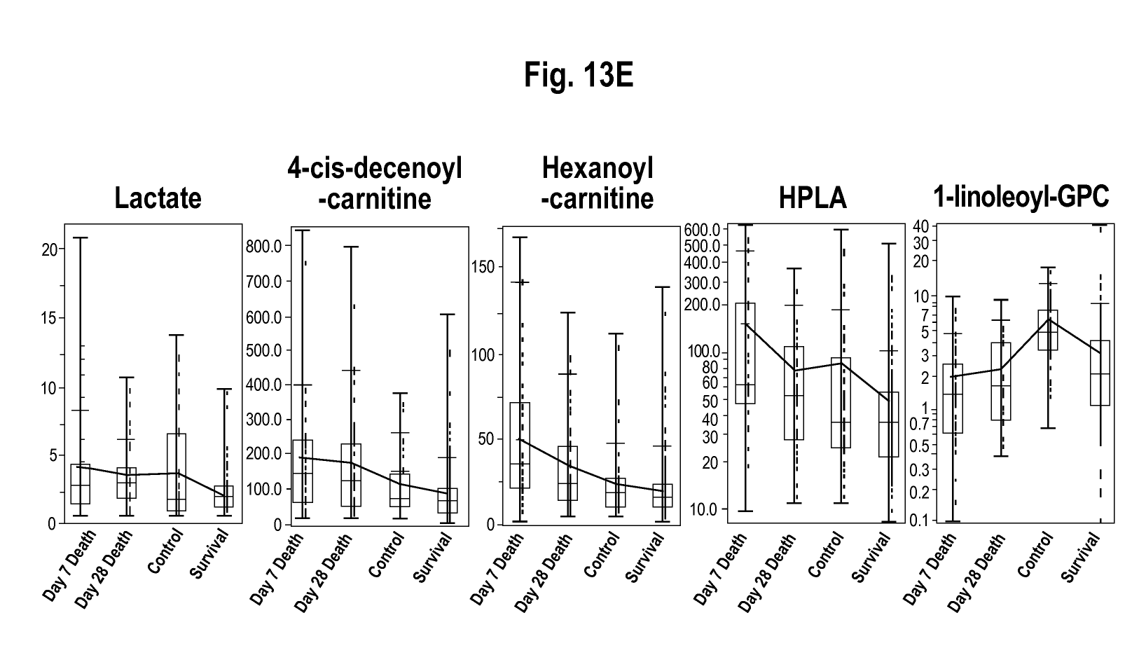

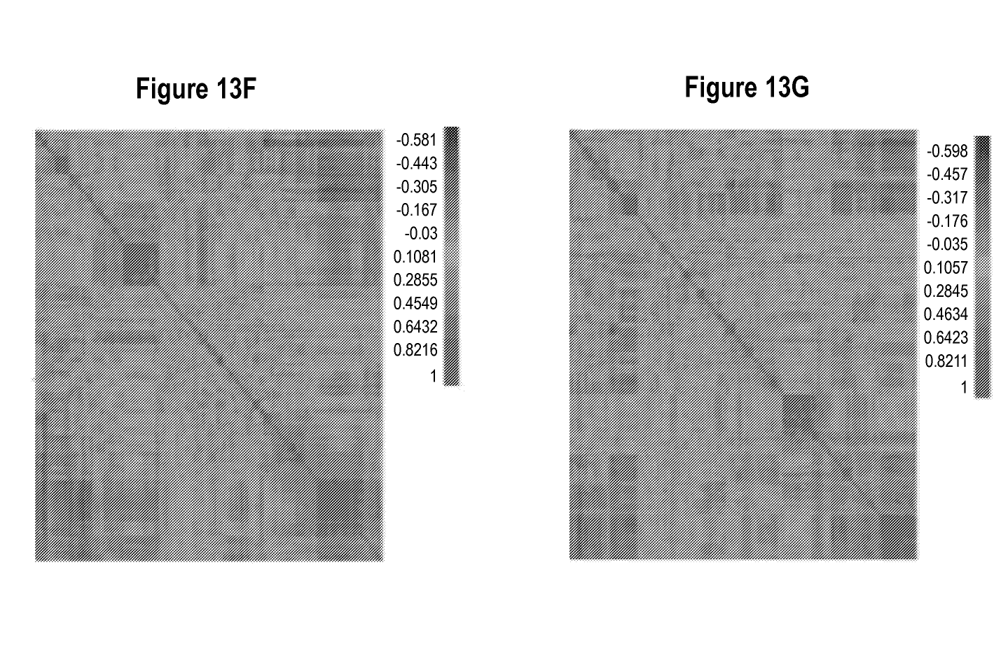

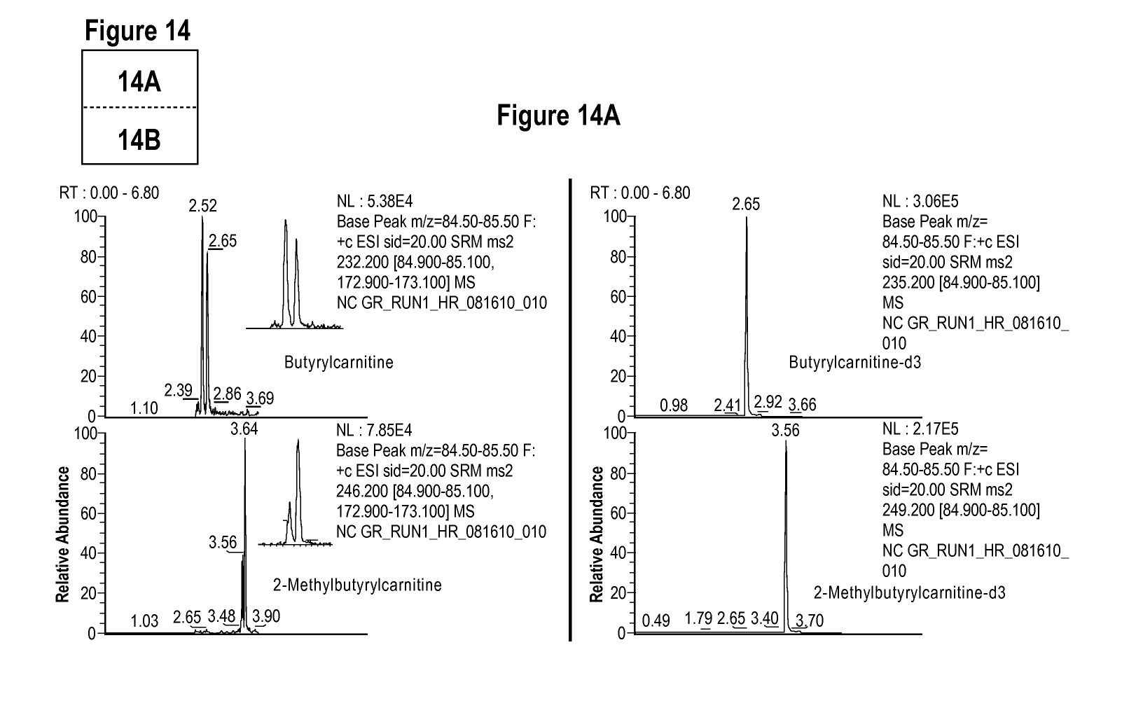

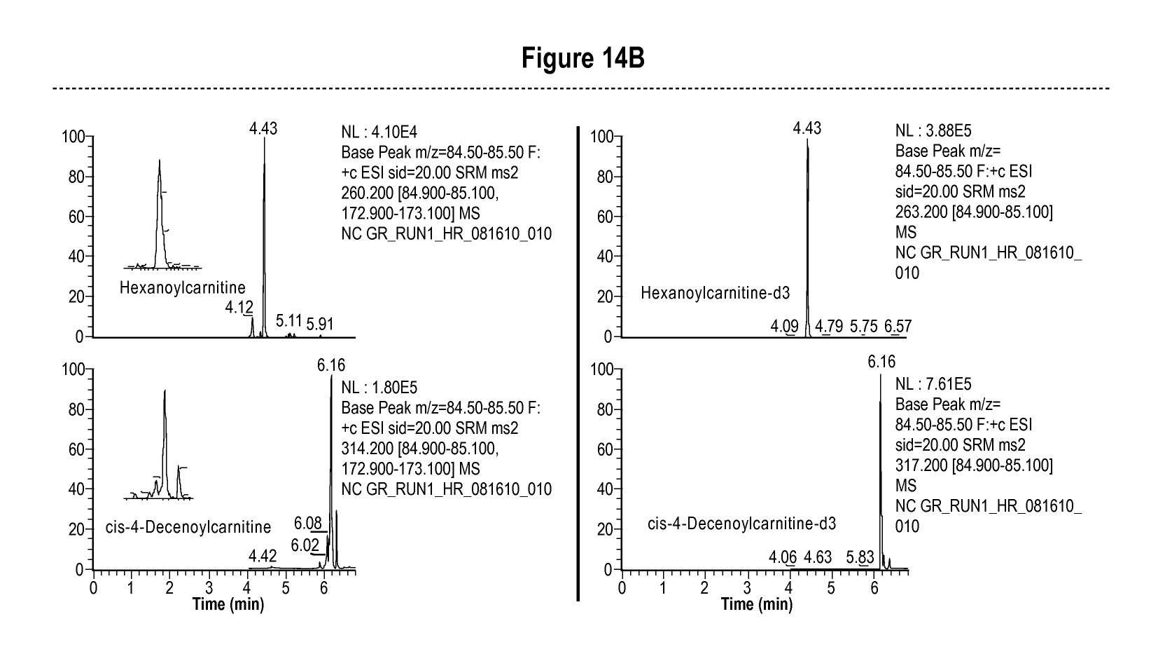

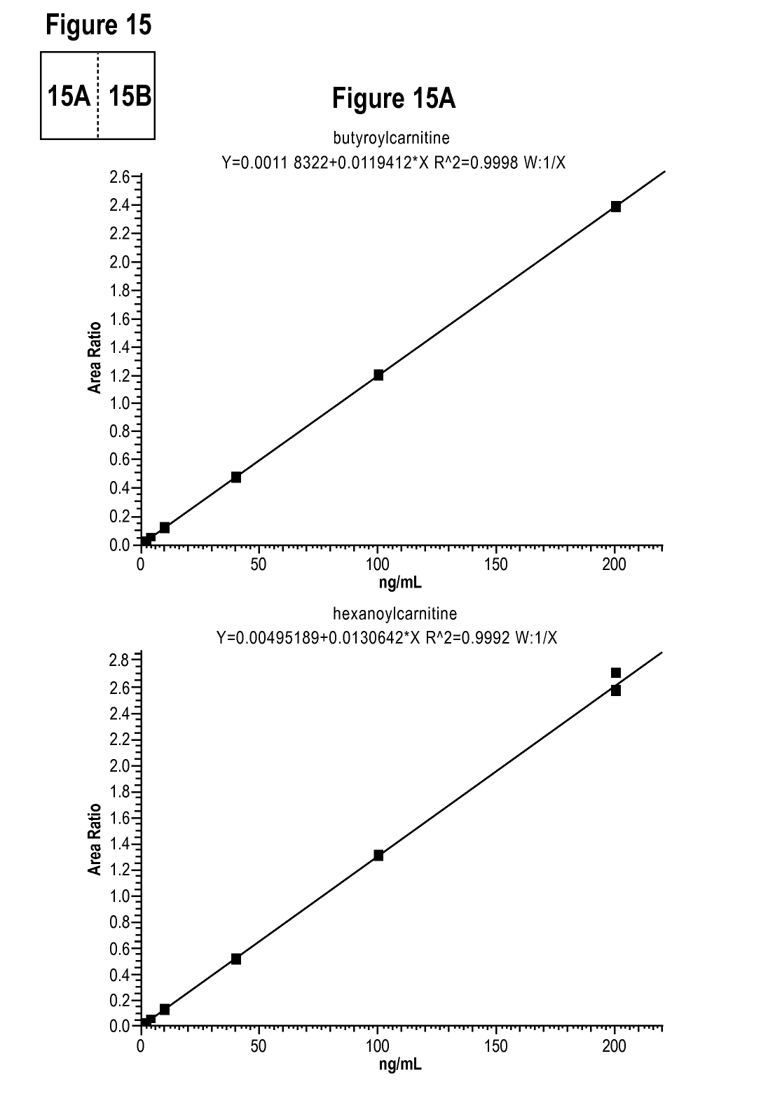

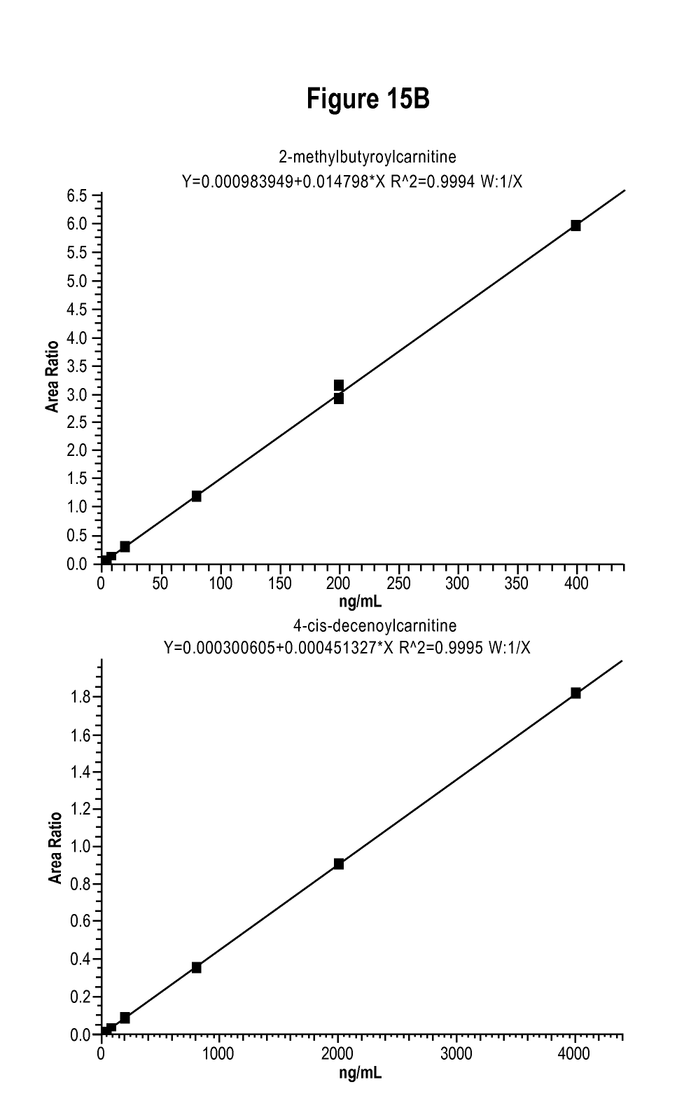

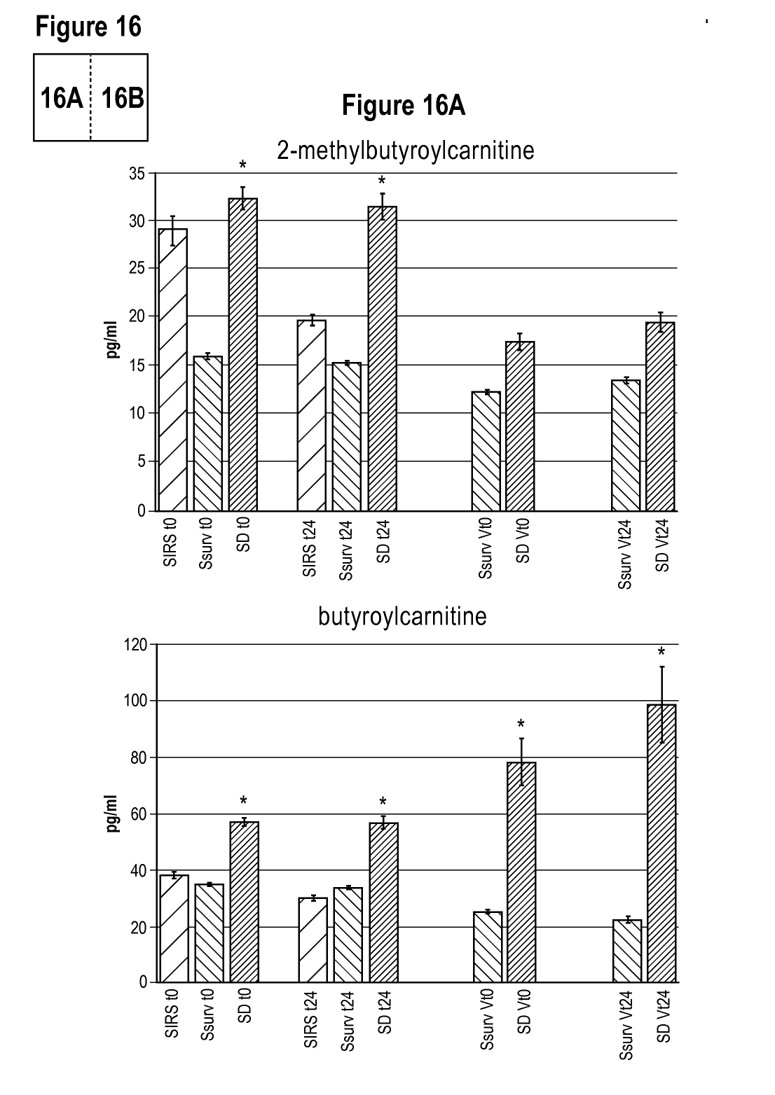

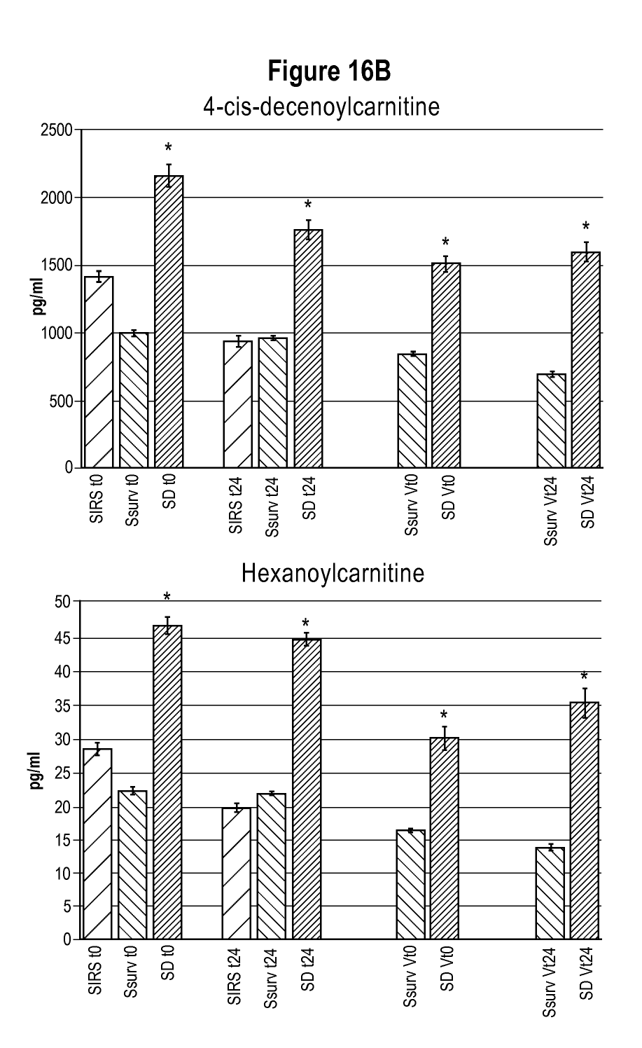

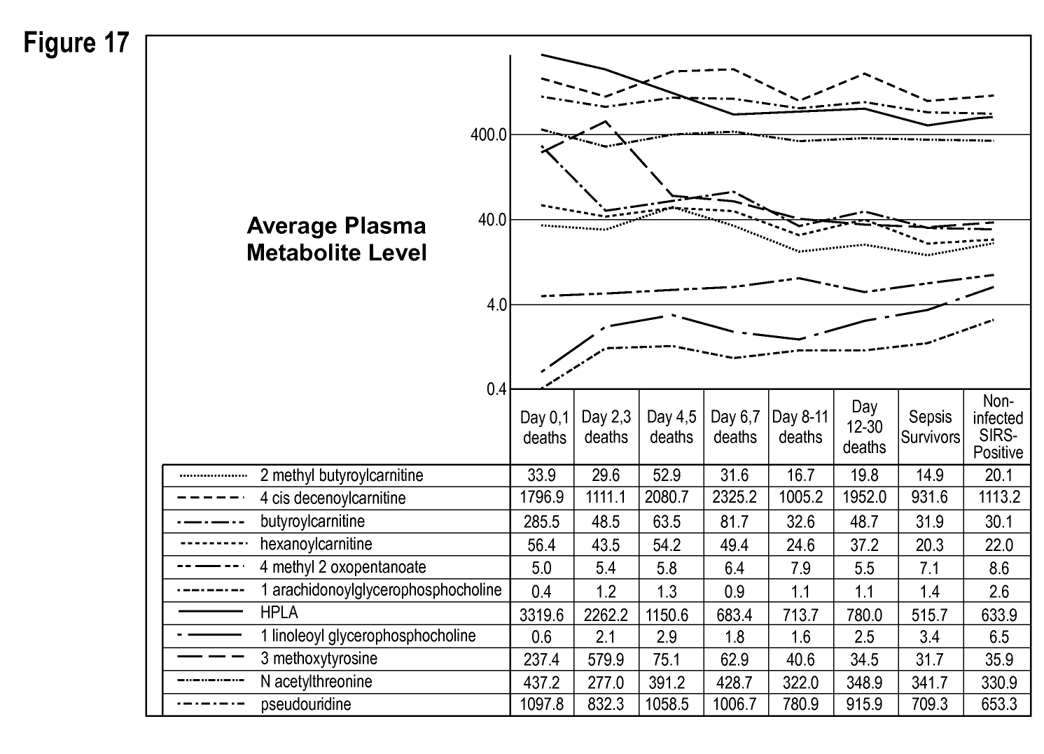

Additional validation was obtained by retesting all 393 samples using targeted, quantitative assays of 11 metabolites representative of the major findings. While inter-individual variability was considerable, the differences between sepsis survivor, sepsis death and control groups were confirmed (FIG. 13b-e, FIGS. 14-17). The average differences between sepsis survivors and deaths increased inversely with time-to-death, suggesting a causal relationship between metabolic perturbation and sepsis death (FIG. 17).

### Example 4

### Plasma Proteomics

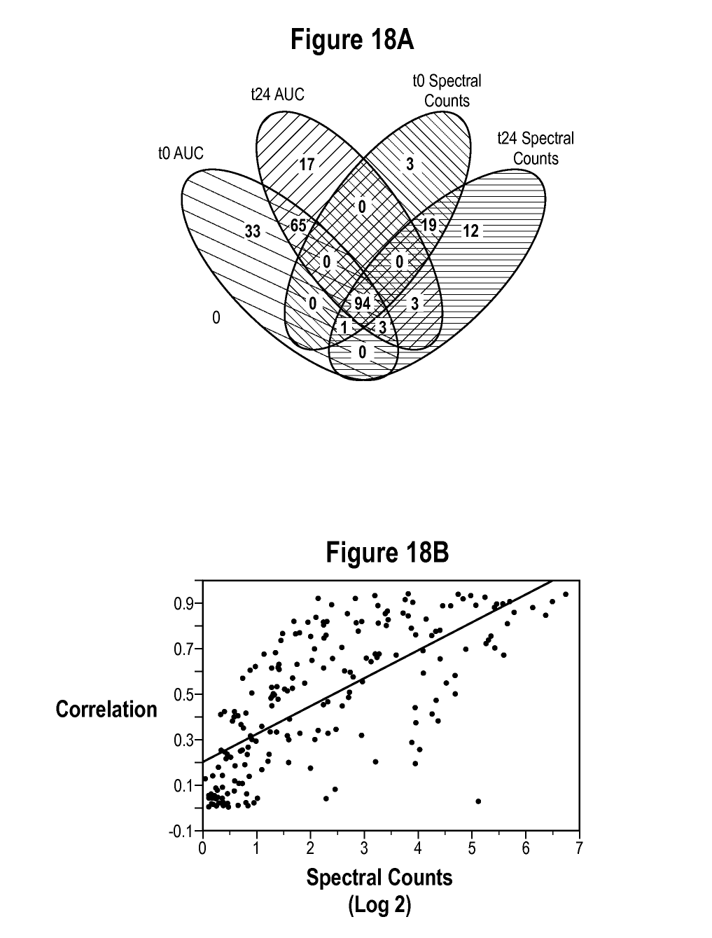

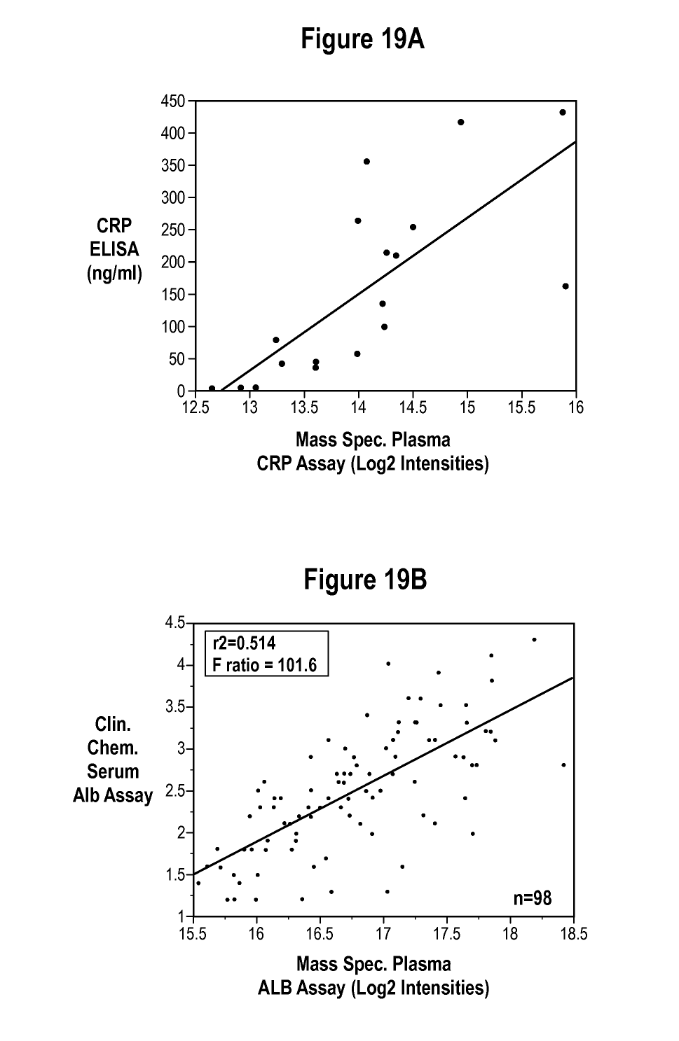

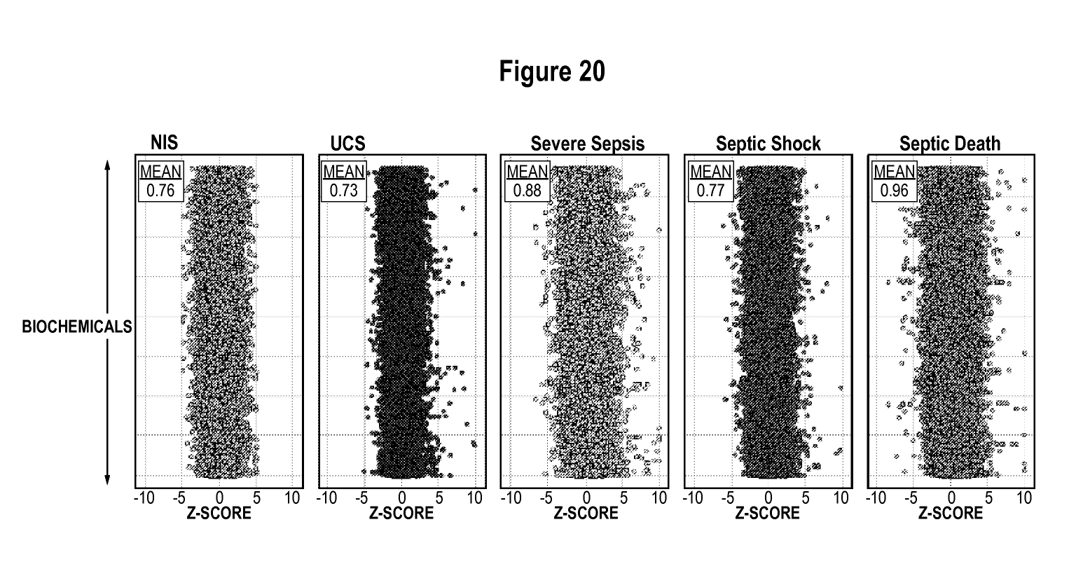

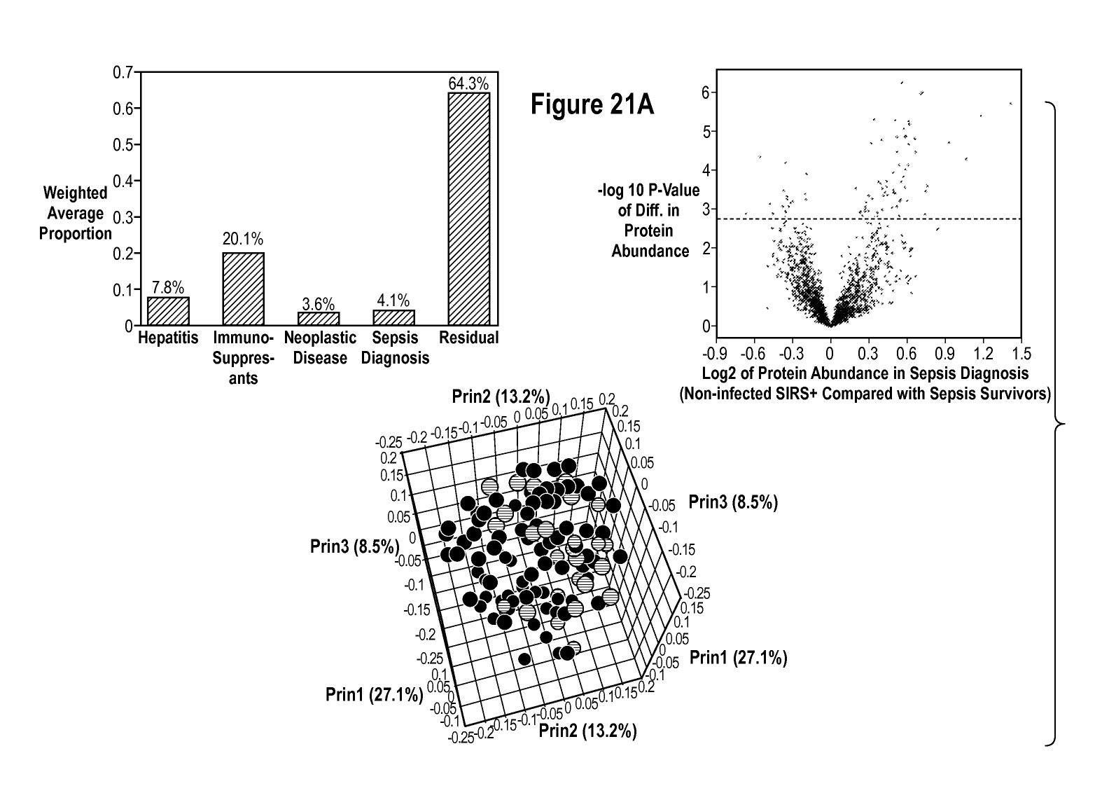

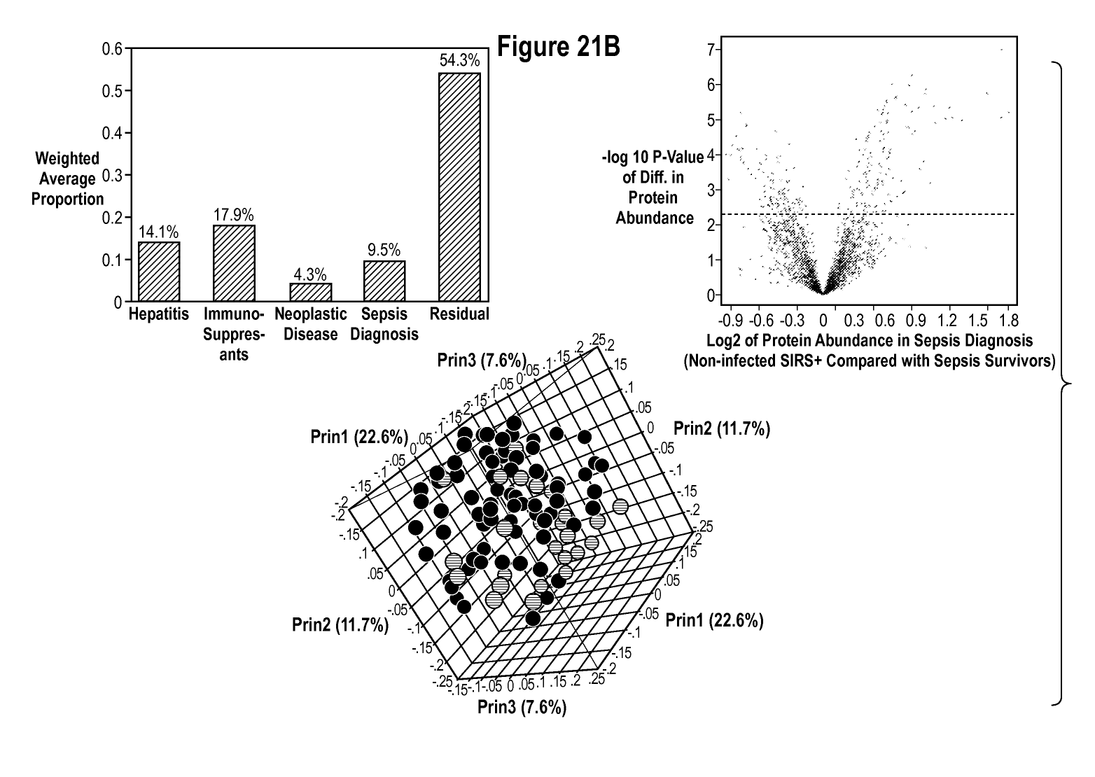

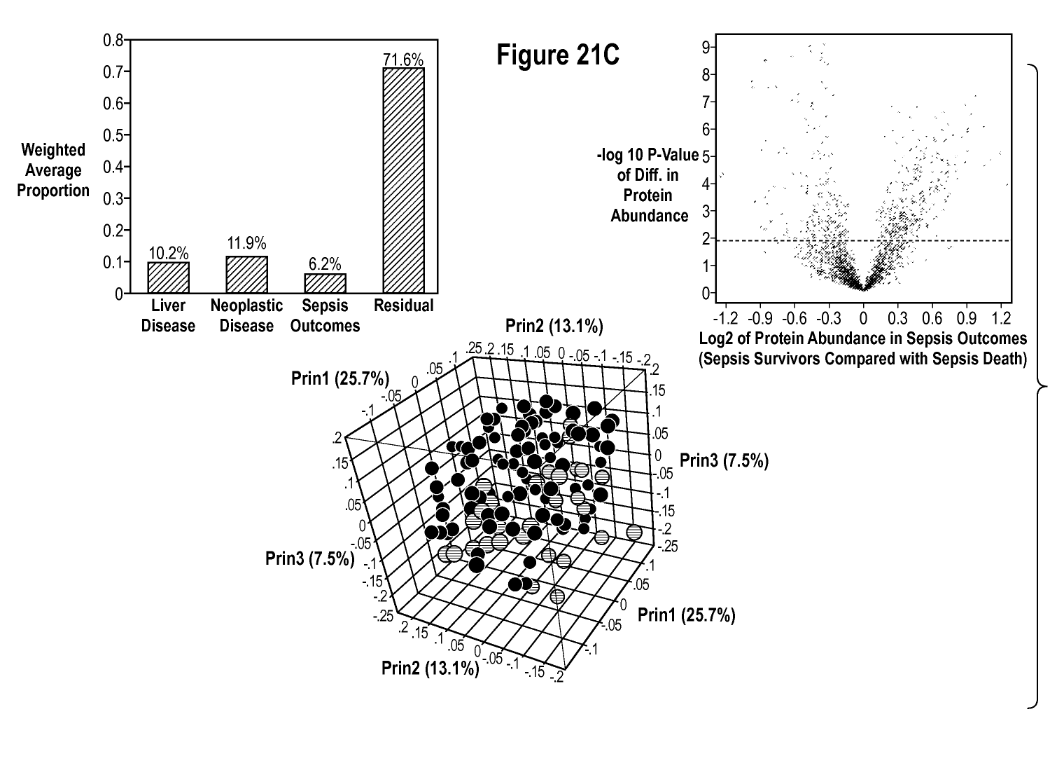

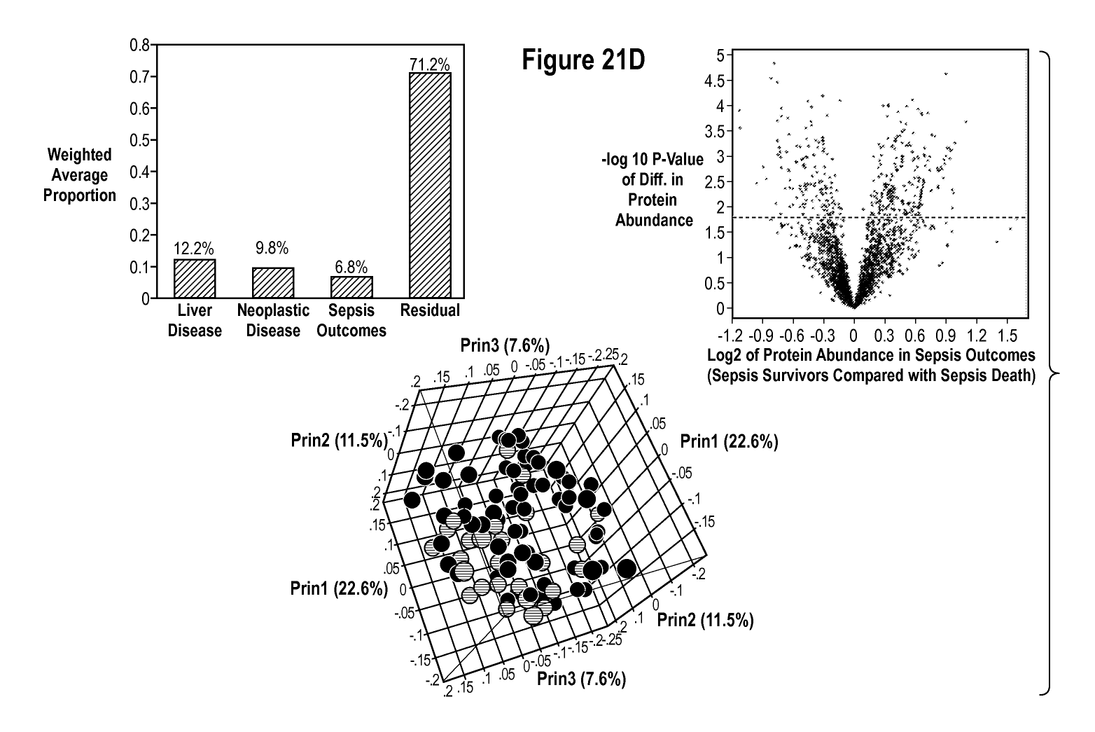

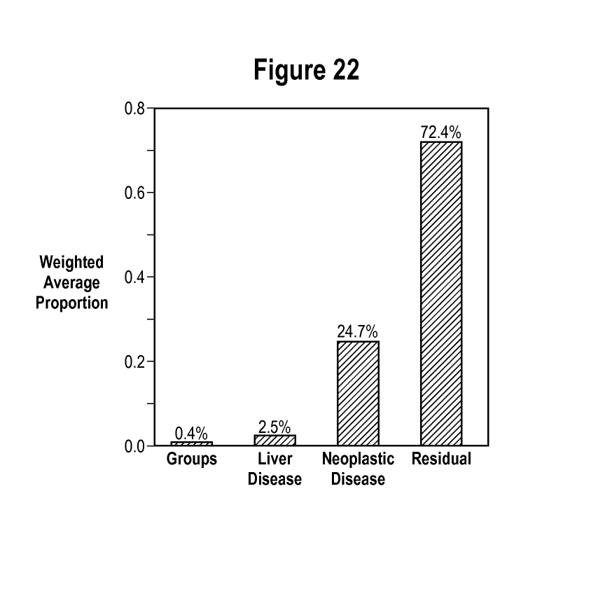

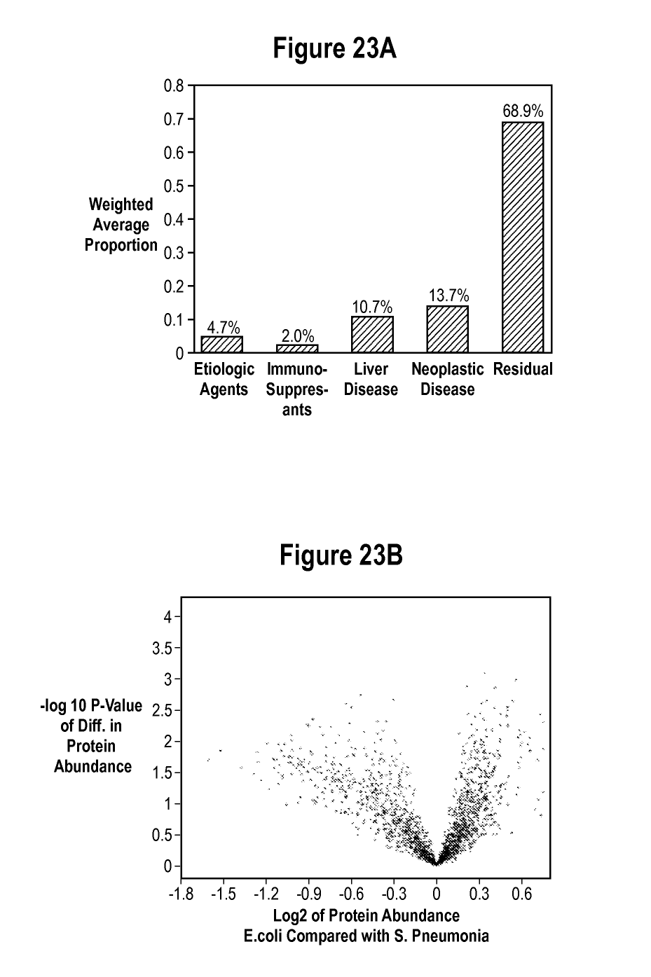

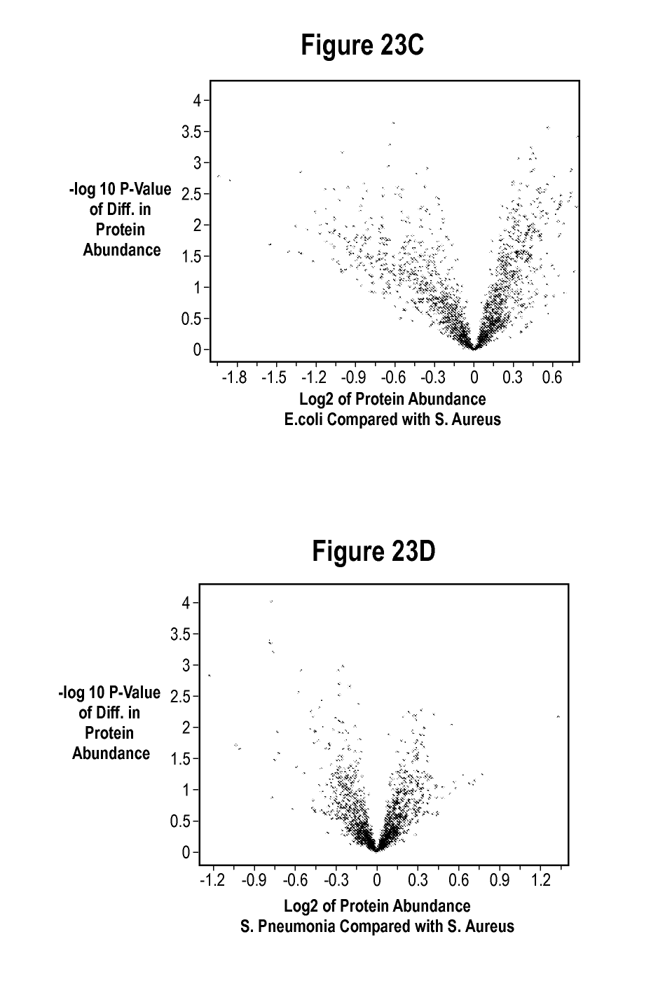

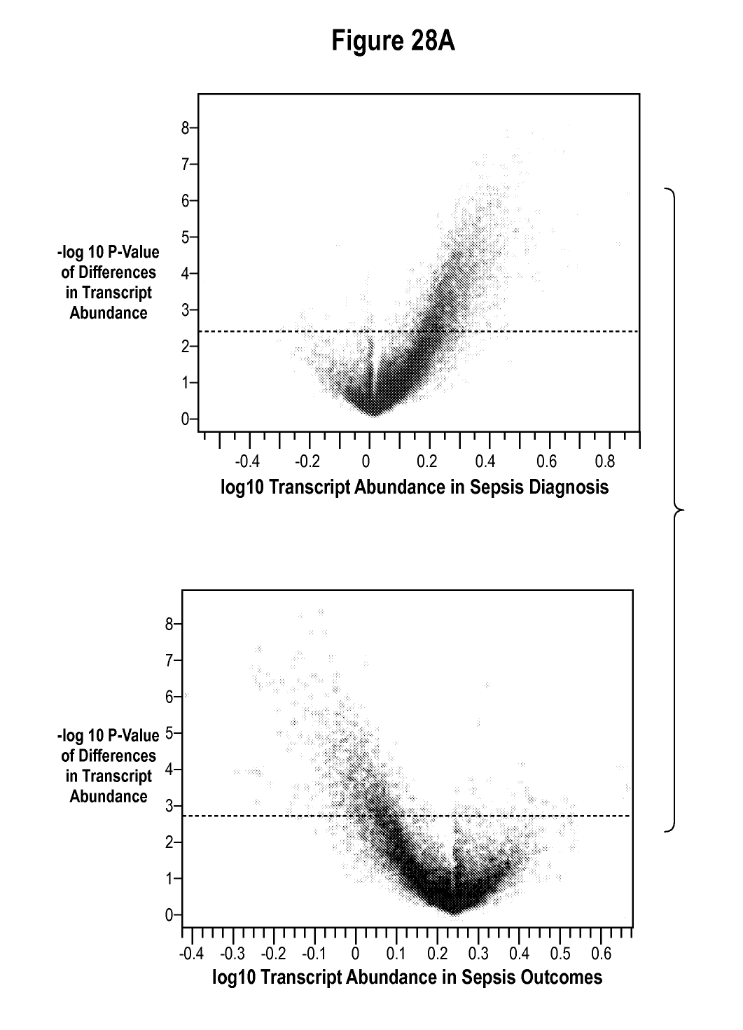

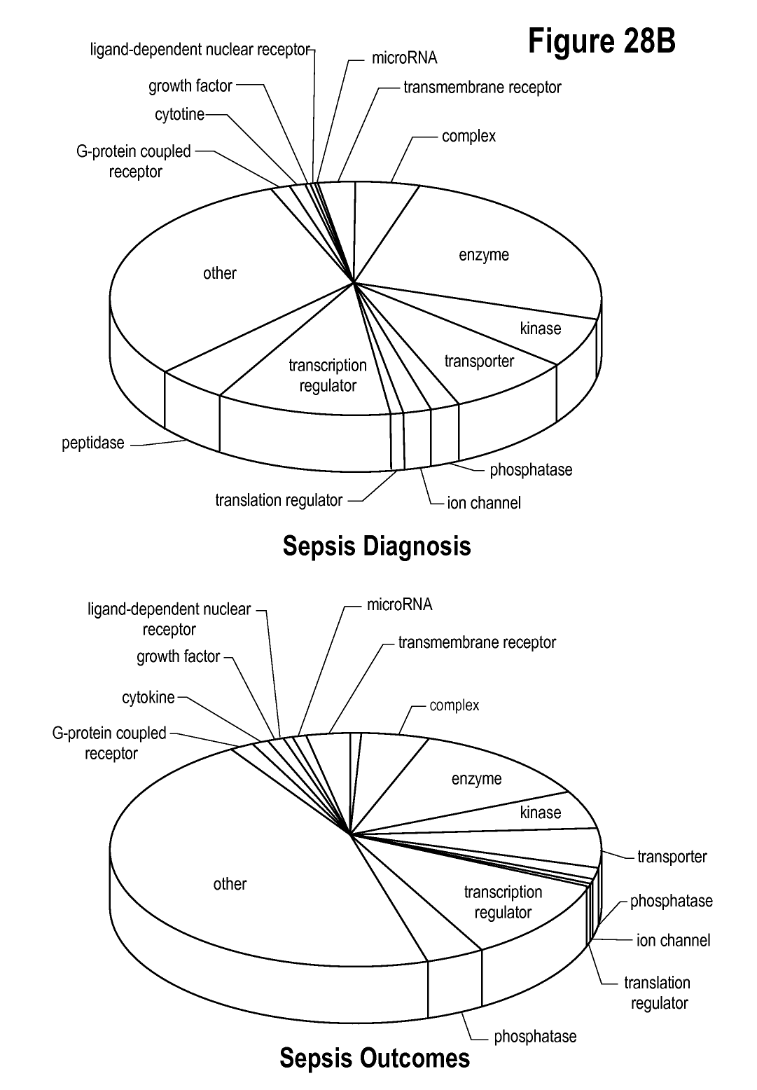

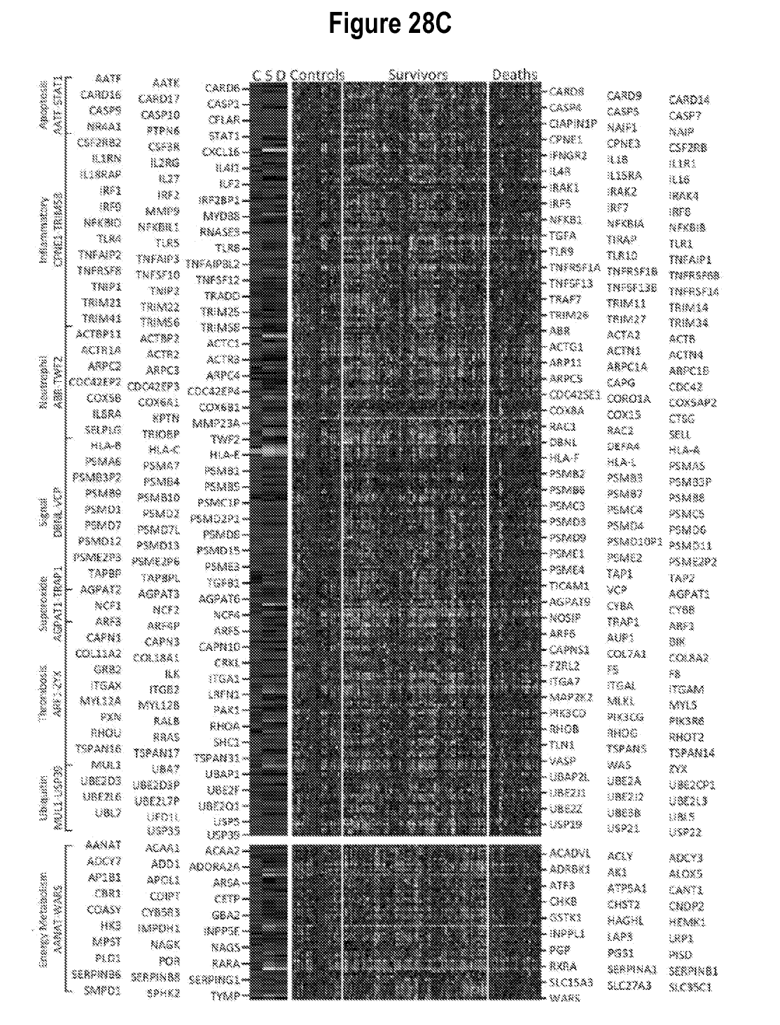

Proteomic analysis of these samples provided an orthogonal survey of host response in sepsis survival and death (FIG. 3). Plasma proteins of high confidence were identified by MS and quantified both by log-transformed quantile-normalized areas-under-the-curve (AUC) of aligned chromatograms after background noise removal, and by spectral counting. In general, cytokines are too small to be detected with high confidence (by more than one peptide) by MS. Following immunodepletion of abundant plasma proteins, 195 and 117 high confidence proteins were measured by the two methods, respectively, of which 101 were detected by both (FIG. 18; Tables 12, 13). For proteins with spectral counts >10, measurements derived from the two methods correlated well (FIG. 18). Despite 23.7% median coefficient of variation of AUC measurements, clinical assays of serum C reactive protein (CRP) and albumin correlated with log-transformed MS values in plasma (FIG. 19), t₀ plasma proteome mScores (averages of the absolute values of Z-scores) showed an identical group progression to that of metabolites (FIG. 20). PCA showed the major determinants of variation in the plasma proteome to be liver disease, immunosuppression/neoplasia, and sepsis group membership, in descending order (FIG. 21). Variability in the plasma proteome was uninfluenced by renal function. Sepsis group effects increased from t₀ to t₂₄. Akin to the metabolome, only a single significant protein difference was found among sepsis survivor subgroups or between infectious agents (FIG. 28, 29).

TABLE 12
Plasma proteins of high confidence identified and quantified by log-transformed,
quantile-normalized AUC of chromatograms after background noise removal.
Proteins were assigned priorities depending on the quality of protein identifi-
cation and whether multiple amino acid sequences were quantified from the same
protein. CV: Coefficient of variation. Only annotated Priority 1 proteins were
retained for analysis.

| | | | Number | Max Absolute | | |
|---|---|---|---|---|---|---|
| Protein | Peptide ID | Multiple | of t₀ | Fold change at | Median CV | Number |
| Priority | Confidence | Sequences | Proteins | t₀ | t₀ Sample | of t₂₄ Proteins |
| 1 | High | Yes | 279 | 2.09 | 23.7% | 195 |
| 2 | High | No | 512 | 2.99 | 40.6% | 584 |
| 3 | Moderate | Yes | 176 | 1.93 | 29.4% | 262 |
| 4 | Moderate | No | 1616 | 3.97 | 41.6% | 1836 |
| Total | | | 2583 | 3.97 | 38.8% | 2877 |

TABLE 13
Plasma proteins detected with high confidence by two MS-based methods
(log-transformed, quantile-normalized AUC of chromatograms after background noise
removal and spectral counting) following immunodepletion of abundant proteins

| t₀ Protein ID | t₂₀ Protein ID | Gene Symbol | Annotation |
|---|---|---|---|
| IPI00022895.7 | IPI00022895.7 | A1BG | α-1-A-glycoprotein |
| IPI00478003.1 | IPI00478003.1 | AZM | α-z-inacrogiobulin |
| IPI00328762.4 | IPI00328762.5 | ABCA13 | ATP-binding cassette sub-family A member 13 |
| IPI00021428.1 | | ACTA1 | Actin, α |
| IPI00008603.1 | IPI00008603.1 | ACTA2 | actin_α 2 |
| IPI00021439.1 | IPI00021439.1 | ACT6 | actin β |
| IPI00003269.1 | IPI00003269.1 | ACTBL2 | actin_β-like 2 |
| IPI00020019.1 | IPI00020019.1 | ADIPOQ | adiponectin_ClQ and collagen domain containing |
| | IPI00004344.1 | AFF4 | AF4/FMB2 family_member 4 |
| IPI00019943.1 | IPI00019943.1 | AFM | afamin |
| IPI00022443.1 | IPI00022443.1 | AFP | α-fetoprotein |
| 15079348 | IPI00032220.3 | AGT | engiotensinogen (serpin peptidase inhibitor_cladeA_member 8) |
| IPI00022431.1 | IPI00022431.2 | AHSG | α-2-H5-glycoprotein |
| | | ALB | Alburain |
| IPI00022426.1 | IPI00022426.1 | AMBP | α-1-microglobullin/bikunin precurcor |
| IPI00022391.1 | IPI00022391.1 | APCS | amyloid P component_serum |
| 253362 | IPI00021841.1 | APOA1 | Apolipoprotein A-I |
| 671882 | IPI00021854.1 | APOA2 | apolipoprotein A-II |
| IPI00304273.2 | IPI00847179.1 | APOA4 | apolipoprotein A-IV |
| 225311 | IPI00022229.3 | APO8 | Apolipoprotein 8-100 |
| IPI00021855.1 | IPI00021855.1 | APOC1 | apolipoprotein C-I |
| IPI00021856.3 | IPI00021856.3 | APOC2 | apolipoprotein C-II |
| IPI00021857.1 | IPI00021857.1 | APOC3 | apolipoprotein C-III |
| IPI00022731.1 | IPI00022731.1 | APOC4 | apolipoprotein C-IV |
| IPI00006662.1 | IPI00006662.1 | APOD | apolipoprotein D |
| 15826311 | IPI00021842.1 | APOE | apolipoprotein E |
| | | APOF | Apolipoprotein F |
| IPI00298828.3 | IPI00298828.3 | APOH | apolipoprotein H (β-2-glycoprotein I) |
| IPI00186903.3 | IPI00186903.4 | APOL1 | apolipoprotein L_1 |
| IPI00027235.1 | | ATRN | Attractin |
| IPI00166729.4 | IPI00166729.4 | AZGP1 | α-2-glycoprotein 1_zinc-binding |
| IPI00004656.1 | IPI00004656.3 | B2M | β-2-microglobulin |
| IPI00297188.5 | IPI00297188.6 | BAI2 | brain-specific angiogenesis inhibitor 2 |
| IPI00022392.1 | IPI00022392.1 | C1QA | complement component 1_q subcomponent_A chain |
| IPI00477992.1 | IPI00477992.1 | C1QB | complement component 1_q subcomponent_B chain |
| IPI00022394.2 | IPI00022394.2 | C1QC | complement component 1_q subcomponent_C chain |
| IPI00296165.5 | IPI00296165.6 | C1R | complement component 1_r subcomponent |
| IPI00009793.2 | IPI00009793.4 | C1RL | complement component 1_r subcomponent-like |
| IPI00017696.1 | IPI00017696.1 | CLS | complement component 1_s subcomponent |
| IPI00745619.1 | IPI00303963.1 | C2 | complement component 2 |
| IPI00783987.1 | IPI00783987.2 | C3 | complement component 3 |
| 179674 | IPI00892547.1 | C4A | complement component 4A |
| IPI00418163.3 | IPI00418163.3 | C48 | complement component 4B |
| IPI00021727.1 | IPI00021727.1 | C48PA | complement component 4 binding protein_α |
| IPI00025862.1 | IPI00025862.2 | C48PB | complement component 4 binding protein_β |
| 38016947 | IPI00032291.2 | C5 | complement component 5 |
| IPI00009920.2 | IPI00879709.3 | C6 | complement component 6 |
| 179716 | IPI00296608.6 | C7 | Complement component 7 |
| 9016854 | IPI00011252.1 | C8A | complement component 8_α polypeptide |
| IPI00294395.1 | | C8B | Complement component C8 β chain |
| IPI00011261.2 | IPI00011261.2 | C8G | complement component 8_gamma polypeptide |
| IPI00022395.1 | IPI00022395.1 | C9 | complement component 9 |
| IPI00215983.2 | IPI00215983.3 | CA1 | carbonic anhydrase 1 |
| IPI00465436.3 | IPI00465436.4 | CAT | catalase |
| IPI00029260.2 | IPI00029260.2 | CD14 | CD14 molecule |
| IPI00104074.4 | | CD163 | Scavenger receptor cysteine-rich type 1 protein M130 |
| IPI00025204.1 | IPI00025204.1 | CD5L | CD5 molecule-like |
| 40737516 | | CDA5 | C4A6 |
| | IPI00855958.1 | CENPF | centromere protein F_350/400ka (mitosin) |
| IPI00010180.3 | 010180.4 | CES1 | carboxylesterase 1 (monocyte/macrophage serine csterase 1) |
| IPI00019591.1 | IPI00893864.1 | CF8 | complement factor 8 |
| IPI00019579.1 | IPI00165972.3 | CFD | complement factor D (adipain) |
| IPI00029739.4 | IPI00029739.5 | CFH | Complement factor H |
| IPI00006543.2 | IPI00011264.2 | CFHR1 | complement factor H-related 1 |
| IPI00006154.1 | IPI00006154.1 | CFHR2 | complement factor H-related 2 |
| IPI00027507.1 | IPI00027507.1 | CFHR3 | complement factor H-related 3 |
| IPI00291867.3 | IPI00291867.3 | CF1 | complement factor I |
| IPI00012011.5 | | CFL1 | Cofain-1 |
| IPI00021364.4 | IPI00021364.1 | CFP | complement factor properdin |
| IPI00009028.1 | IPI00009028.1 | CLEC38 | C-type lectin domain family 3_member B |
| IPI00291262.3 | IPI00291262.3 | CLU | clusterin |
| IPI00011283.1 | IPI00011783.2 | COL11A2 | collagen_type X1_α2 |
| 179594 | IPI00297646.4 | COL1A1 | collagen_type I_α1 |
| IPI00168920.2 | IPI00168920.3 | COL24A1 | collagen_type XXIV_α1 |
| 930045 | IPI00021033.2 | COL3A1 | collagen_type III_α1 |
| IPI00025418.1 | IPI00025418.2 | COL7A1 | collagen_type VII_α1 |
| 2632189 | IPI00423463.1 | COPB1 | coatomer protein complex_subunit β1 |
| IPI00017601.1 | IPI00017601.1 | CP | ceruloplasmin (ferroxidase) |
| IPI00293057.5 | | CPBZ | Carboxypeptidase B2 |
| IPI00010295.1 | IPI00010295.1 | CPN1 | carboxypeptidase N_polypeptide 1 |
| IPI00479116.1 | IPI00475136.3 | CPN2 | carboxypeptidase N_polypeptide 2 |
| | IPI00011062.1 | CPS1 | carbamoyl-phosphate synthecase 1_mitochondrial |
| IPI00022339.1 | IPI00022389.1 | CRP | C-reactive protein_pentraxin-related |
| IPI00032293.1 | IPI00032293.1 | CST3 | cystatin C |
| IPI00005721.1 | IPI00005721.1 | DEFA1 | betensin_α1 |
| IPI00465045.2 | | DIP2B | Disco-interacting protein 2 homolog g |
| 4758236 | IPI00003351.2 | ECM1 | extracellular matrix protein 1 |
| IPI00019531.1 | IPI00019581.1 | F12 | coagulation factor XII (Hegeman factor) |
| IPI00019588.1 | IPI00019568.1 | F2 | coagulation factor II (thrombin) |
| IPI00010290.1 | IPI00010290.2 | FABP1 | fatty acid-binding protein 1_liver |
| IPI00215746.2 | | FABP4 | fatty acid-binding protein, adipocyte |
| | | FAM135A | Protein FAM135A |
| IPI00218803.2 | IPI00218803.3 | FELN1 | fibulin 1 |
| IPI00242956.3 | IPI00242956.5 | FCGBP | Fe-fragment of IgG binding protein |
| IPI00293925.2 | | FCN3 | Ficolin-3 |
| IPI00021885.1 | IPI00021885.1 | FGA | fibrinogen α chain |
| IPI00298437.3 | IPI00298497.3 | FGB | fibrinogen β chain |
| IPI00021891.5 | IPI00021891.5 | FGG | fibrinogen gamma chain |
| | IPI00289334.1 | FLNB | filamin B_β |
| 51476364 | IPI00022418.1 | FN1 | fibronectin 1 |
| | IPI00375676.3 | FTL | ferritin_light polypeptide |
| IPI00010375.3 | IPI00555812.4 | GC | vitamin D-binding protein |
| IPI00026199.1 | IPI00026199.2 | GPK3 | glutathione peroxidase 3 (plasma) |
| IPI00026314.1 | IPI00026314.1 | GSN | gelsolin (amyloidosis_Finnish type) |
| IPI00465253.4 | IPI00465253.4 | HAU56 | HAUS augrnin-like complex_subunit 5 |
| 27574247 | IPI00410714.5 | HBA1 | hemoglobin_α1 |
| 13195586 | | HBA2 | Hemoglobin subunit α |
| IPI00658153.1 | IPI00654755.3 | HBB | Hemoglobin subunit β |
| IPI00473011.2 | IPI00473011.3 | HBD | hemoglobin_delta |
| | IPI00220706.10 | HBG1 | hemoglobin_gamma A |
| IPI00217473.4 | IPI00217473.5 | HBZ | hemoglobin_zeta |
| 229528 | | HLA-E | HLA class I histocompatibility antigen, α chain E |
| 229271 | IPI00902590.1 | HP | Haptoglubin |
| IPI00477597.1 | IPI00477597.1 | HPR | haptoglubin-related protein |
| IPI00022488.1 | IPI00022488.2 | HPX | hemopexin |
| IPI00022371.1 | IPI00022371.1 | HRG | Histidine-rich glycoprotein |
| | IPI00220362.5 | HSPE1 | heat shock 10 kDa protein 1 (chaperonin 10) |
| IPI00005477.4 | IPI00009477.4 | ICAM2 | Intercellular adhesion molecular |
| | | IGFALS | Insulin-like growth factor-binding protein complex acid lablle subunit |
| IPI00297284.1 | | IGFBP2 | Insulin-like growth factor-binding protein 2 |
| IPI00305380.3 | IPI00305380.3 | IGFBP4 | Insulin-like growth factor binding protein 4 |
| IPI00029235.1 | | IGFBP6 | Insulin-like growth factor-binding protein 6 |
| 34527679 | IPI00386524.3 | IGHA1 | immunogicbulin heavy censtant α1 |
| | | IGHD | Ig delta chain C region |
| 21757089 | | IGHG1 | Ig gamma-1 chain C region |
| | | IGHG2 | Ig gamma-2 chain C region |
| | | IGHG3 | Ig gamma-3 chain C region |
| | | IGHG4 | Ig gamma-4 chain C region |
| | | IGHM | Ig rae chain C region |
| | | IGJ | Immunoglobulin J chain |
| | | IGKC | Ig kappa chain C region |
| | | IGKY1-5 | Ig kappa chain V-1 region HK102 |
| | | IGLC7 | Ig lambda-7 chain C region |
| 1871489 | | IGM | IgM_(Homo_sapiens) |
| 33319112 | | IGVH2 | Ig heavy chain variable region, VH3 family |
| IPI00292530.1 | IPI00292530.1 | ITH1 | inter-α (globulin) inhibitor H1 |
| IPI00305461.2 | IPI00305461.3 | ITH2 | inter-α (globulin) inhibitor H2 |
| IPI00026413.1 | IPI00028413.8 | ITH3 | inter-α (globulin) inhibitor H3 |
| IPI00218192.1 | IPI00218192.3 | ITH4 | inter-α (globulin) inhibitor H4 |
| 1575607 | IPI00479786.5 | KHSRP | KH-type splicing regulatory protein |
| | IPI00827544.1 | KIF19 | kinesin family member 19 |
| IPI00654888.2 | IPI00654888.2 | KLKB1 | kallikrein B_plasma (Fletcher factor)1 |
| IPI00032328.1 | IPI00215894.1 | KNGL | kninogen 1 |
| | | KRT31 | Keratin, type I cuticular Ha1 |
| | | KRT81 | Keratin, type II cuticular Hb1 |
| 31652249 | IPI00032311.4 | LBP | lipopolysaccharide binding protein |
| IPI00299547.4 | IPI00299547.4 | LCN2 | lipocalin 2 |
| IPI00010471.4 | IPI00010471.5 | LCP1 | lympocyte-cytosolic protein 1 (L-plastin) |
| IPI00023673.1 | IPI00023673.1 | LGALS3BP | lectin_galactoside-binding_soluble_3 binding protein |
| IPI00164623.4 | | LOC100133511 | hypothetical protein LOC100133511 |
| IPI00167093.4 | | LOC100293069 | similar to complement factor H-related 1 |
| IPI00061977.1 | | LOC100294459 | similar to immunoglobulin lambda-like polypeptide 1 |
| IPI00736860.2 | | LOC100294460 | similar to immunoglobulin lambda-like polypeptide 2 |
| IPI00029168.1 | IPI00029168.1 | LPA | lipoprotein_Lp(a) |
| 21707947 | IPI00022417.4 | LRG1 | leucine-rich-α-2-glycoprotein 1 |
| IPI00298860.5 | | LTF | Lactotransferrin |
| IPI00020986.2 | IPI00020986.2 | LUM | lumican |
| 3402141 | IPI00019038.1 | LYZ | lysozyme |
| | 126508 | LYZL4 | lysozme like-4 |
| | | MACF1 | Microtuble-actin cross-linking factor 1, isoform 4 |
| IPI00217493.4 | IPI00217493.5 | MB | myoglobin |
| IPI00004373.1 | IPI00004373.1 | MBL2 | mannose-binding lectin Z (protein C) |
| 21756643 | | MCAM | Cell surface glycoprotein MUC18 |
| IPI00306929.7 | IPI00306929.9 | MYO18B | myosin XVIIIB |
| | IPI00423460.3 | NID1 | nidogen 1 |
| IPI00022429.3 | IPI00022429.3 | ORM1 | orosomucoid 1 |
| IPI00020091.1 | IPI00020091.1 | ORM2 | orosomucoid 2 |
| IPI00022295.1 | | PF4V1 | Platelet factor 4 variant |
| IPI00216694.1 | | PFN1 | Profillin-1 |
| IPI00163207.1 | IPI00163207.1 | PGLYRP2 | peptidoglycan recognition protein 2 |
| IPI00004573.1 | IPI00004573.2 | PIGR | polymeric immunoglobulin receptor |
| IPI00306311.8 | IPI00306311.8 | PLEK | pleckstrin |
| 229453 | IPI00019580.1 | PLG | Plasminogen |
| 1262347 | IPI00218732.3 | PON1 | paraoxonase 1 |
| IPI00555900.1 | | POTEK | POTE ankyrin domain family, member K |
| IPI00022445.1 | IPI00022445.1 | PFBP | pro-platelet basic protein (chemokine C-X-C ligand 7) |
| IPI00027350.3 | IPI00027350.2 | PRDX2 | peroxiredoxin 2 |
| IPI00024825.2 | IPI00024825.2 | PRG4 | proteoglycan 4 |
| IPI00294004.1 | | PROS1 | Vitamin K-dependent protein 5 |
| IPI00013179.1 | IPI00013179.1 | PTGDS | Prostaglandin-H2 D-isomerase |
| IPI00025426.1 | IPI00025426.2 | PZP | pregnancy-zone protein |
| IPI00003590.2 | | QSOX1 | Sulfhydryl-oxidase 1 |
| IPI00221325.3 | IPI00221325.3 | RANBP2 | RNA binding protein 2 |
| IPI00411314.2 | IPI00304692.1 | RBMX | RNA binding motif protein_X-linked |
| 2895204 | IPI00022420.3 | RBP4 | retinal binding protein 4_plasma |
| IPI00009027.1 | IPI00009027.1 | REG1A | regenerating islet-derived 1c |
| IPI00014048.1 | IPI00014048.1 | RNASE1 | RNase A family_1 |
| IPI00007047.1 | IPI00007047.1 | S100A8 | S100 calcium binding protein A8 |
| IPI00027462.1 | IPI00027462.1 | S100A9 | S100 calcium binding protein A9 |
| 247142 | IPI00006146.4 | SAA1 | serum amyloid A protein |
| IPI00027191.1 | IPI00027191.1 | SAA3P | serum amyloid A3 |
| IPI00019399.1 | IPI00019399.1 | SAA4 | serum amyloid A4 |
| | IPI00218795.1 | SELL | selectin L |
| IPI00029061.2 | IPI00029061.3 | SEPP1 | selenoprotein P_plasma_1 |
| 223433 | IPI00553177.1 | SERPINA1 | α-1-antitrypsin |
| IPI00007199.4 | IPI00007199.4 | SERPINA10 | serpin peptidase inhibitor_clade A (α-1 antiproteinase)_member 10 |
| 225769 | IPI00550991.3 | SERPINA3 | serpin peptidase inhibitor_clade A (α-1 antiproteinase)_member 3 |
| IPI00027482.1 | IPI00027482.1 | SERPINA6 | serpin peptidase inhibitor_clade A (α-1 antiproteinase)_member 6 |
| IPI00292946.1 | IPI00292946.1 | SERPINA7 | serpin peptidase inhibitor_clade A (α-1 antiproteinase)_member 7 |
| IPI00032179.2 | IPI00032179.3 | SERPINC1 | serpin peptidase inhibitor_clade C (antithrombin)_member 1 |
| 23273330 | IPI00292950.4 | SERPIND1 | serpin peptidase inhibitor_clade D (heparin cofactor)_member 1 |
| 39725934 | IPI00006114.4 | SERPINF1 | serpin peptidase inhibitor_clade F (α-2 antiplasmin)_member 1 |
| IPI00029863.4 | IPI00879231.1 | SERPINF2 | serpin peptidase inhibitor_clade F (α-2 antiplasmin)_member 2 |
| 179621 | IPI00291866.5 | SERPING1 | serpin peptidase inhibitor_clade G (C1 inhibitor)_member 1 |
| | IPI00179016.8 | SETD1A | SET domain containing 1A |
| IPI00023019.1 | IPI00023019.1 | SHBG | sex hormonic binding protein |
| | IPI00011961.1 | SIGLEC1 | sialic acid binding Ig-like lectin 1 sialoadherin |
| IPI00020687.1 | IPI00020687.1 | SPINK1 | serine peptidase inhibitor_Kazal type 1 |
| IPI00550363.2 | IPI00550363.3 | TAGLN2 | transgelin 2 |
| 2815575 | IPI00022463.1 | TF | Serotransferrin |
| IPI00032292.1 | IPI00032292.1 | TIMP1 | TIMP metallopeptidase inhibitor 1 |
| IPI00298994.5 | IPI00298994.5 | TLN1 | talin 1 |
| IPI00180240.2 | | TMSL3 | Thymosin β-4-like protein 3 |
| | IPI00554760.1 | TNR | tenascin R (restrictin_janusin) |
| IPI00010779.3 | | TPM4 | Tropomyosin α-4-chain |
| | | TRANK1 | IPR and ankyrin repeat-containing protein 1 |
| | IPI00413160.4 | TRIOBP | TRIO and F-actin binding protein |
| IPI00023283.3 | IPI00023283.3 | TIN | titin |
| 230651 | IPI00022432.1 | TIR | transthyretin |
| IPI00295413.8 | IPI00807602.1 | ULK4 | unc-51-like kinase 4 |
| 33358191 | IPI00386524.3 | unknown 51 | I_Chain_L Of_Iliv-1_Antibody_215_In_Complex_With_Gp41 |
| 51103537 | | unknown 70 | immunoglobulin variable region VL kappa domain |
| 51103559 | | unknown 72 | immunoglobulin variable region VL kappa domain |
| 896277 | | unknown 8 | immmunoglobulin lambda light chain VLI region |
| | IPI00887739.3 | unknowna111 | similar to complement component C3 |
| | IPI00894523.1 | unknowna126 | POTE ankyrin domain family, member |
| | IPI00930382.1 | unknowna149 | hp2-a |
| | IPI00736860.3 | unknowna60 | immunoglobulin heavy chain |
| | | unknown_d1 | Ig kappa chain V-I-region AG |
| | | unknown_d10 | Ig kappa chain V-III-region SIE |
| | | unknown_d11 | Ig kappa chain V-III-region VG |
| | | unknown_d12 | Ig kappa chain V-III-region VH |
| | | unknown_d13 | Ig kappa chain V-IV-region Len |
| | | unknown_d14 | Ig lambda chain V-I-region WAH |
| | | unknown_d15 | Ig lambda chain V-III-region LOI |
| | | unknown_d16 | JPH1_HUMAN-R |
| | | unknown_d17 | Keratin-B1-like protein |
| | | unknown_d2 | Ig kappa chain V-II region MIL |
| | | unknown_d3 | ZXDB_HUMAN-R |
| | | unknown_d4 | FBX7_HUMAM-R |
| | | unknown_d5 | Ig heavy chain V-III region GAL |
| | | unknown_d6 | Ig kappa chain V-I region EU |
| | | unknown_d7 | Ig kappa chain V-I region WEA |
| | | unknown_d8 | Ig kappa chain V-III region B6 |
| | | unknown_d9 | Ig kappa chain V-III region NG9 |
| | IPI00020037.1 | USF2 | upstream transcription factor 2_c-fos interacting |
| IPI00395488.2 | IPI00395488.2 | VASN | vasorin |
| IPI00027038.1 | IPI00027038.1 | VSIG4 | V-set and immunoglobulin domain containing 4 |
| IPI00298971.1 | IPI00298971.1 | VTN | vitronectin |
| IPI00023014.1 | IPI00023014.1 | VWF | von Willebrand factor |

| t₀ Protein ID | t₂₀ Protein ID | Gene Symbol | t₀ Best Peptide Sequence |
|---|---|---|---|
| IPI00022895.7 | IPI00022895.7 | A1BG | VTLTCVAPLSGVDFQLR |
| IPI00478003.1 | IPI00478003.1 | AZM | AFOPFFVELTMRYSVIR |
| IPI00328762.4 | IPI00328762.5 | ABCA13 | YIYELLN |
| IPI00021428.1 | | ACTA1 | AGFAGDDAPR |
| IPI00008603.1 | IPI00008603.1 | ACTA2 | AGFAGDDAPR |
| IPI00021439.1 | IPI00021439.1 | ACT6 | AGFAGDDAPR |
| IPI00003269.1 | IPI00003269.1 | ACTBL2 | SYELPOGQVITIGNER |
| IPI00020019.1 | IPI00020019.1 | ADIPOQ | GDIGETGVPGAEGPR |
| | IPI00004344.1 | AFF4 | |
| IPI00019943.1 | IPI00019943.1 | AFM | TINPAVDHCCK |
| IPI00022443.1 | IPI00022443.1 | AFP | YIQESQALAK |
| 15079348 | IPI00032220.3 | AGT | SLDFTELDVAAEK |
| IPI00022431.1 | IPI00022431.2 | AHSG | HTFMGVVSLGSPSGEVSHPR |
| | | ALB | |
| IPI00022426.1 | IPI00022426.1 | AMBP | AFIQLWAFDAVK |
| IPI00022391.1 | IPI00022391.1 | APCS | AYSLFSYNTQGR |
| 253362 | IPI00021841.1 | APOA1 | VKDLATVYVDVLK |
| 671882 | IPI00021854.1 | APOA2 | EPCVESLVSQYFQTVTDYGK |
| IPI00304273.2 | IPI00847179.1 | APOA4 | SLAELGGHLDQQVEEFR |
| 225311 | IPI00022229.3 | APO8 | ILSEPINIDALEMR |
| IPI00021855.1 | IPI00021855.1 | APOC1 | TPDVSSALDKLK |
| IPI00021856.3 | IPI00021856.3 | APOC2 | STAAMSTYIGIFTDQVLSVLK |
| IPI00021857.1 | IPI00021857.1 | APOC3 | DALSSVQESQVAQQAR |
| IPI00022731.1 | IPI00022731.1 | APOC4 | DGWQWFWSPSTFR |
| IPI00006662.1 | IPI00006662.1 | APOD | CPNPPYQENFDYNK |
| 15826311 | IPI00021842.1 | APOE | GEVQAMLGQSTEELR |
| | | APOF | |
| IPI00298828.3 | IPI00298828.3 | APOH | ATFGCHDGYSLDGPEEIECTK |
| IPI00186903.3 | IPI00186903.4 | APOL1 | VTEPISAESGEQYER |
| IPI00027235.1 | | ATRN | GDECQLCEVENR |
| IPI00166729.4 | IPI00166729.4 | AZGP1 | YSLTYIYTGLSK |
| IPI00004656.1 | IPI00004656.3 | B2M | DWSFYLLYYTEFTPTEKDEYACR |
| IPI00297188.5 | IPI00297188.6 | BAI2 | ASPGLGEPPPQEANPVYM |
| IPI00022392.1 | IPI00022392.1 | C1QA | SLGFCDTINK |
| IPI00477992.1 | IPI00477992.1 | C1QB | LEQGENVFLQATDK |
| IPI00022394.2 | IPI00022394.2 | C1QC | FNAVLTNPQGDYDTSTGK |
| IPI00296165.5 | IPI00296165.6 | C1R | LPVANPQACENWLR |
| IPI00009793.2 | IPI00009793.4 | C1RL | SGLIGYYSGFGMEMGWLTTELK |
| IPI00017696.1 | IPI00017696.1 | CLS | CVPVCGVPR |
| IPI00745619.1 | IPI00303963.1 | C2 | RHAFILQAVYK |
| IPI00783987.1 | IPI00783987.2 | C3 | DICEEQVNSLPGSITK |
| 179674 | IPI00892547.1 | C4A | VTSADPLDTLGSEGALSPGGVASLLR |
| IPI00418163.3 | IPI00418163.3 | C48 | VTSADPLDTLGSEGALSPGGVASLLR |
| IPI00021727.1 | IPI00021727.1 | C48PA | FSAICQGOGTWSPR |
| IPI00025862.1 | IPI00025862.2 | C48PB | NLCEAMENFMQQLK |
| 38016947 | IPI00032291.2 | C5 | TDAPDLPEENQAR |
| IPI00009920.2 | IPI00879709.3 | C6 | CPINCLLGDFGPWSDCDPCIEK |
| 179716 | IPI00296608.6 | C7 | MPYECGPSLDVCAQDER |
| 9016854 | IPI00011252.1 | C8A | ALDQYLMEFNACT |
| IPI00294395.1 | | C8B | DFGTHYITEAVLGGIYEYTLYMNK |
| IPI00011261.2 | IPI00011261.2 | C8G | YGFCEAADQFHVLDEVR |
| IPI00022395.1 | IPI00022395.1 | C9 | AIEDUINEFSVR |
| IPI00215983.2 | IPI00215983.3 | CA1 | ADGLAVIGVLMK |
| IPI00465436.3 | IPI00465436.4 | CAT | AFYVNVLNEEQR |
| IPI00029260.2 | IPI00029260.2 | CD14 | AFPALTSLOLSDNPGLGER |
| IPI00104074.4 | | CD163 | EAEFGQGTGPIWLNEVK |
| IPI00025204.1 | IPI00025204.1 | CD5L | ELGCGAASGTPSGILYEPPAEK |
| 40737516 | | CDA5 | VGDTLNLNLR |
| | IPI00855958.1 | CENPF | |
| IPI00010180.3 | 010180.4 | CES1 | ESQPLLGTVIDGMLLIK |
| IPI00019591.1 | IPI00893864.1 | CF8 | LLQEGQALEYVCPSGFYPYPVQTR |
| IPI00019579.1 | IPI00165972.3 | CFD | RPDSLQHVLLPVLDR |
| IPI00029739.4 | IPI00029739.5 | CFH | AVYTCNEGYQLLGEINYR |
| IPI00006543.2 | IPI00011264.2 | CFHR1 | ITCTEEGWSPTPK |
| IPI00006154.1 | IPI00006154.1 | CFHR2 | CLDPCVISQEIMEK |
| IPI00027507.1 | IPI00027507.1 | CFHR3 | CYFPYLENGYNQNYGR |
| IPI00291867.3 | IPI00291867.3 | CF1 | TMGYQDFADVVCYTQK |
| IPI00012011.5 | | CFL1 | KEDLYFIFWAPESAPLK |
| IPI00021364.4 | IPI00021364.1 | CFP | SICQEIPGQQSR |
| IPI00009028.1 | IPI00009028.1 | CLEC38 | LDTLAQEVALLK |
| IPI00291262.3 | IPI00291262.3 | CLU | LFDSDPITVTVPVEVSR |
| IPI00011283.1 | IPI00011783.2 | COL11A2 | GEHGPPGPPGPIGPVGQPGAAGADGEPGAR |
| 179594 | IPI00297646.4 | COL1A1 | VLCDDVICDETK |
| IPI00168920.2 | IPI00168920.3 | COL24A1 | NKNRLQLGVQLLPK |
| 930045 | IPI00021033.2 | COL3A1 | GDPGANGLPGAA |
| IPI00025418.1 | IPI00025418.2 | COL7A1 | GDPGVGLPGPR |
| 2632189 | IPI00423463.1 | COPB1 | VSGUWGQGTLVTVSSASPTSPK |
| IPI00017601.1 | IPI00017601.1 | CP | ALYLQYTDETPE |
| IPI00293057.5 | | CPBZ | ASASYYEQYHSLNEIYSWIEFTTER |
| IPI00010295.1 | IPI00010295.1 | CPN1 | IVQLIQDTR |
| IPI00479116.1 | IPI00475136.3 | CPN2 | VVFLNTQLCQFRPDAFGGLPR |
| | IPI00011062.1 | CPS1 | |
| IPI00022339.1 | IPI00022389.1 | CRP | RQDNEIJFWSK |
| IPI00032293.1 | IPI00032293.1 | CST3 | LVGGPMDASVEEESVR |
| IPI00005721.1 | IPI00005721.1 | DEFA1 | IPACIAGER |
| IPI00465045.2 | | DIP2B | DSAVQKELR |
| 4758236 | IPI00003351.2 | ECM1 | NVALVSGDTENAK |
| IPI00019531.1 | IPI00019581.1 | F12 | TILSGAPCOPWASEATYR |
| IPI00019588.1 | IPI00019568.1 | F2 | LAVTTHGLPCLAWASAGAK |
| IPI00010290.1 | IPI00010290.2 | FABP1 | SVTELNGDHTNTMTLGDIVFK |
| IPI00215746.2 | | FABP4 | LVSSENFDDYMK |
| | | FAM135A | |
| IPI00218803.2 | IPI00218803.3 | FELN1 | GYQLSDVDGVTCEDIDECALPTGGHICSYR |
| IPI00242956.3 | IPI00242956.5 | FCGBP | EQGGQGVCLPNYEATCWLWGD |
| IPI00293925.2 | | FCN3 | YGIDWASGR |
| IPI00021885.1 | IPI00021885.1 | FGA | GLIDEVNQDFTNR |
| IPI00298437.3 | IPI00298497.3 | FGB | VYCDMNTENGGWTVIQNR |
| IPI00021891.5 | IPI00021891.5 | FGG | YEASILTHDSSIR |
| | IPI00289334.1 | FLNB | |
| 51476364 | IPI00022418.1 | FN1 | SYTITGLQPGTDYK |
| | IPI00375676.3 | FTL | |
| IPI00010375.3 | IPI00555812.4 | GC | LSNLIK |
| IPI00026199.1 | IPI00026199.2 | GPK3 | FLVGPDGIPIMR |
| IPI00026314.1 | IPI00026314.1 | GSN | QTQYSVLPEFFETPLFK |
| IPI00465253.4 | IPI00465253.4 | HAU56 | LDGTNVAINIPR |
| 27574247 | IPI00410714.5 | HBA1 | VFAHAGEYGAEALER |
| 13195586 | | HBA2 | VGAHAGEYGAEALER |
| IPI00658153.1 | IPI00654755.3 | HBB | SAYTALWGQ |
| IPI00473011.2 | IPI00473011.3 | HBD | VLGAFSDGLADNLK |
| | IPI00220706.10 | HBG1 | |
| IPI00217473.4 | IPI00217473.5 | HBZ | LRVDPVNFK |
| 229528 | | HLA-E | YACZVTHZGLSSPVTK |
| 229271 | IPI00902590.1 | HP | TQGDGVYTLNNEK |
| IPI00477597.1 | IPI00477597.1 | HPR | TEGDGVYTLNDK |
| IPI00022488.1 | IPI00022488.2 | HPX | LYLVQGTQVYVFLTK |
| IPI00022371.1 | IPI00022371.1 | HRG | GGEGTGYFVDFSVR |
| | IPI00220362.5 | HSPE1 | |
| IPI00005477.4 | IPI00009477.4 | ICAM2 | QVILTLQPTLVAVGK |
| | | IGFALS | |
| IPI00297284.1 | | IGFBP2 | GECWCVNPNTGK |
| IPI00305380.3 | IPI00305380.3 | IGFBP4 | EDARPVPQGSCQSELHR |
| IPI00029235.1 | | IGFBP6 | HLOSVLQQLQTEVYR |
| 34527679 | IPI00386524.3 | IGHA1 | SAVQGPPDRDLCGCYSVSSVLPGCAEPWNHGK |
| | | IGHD | |
| 21757089 | | IGHG1 | WQQGNVFSCSVMHEALHDHYTQK |
| | | IGHG2 | |
| | | IGHG3 | |
| | | IGHG4 | |
| | | IGHM | |
| | | IGJ | |
| | | IGKC | |
| | | IGKY1-5 | |
| | | IGLC7 | |
| 1871489 | | IGM | EVQLVESGGGLVKPRG |
| 33319112 | | IGVH2 | LSCVTSGFTFDDHGMTWVR |
| IPI00292530.1 | IPI00292530.1 | ITH1 | LWAYLDQELLAK |
| IPI00305461.2 | IPI00305461.3 | ITH2 | MLADAPPQDPSCCSGALYYGSK |
| IPI00026413.1 | IPI00028413.8 | ITH3 | LVDEDMNSFK |
| IPI00218192.1 | IPI00218192.3 | ITH4 | LDYQEGPPGVEISCWSVEL |
| 1575607 | IPI00479786.5 | KHSRP | AQPPGGGGPGIR |
| | IPI00827544.1 | KIF19 | |
| IPI00654888.2 | IPI00654888.2 | KLKB1 | CLLFSFLPASSINDMEK |
| IPI00032328.1 | IPI00215894.1 | KNGL | LGQSLDCNAEVYVVPWEK |
| | | KRT31 | |
| | | KRT81 | |
| 31652249 | IPI00032311.4 | LBP | GLQYAAQEGLLALQSELLR |
| IPI00299547.4 | IPI00299547.4 | LCN2 | VPLQQNFQDNQFQGK |
| IPI00010471.4 | IPI00010471.5 | LCP1 | VDTDGNGYISFNELNDLFK |
| IPI00023673.1 | IPI00023673.1 | LGALS3BP | ELSEALGQIFDSQR |
| IPI00164623.4 | | LOC100133511 | DICEEQVNSLPGSITK |
| IPI00167093.4 | | LOC100293069 | EIMENYNIALR |
| IPI00061977.1 | | LOC100294459 | GDTFSCMVGHEALPLAFTQE |
| IPI00736860.2 | | LOC100294460 | NQVTLTCLVK |
| IPI00029168.1 | IPI00029168.1 | LPA | TPEYVPNAGLIMNYCR |
| 21707947 | IPI00022417.4 | LRG1 | ENQLEVLEVWLHGLK |
| IPI00298860.5 | | LTF | CSTSPLLEACEFLR |
| IPI00020986.2 | IPI00020986.2 | LUM | SLEYLDLSENQLAR |
| 3402141 | IPI00019038.1 | LYZ | WESGYNTR |
| | 126508 | LYZL4 | |
| | | MACF1 | |
| IPI00217493.4 | IPI00217493.5 | MB | HGATVLTALGGILK |
| IPI00004373.1 | IPI00004373.1 | MBL2 | TEGQFVDLTGNA |
| 21756643 | | MCAM | LSCEASGFR |
| IPI00306929.7 | IPI00306929.9 | MYO18B | TTELKEAEPQGK |
| | IPI00423460.3 | NID1 | |
| IPI00022429.3 | IPI00022429.3 | ORM1 | NWGLSVYADKPETTK |
| IPI00020091.1 | IPI00020091.1 | ORM2 | TLMFGSYLDDEK |
| IPI00022295.1 | | PF4V1 | HITSLEVIK |
| IPI00216694.1 | | PFN1 | TFVNITPAEVGVLVGK |
| IPI00163207.1 | IPI00163207.1 | PGLYRP2 | SLPLLMDSVIQALAELEQK |
| IPI00004573.1 | IPI00004573.2 | PIGR | NADLQVLKPEPELVYEDLR |
| IPI00306311.8 | IPI00306311.8 | PLEK | LPETIDLGALYLSMK |
| 229453 | IPI00019580.1 | PLG | EPLDDVYNTQGASLFSVTK |
| 1262347 | IPI00218732.3 | PON1 | ILLMDLNEEDPTVLELGITGSK |
| IPI00555900.1 | | POTEK | SYELPDGQVITIGNER |
| IPI00022445.1 | IPI00022445.1 | PFBP | GTHCNQVEVIATLK |
| IPI00027350.3 | IPI00027350.2 | PRDX2 | KEGGLGPLNIPLLADVTR |
| IPI00024825.2 | IPI00024825.2 | PRG4 | ITEVWGIPSPIDTVFTR |
| IPI00294004.1 | | PROS1 | HCLVTVEK |
| IPI00013179.1 | IPI00013179.1 | PTGDS | TMLLQPAGSLGSYSYR |
| IPI00025426.1 | IPI00025426.2 | PZP | AFQPFFVELTMPYSVIR |
| IPI00003590.2 | | QSOX1 | LDVPVWDVEATLNFLK |
| IPI00221325.3 | IPI00221325.3 | RANBP2 | ERGIGNVKILR |
| IPI00411314.2 | IPI00304692.1 | RBMX | LTVIMVIP |
| 2895204 | IPI00022420.3 | RBP4 | FSGTWYAMAK |
| IPI00009027.1 | IPI00009027.1 | REG1A | ISCPEGTNAYR |
| IPI00014048.1 | IPI00014048.1 | RNASE1 | QHMDSDSSPSSSSTYCNQMMR |
| IPI00007047.1 | IPI00007047.1 | S100A8 | LLETECPCIYIR |
| IPI00027462.1 | IPI00027462.1 | S100A9 | VIEHIMEDLDTNADK |
| 247142 | IPI00006146.4 | SAA1 | SFFSFLGEAFDGAR |
| IPI00027191.1 | IPI00027191.1 | SAA3P | SGKDPNHFRPAGLPEK |
| IPI00019399.1 | IPI00019399.1 | SAA4 | VYLQGLIDYYLFGNSSTVLEDSK |
| | IPI00218795.1 | SELL | |
| IPI00029061.2 | IPI00029061.3 | SEPP1 | LVYHLGLPFSFLTFPYVEEAIK |
| 223433 | IPI00553177.1 | SERPINA1 | FNKPFVFLMIEQNTK |
| IPI00007199.4 | IPI00007199.4 | SERPINA10 | IFSPFADLSELSATGR |
| 225769 | IPI00550991.3 | SERPINA3 | RLYGSEAFATDFQDSAAAK |
| IPI00027482.1 | IPI00027482.1 | SERPINA6 | NVDFAFSLYK |
| IPI00292946.1 | IPI00292946.1 | SERPINA7 | EGQMESVEAAMSSK |
| IPI00032179.2 | IPI00032179.3 | SERPINC1 | NDNDNIFLSPLSISTAFAMTK |
| 23273330 | IPI00292950.4 | SERPIND1 | GGETAQSADPQWEQLNNK |
| 39725934 | IPI00006114.4 | SERPINF1 | LDLQEINNWVQAQMK |
| IPI00029863.4 | IPI00879231.1 | SERPINF2 | LGNQSPGGQTALK |
| 179621 | IPI00291866.5 | SERPING1 | GVTSYSQIFHSPDLAIR |
| | IPI00179016.8 | SETD1A | |
| IPI00023019.1 | IPI00023019.1 | SHBG | VVLSSGSGPGLDLPLVLGLPLQLK |
| | IPI00011961.1 | SIGLEC1 | |
| IPI00020687.1 | IPI00020687.1 | SPINK1 | IYDPVCGTDGNTYPNECVLCFENR |
| IPI00550363.2 | IPI00550363.3 | TAGLN2 | YGINTTDIFQTVDLWEGK |
| 2815575 | IPI00022463.1 | TF | KSVEEYANCHLAR |
| IPI00032292.1 | IPI00032292.1 | TIMP1 | LQSGTHCLWTDQLLQGSEK |
| IPI00298994.5 | IPI00298994.5 | TLN1 | GVAALTSDPAVQAIVLDTASDVLDK |
| IPI00180240.2 | | TMSL3 | NPLPSKETIEQEK |
| | IPI00554760.1 | TNR | |
| IPI00010779.3 | | TPM4 | AEGDVAALNR |
| | | TRANK1 | |
| | IPI00413160.4 | TRIOBP | |
| IPI00023283.3 | IPI00023283.3 | TIN | SEPIVAR |
| 230651 | IPI00022432.1 | TIR | KAADDTWEPFASGK |
| IPI00295413.8 | IPI00807602.1 | ULK4 | TEHNOTFTR |
| 33358191 | IPI00386524.3 | unknown 51 | ALQLTQSPSSLSASVGDR |
| 51103537 | | unknown 70 | DVVMTQSPLSLAVTFGEPASISCR |
| 51103559 | | unknown 72 | DIVMTQSPLSLAVTFGEPASISCR |
| 896277 | | unknown 8 | SYELTQPPSVSVSPGKTAR |
| | IPI00887739.3 | unknowna111 | |
| | IPI00894523.1 | unknowna126 | |
| | IPI00930382.1 | unknowna149 | |
| | IPI00736860.3 | unknowna60 | |
| | | unknown_d1 | |
| | | unknown_d10 | |
| | | unknown_d11 | |
| | | unknown_d12 | |
| | | unknown_d13 | |
| | | unknown_d14 | |
| | | unknown_d15 | |
| | | unknown_d16 | |
| | | unknown_d17 | |
| | | unknown_d2 | |
| | | unknown_d3 | |
| | | unknown_d4 | |
| | | unknown_d5 | |
| | | unknown_d6 | |
| | | unknown_d7 | |
| | | unknown_d8 | |
| | | unknown_d9 | |
| | IPI00020037.1 | USF2 | |
| IPI00395488.2 | IPI00395488.2 | VASN | LAGLGLQQLDEGLFSR |
| IPI00027038.1 | IPI00027038.1 | VSIG4 | VATLSTLLFKPAVIADSGSYFCTAK |
| IPI00298971.1 | IPI00298971.1 | VTN | DVWGIEGPIDAAFTR |
| IPI00023014.1 | IPI00023014.1 | VWF | LLDLVFLLDGSSR |
| t₀ Protein ID | t₂₀ Protein ID | Gene Symbol | t₂₄ Best Peptide Sequence |
| IPI00022895.7 | IPI00022895.7 | A1BG | CEGPIPDVTFELLR |
| IPI00478003.1 | IPI00478003.1 | AZM | VSNQTLSFFTVLQDVPVR |
| IPI00328762.4 | IPI00328762.5 | ABCA13 | YIYELLN |
| IPI00021428.1 | | ACTA1 | |
| IPI00008603.1 | IPI00008603.1 | ACTA2 | AGFAGDDAPR |
| IPI00021439.1 | IPI00021439.1 | ACT6 | AGFAGDDAPR |
| IPI00003269.1 | IPI00003269.1 | ACTBL2 | VAPDEHPILLTEAPLNPK |
| IPI00020019.1 | IPI00020019.1 | ADIPOQ | SAFSYGLETYVTIPNIYIPIT |
| | IPI00004344.1 | AFF4 | YNPSK |
| IPI00019943.1 | IPI00019943.1 | AFM | LKHELTDEELOSLFTNFANVVDK |
| IPI00022443.1 | IPI00022443.1 | AFP | CCQGQEQEVQFAEEGQK |
| 15079348 | IPI00032220.3 | AGT | SLDFTELDYAAEK |
| IPI00022431.1 | IPI00022431.2 | AHSG | EHAVEGDCDFQLLK |
| | | ALB | |
| IPI00022426.1 | IPI00022426.1 | AMBP | AFIQLWAFDAVK |
| IPI00022391.1 | IPI00022391.1 | APCS | IVLGQEQDSYGGK |
| 253362 | IPI00021841.1 | APOA1 | VKDLATVYVDVLK |
| 671882 | IPI00021854.1 | APOA2 | KASTELVNFLSYSVELGTQPATQ |
| IPI00304273.2 | IPI00847179.1 | APOA4 | LGEVNTYAGDLQK |
| 225311 | IPI00022229.3 | APO8 | LSLESLTSYFSIESSTK |
| IPI00021855.1 | IPI00021855.1 | APOC1 | LKEFGNTLEDK |
| IPI00021856.3 | IPI00021856.3 | APOC2 | STAAMSTYIGIFTOQVLSVLK |
| IPI00021857.1 | IPI00021857.1 | APOC3 | DALSSVQESQVAQQAR |
| IPI00022731.1 | IPI00022731.1 | APOC4 | DGWQWFWSPSTFT |
| IPI00006662.1 | IPI00006662.1 | APOD | CPNPPVQENFDVNK |
| 15826311 | IPI00021842.1 | APOE | GEVQAMLGQSTEELR |
| | | APOF | |
| IPI00298828.3 | IPI00298828.3 | APOH | CSYTEDAQCIDGTIEVPK |
| IPI00186903.3 | IPI00186903.4 | APOL1 | VTEPISAESEEQVER |
| IPI00027235.1 | | ATRN | |
| IPI00166729.4 | IPI00166729.4 | AZGP1 | YSLTVNTGLSK |
| IPI00004656.1 | IPI00004656.3 | B2M | SNFLNCYVSGFHPSQIEVDLLK |
| IPI00297188.5 | IPI00297188.6 | BAI2 | CPPNASGSASR |
| IPI00022392.1 | IPI00022392.1 | C1QA | SLGFCGTTNK |
| IPI00477992.1 | IPI00477992.1 | C1QB | LEQGENVFLOATDK |
| IPI00022394.2 | IPI00022394.2 | C1QC | FNAVLTNPQGDYOTSTGK |
| IPI00296165.5 | IPI00296165.6 | C1R | LVFQQFDLEPSEGCFYDYVK |
| IPI00009793.2 | IPI00009793.4 | C1RL | LGNFPWQAFTSIHGR |
| IPI00017696.1 | IPI00017696.1 | CLS | SNALDIFQTDLTGQK |
| IPI00745619.1 | IPI00303963.1 | C2 | PICLPCTMEANLALR |
| IPI00783987.1 | IPI00783987.2 | C3 | VQLSNDFDEYIMAIEQTIK |
| 179674 | IPI00892547.1 | C4A | GLEEELQFSLGSK |
| IPI00418163.3 | IPI00418163.3 | C48 | GLEEELQFSLGSK |
| IPI00021727.1 | IPI00021727.1 | C48PA | CEWETPEGCEQVLTGR |
| IPI00025862.1 | IPI00025862.2 | C48PB | NLCEAMENFMQQLK |
| 38016947 | IPI00032291.2 | C5 | YIYPLDSLTWIEYWPR |
| IPI00009920.2 | IPI00879709.3 | C6 | CPINCLLGDFGPWSDCDPCIEK |
| 179716 | IPI00296608.6 | C7 | MPYECGPSLDVCAQDER |
| 9016854 | IPI00011252.1 | C8A | AIDEDCSQYEPIPGSQK |
| IPI00294395.1 | | C8B | |
| IPI00011261.2 | IPI00011261.2 | C8G | VQEAHLTEDQIFYFPK |
| IPI00022395.1 | IPI00022395.1 | C9 | GTVIDVTDFVNWASSINDAPVLISQK |
| IPI00215983.2 | IPI00215983.3 | CA1 | HDTSLKPISVSYNPATAR |
| IPI00465436.3 | IPI00465436.4 | CAT | LGPNYLHIPVNVPYR |
| IPI00029260.2 | IPI00029260.2 | CD14 | VLSIAQAHSPAFSCEQVR |
| IPI00104074.4 | | CD163 | |
| IPI00025204.1 | IPI00025204.1 | CD5L | EATLQDCPSGPWGK |
| 40737516 | | CDA5 | |
| | IPI00855958.1 | CENPF | SSGIWENGR |
| IPI00010180.3 | 010180.4 | CES1 | ESOPLLGTVIDGMLLIK |
| IPI00019591.1 | IPI00893864.1 | CF8 | LLQEGOALEYVCPSGFYPYPVQTR |
| IPI00019579.1 | IPI00165972.3 | CFD | RPDSLQHVLLPVLDR |
| IPI00029739.4 | IPI00029739.5 | CFH | CFEGFGIDGPAIAK |
| IPI00006543.2 | IPI00011264.2 | CFHR1 | YKPESQVPTGEVFYYSCEYNFVSPSK |
| IPI00006154.1 | IPI00006154.1 | CFHR2 | ITCAEEGWSPTPK |
| IPI00027507.1 | IPI00027507.1 | CFHR3 | KCYFPYLENGYNQNYGR |
| IPI00291867.3 | IPI00291867.3 | CF1 | TMGYQDFADVVCYTQK |
| IPI00012011.5 | | CFL1 | |
| IPI00021364.4 | IPI00021364.1 | CFP | HCYSIQHCPLK |
| IPI00009028.1 | IPI00009028.1 | CLEC38 | LDTLAQEVALLK |
| IPI00291262.3 | IPI00291262.3 | CLU | EILSVDCSTNNPSQAK |
| IPI00011283.1 | IPI00011783.2 | COL11A2 | GDPGPPGAPGKDGPAGLR |
| 179594 | IPI00297646.4 | COL1A1 | VLCDDVICDETK |
| IPI00168920.2 | IPI00168920.3 | COL24A1 | NKNRLQLGVQLLPK |
| 930045 | IPI00021033.2 | COL3A1 | GPPGINGSPGGK |
| IPI00025418.1 | IPI00025418.2 | COL7A1 | IFSPIREAQASGLNVVML |
| 2632189 | IPI00423463.1 | COPB1 | HNWFDPWGQGTLVTISSASTK |
| IPI00017601.1 | IPI00017601.1 | CP | HYYIAAEEIIWNYAPSGIDIFTK |
| IPI00293057.5 | | CPBZ | |
| IPI00010295.1 | IPI00010295.1 | CPN1 | ELMLQLSEFLCEEFR |
| IPI00479116.1 | IPI00475136.3 | CPN2 | TLNLAQNLLAQLPEELFHPLTSLQTLK |
| | IPI00011062.1 | CPS1 | IAPSFAVESIEGALK |
| IPI00022339.1 | IPI00022389.1 | CRP | GYSIFSYATK |
| IPI00032293.1 | IPI00032293.1 | CST3 | LVGGPMDASVEEEGVR |
| IPI00005721.1 | IPI00005721.1 | DEFA1 | ADEVAAAPEQIAADIPEVVVSLAWDESLAPK |
| IPI00465045.2 | | DIP2B | |
| 4758236 | IPI00003351.2 | ECM1 | NIWRDPALCCYLSPGDEQVNCFNINYLR |
| IPI00019531.1 | IPI00019581.1 | F12 | GRPGPQPWCATTPNFDQDQR |
| IPI00019588.1 | IPI00019568.1 | F2 | IVEGSDAEUGMSPQEVMLFR |
| IPI00010290.1 | IPI00010290.2 | FABP1 | AIGLPEELIQK |
| IPI00215746.2 | | FABP4 | |
| | | FAM135A | |
| IPI00218803.2 | IPI00218803.3 | FELN1 | DIDECESGTHNCLPDFICQNTLGSFR |
| IPI00242956.3 | IPI00242956.5 | FCGBP | APGWDPLCWDECR |
| IPI00293925.2 | | FCN3 | |
| IPI00021885.1 | IPI00021885.1 | FGA | ADSGEGDFLAEGGGVR |
| IPI00298437.3 | IPI00298497.3 | FGB | DNENVVNEYSSELEK |
| IPI00021891.5 | IPI00021891.5 | FGG | YEASILTHDSSIR |
| | IPI00289334.1 | FLNB | PASFAIR |
| 51476364 | IPI00022418.1 | FN1 | SYTTTGLQPGTDYK |
| | IPI00375676.3 | FTL | KINQALLDLHALGSAR |
| IPI00010375.3 | IPI00555812.4 | GC | FMPAAQLPELPDVELPTNK |
| IPI00026199.1 | IPI00026199.2 | GPK3 | FLVGPDGIPIMR |
| IPI00026314.1 | IPI00026314.1 | GSN | AQPVQVAEGSEPDGFQEAGGK |
| IPI00465253.4 | IPI00465253.4 | HAU56 | LDGTNVANIPR |
| 27574247 | IPI00410714.5 | HBA1 | VGAHAGEYGAEALER |
| 13195586 | | HBA2 | |
| IPI00658153.1 | IPI00654755.3 | HBB | VLGAFSDELAHLDNLK |
| IPI00473011.2 | IPI00473011.3 | HBD | VLGAFSDGLAHLDNLK |
| | IPI00220706.10 | HBG1 | VNVEDAGGETLGR |
| IPI00217473.4 | IPI00217473.5 | HBZ | LRVDPVNFK |
| 229528 | | HLA-E | |
| 229271 | IPI00902590.1 | HP | SPVGVQPILNEHTFCAGMSK |
| IPI00477597.1 | IPI00477597.1 | HPR | TEGDGVYTLNDK |
| IPI00022488.1 | IPI00022488.2 | HPX | LLQDEFPGIPSPLDAAVECHR |
| IPI00022371.1 | IPI00022371.1 | HRG | GGEGTGYFVDFSVR |
| | IPI00220362.5 | HSPE1 | GGEIQPVSVK |
| IPI00005477.4 | IPI00009477.4 | ICAM2 | YPTVEPLDSLTLFLER |
| | | IGFALS | |
| IPI00297284.1 | | IGFBP2 | |
| IPI00305380.3 | IPI00305380.3 | IGFBP4 | EDARPVPQGSCQSELHR |
| IPI00029235.1 | | IGFBP6 | |
| 34527679 | IPI00386524.3 | IGHA1 | SAVQGPPDRDLCGCYSVSSVLPGCAEPWNHGK |
| | | IGHD | |
| 21757089 | | IGHG1 | |
| | | IGHG2 | |
| | | IGHG3 | |
| | | IGHG4 | |
| | | IGHM | |
| | | IGJ | |
| | | IGKC | |
| | | IGKY1-5 | |
| | | IGLC7 | |
| 1871489 | | IGM | |
| 33319112 | | IGVH2 | |
| IPI00292530.1 | IPI00292530.1 | ITH1 | LWAYETIQELLAK |
| IPI00305461.2 | IPI00305461.3 | ITH2 | AHVSFKPTVAQQR |
| IPI00026413.1 | IPI00028413.8 | ITH3 | LWAYLTIEQLLEK |
| IPI00218192.1 | IPI00218192.3 | ITH4 | SPEQQETVLDGNLIIR |
| 1575607 | IPI00479786.5 | KHSRP | AGLVIGK |
| | IPI00827544.1 | KIF19 | QIIDDYNL |
| IPI00654888.2 | IPI00654888.2 | KLKB1 | CLLFSFLPASSINDMEK |
| IPI00032328.1 | IPI00215894.1 | KNGL | LGQSLDCNAEVYVVPWEK |
| | | KRT31 | |
| | | KRT81 | |
| 31652249 | IPI00032311.4 | LBP | GLQYAAQEGLLALQSELLR |
| IPI00299547.4 | IPI00299547.4 | LCN2 | SLGLPENHIVFPVPIDQCIDG |
| IPI00010471.4 | IPI00010471.5 | LCP1 | INLARANLFNR |
| IPI00023673.1 | IPI00023673.1 | LGALS3BP | GQWGTVCDNLWDLTDASVVCR |
| IPI00164623.4 | | LOC100133511 | |
| IPI00167093.4 | | LOC100293069 | |
| IPI00061977.1 | | LOC100294459 | |
| IPI00736860.2 | | LOC100294460 | |
| IPI00029168.1 | IPI00029168.1 | LPA | TPEYVPNAGLIMNYCR |
| 21707947 | IPI00022417.4 | LRG1 | ENQLEVLEVWLHGLK |
| IPI00298860.5 | | LTF | |
| IPI00020986.2 | IPI00020986.2 | LUM | LPSGLPVSLLTLYDNNK |
| 3402141 | IPI00019038.1 | LYZ | TPCAVNACHLSCSALLQDNIADAVACAK |
| | 126508 | LYZL4 | GYSLGNWVCAAK |
| | | MACF1 | |
| IPI00217493.4 | IPI00217493.5 | MB | HGATVLTALGGILK |
| IPI00004373.1 | IPI00004373.1 | MBL2 | FQASVATPR |
| 21756643 | | MCAM | |
| IPI00306929.7 | IPI00306929.9 | MYO18B | TTELKEAEPQGK |
| | IPI00423460.3 | NID1 | DLCGCYSVPSVLPGCAEPWNHGK |
| IPI00022429.3 | IPI00022429.3 | ORM1 | NWGLSVYADKPETTK |
| IPI00020091.1 | IPI00020091.1 | ORM2 | NWGLSVYADKPETTK |
| IPI00022295.1 | | PF4V1 | |
| IPI00216694.1 | | PFN1 | |
| IPI00163207.1 | IPI00163207.1 | PGLYRP2 | SLPLLMDSVIQALAELEQK |
| IPI00004573.1 | IPI00004573.2 | PIGR | NADLQVLKPEPELVYEDLR |
| IPI00306311.8 | IPI00306311.8 | PLEK | KSEEEMLFEIITADEVHYFLQAATPK |
| 229453 | IPI00019580.1 | PLG | VILGAHQEVNLEPHVQEIEYSR |
| 1262347 | IPI00218732.3 | PON1 | IFFYDSENPPASEVLR |
| IPI00555900.1 | | POTEK | |
| IPI00022445.1 | IPI00022445.1 | PFBP | GKEESLDSDLYAELR |
| IPI00027350.3 | IPI00027350.2 | PRDX2 | ATAVVDGAFK |
| IPI00024825.2 | IPI00024825.2 | PRG4 | GLPNVVTSAISLPNIR |
| IPI00294004.1 | | PROS1 | |
| IPI00013179.1 | IPI00013179.1 | PTGDS | APEAQVSQPNFQQQK |
| IPI00025426.1 | IPI00025426.2 | PZP | GSGALSFPVESDVAPIAR |
| IPI00003590.2 | | QSOX1 | |
| IPI00221325.3 | IPI00221325.3 | RANBP2 | DTSFLGSDDIGNIDVR |
| IPI00411314.2 | IPI00304692.1 | RBMX | RDVYLSPR |
| 2895204 | IPI00022420.3 | RBP4 | LLNNWDYCADMVGTFTDTEDPAK |
| IPI00009027.1 | IPI00009027.1 | REG1A | RWHWSSGSLVSYK |
| IPI00014048.1 | IPI00014048.1 | RNASE1 | CKPVNTFVHEPLVDVQNVCFQEK |
| IPI00007047.1 | IPI00007047.1 | S100A8 | ALNSIIDVYHK |
| IPI00027462.1 | IPI00027462.1 | S100A9 | VIEHIMEDLDTNADK |
| 247142 | IPI00006146.4 | SAA1 | GPGGAWAAEVISNAR |
| IPI00027191.1 | IPI00027191.1 | SAA3P | SGKDPNHFRPAGLPEK |
| IPI00019399.1 | IPI00019399.1 | SAA4 | VYLQGLIDYYLFGNSSTVLEDSK |
| | IPI00218795.1 | SELL | NKEDCVEIYIK |
| IPI00029061.2 | IPI00029061.3 | SEPP1 | LVYHLGLPFSFLTFPYVEEAIK |
| 223433 | IPI00553177.1 | SERPINA1 | VFSNGADLSGVETTAPLK |
| IPI00007199.4 | IPI00007199.4 | SERPINA10 | IFSPFADLSELSATGR |
| 225769 | IPI00550991.3 | SERPINA3 | RLYGSEAFATDFQDSAAAK |
| IPI00027482.1 | IPI00027482.1 | SERPINA6 | WSAGLTSSQVDLYIPK |
| IPI00292946.1 | IPI00292946.1 | SERPINA7 | SFMLLILER |
| IPI00032179.2 | IPI00032179.3 | SERPINC1 | FATTFYQHLAQSK |
| 23273330 | IPI00292950.4 | SERPIND1 | GGETAQSADPQWEQLNNK |
| 39725934 | IPI00006114.4 | SERPINF1 | SSMSPTTNVLLSPLSVATALSALSGAEQR |
| IPI00029863.4 | IPI00879231.1 | SERPINF2 | LCQDLGPGAFR |
| 179621 | IPI00291866.5 | SERPING1 | LVLLNAMLSAK |
| | IPI00179016.8 | SETD1A | FQGSGAATETAESRR |
| IPI00023019.1 | IPI00023019.1 | SHBG | ALALPPLGLAPILNLWAKPQGR |
| | IPI00011961.1 | SIGLEC1 | GCSPR |
| IPI00020687.1 | IPI00020687.1 | SPINK1 | IYDPVCGTDGNTYPNECVLCFENR |
| IPI00550363.2 | IPI00550363.3 | TAGLN2 | YGINTTDIFQTVDLWEGK |
| 2815575 | IPI00022463.1 | TF | TAGWNIFMGLLYNK |
| IPI00032292.1 | IPI00032292.1 | TIMP1 | LQSGTHCLWTDQLLQGSEK |
| IPI00298994.5 | IPI00298994.5 | TLN1 | GTEWVDPEDPTVIAENELLGAAAAIEAAAK |
| IPI00180240.2 | | TMSL3 | |
| | IPI00554760.1 | TNR | QQSLESSTVDAFTGIDPPK |
| IPI00010779.3 | | TPM4 | |
| | | TRANK1 | |
| | IPI00413160.4 | TRIOBP | SPVGGDAAGQKK |
| IPI00023283.3 | IPI00023283.3 | TIN | VLACNAGGPGEPEAVPGTVK |
| 230651 | IPI00022432.1 | TIR | KAADDTWEPFASGK |
| IPI00295413.8 | IPI00807602.1 | ULK4 | ILCEDPLPPIPKDSSRPK |
| 33358191 | IPI00386524.3 | unknown 51 | |
| 51103537 | | unknown 70 | |
| 51103559 | | unknown 72 | |
| 896277 | | unknown 8 | |
| | IPI00887739.3 | unknowna111 | SNLDEDIIAEENIVSR |
| | IPI00894523.1 | unknowna126 | AGFAGDDAPR |
| | IPI00930382.1 | unknowna149 | SPVGVQPVPILNEHTF |
| | IPI00736860.3 | unknowna60 | DYFPEPVTVSWNSGALTR |
| | | unknown_d1 | |
| | | unknown_d10 | |
| | | unknown_d11 | |
| | | unknown_d12 | |
| | | unknown_d13 | |
| | | unknown_d14 | |
| | | unknown_d15 | |
| | | unknown_d16 | |
| | | unknown_d17 | |
| | | unknown_d2 | |
| | | unknown_d3 | |
| | | unknown_d4 | |
| | | unknown_d5 | |
| | | unknown_d6 | |
| | | unknown_d7 | |
| | | unknown_d8 | |
| | | unknown_d9 | |
| | IPI00020037.1 | USF2 | MLDPGLDPAASATAAAAASHDK |
| IPI00395488.2 | IPI00395488.2 | VASN | LLLLDLSHNSLLALEPGILDTANVEALR |
| IPI00027038.1 | IPI00027038.1 | VSIG4 | VATLSTLLFKPAVIADSGSYFCTAK |
| IPI00298971.1 | IPI00298971.1 | VTN | DVWGIEGPIDAAFTR |
| IPI00023014.1 | IPI00023014.1 | VWF | IGWPNAPILIQDFETLPR |

| | | | | t24 | | AUC | t₀ | t₂₆ |
|---|---|---|---|---|---|---|---|---|
| | | | Priority | Priority | AUC | t₂₄ | Spectral | Spectral |
| t₀ Protein ID | t₂₀ Protein ID | Gene Symbol | Score t₀ | Score | t₀ ID | ID | Count ID | Count ID |
| IPI00022895.7 | IPI00022895.7 | A1BG | 1 | 1 | 1 | 1 | 1 | 1 |
| IPI00478003.1 | IPI00478003.1 | AZM | 1 | 1 | 1 | 1 | 1 | 1 |
| IPI00328762.4 | IPI00328762.5 | ABCA13 | 1 | 2 | 1 | 1 | 0 | 0 |
| IPI00021428.1 | | ACTA1 | 1 | | 1 | 0 | 0 | 0 |
| IPI00008603.1 | IPI00008603.1 | ACTA2 | 1 | 2 | 1 | 1 | 0 | 0 |
| IPI00021439.1 | IPI00021439.1 | ACT6 | 1 | 1 | 1 | 1 | 0 | 0 |
| IPI00003269.1 | IPI00003269.1 | ACTBL2 | 1 | 2 | 1 | 1 | 0 | 0 |
| IPI00020019.1 | IPI00020019.1 | ADIPOQ | 1 | 2 | 1 | 1 | 0 | 0 |
| | IPI00004344.1 | AFF4 | | 1 | 0 | 1 | 0 | 0 |
| IPI00019943.1 | IPI00019943.1 | AFM | 1 | 1 | 1 | 1 | 1 | 1 |
| IPI00022443.1 | IPI00022443.1 | AFP | 1 | 1 | 1 | 1 | 0 | 0 |
| 15079348 | IPI00032220.3 | AGT | 1 | 1 | 1 | 1 | 1 | 1 |
| IPI00022431.1 | IPI00022431.2 | AHSG | 1 | 1 | 1 | 1 | 1 | 1 |
| | | ALB | | | 0 | 0 | 1 | 1 |
| IPI00022426.1 | IPI00022426.1 | AMBP | 1 | 1 | 1 | 1 | 1 | 1 |
| IPI00022391.1 | IPI00022391.1 | APCS | 1 | 1 | 1 | 1 | 1 | 1 |
| 253362 | IPI00021841.1 | APOA1 | 1 | 1 | 1 | 1 | 1 | 1 |
| 671882 | IPI00021854.1 | APOA2 | 1 | 1 | 1 | 1 | 1 | 1 |
| IPI00304273.2 | IPI00847179.1 | APOA4 | 1 | 1 | 1 | 1 | 1 | 1 |
| 225311 | IPI00022229.3 | APO8 | 1 | 1 | 1 | 1 | 1 | 1 |
| IPI00021855.1 | IPI00021855.1 | APOC1 | 1 | 1 | 1 | 1 | 1 | 1 |
| IPI00021856.3 | IPI00021856.3 | APOC2 | 1 | 1 | 1 | 1 | 1 | 1 |
| IPI00021857.1 | IPI00021857.1 | APOC3 | 1 | 1 | 1 | 1 | 1 | 1 |
| IPI00022731.1 | IPI00022731.1 | APOC4 | 1 | 1 | 1 | 1 | 0 | 0 |
| IPI00006662.1 | IPI00006662.1 | APOD | 1 | 1 | 1 | 1 | 1 | 1 |
| 15826311 | IPI00021842.1 | APOE | 1 | 1 | 1 | 1 | 1 | 1 |
| | | APOF | | | 0 | 0 | 1 | 1 |
| IPI00298828.3 | IPI00298828.3 | APOH | 1 | 1 | 1 | 1 | 1 | 1 |
| IPI00186903.3 | IPI00186903.4 | APOL1 | 1 | 1 | 1 | 1 | 1 | 1 |
| IPI00027235.1 | | ATRN | 1 | | 1 | 0 | 0 | 0 |
| IPI00166729.4 | IPI00166729.4 | AZGP1 | 1 | 1 | 1 | 1 | 1 | 1 |
| IPI00004656.1 | IPI00004656.3 | B2M | 1 | 1 | 1 | 1 | 1 | 1 |
| IPI00297188.5 | IPI00297188.6 | BAI2 | 4 | 1 | 1 | 1 | 0 | 0 |
| IPI00022392.1 | IPI00022392.1 | C1QA | 1 | 1 | 1 | 1 | 1 | 1 |
| IPI00477992.1 | IPI00477992.1 | C1QB | 1 | 1 | 1 | 1 | 1 | 1 |
| IPI00022394.2 | IPI00022394.2 | C1QC | 1 | 1 | 1 | 1 | 1 | 1 |
| IPI00296165.5 | IPI00296165.6 | C1R | 1 | 1 | 1 | 1 | 1 | 1 |
| IPI00009793.2 | IPI00009793.4 | C1RL | 2 | 1 | 1 | 1 | 0 | 0 |
| IPI00017696.1 | IPI00017696.1 | CLS | 2 | 1 | 1 | 1 | 0 | 0 |
| IPI00745619.1 | IPI00303963.1 | C2 | 1 | 1 | 1 | 1 | 1 | 1 |
| IPI00783987.1 | IPI00783987.2 | C3 | 1 | 1 | 1 | 1 | 1 | 1 |
| 179674 | IPI00892547.1 | C4A | 1 | 1 | 1 | 1 | 0 | 0 |
| IPI00418163.3 | IPI00418163.3 | C48 | 1 | 1 | 1 | 1 | 1 | 1 |
| IPI00021727.1 | IPI00021727.1 | C48PA | 1 | 1 | 1 | 1 | 1 | 1 |
| IPI00025862.1 | IPI00025862.2 | C48PB | 1 | 1 | 1 | 1 | 0 | 0 |
| 38016947 | IPI00032291.2 | C5 | 1 | 1 | 1 | 1 | 1 | 1 |
| IPI00009920.2 | IPI00879709.3 | C6 | 1 | 1 | 1 | 1 | 1 | 1 |
| 179716 | IPI00296608.6 | C7 | 1 | 1 | 1 | 1 | 1 | 1 |
| 9016854 | IPI00011252.1 | C8A | 1 | 1 | 1 | 1 | 1 | 1 |
| IPI00294395.1 | | C8B | 1 | | 1 | 0 | 0 | 0 |
| IPI00011261.2 | IPI00011261.2 | C8G | 1 | 1 | 1 | 1 | 1 | 1 |
| IPI00022395.1 | IPI00022395.1 | C9 | 1 | 1 | 1 | 1 | 1 | 1 |
| IPI00215983.2 | IPI00215983.3 | CA1 | 1 | 1 | 1 | 1 | 0 | 0 |
| IPI00465436.3 | IPI00465436.4 | CAT | 1 | 1 | 1 | 1 | 0 | 0 |
| IPI00029260.2 | IPI00029260.2 | CD14 | 1 | 1 | 1 | 0 | 1 | 1 |
| IPI00104074.4 | | CD163 | 1 | | 1 | 1 | 0 | 0 |
| IPI00025204.1 | IPI00025204.1 | CD5L | 1 | 1 | 1 | 0 | 0 | 0 |
| 40737516 | | CDA5 | 1 | | 1 | 1 | 0 | 0 |
| | IPI00855958.1 | CENPF | | 1 | 0 | 1 | 0 | 0 |
| IPI00010180.3 | 010180.4 | CES1 | 1 | 1 | 1 | 1 | 0 | 1 |
| IPI00019591.1 | IPI00893864.1 | CF8 | 1 | 1 | 1 | 1 | 1 | 1 |
| IPI00019579.1 | IPI00165972.3 | CFD | 1 | 1 | 1 | 1 | 0 | 0 |
| IPI00029739.4 | IPI00029739.5 | CFH | 1 | 1 | 1 | 1 | 1 | 1 |
| IPI00006543.2 | IPI00011264.2 | CFHR1 | 2 | 1 | 1 | 1 | 0 | 0 |
| IPI00006154.1 | IPI00006154.1 | CFHR2 | 1 | 1 | 1 | 1 | 0 | 0 |
| IPI00027507.1 | IPI00027507.1 | CFHR3 | 1 | 1 | 1 | 1 | 0 | 0 |
| IPI00291867.3 | IPI00291867.3 | CF1 | 1 | 1 | 1 | 1 | 1 | 1 |
| IPI00012011.5 | | CFL1 | 1 | | 1 | 0 | 0 | 0 |
| IPI00021364.4 | IPI00021364.1 | CFP | 1 | 1 | 1 | 1 | 0 | 0 |
| IPI00009028.1 | IPI00009028.1 | CLEC38 | 1 | 1 | 1 | 1 | 1 | 1 |
| IPI00291262.3 | IPI00291262.3 | CLU | 1 | 1 | 1 | 1 | 1 | 1 |
| IPI00011283.1 | IPI00011783.2 | COL11A2 | 3 | 1 | 1 | 1 | 0 | 0 |
| 179594 | IPI00297646.4 | COL1A1 | 2 | 1 | 1 | 1 | 0 | 0 |
| IPI00168920.2 | IPI00168920.3 | COL24A1 | 2 | 1 | 1 | 1 | 0 | 0 |
| 930045 | IPI00021033.2 | COL3A1 | 4 | 1 | 1 | 1 | 0 | 0 |
| IPI00025418.1 | IPI00025418.2 | COL7A1 | 2 | 1 | 1 | 1 | 0 | 0 |
| 2632189 | IPI00423463.1 | COPB1 | 4 | 1 | 1 | 1 | 0 | 0 |
| IPI00017601.1 | IPI00017601.1 | CP | 1 | 1 | 1 | 1 | 1 | 1 |
| IPI00293057.5 | | CPBZ | 1 | | 1 | 0 | 0 | 0 |
| IPI00010295.1 | IPI00010295.1 | CPN1 | 1 | 1 | 1 | 1 | 0 | 0 |
| IPI00479116.1 | IPI00475136.3 | CPN2 | 1 | 1 | 1 | 1 | 1 | 1 |
| | IPI00011062.1 | CPS1 | | 1 | 0 | 1 | 0 | 1 |
| IPI00022339.1 | IPI00022389.1 | CRP | 1 | 1 | 1 | 1 | 1 | 1 |
| IPI00032293.1 | IPI00032293.1 | CST3 | 1 | 1 | 1 | 1 | 1 | 1 |
| IPI00005721.1 | IPI00005721.1 | DEFA1 | 1 | 1 | 1 | 1 | 0 | 0 |
| IPI00465045.2 | | DIP2B | 1 | | 1 | 0 | 0 | 0 |
| 4758236 | IPI00003351.2 | ECM1 | 1 | 1 | 1 | 1 | 0 | 0 |
| IPI00019531.1 | IPI00019581.1 | F12 | 1 | 1 | 1 | 1 | 0 | 0 |
| IPI00019588.1 | IPI00019568.1 | F2 | 1 | 1 | 1 | 1 | 1 | 1 |
| IPI00010290.1 | IPI00010290.2 | FABP1 | 1 | 2 | 1 | 1 | 0 | 0 |
| IPI00215746.2 | | FABP4 | 1 | | 1 | 0 | 0 | 0 |
| | | FAM135A | | | 0 | 0 | 1 | 1 |
| IPI00218803.2 | IPI00218803.3 | FELN1 | 1 | 2 | 1 | 1 | 0 | 0 |
| IPI00242956.3 | IPI00242956.5 | FCGBP | 1 | 1 | 1 | 1 | 1 | 1 |
| IPI00293925.2 | | FCN3 | 1 | | 1 | 0 | 0 | 0 |
| IPI00021885.1 | IPI00021885.1 | FGA | 1 | 1 | 1 | 1 | 1 | 1 |
| IPI00298437.3 | IPI00298497.3 | FGB | 1 | 1 | 1 | 1 | 1 | 1 |
| IPI00021891.5 | IPI00021891.5 | FGG | 1 | 1 | 1 | 1 | 1 | 1 |
| | IPI00289334.1 | FLNB | | 1 | 0 | 1 | 0 | 0 |
| 51476364 | IPI00022418.1 | FN1 | 1 | 1 | 1 | 1 | 1 | 1 |
| | IPI00375676.3 | FTL | | 1 | 0 | 1 | 0 | 1 |
| IPI00010375.3 | IPI00555812.4 | GC | 4 | 1 | 1 | 1 | 1 | 1 |
| IPI00026199.1 | IPI00026199.2 | GPK3 | 1 | 1 | 1 | 1 | 0 | 0 |
| IPI00026314.1 | IPI00026314.1 | GSN | 1 | 1 | 1 | 1 | 1 | 1 |
| IPI00465253.4 | IPI00465253.4 | HAU56 | 2 | 1 | 1 | 1 | 0 | 0 |
| 27574247 | IPI00410714.5 | HBA1 | 1 | 1 | 1 | 1 | 1 | 1 |
| 13195586 | | HBA2 | 1 | | 1 | 0 | 0 | 0 |
| IPI00658153.1 | IPI00654755.3 | HBB | 2 | 1 | 1 | 1 | 1 | 1 |
| IPI00473011.2 | IPI00473011.3 | HBD | 1 | 1 | 1 | 1 | 0 | 0 |
| | IPI00220706.10 | HBG1 | | 1 | 0 | 1 | 0 | 1 |
| IPI00217473.4 | IPI00217473.5 | HBZ | 1 | 1 | 1 | 1 | 0 | 0 |
| 229528 | | HLA-E | 1 | | 1 | 0 | 0 | 0 |
| 229271 | IPI00902590.1 | HP | 4 | 1 | 1 | 1 | 1 | 1 |
| IPI00477597.1 | IPI00477597.1 | HPR | 1 | 1 | 1 | 1 | 1 | 1 |
| IPI00022488.1 | IPI00022488.2 | HPX | 1 | 1 | 1 | 1 | 1 | 1 |
| IPI00022371.1 | IPI00022371.1 | HRG | 1 | 1 | 1 | 1 | 1 | 1 |
| | IPI00220362.5 | HSPE1 | | 1 | 0 | 1 | 0 | 0 |
| IPI00005477.4 | IPI00009477.4 | ICAM2 | 2 | 1 | 1 | 1 | 0 | 0 |
| | | IGFALS | | | 0 | 0 | 0 | 1 |
| IPI00297284.1 | | IGFBP2 | 1 | | 1 | 0 | 0 | 0 |
| IPI00305380.3 | IPI00305380.3 | IGFBP4 | 1 | 2 | 1 | 1 | 0 | 0 |
| IPI00029235.1 | | IGFBP6 | 1 | | 1 | 0 | 0 | 0 |
| 34527679 | IPI00386524.3 | IGHA1 | 1 | 1 | 1 | 1 | 1 | 1 |
| | | IGHD | | | 0 | 0 | 1 | 1 |
| 21757089 | | IGHG1 | 1 | | 1 | 0 | 1 | 1 |
| | | IGHG2 | | | 0 | 0 | 1 | 1 |
| | | IGHG3 | | | 0 | 0 | 1 | 1 |
| | | IGHG4 | | | 0 | 0 | 1 | 1 |
| | | IGHM | | | 0 | 0 | 1 | 1 |
| | | IGJ | | | 0 | 0 | 1 | 1 |
| | | IGKC | | | 0 | 0 | 1 | 1 |
| | | IGKY1-5 | | | 0 | 0 | 0 | 1 |
| | | IGLC7 | | | 0 | 0 | 1 | 1 |
| 1871489 | | IGM | 1 | | 1 | 0 | 0 | 0 |
| 33319112 | | IGVH2 | 1 | | 1 | 0 | 0 | 0 |
| IPI00292530.1 | IPI00292530.1 | ITH1 | 1 | 1 | 1 | 1 | 1 | 1 |
| IPI00305461.2 | IPI00305461.3 | ITH2 | 1 | 1 | 1 | 1 | 1 | 1 |
| IPI00026413.1 | IPI00028413.8 | ITH3 | 1 | 1 | 1 | 1 | 1 | 1 |
| IPI00218192.1 | IPI00218192.3 | ITH4 | 1 | 1 | 1 | 1 | 1 | 1 |
| 1575607 | IPI00479786.5 | KHSRP | 2 | 1 | 1 | 1 | 0 | 0 |
| | IPI00827544.1 | KIF19 | | 1 | 0 | 1 | 0 | 0 |
| IPI00654888.2 | IPI00654888.2 | KLKB1 | 1 | 1 | 1 | 1 | 0 | 0 |
| IPI00032328.1 | IPI00215894.1 | KNGL | 1 | 1 | 1 | 1 | 1 | 1 |
| | | KRT31 | | | 0 | 0 | 0 | 1 |
| | | KRT81 | | | 0 | 0 | 0 | 1 |
| 31652249 | IPI00032311.4 | LBP | 1 | 1 | 1 | 1 | 1 | 1 |
| IPI00299547.4 | IPI00299547.4 | LCN2 | 1 | 1 | 1 | 1 | 0 | 0 |
| IPI00010471.4 | IPI00010471.5 | LCP1 | 1 | 1 | 1 | 1 | 0 | 0 |
| IPI00023673.1 | IPI00023673.1 | LGALS3BP | 1 | 2 | 1 | 1 | 0 | 0 |
| IPI00164623.4 | | LOC100133511 | 1 | | 1 | 0 | 0 | 0 |
| IPI00167093.4 | | LOC100293069 | 1 | | 1 | 0 | 0 | 0 |
| IPI00061977.1 | | LOC100294459 | 1 | | 1 | 0 | 0 | 0 |
| IPI00736860.2 | | LOC100294460 | 1 | | 1 | 0 | 0 | 0 |
| IPI00029168.1 | IPI00029168.1 | LPA | 1 | 1 | 1 | 1 | 1 | 1 |
| 21707947 | IPI00022417.4 | LRG1 | 1 | 1 | 1 | 1 | 1 | 1 |
| IPI00298860.5 | | LTF | 1 | | 1 | 0 | 0 | 0 |
| IPI00020986.2 | IPI00020986.2 | LUM | 1 | 1 | 1 | 1 | 1 | 1 |
| 3402141 | IPI00019038.1 | LYZ | 1 | 1 | 1 | 1 | 0 | 0 |
| | 126508 | LYZL4 | | 1 | 0 | 1 | 0 | 0 |
| | | MACF1 | | | 0 | 0 | 1 | 1 |
| IPI00217493.4 | IPI00217493.5 | MB | 2 | 1 | 1 | 1 | 0 | 1 |
| IPI00004373.1 | IPI00004373.1 | MBL2 | 1 | 2 | 1 | 1 | 0 | 0 |
| 21756643 | | MCAM | 1 | | 1 | 0 | 0 | 0 |
| IPI00306929.7 | IPI00306929.9 | MYO18B | 2 | 1 | 1 | 1 | 0 | 0 |
| | IPI00423460.3 | NID1 | | 1 | 0 | 1 | 0 | 0 |
| IPI00022429.3 | IPI00022429.3 | ORM1 | 1 | 1 | 1 | 1 | 1 | 1 |
| IPI00020091.1 | IPI00020091.1 | ORM2 | 1 | 1 | 1 | 1 | 1 | 1 |
| IPI00022295.1 | | PF4V1 | 1 | | 1 | 0 | 0 | 0 |
| IPI00216694.1 | | PFN1 | 1 | | 1 | 0 | 0 | 0 |
| IPI00163207.1 | IPI00163207.1 | PGLYRP2 | 1 | 1 | 1 | 1 | 1 | 1 |
| IPI00004573.1 | IPI00004573.2 | PIGR | 2 | 1 | 1 | 1 | 0 | 0 |
| IPI00306311.8 | IPI00306311.8 | PLEK | 1 | 1 | 1 | 1 | 0 | 0 |
| 229453 | IPI00019580.1 | PLG | 1 | 1 | 1 | 1 | 1 | 1 |
| 1262347 | IPI00218732.3 | PON1 | 1 | 1 | 1 | 1 | 0 | 0 |
| IPI00555900.1 | | POTEK | 1 | | 1 | 0 | 0 | 0 |
| IPI00022445.1 | IPI00022445.1 | PFBP | 1 | 1 | 1 | 1 | 1 | 1 |
| IPI00027350.3 | IPI00027350.2 | PRDX2 | 1 | 1 | 1 | 1 | 0 | 0 |
| IPI00024825.2 | IPI00024825.2 | PRG4 | 1 | 1 | 1 | 1 | 1 | 1 |
| IPI00294004.1 | | PROS1 | 1 | | 1 | 0 | 0 | 0 |
| IPI00013179.1 | IPI00013179.1 | PTGDS | 1 | 1 | 1 | 1 | 1 | 1 |
| IPI00025426.1 | IPI00025426.2 | PZP | 1 | 1 | 1 | 1 | 1 | 1 |
| IPI00003590.2 | | QSOX1 | 1 | | 1 | 0 | 0 | 0 |
| IPI00221325.3 | IPI00221325.3 | RANBP2 | 3 | 1 | 1 | 1 | 0 | 0 |
| IPI00411314.2 | IPI00304692.1 | RBMX | 4 | 1 | 1 | 1 | 0 | 0 |
| 2895204 | IPI00022420.3 | RBP4 | 2 | 1 | 1 | 1 | 1 | 1 |
| IPI00009027.1 | IPI00009027.1 | REG1A | 2 | 1 | 1 | 1 | 0 | 0 |
| IPI00014048.1 | IPI00014048.1 | RNASE1 | 1 | 2 | 1 | 1 | 0 | 0 |
| IPI00007047.1 | IPI00007047.1 | S100A8 | 1 | 1 | 1 | 1 | 0 | 0 |
| IPI00027462.1 | IPI00027462.1 | S100A9 | 1 | 1 | 1 | 1 | 1 | 1 |
| 247142 | IPI00006146.4 | SAA1 | 1 | 1 | 1 | 1 | 1 | 1 |
| IPI00027191.1 | IPI00027191.1 | SAA3P | 1 | 1 | 1 | 1 | 0 | 0 |
| IPI00019399.1 | IPI00019399.1 | SAA4 | 1 | 1 | 1 | 1 | 1 | 1 |
| | IPI00218795.1 | SELL | | 1 | 0 | 1 | 0 | 0 |
| IPI00029061.2 | IPI00029061.3 | SEPP1 | 2 | 1 | 1 | 1 | 0 | 0 |
| 223433 | IPI00553177.1 | SERPINA1 | 1 | 1 | 1 | 1 | 1 | 1 |
| IPI00007199.4 | IPI00007199.4 | SERPINA10 | 1 | 2 | 1 | 1 | 0 | 0 |
| 225769 | IPI00550991.3 | SERPINA3 | 1 | 1 | 1 | 1 | 1 | 1 |
| IPI00027482.1 | IPI00027482.1 | SERPINA6 | 1 | 1 | 1 | 1 | 0 | 0 |
| IPI00292946.1 | IPI00292946.1 | SERPINA7 | 1 | 1 | 1 | 1 | 1 | 1 |
| IPI00032179.2 | IPI00032179.3 | SERPINC1 | 1 | 1 | 1 | 1 | 1 | 1 |
| 23273330 | IPI00292950.4 | SERPIND1 | 1 | 1 | 1 | 1 | 1 | 1 |
| 39725934 | IPI00006114.4 | SERPINF1 | 1 | 1 | 1 | 1 | 1 | 1 |
| IPI00029863.4 | IPI00879231.1 | SERPINF2 | 1 | 1 | 1 | 1 | 1 | 1 |
| 179621 | IPI00291866.5 | SERPING1 | 1 | 1 | 1 | 1 | 1 | 1 |
| | IPI00179016.8 | SETD1A | | 1 | 0 | 1 | 0 | 0 |
| IPI00023019.1 | IPI00023019.1 | SHBG | 1 | 2 | 1 | 1 | 0 | 0 |
| | IPI00011961.1 | SIGLEC1 | | 1 | 0 | 1 | 0 | 0 |
| IPI00020687.1 | IPI00020687.1 | SPINK1 | 1 | 2 | 1 | 1 | 0 | 0 |
| IPI00550363.2 | IPI00550363.3 | TAGLN2 | 1 | 1 | 1 | 1 | 0 | 0 |
| 2815575 | IPI00022463.1 | TF | 1 | 1 | 1 | 1 | 1 | 1 |
| IPI00032292.1 | IPI00032292.1 | TIMP1 | 1 | 1 | 1 | 1 | 0 | 0 |
| IPI00298994.5 | IPI00298994.5 | TLN1 | 1 | 2 | 1 | 1 | 1 | 1 |
| IPI00180240.2 | | TMSL3 | 1 | | 1 | 0 | 0 | 0 |
| | IPI00554760.1 | TNR | | 1 | 0 | 1 | 0 | 0 |
| IPI00010779.3 | | TPM4 | 1 | | 1 | 0 | 0 | 0 |
| | | TRANK1 | | | 0 | 0 | 1 | 1 |
| | IPI00413160.4 | TRIOBP | | 1 | 0 | 1 | 0 | 0 |
| IPI00023283.3 | IPI00023283.3 | TIN | 1 | 1 | 1 | 1 | 0 | 0 |
| 230651 | IPI00022432.1 | TIR | 1 | 1 | 1 | 1 | 1 | 1 |
| IPI00295413.8 | IPI00807602.1 | ULK4 | 4 | 1 | 1 | 1 | 0 | 0 |
| 33358191 | IPI00386524.3 | unknown 51 | 1 | | 1 | 0 | 0 | 0 |
| 51103537 | | unknown 70 | 1 | | 1 | 0 | 0 | 0 |
| 51103559 | | unknown 72 | 1 | | 1 | 0 | 0 | 0 |
| 896277 | | unknown 8 | 1 | | 1 | 0 | 0 | 0 |
| | IPI00887739.3 | unknowna111 | | 1 | 0 | 1 | 0 | 0 |
| | IPI00894523.1 | unknowna126 | | 1 | 0 | 1 | 0 | 0 |
| | IPI00930382.1 | unknowna149 | | 1 | 0 | 1 | 0 | 0 |
| | IPI00736860.3 | unknowna60 | | 1 | 0 | 1 | 0 | 0 |
| | | unknown_d1 | | | 0 | 0 | 1 | 0 |
| | | unknown_d10 | | | 0 | 0 | 1 | 1 |
| | | unknown_d11 | | | 0 | 0 | 1 | 1 |
| | | unknown_d12 | | | 0 | 0 | 0 | 1 |
| | | unknown_d13 | | | 0 | 0 | 1 | 1 |
| | | unknown_d14 | | | 0 | 0 | 0 | 1 |
| | | unknown_d15 | | | 0 | 0 | 1 | 1 |
| | | unknown_d16 | | | 0 | 0 | 0 | 0 |
| | | unknown_d17 | | | 0 | 0 | 0 | 0 |
| | | unknown_d2 | | | 0 | 0 | 1 | 0 |
| | | unknown_d3 | | | 0 | 0 | 1 | 0 |
| | | unknown_d4 | | | 0 | 0 | 0 | 1 |
| | | unknown_d5 | | | 0 | 0 | 1 | 1 |
| | | unknown_d6 | | | 0 | 0 | 1 | 1 |
| | | unknown_d7 | | | 0 | 0 | 0 | 1 |
| | | unknown_d8 | | | 0 | 0 | 0 | 1 |
| | | unknown_d9 | | | 0 | 0 | 0 | 1 |
| | IPI00020037.1 | USF2 | | 1 | 0 | 1 | 0 | 0 |
| IPI00395488.2 | IPI00395488.2 | VASN | 1 | 2 | 1 | 1 | 1 | 1 |
| IPI00027038.1 | IPI00027038.1 | VSIG4 | 2 | 1 | 1 | 1 | 0 | 0 |
| IPI00298971.1 | IPI00298971.1 | VTN | 1 | 1 | 1 | 1 | 1 | 1 |
| IPI00023014.1 | IPI00023014.1 | VWF | 1 | 1 | 1 | 1 | 1 | 1 |

| | | | t₀ | t₀ | t₂₄ | t₂₄ |
|---|---|---|---|---|---|---|
| | | | Corr. | Spectral | Corr. | Spectral |
| t₀ Protein ID | t₂₀ Protein ID | Gene Symbol | (r²) | Count | (r²) | Count |
| IPI00022895.7 | IPI00022895.7 | A1BG | 0.37 | 15.68 | 0.25 | 16.53 |
| IPI00478003.1 | IPI00478003.1 | AZM | 0.93 | 27.01 | 0.84 | 82.66 |
| IPI00328762.4 | IPI00328762.5 | ABCA13 | | | | |
| IPI00021428.1 | | ACTA1 | | | | |
| IPI00008603.1 | IPI00008603.1 | ACTA2 | | | | |
| IPI00021439.1 | IPI00021439.1 | ACT6 | | | | |
| IPI00003269.1 | IPI00003269.1 | ACTBL2 | | | | |
| IPI00020019.1 | IPI00020019.1 | ADIPOQ | | | | |
| | IPI00004344.1 | AFF4 | | | | |
| IPI00019943.1 | IPI00019943.1 | AFM | 0.81 | 7.23 | 0.74 | 4.74 |
| IPI00022443.1 | IPI00022443.1 | AFP | | | | |
| 15079348 | IPI00032220.3 | AGT | 0.44 | 15.45 | 0.64 | 8.78 |
| IPI00022431.1 | IPI00022431.2 | AHSG | 0.33 | 2.58 | 0.52 | 3.19 |
| | | ALB | 0.89 | 33.86 | | 28.13 |
| IPI00022426.1 | IPI00022426.1 | AMBP | 0.51 | 2.91 | 0.63 | 3.34 |
| IPI00022391.1 | IPI00022391.1 | APCS | 0.57 | 9.65 | 0.50 | 6.60 |
| 253362 | IPI00021841.1 | APOA1 | 0.91 | 28.85 | 0.88 | 24.59 |
| 671882 | IPI00021854.1 | APOA2 | 0.85 | 10.61 | 0.57 | 6.98 |
| IPI00304273.2 | IPI00847179.1 | APOA4 | 0.85 | 13.37 | 0.81 | 9.58 |
| 225311 | IPI00022229.3 | APO8 | 0.90 | 90.44 | 0.90 | 52.75 |
| IPI00021855.1 | IPI00021855.1 | APOC1 | 0.52 | 1.32 | 0.18 | 1.52 |
| IPI00021856.3 | IPI00021856.3 | APOC2 | 0.49 | 2.52 | 0.62 | 1.97 |
| IPI00021857.1 | IPI00021857.1 | APOC3 | 0.47 | 2.63 | 0.45 | 2.43 |
| IPI00022731.1 | IPI00022731.1 | APOC4 | | | | |
| IPI00006662.1 | IPI00006662.1 | APOD | 0.42 | 1.49 | 0.40 | 1.51 |
| 15826311 | IPI00021842.1 | APOE | 0.60 | 6.20 | 0.75 | 4.05 |
| | | APOF | | 1.13 | 0.05 | 1.09 |
| IPI00298828.3 | IPI00298828.3 | APOH | 0.32 | 3.50 | 0.01 | 1.37 |
| IPI00186903.3 | IPI00186903.4 | APOL1 | 0.11 | 1.69 | 0.06 | 1.38 |
| IPI00027235.1 | | ATRN | | | | |
| IPI00166729.4 | IPI00166729.4 | AZGP1 | 0.75 | 15.73 | 0.66 | 12.11 |
| IPI00004656.1 | IPI00004656.3 | B2M | 0.67 | 2.21 | 0.68 | 2.55 |
| IPI00297188.5 | IPI00297188.6 | BAI2 | | | | |
| IPI00022392.1 | IPI00022392.1 | C1QA | 0.21 | 2.33 | 0.24 | 1.35 |
| IPI00477992.1 | IPI00477992.1 | C1QB | 0.19 | 3.02 | 0.22 | 1.37 |
| IPI00022394.2 | IPI00022394.2 | C1QC | 0.23 | 1.75 | 0.22 | 1.43 |
| IPI00296165.5 | IPI00296165.6 | C1R | 0.41 | 1.24 | 0.37 | 1.45 |
| IPI00009793.2 | IPI00009793.4 | C1RL | | | | |
| IPI00017696.1 | IPI00017696.1 | CLS | | | 0.01 | 1.12 |
| IPI00745619.1 | IPI00303963.1 | C2 | 0.02 | 1.76 | 0.00 | 1.19 |
| IPI00783987.1 | IPI00783987.2 | C3 | 0.93 | 106.97 | 0.84 | 14.11 |
| 179674 | IPI00892547.1 | C4A | | | | |
| IPI00418163.3 | IPI00418163.3 | C48 | 0.89 | 48.79 | 0.87 | 43.55 |
| IPI00021727.1 | IPI00021727.1 | C48PA | 0.66 | 8.15 | 0.29 | 4.23 |
| IPI00025862.1 | IPI00025862.2 | C48PB | | | | |
| 38016947 | IPI00032291.2 | C5 | 0.84 | 3.90 | 0.60 | 2.70 |
| IPI00009920.2 | IPI00879709.3 | C6 | 0.53 | 2.60 | 0.35 | 2.13 |
| 179716 | IPI00296608.6 | C7 | 0.73 | 2.74 | 0.35 | 1.69 |
| 9016854 | IPI00011252.1 | C8A | 0.16 | 2.14 | 0.09 | 1.29 |
| IPI00294395.1 | | C8B | | | | |
| IPI00011261.2 | IPI00011261.2 | C8G | 0.16 | 1.70 | 0.02 | 1.31 |
| IPI00022395.1 | IPI00022395.1 | C9 | 0.75 | 4.88 | 0.10 | 1.58 |
| IPI00215983.2 | IPI00215983.3 | CA1 | | | | |
| IPI00465436.3 | IPI00465436.4 | CAT | | | | |
| IPI00029260.2 | IPI00029260.2 | CD14 | 0.25 | 1.78 | 0.24 | 1.31 |
| IPI00104074.4 | | CD163 | | | | |
| IPI00025204.1 | IPI00025204.1 | CD5L | | | | |
| 40737516 | | CDA5 | | | | |
| | IPI00855958.1 | CENPF | | | | |
| IPI00010180.3 | 010180.4 | CES1 | | | 0.32 | 5.00 |
| IPI00019591.1 | IPI00893864.1 | CF8 | 0.47 | 20.31 | 0.59 | 17.11 |
| IPI00019579.1 | IPI00165972.3 | CFD | | | | |
| IPI00029739.4 | IPI00029739.5 | CFH | 0.29 | 14.79 | 0.20 | 9.28 |
| IPI00006543.2 | IPI00011264.2 | CFHR1 | | | | |
| IPI00006154.1 | IPI00006154.1 | CFHR2 | | | | |
| IPI00027507.1 | IPI00027507.1 | CFHR3 | | | | |
| IPI00291867.3 | IPI00291867.3 | CF1 | 0.45 | 5.02 | 0.31 | 2.93 |
| IPI00012011.5 | | CFL1 | | | | |
| IPI00021364.4 | IPI00021364.1 | CFP | | | | |
| IPI00009028.1 | IPI00009028.1 | CLEC38 | 0.16 | 1.24 | 0.02 | 1.25 |
| IPI00291262.3 | IPI00291262.3 | CLU | 0.31 | 7.77 | 0.45 | 5.98 |
| IPI00011283.1 | IPI00011783.2 | COL11A2 | | | | |
| 179594 | IPI00297646.4 | COL1A1 | | | | |
| IPI00168920.2 | IPI00168920.3 | COL24A1 | | | | |
| 930045 | IPI00021033.2 | COL3A1 | | | | |
| IPI00025418.1 | IPI00025418.2 | COL7A1 | | | | |
| 2632189 | IPI00423463.1 | COPB1 | | | | |
| IPI00017601.1 | IPI00017601.1 | CP | 0.80 | 50.54 | 0.75 | 41.53 |
| IPI00293057.5 | | CPBZ | | | | |
| IPI00010295.1 | IPI00010295.1 | CPN1 | | | | |
| IPI00479116.1 | IPI00475136.3 | CPN2 | 0.01 | 1.17 | 0.04 | 1.29 |
| | IPI00011062.1 | CPS1 | | | 0.04 | 2.00 |
| IPI00022339.1 | IPI00022389.1 | CRP | 0.76 | 3.30 | 0.64 | 4.09 |
| IPI00032293.1 | IPI00032293.1 | CST3 | 0.13 | 1.14 | 0.07 | 1.50 |
| IPI00005721.1 | IPI00005721.1 | DEFA1 | | | | |
| IPI00465045.2 | | DIP2B | | | | |
| 4758236 | IPI00003351.2 | ECM1 | | | | |
| IPI00019531.1 | IPI00019581.1 | F12 | | | | |
| IPI00019588.1 | IPI00019568.1 | F2 | 0.05 | 1.12 | 0.08 | 2.20 |
| IPI00010290.1 | IPI00010290.2 | FABP1 | | | | |
| IPI00215746.2 | | FABP4 | | | | |
| | | FAM135A | 0.01 | 1.38 | | 1.31 |
| IPI00218803.2 | IPI00218803.3 | FELN1 | | | | |
| IPI00242956.3 | IPI00242956.5 | FCGBP | 0.45 | 2.44 | 0.26 | 1.78 |
| IPI00293925.2 | | FCN3 | | | | |
| IPI00021885.1 | IPI00021885.1 | FGA | 0.82 | 17.89 | 0.71 | 38.84 |
| IPI00298437.3 | IPI00298497.3 | FGB | 0.69 | 17.31 | 0.73 | 40.20 |
| IPI00021891.5 | IPI00021891.5 | FGG | 0.82 | 11.00 | 0.69 | 29.91 |
| | IPI00289334.1 | FLNB | | | | |
| 51476364 | IPI00022418.1 | FN1 | 0.59 | 6.75 | 0.39 | 3.05 |
| | IPI00375676.3 | FTL | | | 0.51 | 3.00 |
| IPI00010375.3 | IPI00555812.4 | GC | 0.57 | 26.01 | 0.38 | 20.59 |
| IPI00026199.1 | IPI00026199.2 | GPK3 | | | | |
| IPI00026314.1 | IPI00026314.1 | GSN | 0.69 | 4.22 | 0.65 | 5.28 |
| IPI00465253.4 | IPI00465253.4 | HAU56 | | | | |
| 27574247 | IPI00410714.5 | HBA1 | 0.76 | 2.80 | 0.91 | 4.43 |
| 13195586 | | HBA2 | | | | |
| IPI00658153.1 | IPI00654755.3 | HBB | 0.89 | 5.30 | 0.91 | 7.24 |
| IPI00473011.2 | IPI00473011.3 | HBD | | | | |
| | IPI00220706.10 | HBG1 | | | 0.11 | 1.50 |
| IPI00217473.4 | IPI00217473.5 | HBZ | | | | |
| 229528 | | HLA-E | | | | |
| 229271 | IPI00902590.1 | HP | 0.93 | 14.24 | 0.93 | 31.70 |
| IPI00477597.1 | IPI00477597.1 | HPR | 0.60 | 1.86 | 0.04 | 1.17 |
| IPI00022488.1 | IPI00022488.2 | HPX | 0.78 | 21.19 | 0.77 | 20.24 |
| IPI00022371.1 | IPI00022371.1 | HRG | 0.85 | 6.39 | 0.81 | 4.90 |
| | IPI00220362.5 | HSPE1 | | | | |
| IPI00005477.4 | IPI00009477.4 | ICAM2 | | | | |
| | | IGFALS | | | | 1.33 |
| IPI00297284.1 | | IGFBP2 | | | | |
| IPI00305380.3 | IPI00305380.3 | IGFBP4 | | | | |
| IPI00029235.1 | | IGFBP6 | | | | |
| 34527679 | IPI00386524.3 | IGHA1 | 0.82 | 7.82 | 0.19 | 15.48 |
| | | IGHD | | 1.74 | | 1.58 |
| 21757089 | | IGHG1 | 0.02 | 35.33 | | 28.34 |
| | | IGHG2 | | 5.85 | | 7.96 |
| | | IGHG3 | | 4.84 | | 26.77 |
| | | IGHG4 | | 4.00 | | 4.85 |
| | | IGHM | 0.08 | 5.47 | | 15.52 |
| | | IGJ | | 1.71 | | 2.49 |
| | | IGKC | | 24.21 | | 27.50 |
| | | IGKY1-5 | | | | |
| | | IGLC7 | | 11.68 | | 14.73 |
| 1871489 | | IGM | | | | |
| 33319112 | | IGVH2 | | | | |
| IPI00292530.1 | IPI00292530.1 | ITH1 | 0.78 | 7.48 | 0.70 | 6.05 |
| IPI00305461.2 | IPI00305461.3 | ITH2 | 0.79 | 10.68 | 0.55 | 7.84 |
| IPI00026413.1 | IPI00028413.8 | ITH3 | 0.33 | 2.41 | 0.13 | 1.82 |
| IPI00218192.1 | IPI00218192.3 | ITH4 | 0.67 | 48.61 | 0.70 | 43.21 |
| 1575607 | IPI00479786.5 | KHSRP | | | | |
| | IPI00827544.1 | KIF19 | | | | |
| IPI00654888.2 | IPI00654888.2 | KLKB1 | | | | |
| IPI00032328.1 | IPI00215894.1 | KNGL | 0.34 | 5.59 | 0.54 | 3.72 |
| | | KRT31 | | | | 6.00 |
| | | KRT81 | | | | 1.50 |
| 31652249 | IPI00032311.4 | LBP | 0.60 | 4.73 | 0.75 | 3.46 |
| IPI00299547.4 | IPI00299547.4 | LCN2 | | | | |
| IPI00010471.4 | IPI00010471.5 | LCP1 | | | | |
| IPI00023673.1 | IPI00023673.1 | LGALS3BP | | | | |
| IPI00164623.4 | | LOC100133511 | | | | |
| IPI00167093.4 | | LOC100293069 | | | | |
| IPI00061977.1 | | LOC100294459 | | | | |
| IPI00736860.2 | | LOC100294460 | | | | |
| IPI00029168.1 | IPI00029168.1 | LPA | 0.56 | 1.68 | 0.06 | 1.75 |
| 21707947 | IPI00022417.4 | LRG1 | 0.65 | 21.37 | 0.40 | 19.27 |
| IPI00298860.5 | | LTF | | | | |
| IPI00020986.2 | IPI00020986.2 | LUM | 0.83 | 4.31 | 0.61 | 2.66 |
| 3402141 | IPI00019038.1 | LYZ | | | | |
| | 126508 | LYZL4 | | | | |
| | | MACF1 | | 1.02 | 0.00 | 1.80 |
| IPI00217493.4 | IPI00217493.5 | MB | | | 0.30 | 3.00 |
| IPI00004373.1 | IPI00004373.1 | MBL2 | | | | |
| 21756643 | | MCAM | | | | |
| IPI00306929.7 | IPI00306929.9 | MYO18B | | | | |
| | IPI00423460.3 | NID1 | | | | |
| IPI00022429.3 | IPI00022429.3 | ORM1 | 0.90 | 13.69 | 0.78 | 14.94 |
| IPI00020091.1 | IPI00020091.1 | ORM2 | 0.81 | 4.75 | 0.67 | 9.31 |
| IPI00022295.1 | | PF4V1 | | | | |
| IPI00216694.1 | | PFN1 | | | | |
| IPI00163207.1 | IPI00163207.1 | PGLYRP2 | 0.49 | 2.49 | 0.25 | 1.65 |
| IPI00004573.1 | IPI00004573.2 | PIGR | | | | |
| IPI00306311.8 | IPI00306311.8 | PLEK | | | | |
| 229453 | IPI00019580.1 | PLG | 0.23 | 2.34 | 0.31 | 1.88 |
| 1262347 | IPI00218732.3 | PON1 | | | | |
| IPI00555900.1 | | POTEK | | | | |
| IPI00022445.1 | IPI00022445.1 | PFBP | 0.41 | 1.74 | 0.31 | 1.85 |
| IPI00027350.3 | IPI00027350.2 | PRDX2 | | | | |
| IPI00024825.2 | IPI00024825.2 | PRG4 | 0.04 | 1.15 | 0.02 | 1.22 |
| IPI00294004.1 | | PROS1 | | | | |
| IPI00013179.1 | IPI00013179.1 | PTGDS | 0.61 | 2.45 | 0.02 | 1.93 |
| IPI00025426.1 | IPI00025426.2 | PZP | 0.00 | 1.40 | 0.01 | 1.58 |
| IPI00003590.2 | | QSOX1 | | | | |
| IPI00221325.3 | IPI00221325.3 | RANBP2 | | | | |
| IPI00411314.2 | IPI00304692.1 | RBMX | | | | |
| 2895204 | IPI00022420.3 | RBP4 | 0.45 | 4.74 | 0.53 | 2.43 |
| IPI00009027.1 | IPI00009027.1 | REG1A | | | | |
| IPI00014048.1 | IPI00014048.1 | RNASE1 | | | | |
| IPI00007047.1 | IPI00007047.1 | S100A8 | | | | |
| IPI00027462.1 | IPI00027462.1 | S100A9 | 0.81 | 3.28 | 0.57 | 3.21 |
| 247142 | IPI00006146.4 | SAA1 | 0.88 | 9.59 | 0.86 | 10.71 |
| IPI00027191.1 | IPI00027191.1 | SAA3P | | | | |
| IPI00019399.1 | IPI00019399.1 | SAA4 | 0.49 | 2.48 | 0.50 | 1.89 |
| | IPI00218795.1 | SELL | | | | |
| IPI00029061.2 | IPI00029061.3 | SEPP1 | | | | |
| 223433 | IPI00553177.1 | SERPINA1 | 0.92 | 38.51 | 0.88 | 70.31 |
| IPI00007199.4 | IPI00007199.4 | SERPINA10 | | | | |
| 225769 | IPI00550991.3 | SERPINA3 | 0.86 | 55.45 | 0.89 | 44.76 |
| IPI00027482.1 | IPI00027482.1 | SERPINA6 | | | | |
| IPI00292946.1 | IPI00292946.1 | SERPINA7 | 0.14 | 1.30 | 0.07 | 1.72 |
| IPI00032179.2 | IPI00032179.3 | SERPINC1 | 0.50 | 26.02 | 0.54 | 22.90 |
| 23273330 | IPI00292950.4 | SERPIND1 | 0.29 | 1.99 | 0.01 | 1.30 |
| 39725934 | IPI00006114.4 | SERPINF1 | 0.63 | 2.67 | 0.36 | 1.65 |
| IPI00029863.4 | IPI00879231.1 | SERPINF2 | 0.04 | 4.85 | 0.17 | 4.02 |
| 179621 | IPI00291866.5 | SERPING1 | 0.75 | 19.16 | 0.66 | 9.46 |
| | IPI00179016.8 | SETD1A | | | | |
| IPI00023019.1 | IPI00023019.1 | SHBG | | | | |
| | IPI00011961.1 | SIGLEC1 | | | | |
| IPI00020687.1 | IPI00020687.1 | SPINK1 | | | | |
| IPI00550363.2 | IPI00550363.3 | TAGLN2 | | | | |
| 2815575 | IPI00022463.1 | TF | 0.48 | 6.48 | 0.89 | 21.89 |
| IPI00032292.1 | IPI00032292.1 | TIMP1 | | | | |
| IPI00298994.5 | IPI00298994.5 | TLN1 | 0.25 | 1.66 | 0.13 | 1.02 |
| IPI00180240.2 | | TMSL3 | | | | |
| | IPI00554760.1 | TNR | | | | |
| IPI00010779.3 | | TPM4 | | | | |
| | | TRANK1 | 0.00 | 1.07 | | 1.16 |
| | IPI00413160.4 | TRIOBP | | | | |
| IPI00023283.3 | IPI00023283.3 | TIN | | | | |
| 230651 | IPI00022432.1 | TIR | 0.90 | 14.79 | 0.93 | 9.14 |
| IPI00295413.8 | IPI00807602.1 | ULK4 | | | | |
| 33358191 | IPI00386524.3 | unknown 51 | | | | |
| 51103537 | | unknown 70 | | | | |
| 51103559 | | unknown 72 | | | | |
| 896277 | | unknown 8 | | | | |
| | IPI00887739.3 | unknowna111 | | | | |
| | IPI00894523.1 | unknowna126 | | | | |
| | IPI00930382.1 | unknowna149 | | | | |
| | IPI00736860.3 | unknowna60 | | | | |
| | | unknown_d1 | | | | |
| | | unknown_d10 | | | | |
| | | unknown_d11 | | | | |
| | | unknown_d12 | | | | |
| | | unknown_d13 | | | | |
| | | unknown_d14 | | | | |
| | | unknown_d15 | | | | |
| | | unknown_d16 | | | | |
| | | unknown_d17 | | | | |
| | | unknown_d2 | | | | |
| | | unknown_d3 | | | | |
| | | unknown_d4 | | | | |
| | | unknown_d5 | | | | |
| | | unknown_d6 | | | | |
| | | unknown_d7 | | | | |
| | | unknown_d8 | | | | |
| | | unknown_d9 | | | | |
| | IPI00020037.1 | USF2 | | | | |
| IPI00395488.2 | IPI00395488.2 | VASN | 0.05 | 1.19 | 0.04 | 1.09 |
| IPI00027038.1 | IPI00027038.1 | VSIG4 | | | | |
| IPI00298971.1 | IPI00298971.1 | VTN | 0.40 | 1.56 | 0.33 | 4.45 |
| IPI00023014.1 | IPI00023014.1 | VWF | 0.61 | 4.70 | 0.48 | 2.41 |

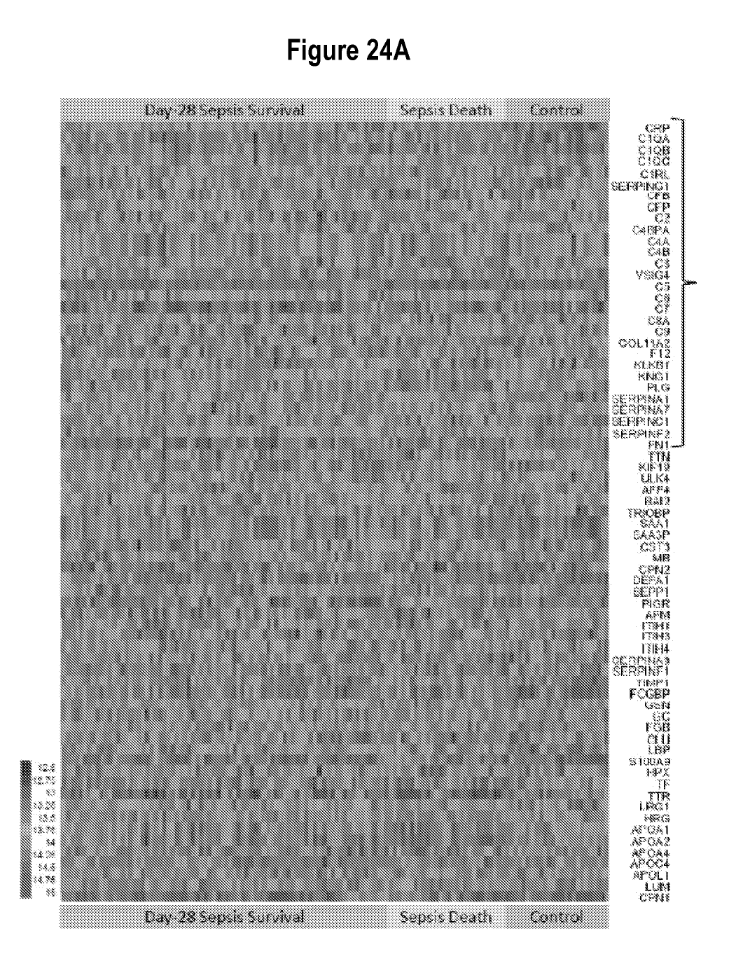

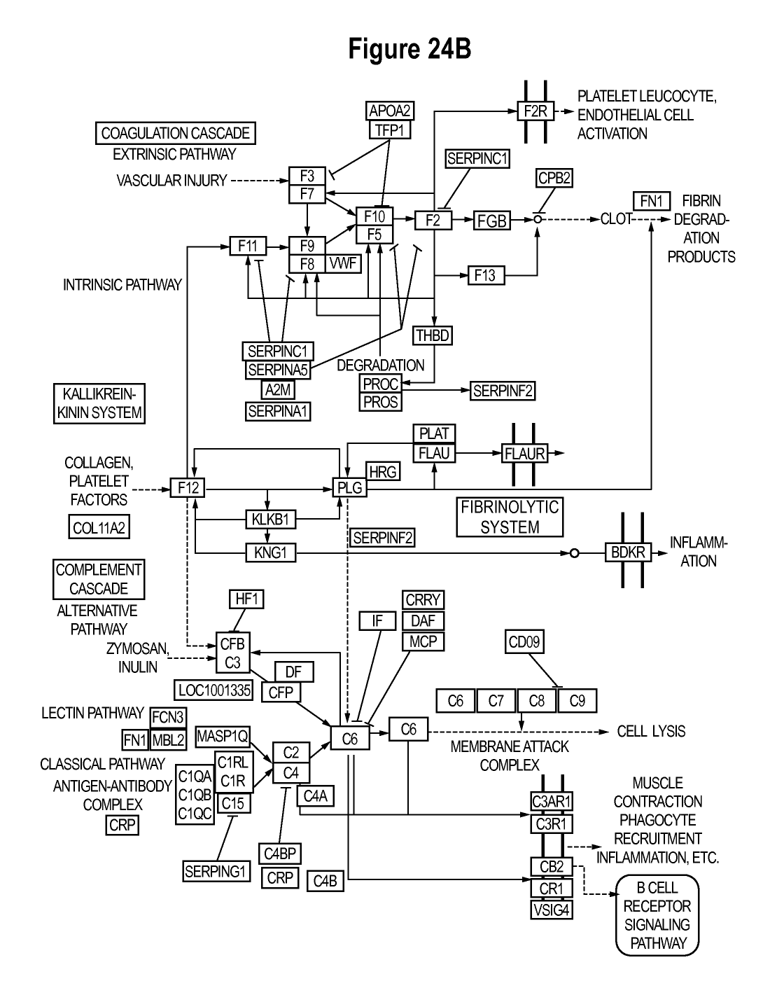

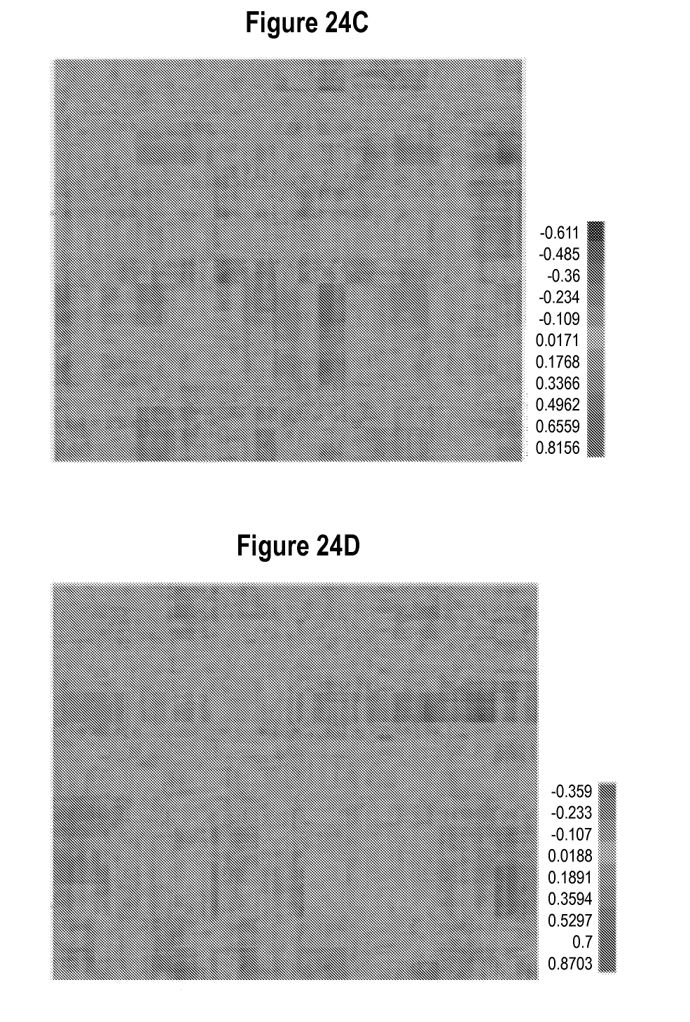

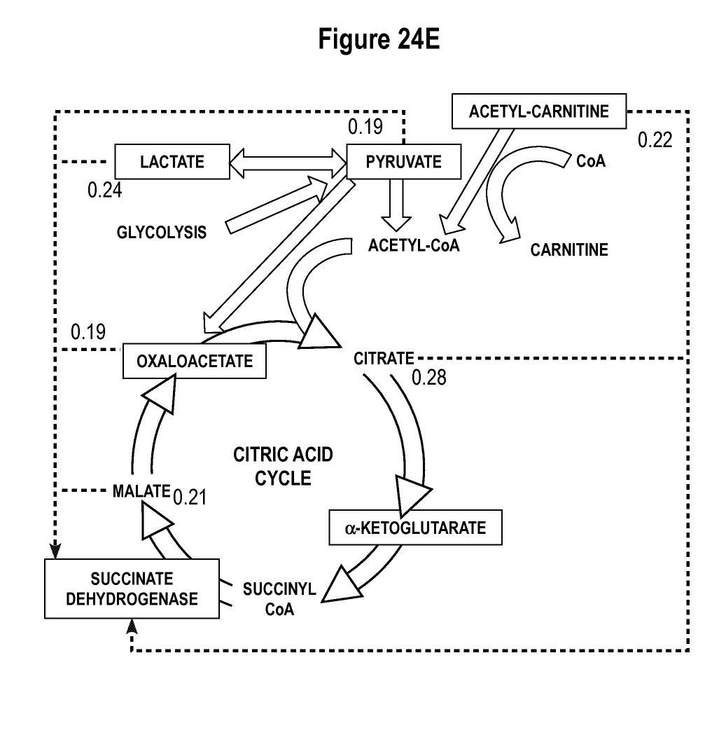

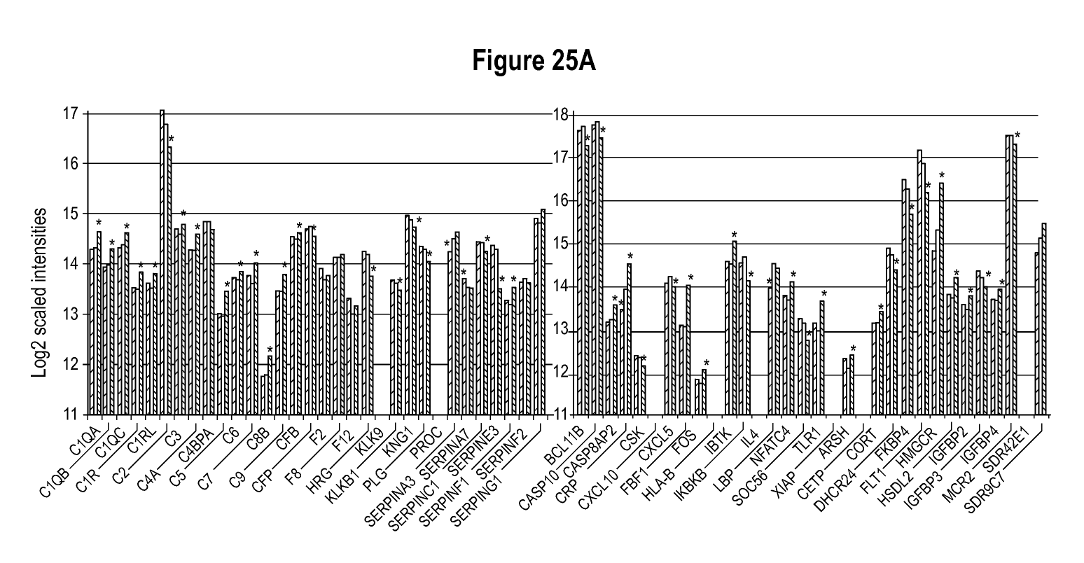

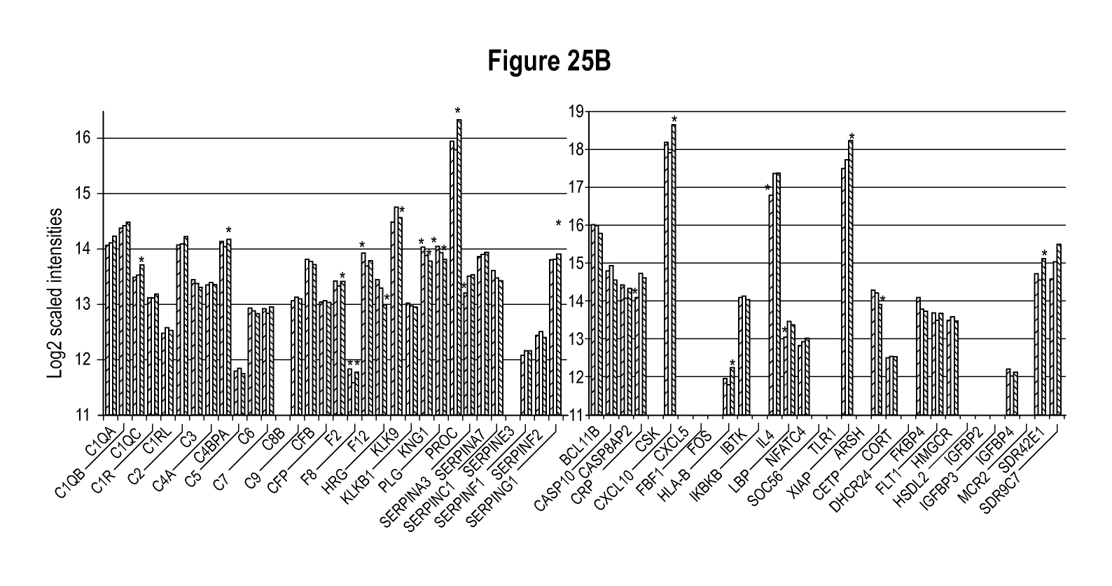

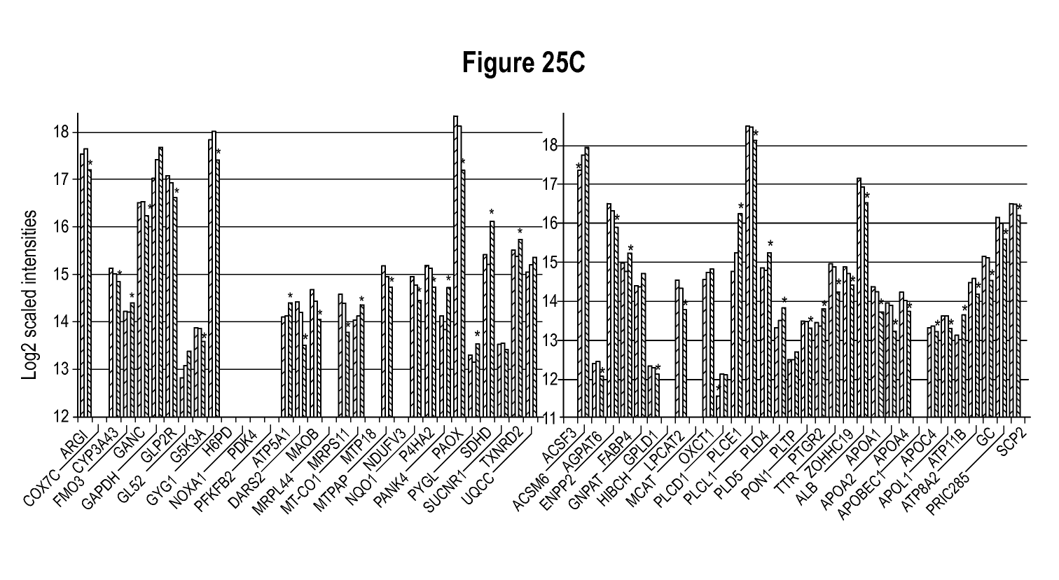

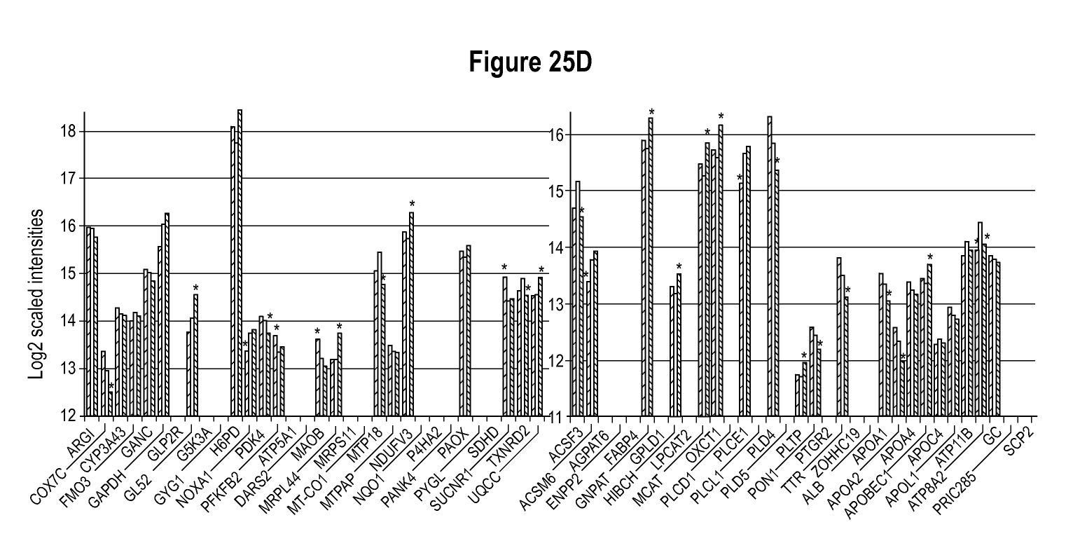

In contrast, sepsis survivors differed from controls in levels of 15 and 23 plasma proteins at t₀ and t₂₄, respectively (stratified ANOVA, FDR 5%; FIG. 24a; Table 14; FIG. 25). 21 of 24 plasma proteins exhibiting significant differences between sepsis survivors and controls at one time point and detected at the other had congruent direction of change. In agreement with previous reports, many inflammatory markers were elevated in sepsis (CRP, lipopolysaccharide binding protein, leucine-rich α2 glycoprotein, serpin peptidase inhibitor 3, serum amyloid A1 and A3 and selenoprotein P; FIG. 24). Prominently decreased were thrombolysis proteins factor XII, plasminogen, kininogen 1 and fibronectin 1. Related to these, serpin peptidase inhibitor 1, which inhibits plasmin and thombin, was increased, also as previously reported.

TABLE 14
Average, log-transformed, scaled, plasma protein concentrations in non-infected, SIRS-positive
patients (controls), sepsis survivors and sepsis deaths at t₀ and t₂₄ in 150 discovery
patients, showing significant differences from sepsis survivors by weighted ANOVAs (denoted*,
5% FDR with the exception of t₂₄ sepsis survival versus death, 10% FDR).

| Gene | | | | | |
|---|---|---|---|---|---|
| Symbol | Annotation | Time | NIS | Survivors | Deaths |
| ACTA1 | Actin α1 | t0 | 13.59 ± 0.02* | 13.40 ± 0.00 | 13.65 ± 0.01* |
| | | t24 | * | * | * |
| ACTA2 | Actin α2 | t0 | 13.34 ± 0.02 | 13.16 ± 0.00 | 13.39 ± 0.01* |
| | | t24 | 12.37 ± 0.01 | 12.25 ± 0.01 | 12.31 ± 0.01 |
| ACTB | Actin β | t0 | 13.42 ± 0.02 | 13.25 ± 0.00 | 13.49 ± 0.01* |
| | | t24 | 12.46 ± 0.01 | 12.38 ± 0.00 | 12.46 ± 0.01 |
| ACTBL2 | Actin β-like 2 | t0 | 13.02 ± 0.02 | 12.80 ± 0.00 | 13.05 ± 0.02* |
| | | t24 | 12.55 ± 0.02 | 12.52 ± 0.01 | 12.61 ± 0.01 |
| LCP1 | Lymphocyte cytosolic protein 1 | t0 | 12.27 ± 0.01 | 12.25 ± 0.00 | 12.48 ± 0.01* |
| | (L-plastin) | t24 | 12.19 ± 0.01 | 12.31 ± 0.00 | 12.46 ± 0.01 |
| TPM4 | Tropomyosin α4 | t0 | 15.27 ± 0.01* | 15.03 ± 0.00 | 15.16 ± 0.01 |
| | | t24 | * | * | * |
| COL11A2 | Collagen XI α2 | t0 | 14.23 ± 0.02 | 14.17 ± 0.01 | 13.82 ± 0.01* |
| | | t24 | 13.50 ± 0.01* | 13.28 ± 0.00 | 13.25 ± 0.01 |
| GSN | gelsolin | t0 | 14.07 ± 0.01* | 13.92 ± 0.00 | 13.99 ± 0.01 |
| | | t24 | 12.65 ± 0.01 | 12.63 ± 0.00 | 12.54 ± 0.01 |
| KIF19 | kinesin 19 | t0 | * | * | * |
| | | t24 | 15.57 ± 0.02* | 15.97 ± 0.01 | 15.87 ± 0.01 |
| TTN | titin | t0 | 14.39 ± 0.01 | 14.52 ± 0.00 | 14.25 ± 0.01* |
| | | t24 | 14.09 ± 0.00 | 14.06 ± 0.00 | 14.04 ± 0.01 |
| MB | myoglobin | t0 | 11.63 ± 0.02 | 11.55 ± 0.00 | 11.70 ± 0.02 |
| | | t24 | 12.29 ± 0.01 | 12.35 ± 0.00 | 12.62 ± 0.01* |
| LUM | lumican | t0 | 14.29 ± 0.02* | 13.93 ± 0.00 | 14.04 ± 0.02 |
| | | t24 | 13.47 ± 0.02 | 13.33 ± 0.00 | 13.46 ± 0.01 |
| NID1 | nidogen 1 | t0 | * | * | * |
| | | t24 | 14.37 ± 0.01 | 14.25 ± 0.00 | 14.44 ± 0.01* |
| PFN1 | Profilin-1 | t0 | 13.98 ± 0.01 | 13.85 ± 0.00 | 13.99 ± 0.01* |
| | | t24 | * | * | * |
| TRIOBP | TRIO and F-actin binding | t0 | * | * | * |
| | protein | t24 | 13.24 ± 0.02 | 13.39 ± 0.00 | 13.60 ± 0.01* |
| POTEK | POTE ankyrin domain family, K | t0 | 13.59 ± 0.02 | 13.43 ± 0.00 | 13.68 ± 0.02* |
| | | t24 | * | * | * |
| RBMX | RNA binding motif protein X- | t0 | 15.11 ± 0.03 | 15.07 ± 0.01 | 15.31 ± 0.03 |
| | linked | t24 | 13.77 ± 0.03 | 13.88 ± 0.01 | 14.29 ± 0.02* |
| AFF4 | AF4/FMR2 family 4 | t0 | * | * | * |
| | | t24 | 15.10 ± 0.02 | 15.05 ± 0.01 | 15.36 ± 0.02* |
| AFM | afamin | t0 | 13.94 ± 0.01 | 13.79 ± 0.00 | 13.55 ± 0.01* |
| | | t24 | 13.32 ± 0.01* | 13.08 ± 0.01 | 12.99 ± 0.01 |
| AHSG | α-2-HS-glycoprotein | t0 | 14.61 ± 0.01 | 14.46 ± 0.01 | 14.01 ± 0.02* |
| | | t24 | 14.20 ± 0.01* | 13.85 ± 0.01 | 13.76 ± 0.02 |
| APOA1 | Apolipoprotein A-I | t0 | 14.39 ± 0.02 | 14.25 ± 0.01 | 13.78 ± 0.01* |
| | | t24 | 13.56 ± 0.02 | 13.37 ± 0.01 | 13.06 ± 0.01* |
| APOA2 | apolipoprotein A-II | t0 | 13.99 ± 0.03 | 13.91 ± 0.01 | 13.25 ± 0.01* |
| | | t24 | 12.58 ± 0.02 | 12.35 ± 0.01 | 12.00 ± 0.01* |
| APOA4 | apolipoprotein A-IV | t0 | 14.20 ± 0.02 | 14.05 ± 0.01 | 13.77 ± 0.02* |
| | | t24 | 13.40 ± 0.01 | 13.27 ± 0.00 | 13.19 ± 0.01 |
| APOC4 | apolipoprotein C-IV | t0 | 13.35 ± 0.01 | 13.40 ± 0.00 | 13.23 ± 0.01* |
| | | t24 | 12.28 ± 0.02 | 12.37 ± 0.01 | 12.31 ± 0.01 |
| APOL1 | apolipoprotein L1 | t0 | 13.64 ± 0.01 | 13.63 ± 0.00 | 13.32 ± 0.01* |
| | | t24 | 12.95 ± 0.01 | 12.82 ± 0.00 | 12.75 ± 0.01 |
| SERPINA7 | thyroxine-binding globulin | t0 | 13.71 ± 0.01* | 13.54 ± 0.00 | 13.52 ± 0.01 |
| | | t24 | 13.83 ± 0.01 | 13.89 ± 0.00 | 13.93 ± 0.01 |
| FABP4 | Fatty acid-binding protein 4 | t0 | 15.01 ± 0.02 | 14.79 ± 0.01 | 15.24 ± 0.02* |
| | | t24 | * | * | * |
| ORM1 | orosomucoid 1 | t0 | 14.42 ± 0.02 | 14.71 ± 0.01 | 14.88 ± 0.02 |
| | | t24 | 13.63 ± 0.01 | 13.80 ± 0.00 | 14.03 ± 0.01* |
| TTR | transthyretin | t0 | 14.98 ± 0.02 | 14.90 ± 0.01 | 14.28 ± 0.02* |
| | | t24 | 13.83 ± 0.02 | 13.50 ± 0.01 | 13.12 ± 0.01* |
| TF | Transferrin | t0 | 14.17 ± 0.01 | 14.01 ± 0.00 | 13.88 ± 0.01* |
| | | t24 | 13.95 ± 0.02* | 13.52 ± 0.01 | 13.39 ± 0.01 |
| GC | Vitamin D-binding protein | t0 | 15.17 ± 0.01 | 15.15 ± 0.00 | 14.56 ± 0.03* |
| | | t24 | 13.87 ± 0.01 | 13.83 ± 0.00 | 13.76 ± 0.01 |
| HPX | hemopexin | t0 | 14.82 ± 0.01 | 14.88 ± 0.00 | 14.68 ± 0.01* |
| | | t24 | 13.84 ± 0.01 | 13.87 ± 0.00 | 13.76 ± 0.01 |
| MBL2 | mannose binding lectin 2 | t0 | 13.19 ± 0.01 | 13.16 ± 0.00 | 13.23 ± 0.01 |
| | | t24 | 13.17 ± 0.02 | 13.11 ± 0.01 | 13.59 ± 0.02* |
| C1QA | complement C1q A chain | t0 | 14.29 ± 0.01 | 14.35 ± 0.00 | 14.67 ± 0.01* |
| | | t24 | 14.06 ± 0.01 | 14.09 ± 0.00 | 14.19 ± 0.01 |
| C1QB | complement C1q B chain | t0 | 13.97 ± 0.01 | 13.99 ± 0.00 | 14.31 ± 0.01* |
| | | t24 | 14.37 ± 0.02 | 14.40 ± 0.00 | 14.45 ± 0.01 |
| C1QC | complement C1q C chain | t0 | 14.33 ± 0.01 | 14.40 ± 0.00 | 14.62 ± 0.01* |
| | | t24 | 13.48 ± 0.02 | 13.51 ± 0.00 | 13.67 ± 0.01* |
| C1R | complement 1r | t0 | 13.54 ± 0.01 | 13.51 ± 0.00 | 13.82 ± 0.01* |
| | | t24 | 13.11 ± 0.01 | 13.11 ± 0.00 | 13.17 ± 0.01 |
| C1RL | complement C1r-like | t0 | 13.61 ± 0.01 | 13.55 ± 0.00 | 13.82 ± 0.01* |
| | | t24 | 12.49 ± 0.01 | 12.56 ± 0.00 | 12.53 ± 0.01 |
| C2 | complement 2 | t0 | 17.06 ± 0.02 | 16.80 ± 0.01 | 16.36 ± 0.02* |
| | | t24 | 14.06 ± 0.01 | 14.09 ± 0.00 | 14.18 ± 0.01 |
| C3 | complement C3 | t0 | 14.70 ± 0.01 | 14.61 ± 0.00 | 14.80 ± 0.02* |
| | | t24 | 13.42 ± 0.01 | 13.35 ± 0.00 | 13.29 ± 0.01 |
| VSIG4 | V-set & immunoglobulin | t0 | 12.41 ± 0.01 | 12.48 ± 0.00 | 12.53 ± 0.01 |
| | domain containing 4 | t24 | 12.30 ± 0.02 | 12.23 ± 0.01 | 12.49 ± 0.02* |
| LOC- | Complement C3-like | t0 | 14.71 ± 0.01 | 14.61 ± 0.00 | 14.81 ± 0.02* |
| 10013351 | | t24 | * | * | * |
| C4A | complement C4A | t0 | 14.30 ± 0.01 | 14.26 ± 0.00 | 14.60 ± 0.02* |
| | | t24 | 13.35 ± 0.01 | 13.37 ± 0.00 | 13.33 ± 0.01 |
| C4B | complement C4B | t0 | 14.36 ± 0.01 | 14.31 ± 0.00 | 14.66 ± 0.02* |
| | | t24 | 13.38 ± 0.01 | 13.40 ± 0.00 | 13.36 ± 0.01 |
| C4BPA | complement C4 binding protein | t0 | 14.88 ± 0.01 | 14.87 ± 0.00 | 14.73 ± 0.01 |
| | α | t24 | 14.11 ± 0.01 | 14.03 ± 0.00 | 14.16 ± 0.01* |
| C5 | complement C5 | t0 | 13.04 ± 0.01 | 12.98 ± 0.00 | 13.45 ± 0.02* |
| | | t24 | 11.79 ± 0.01 | 11.84 ± 0.00 | 11.74 ± 0.01 |
| C6 | complement C6 | t0 | 13.74 ± 0.01 | 13.70 ± 0.00 | 13.86 ± 0.01* |
| | | t24 | 12.93 ± 0.01 | 12.86 ± 0.00 | 12.84 ± 0.01 |
| C7 | Complement C7 | t0 | 13.77 ± 0.02 | 13.61 ± 0.00 | 14.04 ± 0.01* |
| | | t24 | 12.91 ± 0.01 | 12.84 ± 0.00 | 12.94 ± 0.01 |
| C8A | complement C8α | t0 | 14.01 ± 0.01 | 14.04 ± 0.00 | 14.12 ± 0.01 |
| | | t24 | 13.46 ± 0.01* | 13.60 ± 0.00 | 13.55 ± 0.01 |
| C8B | Complement C8β | t0 | 11.78 ± 0.02 | 11.81 ± 0.01 | 12.18 ± 0.02* |
| | | t24 | * | * | * |
| C9 | complement C9 | t0 | 13.49 ± 0.01 | 13.47 ± 0.00 | 13.81 ± 0.02* |
| | | t24 | 13.06 ± 0.01 | 13.12 ± 0.00 | 13.09 ± 0.01 |
| CFB | complement factor B | t0 | 14.56 ± 0.01 | 14.50 ± 0.00 | 14.64 ± 0.01* |
| | | t24 | 13.79 ± 0.01 | 13.78 ± 0.00 | 13.70 ± 0.01 |
| CFP | complement factor properdin | t0 | 14.72 ± 0.01 | 14.74 ± 0.00 | 14.55 ± 0.01* |
| | | t24 | 13.02 ± 0.01 | 13.04 ± 0.00 | 13.02 ± 0.01 |
| CPN1 | carboxypeptidase N 1 | t0 | 13.71 ± 0.01 | 13.67 ± 0.00 | 13.83 ± 0.01* |
| | | t24 | 11.98 ± 0.02 | 12.00 ± 0.00 | 12.03 ± 0.01 |
| CPN2 | carboxypeptidase N 2 | t0 | 12.65 ± 0.01 | 12.67 ± 0.00 | 12.78 ± 0.01 |
| | | t24 | 13.20 ± 0.01 | 13.34 ± 0.00 | 13.19 ± 0.01* |
| CD163 | Scavenger receptor cysteine- | t0 | 13.10 ± 0.01 | 13.07 ± 0.00 | 13.27 ± 0.01* |
| | rich type 1 protein M130 | t24 | * | * | * |
| CLU | clusterin | t0 | 14.45 ± 0.01* | 14.28 ± 0.00 | 14.35 ± 0.01 |
| | | t24 | 14.17 ± 0.01* | 14.05 ± 0.00 | 13.92 ± 0.01 |
| CST3 | cystatin C | t0 | 14.38 ± 0.02 | 14.35 ± 0.00 | 14.64 ± 0.01* |
| | | t24 | 12.87 ± 0.01 | 12.88 ± 0.00 | 13.04 ± 0.01* |
| FCN3 | Ficolin-3 | t0 | 44.50 ± 0.01* | 14.33 ± 0.00 | 14.49 ± 0.01* |
| | | t24 | * | * | * |
| IGFBP2 | Insulin-like growth factor- | t0 | 13.60 ± 0.01 | 13.48 ± 0.00 | 13.77 ± 0.01* |
| | binding protein 2 | t24 | * | * | * |
| IGFBP4 | insulin-like growth factor | t0 | 13.70 ± 0.01 | 13.68 ± 0.00 | 13.96 ± 0.01* |
| | binding protein 4 | t24 | 12.18 ± 0.02 | 12.05 ± 0.01 | 12.12 ± 0.02 |
| ITIH1 | inter-α (globulin) inhibitor H1 | t0 | 14.03 ± 0.01 | 13.95 ± 0.00 | 13.93 ± 0.01 |
| | | t24 | 13.09 ± 0.01 | 13.02 ± 0.00 | 12.89 ± 0.01* |
| ITIH3 | inter-α (globulin) inhibitor H3 | t0 | 13.79 ± 0.01 | 13.89 ± 0.00 | 14.07 ± 0.01* |
| | | t24 | 12.76 ± 0.01 | 12.86 ± 0.00 | 12.86 ± 0.01 |
| ITIH4 | inter-α (globulin) inhibitor H4 | t0 | 14.17 ± 0.01 | 14.16 ± 0.00 | 14.29 ± 0.01* |
| | | t24 | 13.64 ± 0.01 | 13.69 ± 0.00 | 13.74 ± 0.01 |
| SERPINA1 | α-1-antitrypsin | t0 | 13.76 ± 0.01 | 14.02 ± 0.01 | 14.14 ± 0.01 |
| | | t24 | 13.58 ± 0.01* | 13.87 ± 0.00 | 14.03 ± 0.01 |
| SERPINC1 | serpin peptidase inhibitor C1 | t0 | 14.71 ± 0.01 | 14.68 ± 0.00 | 14.51 ± 0.01* |
| | | t24 | 13.56 ± 0.01 | 13.47 ± 0.00 | 13.41 ± 0.01 |
| SERPINF1 | serpin peptidase inhibitor F1 | t0 | 13.39 ± 0.01 | 13.31 ± 0.00 | 13.63 ± 0.01* |
| | | t24 | 12.06 ± 0.01 | 12.15 ± 0.00 | 12.14 ± 0.01 |
| SERPINF2 | serpin peptidase inhibitor F2 | t0 | 13.65 ± 0.01 | 13.72 ± 0.00 | 13.62 ± 0.01* |
| | | t24 | 12.44 ± 0.01 | 12.49 ± 0.00 | 12.39 ± 0.01* |
| SERPING1 | serpin peptidase inhibitor G1 | t0 | 14.91 ± 0.01 | 14.84 ± 0.00 | 15.08 ± 0.01* |
| | | t24 | 13.79 ± 0.01 | 13.80 ± 0.00 | 13.89 ± 0.01 |
| KLKB1 | kallikrein B1 | t0 | 13.68 ± 0.01 | 13.61 ± 0.00 | 13.49 ± 0.01* |
| | | t24 | 13.00 ± 0.01 | 12.96 ± 0.00 | 12.95 ± 0.01 |
| KNG1 | kininogen 1 | t0 | 14.99 ± 0.01 | 14.89 ± 0.00 | 14.75 ± 0.01* |
| | | t24 | 14.00 ± 0.01* | 13.87 ± 0.00 | 13.76 ± 0.01* |
| PLG | Plasminogen | t0 | 14.36 ± 0.01 | 14.30 ± 0.00 | 14.07 ± 0.01* |
| | | t24 | 14.02 ± 0.01* | 13.92 ± 0.00 | 13.79 ± 0.00* |
| HRG | Histidine-rich glycoprotein | t0 | 14.25 ± 0.01 | 14.21 ± 0.01 | 13.77 ± 0.01* |
| Histidine-rich | | t24 | 13.43 ± 0.02 | 13.28 ± 0.01 | 12.98 ± 0.01* |
| glycoprotein | | | | | |
| F12 | coagulation factor XII | t0 | 13.92 ± 0.01* | 13.73 ± 0.00 | 13.79 ± 0.01 |
| | | t24 | 13.91 ± 0.01* | 13.69 ± 0.00 | 13.76 ± 0.01 |
| F2 | coagulation factor II | t0 | 14.13 ± 0.01 | 14.15 ± 0.00 | 14.20 ± 0.01 |
| | | t24 | 13.41 ± 0.01 | 13.32 ± 0.00 | 13.40 ± 0.01* |
| FGB | fibrinogen β | t0 | 14.46 ± 0.01 | 14.66 ± 0.00 | 14.43 ± 0.01 |
| | | t24 | 14.28 ± 0.01* | 14.42 ± 0.00 | 14.35 ± 0.01 |
| FN1 | fibronectin 1 | t0 | 14.14 ± 0.01 | 13.95 ± 0.00 | 13.74 ± 0.01* |
| | | t24 | 13.47 ± 0.01* | 13.25 ± 0.00 | 13.40 ± 0.01 |
| LGALS3BP | lectin galactoside-binding-3 | t0 | 12.70 ± 0.01 | 12.61 ± 0.00 | 12.88 ± 0.01* |
| | binding protein | t24 | 11.60 ± 0.02 | 11.46 ± 0.00 | 11.61 ± 0.02 |
| LRG1 | leucine-rich α-2-glycoprotein 1 | t0 | 14.01 ± 0.01* | 14.31 ± 0.00 | 14.27 ± 0.01 |
| | | t24 | 13.58 ± 0.02* | 13.85 ± 0.00 | 13.90 ± 0.01 |
| LYZL4 | lysozyme-like 4 | t0 | * | * | * |
| | | t24 | 14.01 ± 0.01* | 14.18 ± 0.00 | 14.24 ± 0.01 |
| FCGBP | Fc fragment of IgG binding | t0 | 13.66 ± 0.01 | 13.60 ± 0.00 | 13.86 ± 0.01* |
| | protein | t24 | 13.45 ± 0.01* | 13.65 ± 0.00 | 13.74 ± 0.01 |
| LBP | lipopolysaccharide binding | t0 | 13.99 ± 0.01* | 14.54 ± 0.01 | 14.46 ± 0.02 |
| | protein | t24 | 13.04 ± 0.01* | 13.46 ± 0.01 | 13.38 ± 0.01 |
| PGLYRP2 | peptidoglycan recognition | t0 | 12.87 ± 0.01 | 12.70 ± 0.00 | 12.76 ± 0.01 |
| | protein 2 | t24 | 12.05 ± 0.01* | 11.85 ± 0.00 | 11.65 ± 0.01* |
| PIGR | polymeric immunoglobulin | t0 | 14.92 ± 0.01 | 14.70 ± 0.01 | 14.78 ± 0.02 |
| | receptor | t24 | 12.90 ± 0.02 | 12.74 ± 0.00 | 13.02 ± 0.01* |
| CRP | C-reactive protein | t0 | 13.45 ± 0.02* | 13.92 ± 0.01 | 14.51 ± 0.03* |
| | | t24 | 14.09 ± 0.02* | 14.73 ± 0.01 | 14.58 ± 0.02 |
| SAA1 | Serum amyloid A1 | t0 | 13.26 ± 0.01* | 13.83 ± 0.01 | 13.77 ± 0.02 |
| | | t24 | 12.47 ± 0.02* | 13.27 ± 0.01 | 12.96 ± 0.02 |
| SAA3P | serum amyloid A3 | t0 | 13.38 ± 0.01* | 13.96 ± 0.01 | 13.92 ± 0.02 |
| | | t24 | 12.91 ± 0.03* | 13.86 ± 0.01 | 13.58 ± 0.03 |
| DEFA1 | defensin α1 | t0 | 13.29 ± 0.03 | 13.34 ± 0.01 | 13.97 ± 0.04* |
| | | t24 | 10.71 ± 0.01 | 10.96 ± 0.01 | 11.12 ± 0.02 |
| SERPINA3 | serpin peptidase inhibitor A3 | t0 | 14.25 ± 0.01* | 14.52 ± 0.00 | 14.63 ± 0.01 |
| | | t24 | 13.19 ± 0.01* | 13.50 ± 0.00 | 13.52 ± 0.01 |
| PON1 | paraoxonase 1 | t0 | 13.54 ± 0.01 | 13.52 ± 0.00 | 13.34 ± 0.01* |
| | | t24 | 12.59 ± 0.01 | 12.46 ± 0.00 | 12.21 ± 0.01* |
| QSOX1 | Sulfhydryl oxidase 1 | t0 | 13.26 ± 0.01 | 13.21 ± 0.00 | 13.41 ± 0.01* |
| | | t24 | * | * | * |
| RBMX | RNA binding motif protein X- | t0 | 15.11 ± 0.03 | 15.07 ± 0.01 | 15.31 ± 0.03 |
| | linked | t24 | 13.77 ± 0.03 | 13.88 ± 0.01 | 14.29 ± 0.02* |
| S100A9 | S100 calcium binding protein | t0 | 13.22 ± 0.02 | 13.38 ± 0.01 | 13.57 ± 0.02 |
| | A9 | t24 | 12.29 ± 0.01 | 12.53 ± 0.01 | 12.92 ± 0.02* |
| SEPP1 | selenoprotein P1 | t0 | 13.23 ± 0.01* | 12.96 ± 0.00 | 12.84 ± 0.02 |
| | | t24 | 12.77 ± 0.01 | 12.64 ± 0.00 | 12.70 ± 0.01 |
| TIMP1 | TIMP metallopeptidase | t0 | 13.81 ± 0.01 | 13.80 ± 0.00 | 13.92 ± 0.01 |
| | inhibitor 1 | t24 | 13.54 ± 0.01* | 13.68 ± 0.00 | 13.89 ± 0.01* |
| BAI2 | brain-specific angiogenesis | t0 | 13.26 ± 0.02 | 13.48 ± 0.01 | 13.50 ± 0.02 |
| | inhibitor 2 | t24 | 14.50 ± 0.02* | 14.90 ± 0.01 | 15.00 ± 0.02 |
| MCAM | Cell surface glycoprotein | t0 | 14.78 ± 0.02 | 14.69 ± 0.01 | 14.30 ± 0.01* |
| | MUC18 | t24 | * | * | * |
| ULK4 | unc-51-like kinase 4 | t0 | 15.07 ± 0.02 | 15.16 ± 0.00 | 14.80 ± 0.02* |
| | | t24 | 14.57 ± 0.03 | 14.65 ± 0.01 | 14.60 ± 0.02 |
| CDA6 | C4A6 | t0 | 14.33 ± 0.01 | 14.42 ± 0.00 | 14.62 ± 0.01* |
| | | t24 | * | * | * |

Akin to the metabolome, the plasma proteome disclosed a dichotomous host response in sepsis survivors and deaths (64 and 27 protein differences at t₀ and t₂₄, respectively; FIG. 24a; FIG. 25; Table 14). Unlike the metabolome, however, the proteomic variance associated with outcome did not increase as death approached. There was strong concordance between time points: 50 of 66 plasma proteins with significant survivor-death differences had congruent changes at the other time point. 22 complement cascade proteins were increased in sepsis deaths, while 8 thrombolysis proteins were decreased and 3 were increased (FIG. 24b), consistent with previous reports. Of relevance to increased fatty acids and carnitine esters in sepsis death were decreased levels of nine fatty acid transport proteins (apolipoproteins AI, AII, AIV, L1, CIV, transthyretin, hemopexin, afamin and α-2-HS-glycoprotein). A material negative finding was an absence of increase in plasma levels of large intracellular proteins, indicative of an absence of gross tissue necrosis or injury.

### Example 5

### Blood Transcriptomics

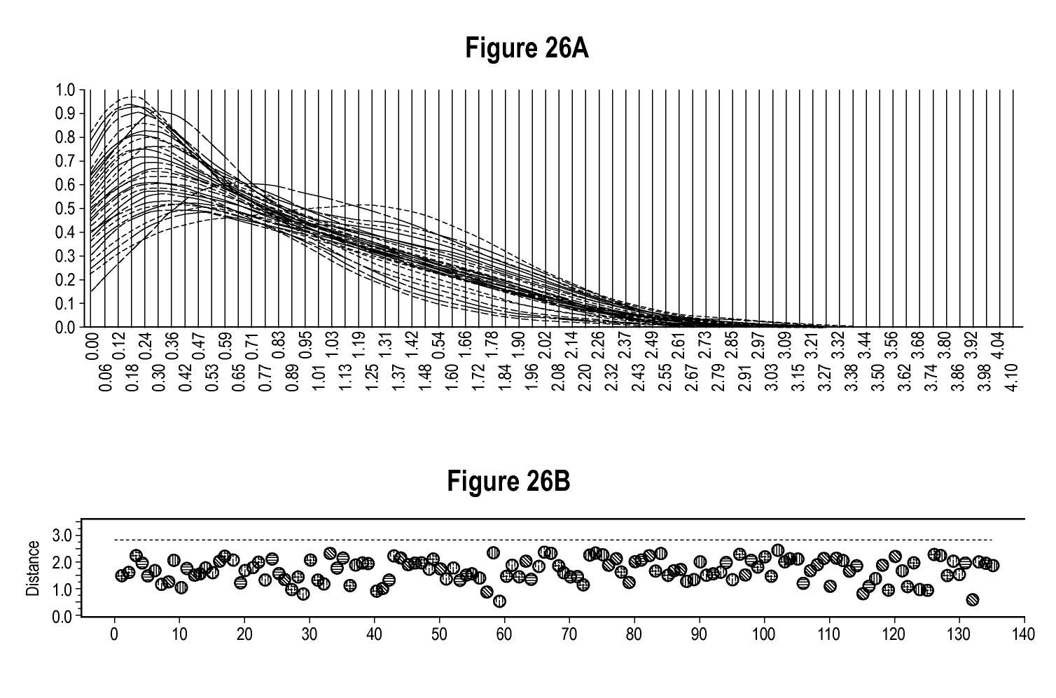

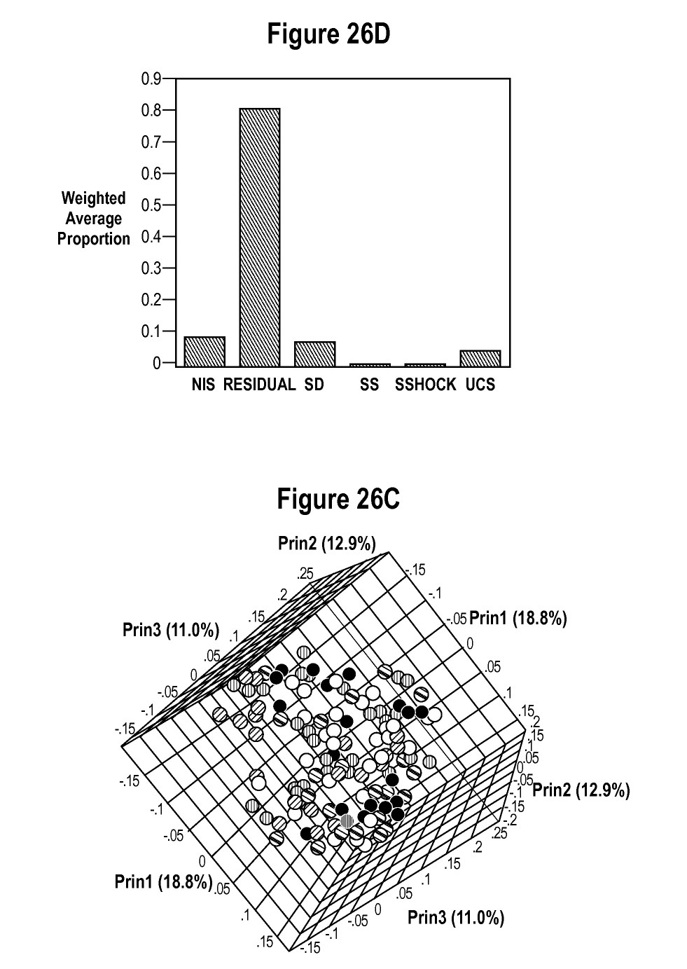

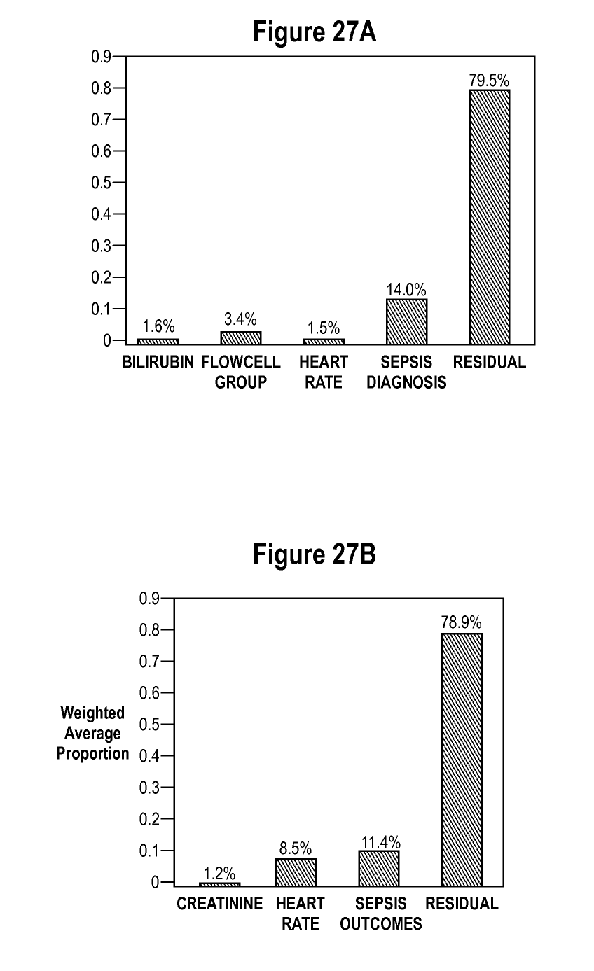

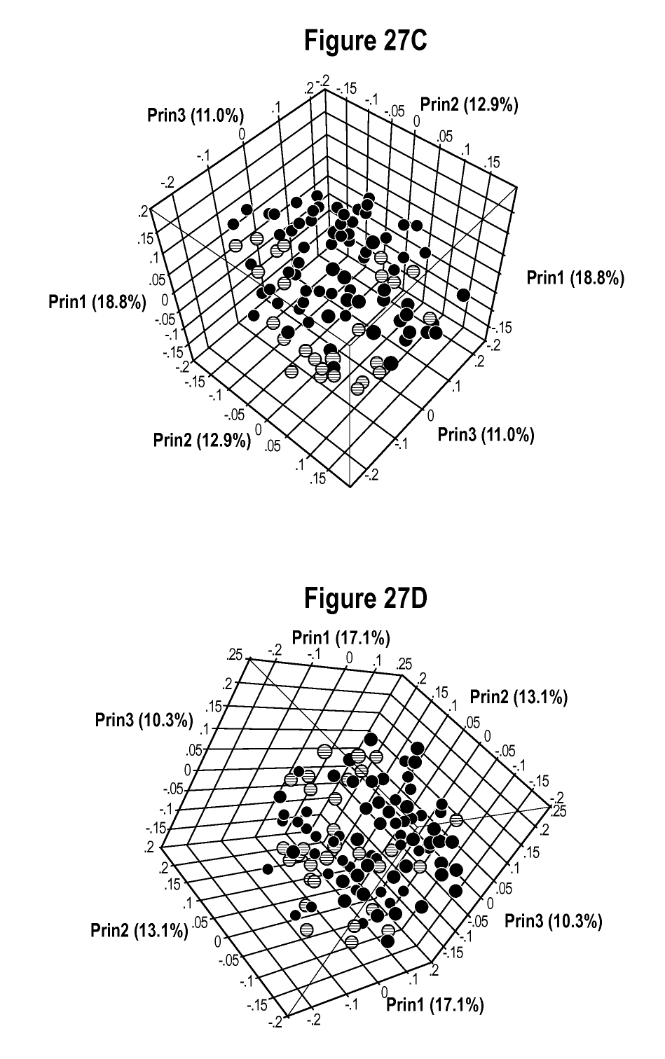

Transcription in venous blood of patients at ED arrival was evaluated by sequencing mRNA from the discovery cohort at t₀ (FIG. 3), which yields both absolute mRNA molecule counts of analytic superiority to ratiometric approaches, and coding nucleotide variants^{40,41}. Blood was collected in PaxGene tubes, preserving in vivo transcript levels but preventing isolation of specific cell sets. Neither leukocyte count nor RNA yield differed significantly between controls, sepsis survivors and deaths. ˜600 million, 54-nucleotide mRNA sequences from each subject were aligned to the human genome, yielding relative levels of transcription of 32,359 genes in blood (of which 18,618 mRNAs were detected in >50% of subjects; data not shown). While sepsis group membership accounted for ˜20% of variance in gene expression, only 3.7% was attributable to sepsis survivor subgroups, in accord with the plasma proteome and metabolome (FIGS. 26 and 27).

Differences in transcript abundance between sepsis survivors and controls and sepsis survivors and deaths were strikingly skewed (FIG. 28a). 3,128 transcripts were significantly increased and 54 decreased in sepsis survivors (compared with controls, stratified ANOVA, FDR 5% data not shown). In contrast, 1,326 transcripts were significantly decreased and only 64 were increased in sepsis deaths (compared with survivors; data not shown). Relevant to this shift in transcription was significantly altered expression of 29 transcriptional regulatory genes, of which 23 were decreased in sepsis death (including FOXO3, oncogenes jun B, jun D and v-maf, two Kruppel-like transcription factors, three enhancer binding proteins (C/EBP), a cyclin-dependent kinase-associated gene, three splicing factors and seven other DNA binding proteins). C/EBP-α binding activity has previously been shown to decrease in sepsis death. Additionally, several RNA polymerase transcripts (POLRMTL, POLR2E and POLR2J) and TATA box binding proteins (TAF10, TAF6 and TAF1C) were decreased in sepsis death. Six transcriptional regulatory genes were increased in sepsis death, including transcription factors Sp3 and E74-like factor 2 and nuclear receptor coactivator 2 (TIF2, SRC2). An additional factor in the shifts in mRNA abundance sepsis survivors and deaths was increased RNase3 transcripts in sepsis death, and decreased RNAse inhibitor (RNH1) transcripts.

Other prominent functional classes that differed in mRNA abundance in sepsis outcome were kinases, transporters, and peptidases (FIG. 28b); prominent networks or pathways were apoptosis, inflammation, neutrophil genes, signal transduction, superoxide metabolism, thrombosis/thrombolysis, ubiquitin system and metabolism (FIG. 28b).

Transcriptome differences suggested elevation of metabolic rate in sepsis survivors: RNAs for 41 nuclear-encoded mitochondrial proteins were significantly increased in sepsis survivors (compared with controls) and 15 were decreased in sepsis death (FIG. 28c). In addition, RNAs for 29 enzymes involved in glycolysis, gluconeogenesis, citric acid cycle, FA β-oxidation, oxidative phosphorylation and mitochondrial transport were significantly increased in survivors (compared to controls), while 32 were decreased in sepsis death. For example, fructose-1,6-bisphosphatase 1, which regulates gluconeogenesis, was significantly elevated in sepsis survivors and depressed in sepsis deaths. Relevant mRNAs that were decreased in sepsis death were FA transport proteins (carnitine acyltransferase, carnitine palmitoyltransferase 1B [CPT1B], SLC27A3, and malonyl CoA:ACP acyltransferase) and FA β-oxidation enzymes (pantothenate kinase 4, CoA synthase and mitochondrial enoyl CoA hydratase 1). Decreased CPT1 and CoA synthase have previously been documented in sepsis.

Transcription of innate immune effectors was markedly different in sepsis survivors and deaths (FIG. 28c): mRNAs for 10 interferon-induced genes, 12 tumor necrosis factor superfamily ligands and receptors and 8 apoptosis genes were decreased in deaths. Of particular note, lymphotoxin β was substantially decreased in sepsis death. Also reduced in sepsis death were toll-like receptor 9, toll interacting protein and toll-like receptor adaptor molecule 1 (TICAM1, TRIF). In murine viral endocarditis, TICAM1 deficiency is associated with 100% mortality. The sole gene related to innate immunity upregulated in sepsis death was tyrosylprotein sulfotransferase 1, which increases interleukin-6 production by LPS-treated macrophages.

Finally, among the small number of mRNAs that were significantly increased in sepsis death were six involved in coagulation and endothelial cell adhesion (angiopoietin-like 2, thrombin receptor-like 2, glycophorin B, kallikrein-related peptidase 8, lymphatic vessel endothelial hyaluronic receptor 1 and PFTAIRE protein kinase). Together with complement regulator CD59, which was decreased in sepsis death, these transcriptional changes agreed with the observed perturbation in thrombolysis and complement proteins in sepsis deaths (FIG. 24b). Non-congruent were SERPINA1, SERPING1 and five complement component proteins (significantly elevated in plasma in sepsis death, but not in blood mRNA), likely reflecting primacy of synthesis by the liver rather than leucocytes.

Common and rare expressed genetic variants that might underpin the molecular differences in sepsis survivors and deaths were sought. Variants were identified and genotyped in peripheral blood transcripts from 142 subjects at nucleotides with adequate coverage (present in ≧4 reads of Q≧20 and >14% reads), as described. Variant genotypes and collapsed variant genotypes within a gene were tested for association with 28-day survival or death under the common disease:common variant and common disease:rare variant hypotheses. Of 384,283 nuclear and mitochondrial mRNA variants, none showed a significant association. However, combined variants in 20 genes showed significant associations with outcome (−log₁₀(p) value≦32; Hotelling T-squared test or regression analysis of principal components representing the combined variants), were observed in at least 60 samples and had at least moderately altered odd ratios in survivor:death and sepsis survivor:death comparisons (Table 6). Several of these genes were plausible functional candidates for risk of adverse sepsis outcome: 4 encoded mitochondrial proteins and 9 exhibited altered mRNA levels in sepsis survival and death. Notably, subunits α2 and β8 of NADH dehydrogenase 1, a component of the mitochondrial electron transport chain, had excess variants in sepsis deaths.

### Expressed Genetic Variants

Common and rare expressed genetic variants that might underpin the molecular differences in sepsis survivors and deaths were sought. Variants were identified and genotyped in peripheral blood transcripts from 142 subjects at nucleotides with adequate coverage (present in ≧4 reads of Q≧20 and >14% reads), as described. Variant genotypes and collapsed variant genotypes within a gene were tested for association with 28-day survival or death under the common disease:common variant and common disease:rare variant hypotheses⁴⁹. Of 384,283 nuclear and mitochondrial mRNA variants, none showed a significant association. However, combined variants in 20 genes showed significant associations with outcome (−log₁₀(p) value≦32; Hotelling T-squared test or regression analysis of principal components representing the combined variants), were observed in at least 60 samples and had at least moderately altered odd ratios in survivor:death and sepsis survivor:death comparisons (Table 6). Several of these genes were plausible functional candidates for risk of adverse sepsis outcome: 4 encoded mitochondrial proteins and 9 exhibited altered mRNA levels in sepsis survival and death. Notably, subunits α2 and 138 of NADH dehydrogenase 1, a component of the mitochondrial electron transport chain, had excess variants in sepsis deaths.

### Example 6

### Integration of Disparate Datasets

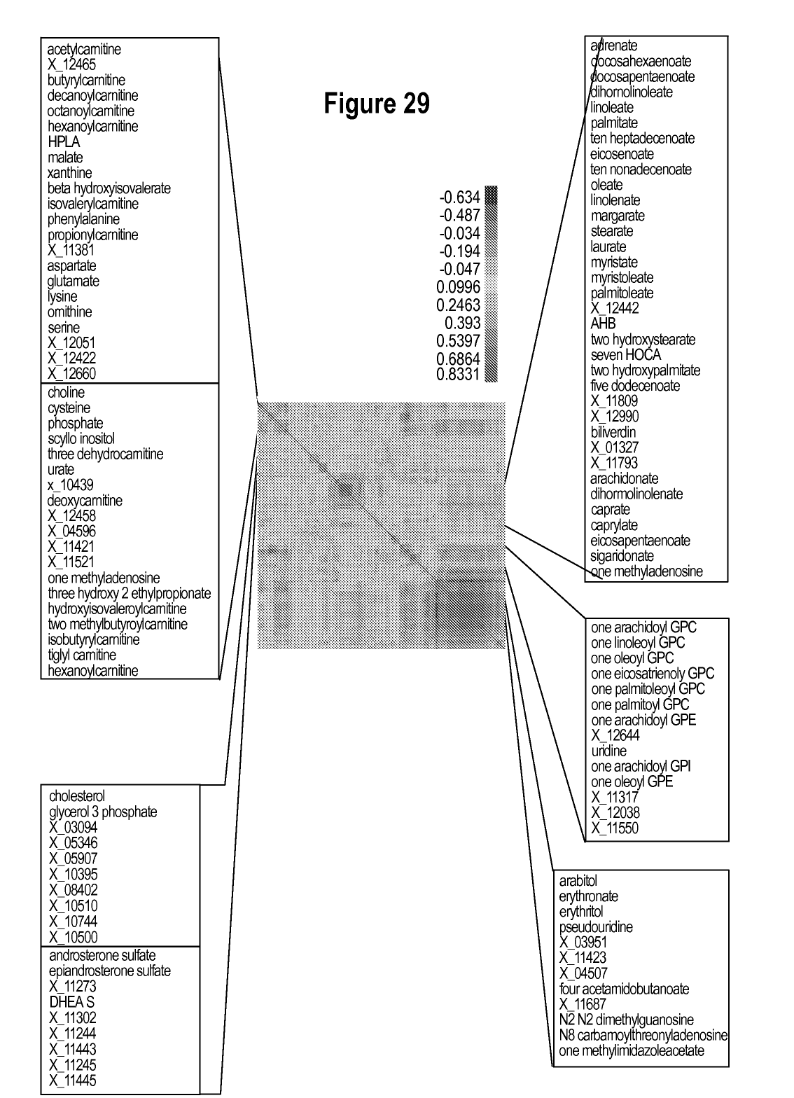

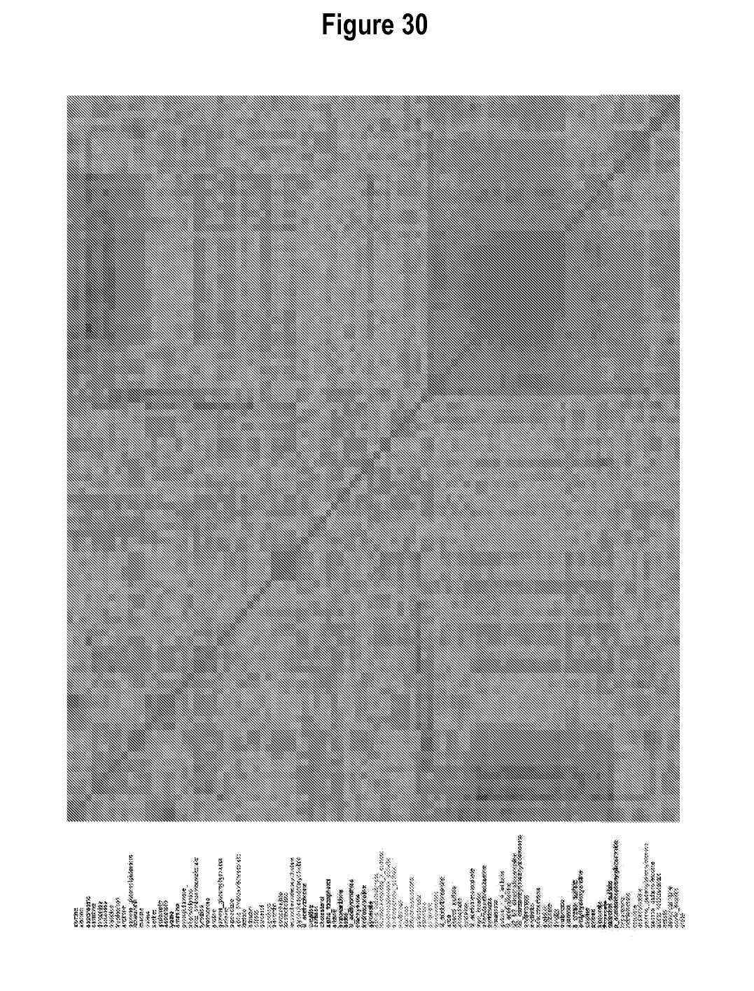

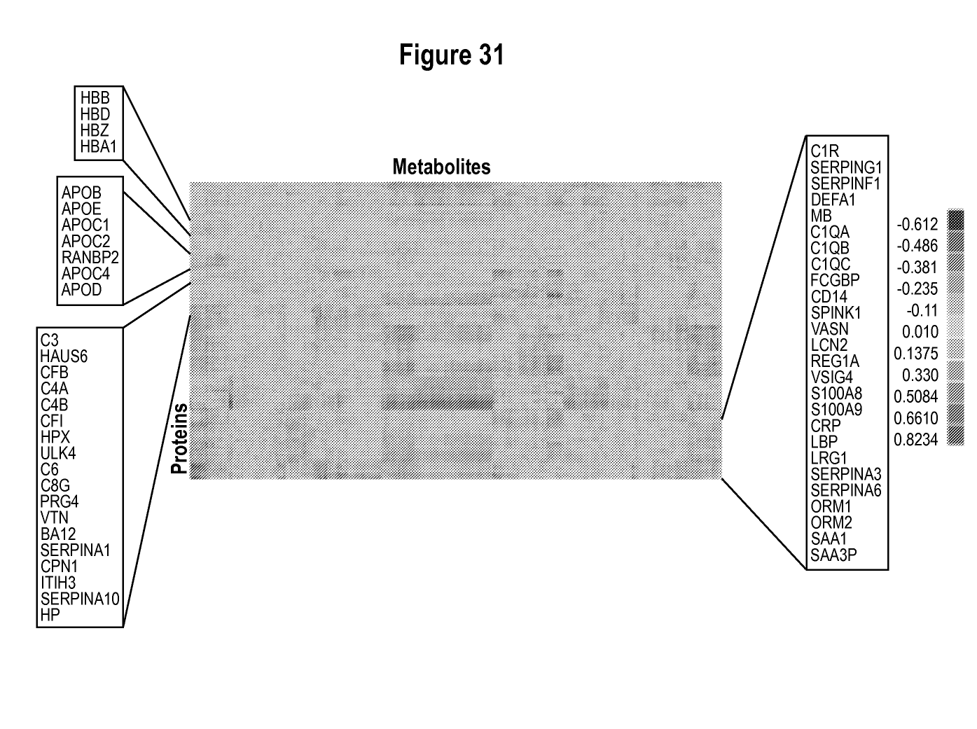

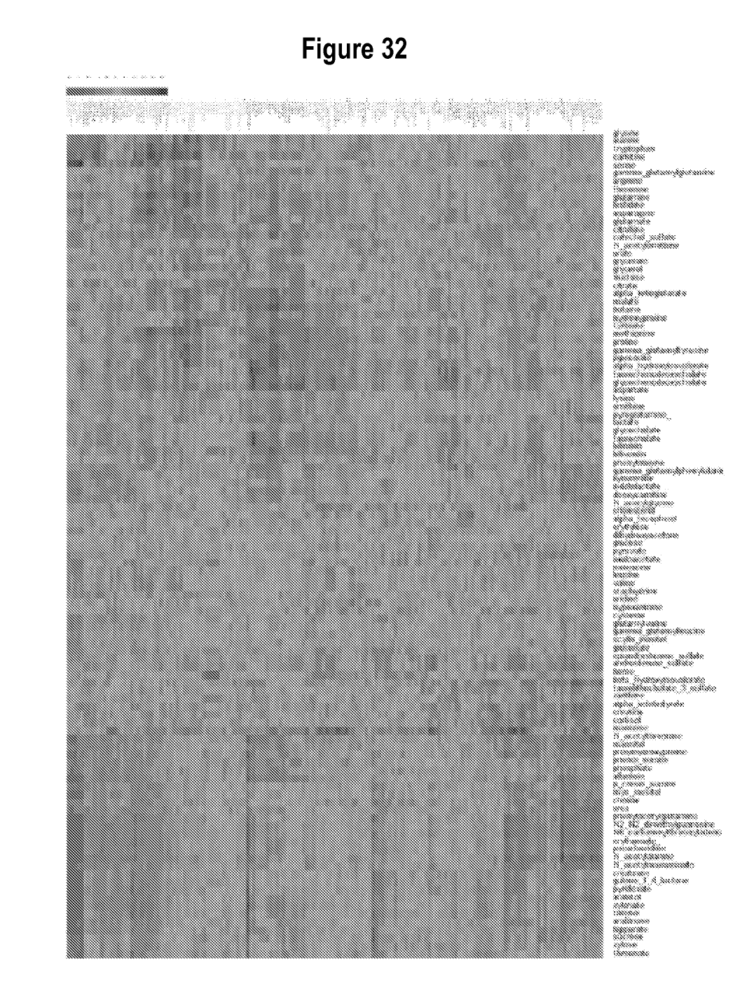

Surveys of the plasma proteome and metabolome were also integrated by global cross-correlations and hierarchical clustering of correlations (FIG. 13 f, g; 24 c,d). Biochemical class membership was largely recapitulated in correlation co-clustering hierarchies (FIG. 13 f, g; 24 c, d; FIGS. 29-32): For example, 7 acyl-carnitines were nearest neighbors at t₀, as were 5 androgenic steroids, 11 acyl-GPCs and acyl-GPEs, 5 bile acids, 16 FAs, 12 amino acid metabolites and the group lactate-citrate-glycerol-pyruvate-oxaloacetate (FIG. 29). Likewise, functionally or structurally related proteins co-clustered, such as 4 hemoglobin isoforms, 9 complement components, and 10 apolipoproteins (FIG. 13 f, g; FIGS. 29, 30). Importantly, class membership of several unannotated biochemicals imputed by co-cluster hierarchies was confirmed by structural determination: Unannotated biochemicals X-11302, X-11245 and X-11445 co-clustered with DHEAS, androsterone sulfate and epiandrosterone sulfate and were determined to be sulfated pregnenolone-related steroids (pregnen-steroid monosulfate, pregnen-diol disulfate and 5α-pregnan-3β, 20α-diol disulfate, respectively); X-11421 co-clustered with 8 medium chain acyl-carnitines and was determined to be cis-4-decenoylcarnitine; X-12465 co-clustered with acetyl- and propionyl-carnitine and was determined to be 3-hydroxybutyrylcarnitine (FIGS. 14, 29).

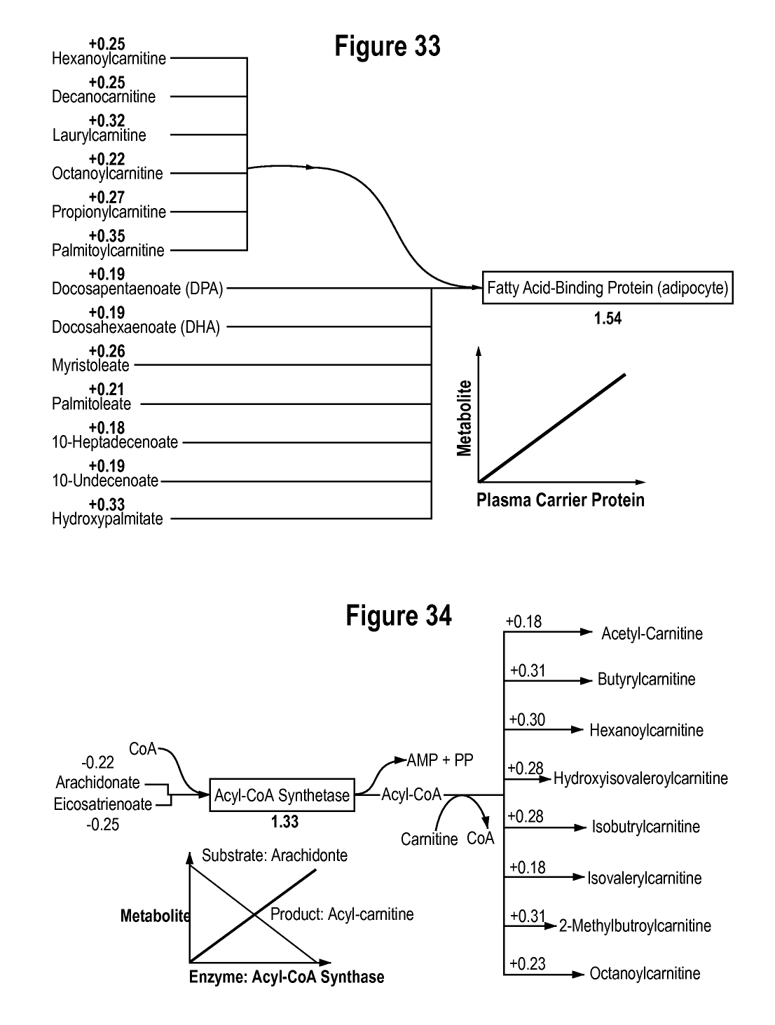

4,106 of 53,784 plasma protein-metabolite correlations were concordant at t₀ and t₂₄ and statistically significant (Bonferroni-corrected log₁₀ p-value<−6.03; data not shown). These included known mass action kinetic models of catalysis or physicochemical complex assembly: Ribonuclease A1 correlated with 12 downstream products of its action (N6-carbamoylthreonyladenosine, N2,N2-dimethylguanosine, pseudouridine, arabitol, arabinose, erythritol, erythronate, gulono-1,4-lactone, allantoin, phosphate, xylonate and xylose). Hemoglobin subunits α1, β, δ and ζ correlated with the component heme, allosteric effector adenosine-5-monophosphate and degradation product xanthine. Subunit D of succinate dehydrogenase (SDHD, a high confidence protein identification supported by a single peptide) correlated with 3 downstream citric acid cycle intermediates (L-malate, oxaloacetate and citrate; FIG. 13e). Several acyl-carnitines/FAs correlated with their plasma transporter fatty acid binding proteins (FABP1 and FABP4, FIG. 33). Two fatty acid substrates correlated inversely with Acyl-CoA Synthase (ACSM6, another high confidence protein identification supported by a single peptide), which catalyzes attachment of fatty acids to CoA for β-oxidation (FIG. 34).

Co-cluster hierarchies and correlations also suggested novel reaction models: Thus, SDHD correlated with pyruvate, lactate and acetyl-carnitine, suggesting novel regulation of the citric acid cycle (FIG. 13e), which has some experimental support. Another plausible model was suggested by correlations of ACSM6 with 9 acyl-carnitines (FIG. 34). ACSM6 acts upstream of carnitine esterification, which mediates mitochondrial FA import. The generalizability and verification of these novel models will require quantitative measurements and confirmation of co-localization in cellular compartments.

Only 3 plasma proteins or metabolites correlated significantly with blood transcripts: levels of fatty acid binding protein 1 and S100A9 correlated with their respective mRNAs (Pearson coefficients 0.49; −log₁₀p=9.0 and 8.8, respectively). Uridine phosphorylase 1 mRNA correlated inversely with plasma uridine (r²=−0.48, −log₁₀p=8.7), consistent with their enzyme-substrate relationship. The paucity of mRNA correlations likely reflects the small effect of blood cells to MS-detected plasma protein and metabolite levels, relative to liver and muscle.

### Example 7

### Biomarker Validation and Applications

The goal of the current study was to identify markers for prompt and objective determination of prognosis in individual sepsis patients in order to tailor treatment dynamically. Since such markers have been sought for decades, an innovative approach, with three premises, was taken. Firstly, comprehensive, hypothesis-agnostic description of the molecular antecedents of survival and death was posited to yield new, unbiased insights. Secondly, holistic integration of metabolomic, proteomic, transcriptomic and genetic data was posited to permit identification of signals undetected or obscured by false discovery cutoffs in single datasets. Thirdly, co-occurrence and correlation of networks and pathways in orthogonal datasets was posited to help identify and prioritize causal molecular mechanisms. Therefore, findings identified in individual datasets by statistically significant group differences in discovery and replication cohorts were prioritized by: 1). assembly into networks, pathways or biochemical families; 2). temporal confirmation or evolution of changes; 3). network and pathway corroboration in orthogonal datasets; and 4). cross correlations, hierarchical co-clustering and assembly of mass action kinetic models of catalysis or physicochemical complexes. Finally, prognostic biomarker candidates were chosen to reflect underpinning molecular mechanisms, rather than by ability to partition accurately.

An integrated systems survey revealed sepsis to be a complex, heterogeneous and dynamic pathologic state and yielded new insights into molecular mechanisms of survival or death that may enable predictive differentiation and individualized patient treatment. There were both negative and positive material findings.

The major negative finding was that the plasma metabolome, proteome and transcriptome did not differ between uncomplicated sepsis, day 3 severe sepsis, day 3 septic shock nor between infections with S. pneumoniae, S. aureus or E. coli. There were no plasma metabolic or proteomic differences between these groups either at time of presentation for care or at t₂₄. Thus, sepsis survivors represented a molecular continuum, irrespective of imminent clinical course or etiology. It should be noted, however, that MS-based proteome analysis was insensitive for measurement of low molecular weight proteins, such as cytokines, which are known to differ between etiologic agents. Importantly, all datasets refuted the concept that the discrete clinical stages of progression from uncomplicated sepsis to severe sepsis to septic shock have a unifying molecular basis. The molecular homogeneity of uncomplicated sepsis, severe sepsis and septic shock was remarkable, challenging the traditional notion of a temporal or molecular pyramid of sepsis progression (FIG. 3a). While surprising, this does not alter the importance of early achievement of effective compartmental concentrations of appropriate antibiotics nor the known differences in mortality between etiologic agents or sites of infection.

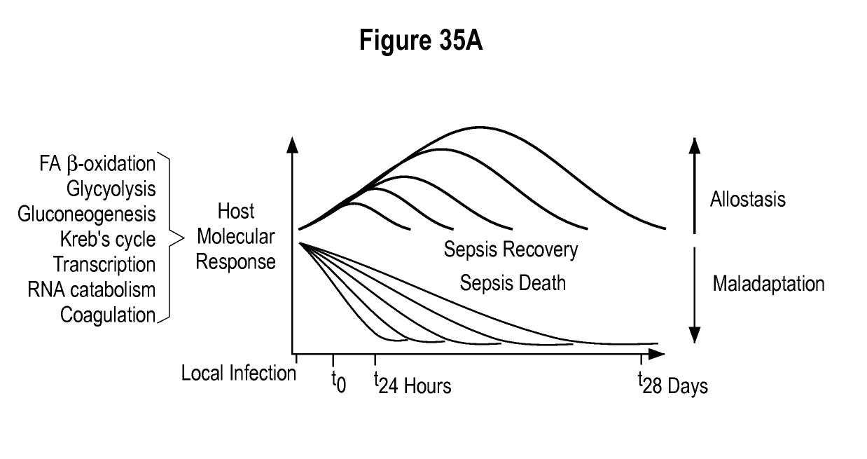

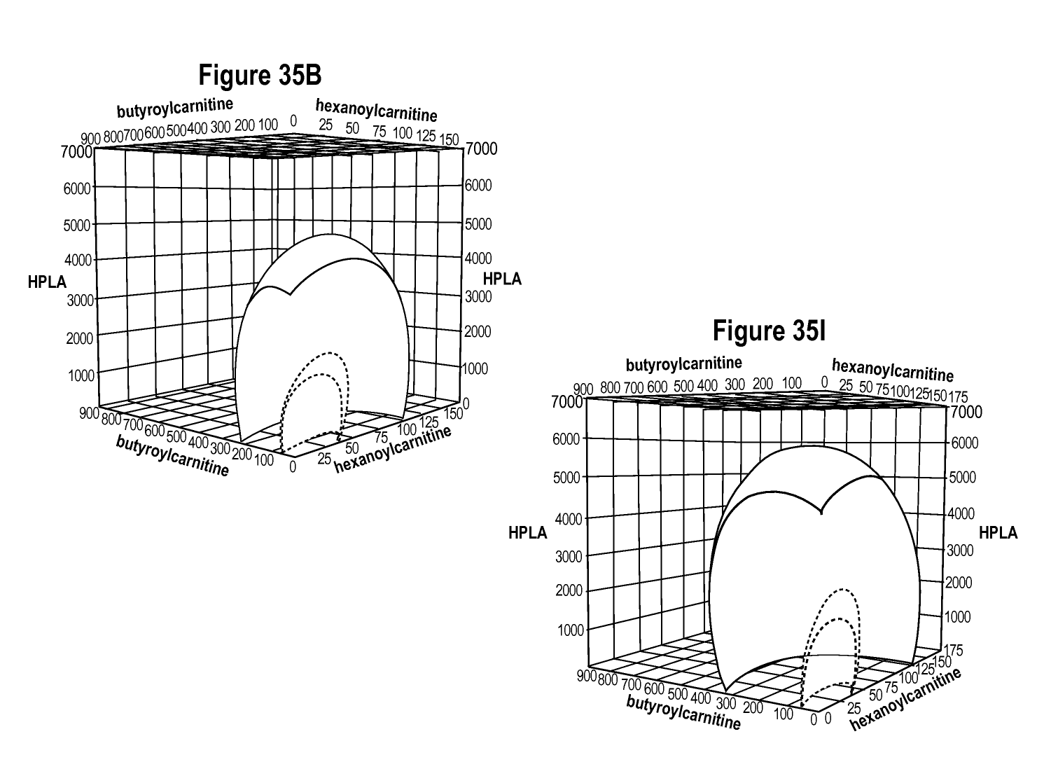

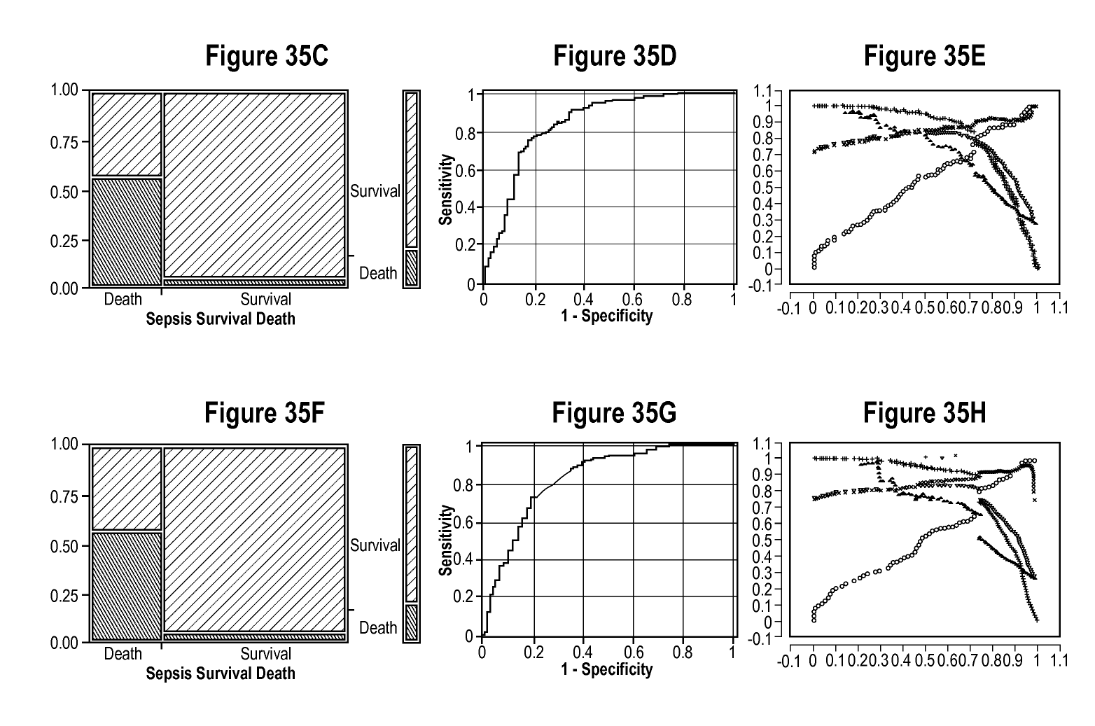

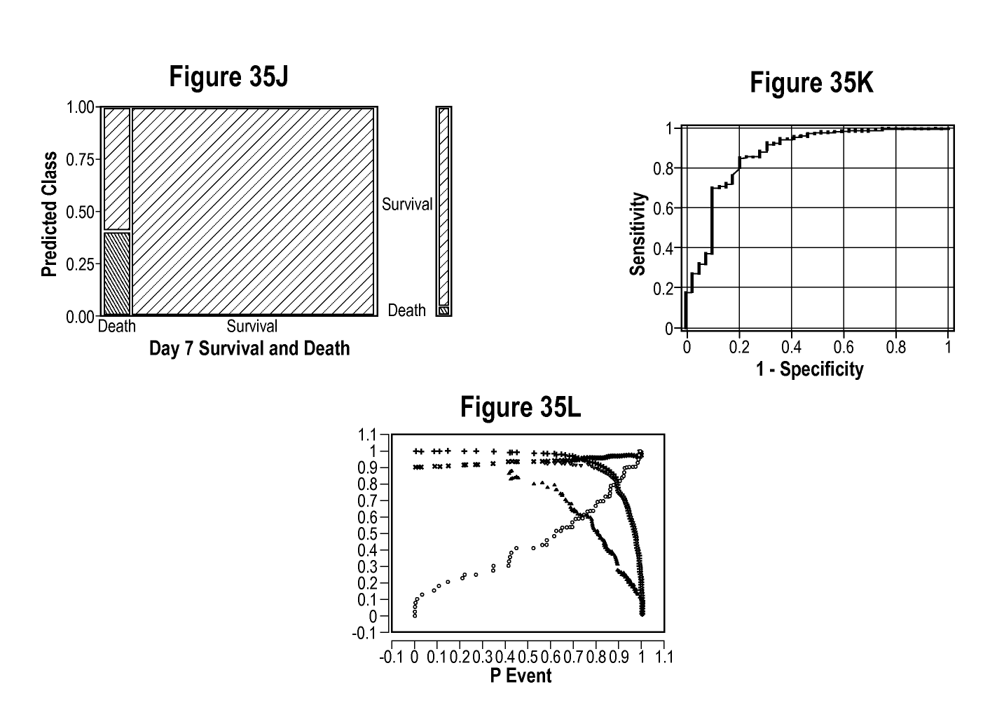

The major positive finding was that the vast majority of host molecular responses were directly opposite in sepsis survivors and deaths (FIG. 35a). This was evident at time of presentation, increased at t₂₄ and became more pronounced as time-to-death decreased. It was observed in the plasma metabolome, proteome and transcriptome. It was true both of mean values of individual analytes, even after inclusion of renal and hepatic as fixed effects, and globally, as assessed by Z-scores, mScores, variance components and global cross-correlations. Divergent host responses were highly conserved temporally, both by global measures, such as Kullback-Liebler distances, and at the level of individual analyte classes, networks and pathways. Thus, there appears to be a remarkable dichotomy in host molecular response to sepsis, reflecting allostasis in survivors, and maladaption in non-survivors.

Prominent in the disparate molecular phenotype of sepsis survival and death was altered fatty acid metabolism: Plasma levels of 6 carnitine esters were decreased in sepsis survivors, relative to controls. In contrast, 16 carnitine esters and 4 FA were elevated in sepsis deaths. Corroborating the metabolic changes were decreases in mRNAs encoding carnitine acyltransferase, carnitine palmitoyltransferase 1B, SLC27A3, malonyl CoA:ACP acyltransferase and the FA β-oxidation enzymes pantothenate kinase 4, CoA synthase and mitochondrial enoyl CoA hydratase 1 in sepsis death. 9 fatty acid transport proteins were decreased in sepsis death, while plasma levels of two fatty acid binding proteins correlated with acyl-carnitine and FA levels. Some of these have been previously reported. Several transcriptional regulatory genes that control fatty acid metabolism were also decreased in sepsis death, including FOXO3, KLF2, C/EBP-α and -β, while TIF2 (NCOA2) was increased. TIF2 is an energy rheostat, which is activated in states of energy depletion, depresses uncoupling protein 3, and increases fat absorption from the gut. Thus, TIF2 up-regulation may represent a maladaptive host response in sepsis death, further elevating plasma lipids that are already increased by impaired β-oxidation. Together, these findings indicate a defect in FA β-oxidation in sepsis death, particularly at the level of the mitochondrial shuttle. Carnitine esterification commits FAs irreversibly to β-oxidation and mitochondrial import of carnitine esters is rate limiting in FA β-oxidation. Acyl-carnitines of all FA lengths were elevated and several shuttle enzymes were affected. A causal role for acylcarnitines in sepsis death is suggested by the finding that micromolar amounts cause ventricular dysfunction. Furthermore, Mendelian mutations of acylcarnitine metabolism induce similar metabolic derangements and high rates of sudden death.

Glycolysis, gluconeogenesis and the citric acid cycle also differed prominently in sepsis survivors and deaths. Plasma values of citrate, malate, glycerol, glycerol 3-phosphate, phosphate and glucogenic and ketogenic amino acids were decreased in sepsis survivors, relative to controls. In contrast, citrate, malate, pyruvate, dihydroxyacetone, lactate, phosphate and gluconeogenic amino acids were increased in sepsis deaths. A corroborating proteomic change was subunit D of succinate dehydrogenase, whose level correlated with the downstream citric acid cycle intermediates malate, oxaloacetate and citrate and with lactate, pyruvate and acetyl-carnitine. Corroborating maladaptive transcriptome changes in sepsis deaths were decreased fructose-1, 6-bisphosphatase 1, hexokinase 3, glucosidase, glycogen synthase kinase, NAD kinase and NAD synthase 1. A parsimonious explanation of these findings was that sepsis survivors mobilized energetic substrates and utilized these in aerobic catabolism completely, while those who would die failed to do so. One clinical corroboration was significantly lower core temperature in sepsis deaths than survivors.

Several lines of evidence support the primacy of metabolism as a determinant of sepsis outcome: Structural studies show mitochondrial derangements, decreased mitochondrial number and reduced substrate utilization in sepsis death, and progressive drop in total body oxygen consumption with increasing severity of sepsis. An early indicator of sepsis outcomes is mitochondrial biogenesis. Finally, sepsis-induced multiple organ failure occurs despite minimal cell death and recovery is rapid in survivors, ruling out irreversible mechanisms. Alternatively, the differences observed in corticoid levels in sepsis survivors and nonsurvivors may betoken neuro-hormonal control of disparate metabolic responses to sepsis. While levels of unbound metabolites in plasma reflect tissue concentrations, values may not be in linear relationship with tissues. Nevertheless, long experience with clinical chemistry predicated on plasma values.

The immediacy of the metabolic dichotomy in sepsis suggested a pre-existing susceptibility and potentially indicated a unifying risk factor. Survivors and deaths did not differ significantly in medication prior to enrollment. However, nucleotide variants in 20 genes showed evidence as risk factors for adverse outcome. The functions of these genes concurred with the molecular differences between sepsis survival and death: SLC16A13 transports lactate and pyruvate; vitamin K epoxide reductase complex, subunit 1, is important for blood clotting; CCAAT/enhancer binding proteins is important in granulocyte maturation and response to TNFα; NADH dehydrogenase 1 α2 and β8 are components of the mitochondrial electron transport chain. The relationships between these variants and the survival/death molecular phenotypes remain unknown.

In summary, an integrated systems survey revealed new and surprising insights into molecular mechanisms of sepsis survival and death. The current study examined community-acquired sepsis in adults in detail, and mainly caused by Streptococcus pneumoniae (and thereby lobar pneumonia), Escherichia coli (and thereby urosepsis) and Staphylococcus aureus (and thereby skin, soft tissue, and catheter associated infections). Additional longitudinal investigation of the host metabolic response to sepsis is needed to address more fully the temporal dynamics and breadth of relevance of this dichotomy in community-acquired infection. New proteomic technologies are available with greater sensitivity than those used herein. Ideally, liver or muscle tissue would be examined concomitantly with blood in order to confirm the relevance of the latter. Additional studies are needed to evaluate the applicability of these findings to nosocomial sepsis, pediatric sepsis, neonatal sepsis, other patient populations and other etiologic agents. Investigation of the relevance of host metabolic dichotomy to other SIRS-inducing conditions, such as trauma, hyperthermia and drug-induced mitochondrial damage, is also warranted.

Finally, prognostic biomarker models derived from the molecular events and mechanisms elucidated in sepsis survival and death were developed. For practical reasons, a homogeneous biomarker panel was sought, rather than combinations of protein, metabolite and RNA measurements. In general, biomarker panels have had disappointing rates of replication. Reasons include data overfitting, reliance on cross-validation rather than independent validation, recruitment at single sites and dependence on single analytic platforms or statistical methods. We sought to obviate these by development of sparse panels, recruitment at three sites, use of two metabolite measurement techniques, replication in an independent CAPSOD cohort, and evaluation of a wide variety of statistical approaches. Numerous combinations of seven or eight of fifteen metabolites and clinical parameters were effective in prediction. A final model employed logistic regression of values of MAP, hexanoylcarnitine, Na⁺, creatinine, pseudouridine, HPLA and 3-methoxytyrosine. The factors in this model all reflected the observed dichotomy in host response and/or have previously shown utility in sepsis outcome prediction. The model predicted 7-day all cause survival/death with an AUC of 0.88 and 99% accuracy, assuming a 10% prior probability of death. All cause survival/death (confirmed sepsis and patients presenting with sepsis but subsequently shown to have a non-infectious SIRS etiology) matched precisely the clinical scenario encountered in ED patients. The performance of this model was approximately 10% better than those obtained in the same patients by capillary lactate, SOFA or APACHE II scores, the current gold standards for prognostic assessment in sepsis. Independent replication studies are needed, as are finalization of markers and parameters and additional assay development. As with many current disease severity markers, the panel is likely to be especially useful when used serially in individual patients. Ideally, the panel should be deployed on device that will be at point-of-care or hospital-based and with time-to-result of about an hour. With additional development, this panel may meet the immense need for prompt determination of sepsis prognosis in individuals to guide targeting of intensive treatments and, thereby, to improve outcomes.

In the interim, it will be possible to use some of the markers of the molecular phenotypes of sepsis as pharmacogenetic indicators. Key questions are whether the observed molecular phenotype of death is universal and is it reversible. The vast majority of the CAPSOD sepsis deaths had received early goal-directed therapy (EGDT). Possibly, inclusion of assessment of the death phenotype could allow individualization of EGDT. None of the sepsis deaths had received activated protein C. The molecular phenotype of death included broad changes in complement, coagulation and fibrinolytic system components, suggesting a specific role for activated protein C in the treatment of these patients. It will be very interesting to evaluate the effect on the death phenotype of experimental sepsis therapies such as succinate or acetylcarnitine supplementation, intensive glycemic control or enhancement of mitochondrial biogenesis.

Finally, global and temporal correlation of metabolome, proteome and transcriptome data from relevant biological fluids and well-phenotyped patient groups seems broadly suitable for expanding our understanding of intermediary metabolism, particularly with respect to poorly annotated analytes, and for characterization of homogeneous subgroups in complex traits. Combinations of transcriptome, proteome, metabolome and genetic data may establish multi-dimensional molecular models of other complex diseases that could provide insights into network responses to intrinsic and/or extrinsic perturbation.

1. A method for determining the severity of a sepsis infection in a patient comprising, determining in the patient; the patient's age, mean arterial pressure, hematocrit, patient temperature, and the concentration of at least one metabolite that is predictive of sepsis severity by obtaining a blood sample from said patient and determining the concentration of the metabolite in the patient's blood; and
determining the severity of sepsis infection by analyzing the measured values in a weighted logistic regression equation.

2. The method for determining the severity of a sepsis infection in a patient of claim 1, further comprising determining the concentration of one or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate, such that the accuracy of the panel in predicting day 7 sepsis survival in a known test patient population pool is about 99% or more.

3. The method for determining the severity of a sepsis infection in a patient of claim 1, further comprising measuring the concentration of two or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

4. The method for determining the severity of a sepsis infection in a patient of claim 1, further comprising measuring the concentration of three or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

5. A method for determining the severity of a sepsis infection in a patient of claim 1, further comprising measuring the concentration of four or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

6. The method for determining the severity of a sepsis infection in a patient of claim 1, further comprising measuring the concentration of five or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

7. The method for determining the severity of a sepsis infection in a patient of claim 1, further comprising measuring the concentration of six or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

8. The method for determining the severity of a sepsis infection in a patient of claim 1, further comprising measuring the concentration of seven or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

9. The method for determining the severity of a sepsis infection in a patient of claim 1, further comprising measuring the concentration of eight or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

10. The method for determining the severity of a sepsis infection in a patient of claim 1, further comprising measuring the concentration of nine or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

11. A method for determining the severity of a sepsis infection in a patient of claim 1, further comprising measuring the concentration of ten or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

12. The method for determining the severity of a sepsis infection in a patient of claim 1, further comprising measuring the concentration of eleven or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

13. The method for determining the severity of a sepsis infection in a patient of claim 1, further comprising measuring the concentration of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

14. The method for determining the severity of a sepsis infection in a patient of claim 1, wherein the blood sample for the analysis is taken when the patient arrives for treatment.

15. The method for determining the severity of a sepsis infection in a patient of claim 1, wherein the blood sample for the analysis is taken when the patient arrives for treatment and again approximately 1 day later.

16. A panel of clinical and metabolomics biomarker classifiers adapted to predict the severity of a sepsis infection in a patient comprising the patient's age, mean arterial pressure, hematocrit, temperature, and the concentration of a metabolite that is predictive of sepsis severity.

17. The panel of clinical markers of claim 16, further comprising the concentration of one or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate, such that the accuracy of the panel in predicting day 7 sepsis survival in a known test patient population pool is about 99% or more.

18. The panel of clinical markers of claim 16, further comprising the concentration of two or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

19. The panel of clinical markers of claim 16, further comprising the concentration of three or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

20. The panel of clinical markers of claim 16, further comprising the concentration of four or more metabolites selected from the group of metabolite markers consisting of 2-methylbutyrylcarnitine, 4-cis-decenoylcarnitine, butyrylcarnitine, hexanoylcarnitine, 4-methyl-2-oxopentanoate, 1-arachidonoylglycerophosphocholine, 1-linoleoylglycerophosphocholine, 3-(4-hydroxyphenyl)lactate (HPLA), 3-methoxytyrosine, n-acetylthreonine, pseudouridine and lactate.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)
